(19) United States
(12) Patent Application Publication
(10) Pub. No.: US 2009/0191610 A1
(43) Pub. Date: Jul. 30, 2009

(54) Microorganisms With Increased Efficiency for Methionine Synthesis

(75) Inventors: Oskar Zelder, Speyer (DE); Andrea Herold, Ketsch (DE); Corinna Klopprogge, Mannheim (DE); Hartwig Schroder, Nussloch (DE); Stefan Haefner, Speyer (DE); Elmar Heinzle, Saarbrucken (DE); Christoph Wittmann, Saarbrucken (DE); Jens Kroemer, Queensland (AU); Janice G. Pero, Lexington, MA (US); R. Rogers Yocum, Lexington, MA (US); Thomas A. Patterson, North Attleboro, MA (US); Mark Williams, Revere, MA (US); Theron Herman, Kinnelon, NJ (US)

Correspondence Address: LAHIVE & COCKFIELD, LLP;FLOOR 30, SUITE 3000, ONE POST OFFICE SQUARE, BOSTON, MA, 02109, US

(73) Assignee: Evonik Degussa GmbH, Essen (DE)

(21) Appl. No.: 11/989,369

(22) PCT Filed: Aug. 18, 2006
(86) PCT No.: PCT/EP2006/065460

§ 371 Date: Nov. 21, 2008

(30) Foreign Application Priority Data
Aug. 18, 2005 (EP) 05107609.9
May 24, 2006 (EP) 06114543.9

(51) Int. Cl. C12N 1/21; C12N 15/10; C12N 1/19; G01N 33/48; C12N 5/10

(52) U.S. Cl. 435/252.32; 435/471; 435/468; 435/419; 435/252.33; 435/254.2; 702/19

(57) ABSTRACT

The present invention relates to methods for the production of microorganisms with increased efficiency for methionine synthesis, microorganisms with increased efficiency for methionine synthesis, and methods for determining the optimal metabolic flux for organisms with respect to methionine synthesis.

### FIELD OF THE INVENTION

The invention lies in the field of fine chemicals being produced by organisms. Particularly, the present invention concerns methods for the production of microorganisms with increased efficiency for methionine synthesis. The present invention also concerns microorganisms with increased efficiency for methionine synthesis. Furthermore, the present invention concerns methods for determining the optimal metabolic flux for organisms with respect to methionine synthesis.

### TECHNOLOGICAL BACKGROUND

Amino acids are used for different purposes, one field of application being the use as food additives in the food of human and animals. Methionine is an essential amino acid that has to be ingested with food. Besides being essential for protein biosynthesis, methionine serves as a precursor for different metabolites such as glutathione, S-adenosyl methionine and biotine. It also acts as a methyl group donor in various cellular processes.

Currently, worldwide annual production of methionine is about 500,000 tons. Methionine is the first limiting amino acid in livestock of poultry feed and due to this, mainly applied as feed supplement. In contrast to other industrial amino acids, methionine is almost exclusively applied as a racemate produced by chemical synthesis (DE 190 64 05). As animals can metabolise both stereoisomers of methionine, direct feed of the chemically produced racemic mixture is possible (Dello and Lewis (1978) Effect of Nutrition Deficiencies in Animals: Amino Acids, Rechgigl (Ed.) CRC Handbook Series in Nutrition and Food, 441-490).

However, there is still a great interest in replacing the existing chemical production by a biotechnological process. This is due to the fact that at lower levels of supplementation L-methionine is a better source of sulfur amino acids than D-methionine (Katz & Baker, (1975) Poult. Sci., 545, 1667-74). Moreover, the chemical process uses rather hazardous chemicals and produces substantial waste streams. An efficient biotechnological process could avoid all these disadvantages of chemical production.

For other amino acids such as glutamate, lysine, threonine and tryptophane, it has been known to produce them using fermentation methods. For these purposes, certain microorganisms such as Escherichia coli (E. coli) and Corynebacterium glutamicum (C. glutamicum) have proven to be particularly suited The production of amino acids by fermentation also has the particular advantage that only L-amino acids are produced and that environmentally problematic chemicals such as solvents, etc. which are used in chemical synthesis are avoided. However, fermentative production of methionine by microorganisms will only be an alternative to chemical synthesis if it allows for the production of methionine on a commercial scale at a price comparable to that of chemical production.

In the past, there have been attempts to use microorganisms such as E. coli and C. glutamicum for production of sulfur-containing compounds that are commonly also designated as fine chemicals. These methods included classical strain selection by mutagenesis as well as optimisation of the cultivation conditions, e.g. steering, provision of oxygen, composition of cultivation media, etc. (Kumar et al. (2005) Biotechnology Advances, 23, 41-61).

One of the reasons that fermentative production of methionine in microorganisms has not yet proven to be economically interesting probably results from the peculiars of the biosynthesis and metabolic pathways that lead to methionine. In general, the basic metabolic pathways leading to methionine synthesis in organisms such as E. coli and C. glutamicum are well known (e.g. Voet and Voet (1995) Biochemistry, 2^{nd} edition, Jon Wiley &Sons, Inc and http://www.genomejp/kegg/metabolism.html). However, the details of biosynthesis of methionine in C. glutamicum and E. coli is subject to intensive research and have recently been reviewed in Rückert et al. (Rückert et al. (2003), J. of Biotechnology, 104, 213-228) and Lee et al. (Lee et al. (2003), Appl. Microbiol. Biotechnol., 62, 459-467).

A key step in the biosynthesis of methionine is the incorporation of sulfur into the carbon backbone. The sulfur source regularly is sulfate and has to be taken up by the microorganisms. The microorganisms then have to activate and reduce the sulfate. These steps require an energy input of 7 mol ATP and 8 mol NADPH per molecule methionine (Neidhardt et al. (1990) Physiology of the bacterial cell: a molecular approach, Sunderland, Mass., USA, Sinauer Associates, Inc.) Thus, methionine is the one amino acid with respect to which a cell has to provide the most energy.

As a consequence thereof, methionine-producing microorganisms have evolved metabolic pathways that are under strict control with respect to the rate and amount of methionine synthesis (Neidhardt F. C. (1996) E. coli and S. typhimurium, ASM Press Washington). These regulation mechanisms include e.g. feedback control mechanisms, i.e. methionine producing metabolic pathways are down-regulated with respect to their activity once the cell has produced sufficient amounts of methionine. Approaches of the prior art for obtaining microorganisms which can be used for industrial scale production of methionine by microorganisms mainly focussed on overcoming the above-mentioned control mechanisms by identifying genes that are involved in the biosynthesis of methionine. These genes were then either over-expressed or repressed, depending on their respective function with the ultimate goal of increasing the amount of methionine produced. In this context, the amount of methionine has been defined either as the amount methionine obtained per amount cell mass or as the amount methionine obtained per time and volume (space-time-yield) or as a combination of both factors that is cell mass and space-time-yield.

For example, WO 02/10209 describes the over-expression or repression of certain genes in order to increase the amount of methionine produced. Recently, Rey et al. (Rey et al. (2003), J. Biotechnol., 103, 51-65,) identified the transcriptional repressor McbR that controls expression of genes involved in the biosynthesis of methionine such as metY (coding for O-acetyl-L-homoserinesulfhydrylase), metK (coding for S-adenosyl-methionine synthetase), hom (coding for homoserinedehydrogenase), cysK (coding for L-cysteine synthase), cysI (coding for NADPH-dependent sulphite reductase) and ssuD (coding for alkane sulfonate monooxygenase).

Even though these approaches allowed for the construction of microorganism strains which produced more methionine compared to the wild type with the methionine amount being calculated per cell mass or per time and volume (space-time yield), no industrially competitive methionine over-producing organism has been described so far (Mondal et al. (1996) Folia Microbiol. (Praha), 416, 465-72, (Kumar et al. (2005) Biotechnology Advances, 23, 41-61).

### SUMMARY OF THE INVENTION

it has been found that the amount of methionine produced by an organism which typically is calculated as the amount of methionine per kilogram cell mass or per time and volume is not a sufficient indicator of whether a methionine-producing organism may be considered as an economically interesting and commercially viable alternative to chemical production of this amino acid. Rather, in order to be an economically interesting alternative for the chemical synthesis method, a methionine-producing organism with high efficiency is required, i.e. an organism that provides for a high space-time yield of methionine on the basis of the energy input of the production system which may be represented by the amount or input of a carbon source such as glucose that is being consumed for the production of methionine.

Thus, when deciding whether a methionine-producing organism may be considered as an alternative to chemical synthesis, the key parameter shall not be the amount of methionine produced per weight cell mass, but the efficiency, i.e. the molar amount of methionine produced per amount energy input consumed by the system e.g. in the form of glucose.

In this context, it has further been found that in order to produce methionine at a high efficiency in a microorganism, the metabolic pathways of the organism that contribute directly or indirectly to methionine synthesis have to be used in an optimal way with respect to methionine synthesis. Thus, for efficient production of methionine by an organism, the metabolic flux through the metabolic pathways has to be modified. Modification may not only be required for those pathways that are directly involved in the synthesis of the methionine backbone, but also of those pathways that provide additional substrates such as sulfur atoms in different oxidative states, nitrogen in the reduced state such as ammonia, further carbon precursors including C1-carbon sources such as serine, glycine and formate, precursors of methionine and different metabolites of tetrathydrofolate which is substituted with carbon at N5 and or N10. In addition energy e.g. in the form of reduction equivalents such as NADH, NADPH, FADH2 can be involved in the pathways leading to methionine. Thus, a microorganism which produces methionine very efficiently may require a high metabolic flux through the pathways that lead to the construction of methionine and that provide precursors thereof, but may require only low metabolic fluxes through biosynthesis pathways of e.g. other amino acids.

It is therefore an object of the present invention to identify the optimal metabolic flux through the pathways involved directly or indirectly in methionine synthesis in order to identify potential organisms which may be very efficient in methionine synthesis.

A further object of the present invention is to provide methods which allow to predict the ideal metabolic flux through the various metabolic pathways of an organism for methionine synthesis in order to achieve efficient methionine biosynthesis.

A further object of the present invention is to provide methods for obtaining organisms which have an increased efficiency in methionine synthesis.

The present invention also aims at organisms that are more efficient with respect to methionine synthesis.

These and other objects, as they will become apparent from the ensuing description, are solved by the subject matter as defined in the independent claims. The dependent claims relate to some of the embodiments contemplated by the invention.

In the course of the present invention a metabolic pathway analysis, also referred to as elementary flux mode analysis or extreme pathway analysis, was used to study the metabolic properties of organisms with respect to methionine synthesis. While the above metabolic pathway analysis has been described in the prior art for other cellular systems Papin et al. (2004) Trends Biotechnol 228, 400-405; Schilling et al. (2000) J. Theor. Biol., 2033, 229-248; Schuster et al. (1999) Trends Biotechnol. 172, 53-60), this type of analysis has not been considered with respect to efficiency of methionine production in organisms such as C. glutamicum and E. coli. Metabolic pathway analysis commonly allows the calculation of a solution space that contains all possible steady-state flux distributions of a metabolic network. Hereby, the stoichiometry of the metabolic network studied, including energy, precursors as well as co-factor requirements are fully considered.

In the present invention, this elementary flux mode analysis was carried out for the first time with respect to the efficiency of methionine production by comparing the metabolic networks of major industrial amino acid producers such as C. glutamicum and E. coli. For this purpose, biochemical reaction models were constructed for C. glutamicum and E. coli (see below). The models comprised all relevant routes of sulfur metabolism involving all pathways linked to methionine production. These models were constructed from current biochemical knowledge of the organisms investigated (see below). On the basis of these models, the optimal metabolic flux through the various pathways was calculated in order to predict which pathways should be used more or less intensively in order to increase efficiency of methionine production.

By calculating these models, a model organism was obtained which for a given set of conditions including the presence of external metabolites such as the carbon source and the sulfur source would be optimal for methionine production.

The present invention thus concerns a method for designing an organism with increased efficiency for methionine synthesis. This method comprises the steps of describing or parameterizing an initial methionine synthesizing organism by means of a plurality of parameters, which are obtained on the basis of pre-known metabolic pathways related to methionine synthesis and which relate to the metabolic flux through the reaction of these pathways, and then determining an organism with increased efficiency for methionine synthesis by modifying at least one of the plurality of said parameters and/or introducing at least one further such parameter in such a manner as to increase the efficiency of methionine synthesis compared to the efficiency of methionine synthesis of the initial methionine synthesizing organism. Using this method, it is thus possible to predict a theoretical organism which should allow for efficiency methionine synthesis. The detailed performance of the method is described later on.

For the purposes of the invention, these parameters were defined in relation to the single reactions of the metabolic network considered. Thus, the parameters for optimisation were defined in relation to the existence of a reaction in the organism employed, the stoichiometry of a reaction and the reversibility of the reaction. As a consequence the parameters relate to the metabolic flux through the various reactions of the network.

The present invention also relates to a device for designing an initial organism with increased efficiency for methionine synthesis, the device comprising a processor adapted to carry out the above-mentioned method steps for predicting optimised pathways for an organism with increased methionine synthesis.

The invention further relates to a computer-readable medium in which a computer program for designing an organism with increased efficiency for methionine synthesis is stored. The computer-readable medium which when being executed by a processor is adapted to carry out the above-mentioned method steps for designing a theoretically optimised organism with increased efficiency of methionine synthesis.

The invention further relates to a program element of designing an organism with increased efficiency for methionine synthesis which, when being executed by a processor, is adapted to carry out the above-mentioned method steps.

The invention also relates to methods for producing organisms with increased efficiency of methionine synthesis which make use of the above-mentioned predictions by genetically modifying a wild type organism in order to influence the metabolic flux of that organism such that it more resembles the predictions of the above-mentioned methods. This may be achieved by genetically modifying the organism such that the metabolic flux through a certain reaction pathway is increased and/or decreased. Genetic modifications may be introduced by recombinant DNA technology. In addition this may be also achieved by other techniques such as but not limited to mutation and selection processes such as chemical or UV mutagenesis and subsequent selection by growth on substrate analoga containing media, leading to resistant strains with improved characteristics.

The invention also relates to methods for producing organisms with increased efficiency of methionine synthesis which make use of the above-mentioned predictions by genetically modifying an organism which is not a wild type organism, but which has already been genetically modified before, preferably to produce methionine at an increased mass and/or time-space yield. Such organisms may be organisms which are known as methionine overproducers and include e.g. organisms in which genes for sulfate assimilation, genes for cysteine biosynthesis and genes for methionine synthesis as well as genes for conversion of oxaloacetate to aspartate semialdehyde are overexpressed. In such organisms which have been already genetically modified the above-mentioned predictions as regards increased efficiency of methionine synthesis may be implemented in order to influence the metabolic flux of that organism such that it more resembles the predictions of the above-mentioned methods. This may be achieved by genetically modifying the organism such that the metabolic flux through a certain reaction pathway is increased and/or decreased. Genetic modifications may be introduced by recombinant DNA technology. In addition this may be also achieved by other techniques such as but not limited to mutation and selection processes such as chemical or UV mutagenesis and subsequent selection by growth on substrate analoga containing media, leading to resistant strains with improved characteristics.

It has surprisingly been found that the theoretic predictions which are obtained with respect to a wild type organism can be used to increase efficiency of methionine synthesis also in an organism which already carries mutations e.g. in pathways relating to methionine synthesis or e.g. accessory pathways relating thereto. Thus, it seems not necessary that theoretic predictions are calculated on the basis of the respective starting organism but that theoretic predictions obtained for a wild type organism may be sufficient. However, the present invention certainly also considers an embodiment in which an optimal metabolic flux is calculated on the basis of an initial organism which already provides some of the above mentioned mutations so that the predictions may be used to further genetically modify the organism.

Particularly, the present invention relates to methods for producing microorganisms of the genus Corynebacterium and Escherichia with increased efficiency of methionine production which comprises the steps of increasing and/or introducing the metabolic flux through pathways that have been used for constructing the above-mentioned model. These methods may additionally include the steps of at least partially decreasing the metabolic flux through the above-mentioned pathways.

The present invention also relates to organisms with an increased efficiency of methionine synthesis which are obtainable by any of the above-mentioned methods. Further, the present invention relates to the use of such organism for producing methionine and for methods of producing methionine by cultivating the above-mentioned organisms and isolating methionine.

### BRIEF DESCRIPTION OF THE FIGURES

FIG. 1 shows a stoichiometric reaction network of a C. glutamicum wild type organism that was used for elementary flux mode analysis.

FIG. 2 shows the metabolic pathway analysis of C. glutamicum and E. coli for methionine synthesis.

FIG. 3 shows the metabolic flux distribution of a C. glutamicum wild type organism with maximal theoretical yield of methionine.

FIG. 4 shows the metabolic flux distribution of an E. coli wild type organism with maximal theoretical yield of methionine.

FIG. 5 shows the metabolic pathway analysis of C. glutamicum for methionine synthesis with different carbon and sulfur sources.

FIGS. 6 to 9 show various vectors which are used in the embodiment examples.

FIG. 10 shows one optimized metabolic flux distribution of a C. glutamicum strain in which additional metabolic pathways have been included.

### DETAILED DESCRIPTION OF THE PRESENT INVENTION

Before describing in detail how the above-mentioned method may be carried out in order to identify a theoretical optimised organism with increased efficiency of methionine synthesis, the following definitions are given.

The term “efficiency of microorganism synthesis” describes the carbon yield of methionine. This efficiency is calculated as a percentage of the energy input which entered the system in the form of a carbon substrate. Throughout the invention this value is given in percent values ((mol methionine) (mol carbon substrate)⁻¹×100) unless indicated otherwise.

The term “increased efficiency of methionine synthesis” relates to a comparison between an organism that has been theoretically modelled by the above-mentioned methods and which has a higher efficiency of methionine synthesis compared to the initial model organism that was used for parameterizing.

The term “increased efficiency of methionine synthesis” may also describe the situation in which an organism that has been e.g. genetically modified provides an increased efficiency of methionine synthesis compared to the respective starting organism.

The term “metabolic pathway” relates to a series of reactions that are part of the metabolic network that is used in the above-mentioned theoretical model for designing an organism with improved methionine synthesis.

The term “metabolic pathway” also describes a series of reactions which take place in a real organism. A metabolic pathway may comprise a well-known series of reactions as these are known from standard textbooks such as e.g. respiratory chain, glycosylation, tricarboxylic acid cycle, etc. Alternatively, metabolic pathways may be defined separately for the purposes of the present invention.

The term “metabolic flux” describes the amount of energy input that is fed into the system, e.g. in the form of a carbon source such as glucose and which passes through the reactions of the metabolic network of an organism or of the above-mentioned theoretical model. Every reaction of the network will usually contribute to the overall metabolic flux. As a consequence, a metabolic flux may be assigned to every reaction of the network. As elementary flux modes are calculated on the basis of the stoichiometry of the various reactions of the network model, fluxes are typically given as relative molar values, normalized to the energy uptake rate which is measured in the form of glucose, i.e. fluxes are given in mol (substance)×(mol glucose)⁻¹×100).

The term “modified metabolic flux” relates to a situation in which the metabolic flux through a certain reaction or a metabolic pathway of an organism that has been genetically modified, is increased or decreased compared to the starting organism. This term also relates to the situation where, in accordance with the above-mentioned theoretical method of determining or designing an optimised organism for methionine synthesis, the theoretical metabolic flux through a certain reaction or metabolic pathway of the metabolic network is increased or decreased by changing the parameters of the theoretical metabolic network.

If in the context of the present invention use is made of the term “approximating the metabolic flux”, this relates to genetically modifying organisms in order to increase and/or decrease and/or introduce the metabolic flux through the pathways of methionine synthesis which have been used for constructing the above-mentioned theoretical model. As the genetic modifications are selected on the basis of the predictions of the above-mentioned model, the metabolic flux of the genetically modified organisms, in comparison to the respective starting organism, should resemble more closely the metabolic flux of the above-mentioned optimized model.

The terms “express”, “expressing”, “expressed” and “expression” refer to expression of a gene product (e.g., a biosynthetic enzyme of a gene of a pathway or reaction defined and described in this application) at a level that the resulting enzyme activity of this protein encoded for or the pathway or reaction that it refers to allows metabolic flux through this pathway or reaction in the organism in which this gene/pathway is expressed in. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

The terms “overexpress”, “overexpressing”, “overexpressed” and “overexpression” refer to expression of a gene product (e.g. a methionine biosynthetic enzyme or sulfate reduction pathway enzyme or cysteine biosynthetic enzyme or a gene or a pathway or a reaction defined and described in this application) at a level greater than that present prior to a genetic alteration of the starting microorganism. In some embodiments, a microorganism can be genetically altered (e.g., genetically engineered) to express a gene product at an increased level relative to that produced by the starting microorganism. Genetic alteration includes, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins). Examples for the overexpression of genes in organisms such as C. glutamicum can be found in Eikmanns et al (Gene. (1991) 102, 93-8).

In some embodiments, a microorganism can be physically or environmentally altered to express a gene product at an increased or lower level relative to level of expression of the gene product by the starting microorganism. For example, a microorganism can be treated with or cultured in the presence of an agent known or suspected to increase transcription of a particular gene and/or translation of a particular gene product such that transcription and/or translation are enhanced or increased.

Alternatively, a microorganism can be cultured at a temperature selected to increase transcription of a particular gene and/or translation of a particular gene product such that transcription and/or translation are enhanced or increased.

The terms “deregulate,” “deregulated” and “deregulation” refer to alteration or modification of at least one gene in a microorganism, wherein the alteration or modification results in increasing efficiency of methionine production in the microorganism relative to methionine production in absence of the alteration or modification. In some embodiments, a gene that is altered or modified encodes an enzyme in a biosynthetic pathway, such that the level or activity of the biosynthetic enzyme in the microorganism is altered or modified. In some embodiments, at least one gene that encodes an enzyme in a biosynthetic pathway is altered or modified such that the level or activity of the enzyme is enhanced or increased relative to the level in presence of the unaltered or wild type gene. In some embodiments, the biosynthetic pathway is the methionine biosynthetic pathway. In other embodiments, the biosynthetic pathway is the cysteine biosynthetic pathway. Deregulation also includes altering the coding region of one or more genes to yield, for example, an enzyme that is feedback resistant or has a higher or lower specific activity. Also, deregulation further encompasses genetic alteration of genes encoding transcriptional factors (e.g., activators, repressors) which regulate expression of genes in the methionine and/or cysteine biosynthetic pathway.

The phrase “deregulated pathway or reaction” refers to a biosynthetic pathway or reaction in which at least one gene that encodes an enzyme in a biosynthetic pathway or reaction is altered or modified such that the level or activity of at least one biosynthetic enzyme is altered or modified. The phrase “deregulated pathway” includes a biosynthetic pathway in which more than one gene has been altered or modified, thereby altering level and/or activity of the corresponding gene products/enzymes. In some cases the ability to “deregulate” a pathway (e.g., to simultaneously deregulate more than one gene in a given biosynthetic pathway) in a microorganism arises from the particular phenomenon of microorganisms in which more than one enzyme (e.g., two or three biosynthetic enzymes) are encoded by genes occurring adjacent to one another on a contiguous piece of genetic material termed an “operon.” In other cases, in order to deregulate a pathway, a number of genes must be deregulated in a series of sequential engineering steps.

The term “operon” refers to a coordinated unit of genetic material that contains a promoter and possibly a regulatory element associated with one or more, preferably at least two, structural genes (e.g., genes encoding enzymes, for example, biosynthetic enzymes). Expression of the structural genes can be coordinately regulated, for example, by regulatory proteins binding to the regulatory element or by anti-termination of transcription. The structural genes can be transcribed to give a single mRNA that encodes all of the structural proteins. Due to the coordinated regulation of genes included in an operon, alteration or modification of the single promoter and/or regulatory element can result in alteration or modification of each gene product encoded by the operon. Alteration or modification of a regulatory element includes, but is not limited to, removing endogenous promoter and/or regulatory element(s), adding strong promoters, inducible promoters or multiple promoters or removing regulatory sequences such that expression of gene products is modified, modifying the chromosomal location of the operon, altering nucleic acid sequences adjacent to the operon or within the operon such as a ribosome binding site, codon usage, increasing copy number of the operon, modifying proteins (e.g., regulatory proteins, suppressors, enhancers, transcriptional activators and the like) involved in transcription of the operon and/or translation of the gene products of the operon, or any other conventional means of deregulating expression of genes routine in the art (including, but not limited to, use of antisense nucleic acid molecules, for example, to block expression of repressor proteins).

In some embodiments, recombinant microorganisms described herein have been genetically engineered to overexpress a bacterially derived gene or gene product. The terms “bacterially-derived” and “derived-from bacteria” refer to a gene which is naturally found in bacteria or a gene product which is encoded by a bacterial gene.

The term “organism” for the purposes of the present invention refers to any organism that is commonly used of the production of amino acids such as methionine. In particular, the term “organism” relates to prokaryotes, lower eukaryotes and plants.

A preferred group of the above-mentioned organisms comprises actino bacteria, cyano bacteria, proteo bacteria, Chloroflexus aurantiacus, Pirellula sp. 1, halo bacteria and/or methanococci, preferably coryne bacteria, myco bacteria, streptomyces, salmonella, Escherichia coli, Shigella and/or Pseudomonas. Particularly preferred microorganisms are selected from Corynebacterium glutamicum, Escherichia coli, microorganisms of the genus Bacillus, particularly Bacillus subtilis, and microorganisms of the genus Streptomyces.

The term “initial organism” is used to describe the organism and the metabolic network that has been used for assigning the initial set of parameters for the above-mentioned model according to independent claim 1.

The term “starting organism” refers to the organism which is used for genetic modification to increase affiance of methionine production. A starting organism may either be a wild type organism or an organism which already carries mutations. The starting organism can be identical to the initial organism. Starting organisms may e.g. be methionine overproducers.

The term “wild type organism” relates to an organism that has not been genetically modified. The term methionine overproducer relates to an organism that has been altered either by genetic manipulation, by mutation and selection or by any other known method and which overproduces more methionine than the wild type strain which was used to obtain an methionine overproducer.

The organisms of the present invention may, however, also comprise yeasts such as Schizosaccharomyces pombe or cerevisiae and Pichia pastoris.

Plants are also considered by the present invention for the production of amino acids. Such plants may be monocots or dicots such as monocotyledonous or dicotyledonous crop plants, food plants or forage plants. Examples for monocotyledonous plants are plants belonging to the genera of avena (oats), triticum (wheat), secale (rye), hordeum (barley), oryza (rice), panicum, pennisetum, setaria, sorghum (millet), zea (maize) and the like.

Dicotyledonous crop plants comprise inter alias cotton, leguminoses like pulse and in particular alfalfa, soybean, rapeseed, tomato, sugar beet, potato, ornamental plants as well as trees. Further crop plants can comprise fruits (in particular apples, pears, cherries, grapes, citrus, pineapple and bananas), oil palms, tea bushes, cacao trees and coffee trees, tobacco, sisal as well as, concerning medicinal plants, rauwolfia and digitalis. Particularly preferred are the grains wheat, rye, oats, barley, rice, maize and millet, sugar beet, rapeseed, soy, tomato, potato and tobacco. Further crop plants can be taken from U.S. Pat. No. 6,137,030.

The term “metabolite” refers to chemical compounds that are used in the metabolic pathways of organisms as precursors, intermediates and/or end products. Such metabolites may not only serve as chemical building units, but may also exert a regulatory activity on enzymes and their catalytic activity. It is known from the literature that such a metabolites may inhibit or stimulate the activity of enzymes (Stryer, Biochemistry, (1995) W.H. Freeman & Company, New York, N.Y.).

For the purposes of the present invention, the term “external metabolite” comprises substrates such as glucose, sulfate, thiosulfate, sulfite, sulfide, ammonia, phosphate, metal ions such as Fe2+Mn 2+Mg2+, Co2+MoO2+ and oxygen etc. In certain embodiments (external) metabolites comprise so-called C1-metabolites. These latter metabolites can function as e.g. methyl donors and comprise compounds such as formate, methanol, formaldehyde, methanethiol, dimethyldisulfide etc.

The term “products” comprises methionine, cysteine, glycine, lysine, trehalose, biomass, CO₂, etc.

Before describing the invention with respect to its particular embodiments, a general overview is given as to how the predictions by elementary flux analysis were obtained.

The elementary flux analysis starts with the formulation and implementation of all metabolic reactions relevant for growth and methionine production. The required information can be collected from public databases such as KEGG (http://www.genome.jp/kegg/) and others. The model is then set up accordingly and reflects the natural potential of the wild type organism and serves as the starting point for further development of methionine overproducing model strains. For obtaining an initial model, biochemical reaction models were constructed for methionine synthesis. For this purpose, models were constructed which comprise all relevant routes of central carbon and sulfur metabolism involving all relevant pathways linked to methionine production as they are known from the literature. If a pathway for a certain organism, such as e.g. E. coli, is known to not be present in another organism such as C. glutamicum, the organism's specific pathway reactions were only considered in the model for that specific organism and left out for the other organisms when constructing the model for the initial organism. After an initial model has been obtained, pathways from other organisms which are known to not occur in the model organism may then be considered, i.e. introduced, for further optimisation. The different biochemical reactions that contribute to a metabolic network may be obtained e.g. from standard textbooks, the scientific literature or Internet links such as http://www.genomejp/kegg/metabolism.html.

An elementary flux mode analysis was then performed as described in the literature (see e.g. Papin et al. (2004) vide supra, Schilling et al. (2000) vide supra, Schuster et al. (1999) vide supra). The elementary flux modes are calculated on the basis of the stoichiometry of the various reactions. The specific kinetics of each reaction are usually not taken into consideration.

As constructed, a metabolic network typically comprises a lot of pathway cycles and reversible reactions. Various pathway routes may thus be taken in order to arrive at a compound such as methionine. Thus, depending on which route is taken, the energy requirements for production of the same compound may change within the same network. As a consequence, if the various reactions of a network are described by parameters and put into an algorithm such as the METATOOL software (Pfeiffer et al. (1999) Bioinformatics, 153, 251-257; Schuster et al. (1999) vide supra), the network can be modified and optimised in order to identify the route which allows for the most efficient synthesis of methionine.

For the purposes of the present invention, the metabolic pathway analysis was carried out using the program METATOOL. The version used for the present invention (meta 4.0.1_double.exe) is available on the Internet at http://www.biozentrum.uni-wuerzburg.de/bioinformatik/computing/metatool/pinguin.biologie.uni-jena.de/bioinformatik/networks/. The mathematical details of the algorithm are described by Pfeiffer et al. (Pfeiffer et al. (1999) vide supra). If the metabolic pathway analysis is carried out using the METATOOL program, several hundreds of elementary flux modes result for each situation investigated. For each of these flux modes the carbon yields of methionine were, as indicated above, calculated as percentage of the carbon that entered the system as substrate. For the various flux modes the carbon yield of biomass may be calculated as percentage of the energy that entered the system in the form of carbon substrate. This parameter may thus be calculated as ((mol biomass)(mol substrate)⁻¹×100). Co-substrates other than glucose, such as formate, formaldehyde, methanol, methanethiol or its dimer dimethyldisulfide may also be considered correspondingly. The comparative analysis of all such elementary flux modes that are obtained for a certain network scenario then allows the determination of the theoretical maximum efficiency for methionine synthesis.

In this way, one obtains a theoretical prediction of the optimal metabolic flux through the metabolic network of an organism which should have an optimal efficiency for methionine synthesis. The details of such a theoretical metabolic flux analysis is described in the experimental section.

The method of theoretically determining or designing such an organism with increased efficiency for methionine synthesis constitutes the subject matter of independent claim 1.

The theoretical predictions which are obtained by these methods may then be put into practise by genetically modifying the respective organism in order to enhance or reduce metabolic flux through those pathways identified by the prediction model. Surprisingly, the theoretic predictions can also be put into practice according to the predictions of the model by genetically altering a starting organism, which is not identical with the initial organism. Such starting organism may thus not be a wild type organism, but organisms which are already genetically modified. In one embodiment, the starting organism may be e.g. a methionine overproducer, i.e. a genetically modified organism which is already known to produce more methionine than the respective wild type organism. Even though the theoretic predictions have not been calculated for such a methionine overproducer, they still allow constructing genetically modified organisms on the basis of the methionine overproducer which provide an increased efficiency of methionine synthesis.

If, e.g. the theoretical predictions imply that methionine synthesis is most efficient if the metabolic flux through the pentose phosphate pathway (PPP) is increased, an organism is genetically modified to that purpose. This could be done, e.g. by increasing the amount and/or activity of enzymes that catalyse certain steps of the PPP in order to channel more metabolic flux through this pathway compared to a genetically unmodified organism that is cultivated under otherwise exactly the same conditions. The flux into the PPP may also be enhanced by e.g. down-regulating the enzymatic activity in an irreversible reaction of another parallel pathway that redirects the metabolic flux into the PPP. The flux through the PPP may also be enhanced by introducing specific mutations into genes coding for proteins that are involved in PPP cycle enzymes such as mutations in the pyruvate carboxylase as described by Onishi et al. (Appl Microbiol Biotechnol. (2002), 58, 217-23). These altered genes contain mutations compared to the genes derived from so-called wild type strains. These mutations may lead to altered enzymatic activity or sensitivity towards molecular feedback inhibitors.

Correspondingly, if the theoretical model requires a reduction of the metabolic flux to the pentose phosphate pathway, the amount and/or activity of enzymes of this pathway may be reduced.

Metabolic flux analysis may also be used to transfer results generated for one organism to another. Thus, if it is found by elementary flux mode analysis that in e.g. E. coli a certain pathway with increased activity is crucial for efficient methionine synthesis, and if this pathway is obviously not used or not present in another organism such as C. glutamicum, this pathway may be introduced into the respective organism by introducing the genes that code for the enzymatic activities of this pathway into the respective organism. By that approach, it may not only be possible to optimise microorganisms with respect to methionine synthesis by optimising their endogenous metabolic pathways, but also to introduce an exogenous metabolic pathway in order to further enhance methionine synthesis and/or increase synthesis efficiency.

In view of this situation, the present invention also relates to a method for producing an organism being selected from the group of prokaryotes, lower eukaryotes and plants with increased efficiency of methionine synthesis compared to the starting organism which comprises the steps of:
a. carrying out the above-mentioned elementary flux mode analysis to obtain a theoretical prediction about the optimal metabolic flux distribution in an organism that is optimised with respect to methionine synthesis, and
b. genetically modifying an organism in a manner to modify existing metabolic pathways in the organism such that the metabolic flux of the organism is approximated to the theoretical model of step a) compared to the starting organism and/or
c. genetically modifying an organism in a manner to introduce exogenous metabolic pathways into the organism such that the metabolic flux of the organism is approximated to the theoretical model of step a) compared to the starting organism and/or
d. providing external metabolites in amounts sufficient to channel the metabolic flux through the metabolic pathways of b) and c).

A further aspect of the present invention relates to a method which puts the theoretical predictions of flux distribution in an organism being optimised for methionine synthesis into practise by producing an organism which is selected from the group of prokaryotes, lower eukaryotes and plants by:
modifying the metabolic flux through at least one of the following metabolic pathways by genetic modification of the organisms:
   phosphotransferase system (PTS) and/or
   pentose phosphate pathway (PPP) and/or
   glycolysis (EMP) and/or
   tricarboxylic acid cycle (TCA) and/or
   glyoxylate shunt (GS) and/or
   anaplerosis (AP) and/or
   respiratory chain (RC) and/or
   sulfinur assimilation (SA) and/or
   methionine synthesis (MS) and/or
   serine/cysteine/glycine synthesis (SCGS) and/or
   glycine cleavage system (GCS) and/or
   transhydrogenase conversion (THGC) and/or
   pathway 1 (P1) and/or
   pathway 2 (P2) and/or
   pathway 3 (P3) and/or
   pathway 4 (P4) and/or
   pathway 5 (P5) and/or
   pathway 6 (P6) and/or
   pathway 7 (P7), and/or
introducing a metabolic flux through at least one of the following exogenous metabolic pathways by genetic modification of the organisms:
   glycine cleavage system (GCS) and/or
   transhydrogenase conversion (THGC) and/or
   thiosulfate reductase system (TRS) and/or
   sulfate reductase system (SARS) and/or
   sulfite reductase system (SRS) and/or
   formate converting system (FCS) and/or
   methanethiol converting system (MCS), and/or
cultivating the organisms in the presence of:
   a. sulfate and/or
   b. sulfite and/or
   c. sulfide and/or
   d. thiosulfate and/or
   e. organic sulfur containing compounds and/or
   f. C1-metabolites such as formate, formaldehyde, methanol, methanethiol or its dimer dimethyldisulfide.

The organisms that have been genetically modified in order to put the predictions as to a model organism with increased efficiency of methionine biosynthesis into practise are also an object of the present invention.

As mentioned above, for the calculation of the optimal metabolic flux through a metabolic network for methionine synthesis, the organism's specific metabolic pathways leading to this amino acid are used. Furthermore, the specific stoichiometries of the specific organisms have to be considered for each metabolic network constructed. The stoichiometries may be taken from the above-mentioned sources.

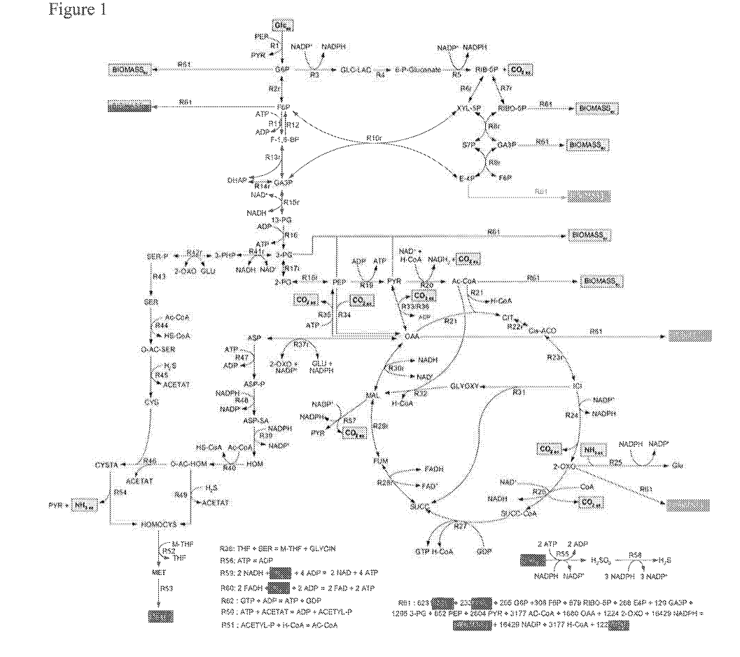

Even though such metabolic networks may differ between organisms such as E. coli to C. glutamicum, FIG. 1 shows a set of reactions that was used for calculating the initial metabolic network using C. glutamicum as an example. As this set is only a minimal set of reactions for a metabolic network contributing to methionine synthesis, additional pathways were regarded for other organisms such as E. coli if their existence was known. However, if reference is made below generally to a certain metabolic pathway or a specific enzyme, these general references all relate to the reactions shown in FIG. 1 unless otherwise indicated. This seems justified because these reactions were largely identical in E. coli and C. glutamicum. For the purposes of the present invention, the various reactions are grouped into the following pathway groups:
phosphotransferase system (PTS)
pentose phosphate pathway (PPP)
glycolysis (EMP)
tricarboxylic acid cycle (TCA)
glyoxylate shunt (GS)
anaplerosis (AP)
respiratory chain (RC)
sulfinur assimilation (SA)
methionine synthesis (MS)
serine/cysteine/glycine synthesis (SCGS)
glycine cleavage system (GCS)
transhydrogenase conversion (THGC)
pathway 1 (P1)
pathway 2 (P2)
pathway 3 (P3)
pathway 4 (P4)
pathway 5 (P5)
pathway 6 (P6)
pathway 7 (P7)
thiosulfate reductase system (TRS)
sulfate reductase system (SARS)
sulfite reductase system (SRS)
formate converting system (FCS)
methanethiol converting system (MCS).

The single pathways may be subdivided into the following reactions which are catalysed by enzymes designated Rn. Abbreviations are used to define these reactions. The way that these definitions are to be understood for the purposes of the invention is explained with respect to the phosphotransferase system. This explanation also applies to the other reactions.

For the purposes of the present invention, the phosphotransferase system (PTS) comprises the reaction of external glucose to glucose-6-phosphate (G6P). This reaction is catalysed by enzyme R1 which is phosphotransferase. This enzyme uses phosphoenolpyruvate as a phosphor-group donor (see FIG. 1). For the purposes of the invention this reaction is described as:
R1 in order to produce more G6P

The single reactions of the various above-mentioned pathways are thus defined with respect to the enzymes that catalyse the reaction and the products resulting from the reactions. Whether or not such a reaction may require energy input in the form of ATP, NADH and/or NADPH or other co-factors is not indicated, but may be taken from FIG. 1. The specific stoichiometry is also not indicated, as this may vary from organism to organism. In general, the educts and energy input of the reaction are also not indicated. These data may be taken from standard textbooks or scientific publications on the various organisms.

For the purposes of the present invention, the pentose phosphate pathway is characterized by the following reactions:
R3 in order to produce GLC-LAC
R4 in order to produce 6-P-Gluconate
R5 in order to produce RIB-5P
R6 in order to produce XYL-5P
R7 in order to produce RTBO-5P
R8 in order to produce S7P and GA3P
R9 in order to produce E4p and F6P
R10 in order to produce F6P and GA3P
R2 in order to produce G6P

For the purposes of the present invention, the glycolysis pathway (EMP) is characterized by the following reactions:
R11 in order to produce F-1,6-BP
R13 in order to produce DHAP and GA3P
R14 in order to produce GA3P
R15 in order to produce 1,3-PG
R16 in order to produce 3-PG
R17 in order to produce 2-PG
R18 in order to produce PEP
R19 in order to produce Pyr

For the purposes of the present invention, the tricarboxylic acid cycle (TCA) is defined by the following reactions:
R20 in order to produce Ac-CoA
R21 in order to produce CIT
R22 in order to produce Cis-ACO
R23 in order to produce ICI
R24 in order to produce 2-OXO
R26 in order to produce SUCC-CoA
R27 in order to produce SUCC
R28 in order to produce FUM
R29 in order to produce MAL
R30 in order to produce OAA

For the purposes of the present invention, the glyoxylate shunt (GS) pathway is defined by the following reactions:
R21 in order to produce CIT
R22 in order to produce Cis-ACO
R23 in order to produce ICI
R31 in order to produce GLYOXY and SUCC
R32 in order to produce MAL
R28 in order to produce FUM
R29 in order to produce MAL
R30 in order to produce OAA

For the purposes of the present invention, the anaplerosis (AP) pathway is defined by the following reactions:
R34 in order to produce OAA
R33/R36 in order to produce OAA

For the purposes of the present invention, the respiratory chain (RC) is defined by the following reactions:
R59 catalysing: 2NADH+O2ₑₓ+4ADP=2NAD+4ATP
R60 catalysing: 2FADH+O2ₑₓ+2 ADP=2FAD+2ATP

For the purposes of the present invention, the sulfur assimilation pathway (SA) is defined by the following reactions:
R55 in order to produce H₂SO₃
R58 in order to produce H₂S

For the purposes of the present invention, the methionine synthesis pathway (MS) is defined by the following reactions:
R37 in order to produce Asp
R47 in order to produce ASP-P
R48 in order to produce ASP-SA
R39 in order to produce HOM
R40 in order to produce O-AC-HOM
R46 in order to produce CYSTA
R49 in order to produce HMOCYS
R54 in order to produce HOMOCYS
R52 in order to produce MET
R53 in order to produce METₑₓ

For the purposes of the present invention, the serine/cysteine/glycine synthesis (SCGS) pathway is defined by the following reactions:
R41 in order to produce 3-PHP R42 in order to produce SER-P
R43 in order to produce SER
R44 in order to produce O-AC-SER
R45 in order to produce CYS
R38 in order to produce M-THF and Glycineₑₓ

For the purposes of the present invention, pathway 1 (P1) comprises the following reactions:
R25 in order to produce GLU

For the purposes of the present invention, pathway 2 (P2) comprises the following reactions:
R33/R36 in order to produce OAA
R30 in order to produce MAL
R57 in order to produce PYR+CO₂

For the purposes of the present invention, pathway 3 (P3) comprises the following reaction:
R56 catalysing: ATP=ADP

For the purposes of the present invention, pathway 4 (P4) comprises the following reactions:
R62 catalysing: GTP+ADP=ATP+GDP

For the purposes of the present invention, pathway 5 (P5) is defined by the following reactions:
R50 catalysing: ATP+acetate=ADP+acetyl P

For the purposes of the present invention, pathway 6 (P6) comprises the following reactions:
R51 catalysing: acetyl P+HCoA=acetyl CoA

For the purposes of the present invention, pathway 7 (P7) comprises the following reaction:
R61 catalysing: 6231 NH₃ₑₓ+233 SO₄ₑₓ+205 G6P+308 F6P+879 RIBO-5P+268E4P+129GA3P+1295 3-PG+652PEP+2604PYR+3177AC-CoA+1680 OAA+1224 2-OXO+16429 NADPH=BIOMASSₑₓ+16429 NADP+3177H-CoA+1227 CO₂ₑₓ

As set out above, the stoichiometry will vary from organism to organism and may be taken from the literature or the above-mentioned Internet pages. Furthermore, the metabolic network of certain organisms such as E. coli or C. glutamicum may comprise additional reaction pathways.

Such additional pathways, as they are used for the purposes of the present invention, include:
glycine cleavage system (GCS) and/or
transhydrogenase conversion (THGC) and/or
thiosulfate reductase system (TRS) and/or
sulfate reductase system (SARS) and/or
sulfite reductase system (SRS) and/or
formate converting system (FCS) and/or
methanethiol converting system (MCS)

For the purposes of the present invention, the glycine cleavage system (GCS) comprises the following reactions:
R71: in order to produce M-HPL
R72: in order to produce Methylene-THF

The person skilled in the art is well aware that the reactions of R71 and R72 are catalysed by at least three proteins, namely gcvH, P and T (see Tables 1 and 2). gcvP catalyses the decarboxylation of glycine to CO₂ and an aminomethyl group, while GcvH is a carrier of the aminomethyl-group (R71). A description of the glycine cleavage system can be found in Neidhardt F. C. (1996) E. coli and S. typhimurium, ASM Press Washington. gcvT is involved in the transfer of the C1 unit from the H-protein to tetrahydrofolate and the release of NH₃ (R72). The reaction is then typically completed by the fourth subunit which is lipoamide dehydrogenase. The lpda encoded lipoamide dehydrogenase functions as the electron transfer from NAD to NADH. This dehydrogenase is borrowed from the multi-subunit pyruvate dehydrogenase and is commonly called lpdA. For the purposes of the present invention the GCS may thus be summarized as:
R71/72: in order to produce Methylene-THF

For the purposes of the present invention, the GCS can optionally also comprise the additional following reaction:
R78: in order to produce Methyl-THF

Strictly speaking, R78 does not belong to the GCS as it only serves to provide Methyl-THF. However, in organisms in which R78 is not present, R78 may be implemented together with the other reactions of the GCS. In organisms in which R78 is already present, this may not be necessary.

For the purposes of the present invention, the transhydrogenase conversion system (THGC) comprises the following reaction:
R70: in order to produce NADPH

For the purposes of the present invention, the THGC may also comprise the following reaction:
R81: in order to produce NADPH

While R70 may for example be a cytosolic Transhydrogenase, R81 may e.g. be a transmembrane Transhydrogenase.

For the purposes of the present invention, the thiosulfate reductase system (TRS) comprises the following reactions:
R73: in order to metabolise thiosulfate into sulfide and sulfite

For the purposes of the present invention, the TRS may additionally comprise:
R82: in order to import extracellular H₂S₂O₃ into the cell and/or
R45a: in order to produce more S-Sulfocysteine and/or
R49: in order to produce more S-Sulfocysteine.

R45a and/or R49 convert Thiosulfate into S-Sulfo-Cysteine and thus belong to the SRS.

For the purposes of the present invention, the sulfate reductase system (SARS) comprises the following reaction:
R80: in order to metabolise sulfate into sulfite

For the purposes of the present invention, the sulfite reductase system (SRS) comprises the following reaction:
R74 in order to metabolise sulfite into sulfide

For the purposes of the present invention, the formate converting system (FCS) comprises the following reactions:
R75: in order to produce 10-formyl-THF
R76: in order to produce Methylene-THF
R78: in order to produce Methyl-THF

For the purposes of the present invention, the methanethiol converting system (MCS) comprises the following reactions:
R77 in order to methyl-sulfhydrylate O-Acetyl-homoserine with methanethiol

For the purposes of the present invention, pathway 8 (P8) comprises the following reaction:
R79 in order to degrade formyl-THF to formate and tetrahydrofolate

In the following table specific examples are given for the above-mentioned enzymes.

Further reactions can be found in the overview of reactions given further below.

TABLE 1

| Number | Enzyme | Gene bank accession number | Organism |
|---|---|---|---|
| R1 | Phospho-transferase | Cgl1360, Cgl1936, Cgl2642 | Corynebacterium |
| | system | | glutamicum |
| R2 | G6P-isomerase | Cgl0851 | Corynebacterium |
| | | | glutamicum |
| R3 | G6P-Dehydrogenase, | Cgl1576, BAB98969) | Corynebacterium |
| | OPCA protein | Cgl1577 | glutamicum and |
| | | | others) |
| R4 | Lactonase | Cgl1578 | Corynebacterium |
| | | | glutamicum |
| R5 | Gluconate-Dehydrogenase | Cgl1452, BAB98845 | Corynebacterium |
| | | | glutamicum and |
| | | | others) |
| R6 | Ribulose-5-P-epimerase | Cgl1598 | Corynebacterium |
| | | | glutamicum |
| R7 | Ribose-5-P-isomerase | Cgl2423 | Corynebacterium |
| | | | glutamicum |
| R8 | Transketolase 1 | Cgl1574, YP_225858 - | Corynebacterium |
| | | | glutamicum and |
| | | | others |
| R9 | Transaldolase | Cgl1575 | Corynebacterium |
| | | | glutamicum and |
| | | | others |
| R10 | Transketolase 2 | Cgl1574 | Corynebacterium |
| | | | glutamicum and |
| | | | others |
| R11 | Phosphofructokinase | Cgl1250 | Corynebacterium |
| | | | glutamicum |
| R12 | Fructosebisphosphatase | Cgl1058 | Corynebacterium |
| | | Cgl1909 | glutamicum |
| | | Cgl2095 | and others |
| R13 | Fructosebisphosphate- | Cgl2770 | Corynebacterium |
| | aldolase | | glutamicum |
| R14 | Triosephosphate- | Cgl1586 | Corynebacterium |
| | isomerase | | glutamicum |
| R15 | 3-phospho glycerate- | Cgl1587 | Corynebacterium |
| | Kinase | | glutamicum |
| R16 | PG-kinase | Cgl2080 | Corynebacterium |
| | | | glutamicum |
| R17 | PG-mutase | Cgl0438 | Corynebacterium |
| | | | glutamicum |
| R18 | PEP-hydratase | Cgl0974 | Corynebacterium |
| | | | glutamicum |
| R19 | PYR-kinase | Cgl2089 | Corynebacterium |
| | | | glutamicum |
| R20 | PYR-Dehydrogenase | Cgl2248 | Corynebacterium |
| | | Cgl2610 | glutamicum |
| | | Cgl1271 | |
| | | Cgl1129 | |
| | | Cgl0097 | |
| | | Cgl0162 | |
| R21 | CIT-Synthase | Cgl0659 | Corynebacterium |
| | | | glutamicum |
| R22 | ACO-Hydrolase | Cgl1315 | Corynebacterium |
| | | Cgl1540 | glutamicum |
| R23 | Aconitase | Cgl1315 | Corynebacterium |
| | | | glutamicum |
| R24 | Isocitrate-Dehydrogenase | Cgl1286 | Corynebacterium |
| | | Cgl0664 | glutamicum |
| R25 | Glutamate- | Cgl2079 | Corynebacterium |
| | Dehydrogenase | | glutamicum |
| R26 | 2-OXO-Dehydrogenase | Cgl1129 | Corynebacterium |
| | | | glutamicum |
| R27 | SUCC-CoA-synthase | Cgl2565 | Corynebacterium |
| | | | glutamicum |
| R28 | SUCC-Dehydrogenase | Cgl0370 | Corynebacterium |
| | | | glutamicum |
| R29 | Fumarase | Cgl1010 | Corynebacterium |
| | | | glutamicum |
| R30 | MAL-Dehydrogenase | Cgl2380 | Corynebacterium |
| | | | glutamicum |
| R31 | ICI-Lyase | Cgl0097 Cgl2331 | Corynebacterium |
| | | | glutamicum |
| R32 | MAL-Synthase | Cgl2329 | Corynebacterium |
| | | | glutamicum |
| R33 | PYR-Carboxylase | Cgl0689 | Corynebacterium |
| | | | glutamicum |
| R34 | PEP-Carboxylase | Cgl1585 | Corynebacterium |
| | | | glutamicum |
| R35 | PEP-Carboxykinase | Cgl2863 | Corynebacterium |
| | | | glutamicum |
| R36 | OAA-Decarboxylase | CE0804 | Corynebacterium |
| | | | glutamicum |
| R37 | ASP-Transaminase | Cgl0240 | Corynebacterium |
| | | | glutamicum |
| R38 | M-THF synthesis 1 | Cgl0860 Cgl1600 Cgl0382 Cgl0861 | Corynebacterium |
| | | Cgl0648 Cgl2171 Cgl0881 Cgl0996 | glutamicum |
| | | Cgl1972 | |
| R39 | HOM-Dehydrogenase | Cgl1183 | Corynebacterium |
| | | | glutamicum |
| R40 | HOM-Transacetylase | Cgl0652 | Corynebacterium |
| | | | glutamicum |
| R41 | PG-Dehydrogenase | Cgl1284 | Corynebacterium |
| | | | glutamicum |
| R42 | Phosphoserine- | Cgl0828 | Corynebacterium |
| | transaminase | | glutamicum |
| R43 | Phosphoserine- | Cgl0299 | Corynebacterium |
| | phosphatase | | glutamicum |
| R44 | Serine-Transacetylase | Cgl2563 | Corynebacterium |
| | | | glutamicum |
| R45 | Cysteine-Synthase | Cgl2136 Cgl0653 | Corynebacterium |
| | | | glutamicum |
| R45a | S-Sulfo-Cysteine- | Cgl2136 Cgl0653 | Corynebacterium |
| | Synthase | | glutamicum |
| R46 | Cystathionine-Synthase | Cgl2446 | Corynebacterium |
| | | | glutamicum |
| R47 | Aspartokinase | Cgl0251 | Corynebacterium |
| | | | glutamicum |
| R48 | ASP-P-Dehydrogenase | Cgl0252 | Corynebacterium |
| | | | glutamicum |
| R49 | O-Ac-HOM | Cgl0653 | Corynebacterium |
| | Sulfhydrylase | | glutamicum |
| R50 | Acetat-Kinase | Cg3047 | Corynebacterium |
| | | | glutamicum |
| R51 | Phosphotransacetylase | Cg3048 | Corynebacterium |
| | | | glutamicum |
| R52 | MET-Synthase (MetE/H) | Cgl1507 Cgl1139 | Corynebacterium |
| | | | glutamicum |
| R53 | Methionine Exporter | YP_224558 | Corynebacterium |
| | | CAF18830 | glutamicum |
| R54 | Cystathionine-Lyase | Ceg2536 | Corynebacterium |
| | | | glutamicum |
| R55 | ATP-Sulfurylase | Cgl2814 | Corynebacterium |
| | | | glutamicum |
| R56 | ATP-Hydrolysis | Not applicable | spontaneous |
| | | | reaction |
| R57 | Malic enzyme | Cgl3007 | Corynebacterium |
| | | | glutamicum |
| R58 | Sufite-Reductase | Cg3118 | Corynebacterium |
| | | | glutamicum |
| R59 | Respiratory chain 1 | Cgl1212 Cgl1210 Cgl1211 Cgl1209 | Corynebacterium |
| | | Cgl1213 Cgl1207 Cgl1206 Cgl1208 | glutamicum |
| R60 | Respiratory chain 2 | Cgl1212 Cgl1210 Cgl1211 Cgl1209 | Corynebacterium |
| | | Cgl1213 Cgl1207 Cgl1206 Cgl1208 | glutamicum |
| R61 | Biomass formation | | Sum equation |
| R62 | GTP-ATP-Phospho- | Cg2603 | Corynebacterium |
| | transferase | | glutamicum |
| R70 | Transhydrogenase | AAC76944, NP_214669, NP_334574 | E. coli and |
| | | | others |
| R71 | Glycine decarboxylase P | Q8FE66, P0A6T9 | E. coli and |
| | protein | | others |
| | H protein | | |
| R72 | Aminomethyltransferase | CAA52144.1, P0A6T9 | E. coli and |
| | T protein | | others |
| | H protein | | |
| R71/72 | Glycine clevage system | CAA52144, Q8FE66, P0A9P0, P0A6T9 | E. coli and |
| | GcvP, H, T lpda | | others |
| R73 | Thiosulfate Reductase | NP_461008, NP_461009, NP_461010 | Salmonella |
| | consisting of 3 subunits | | typhimurium and |
| | | | others |
| R74 | anaerobic Sulfite | AAL21442, AAL21443, NP_804181 | Salmonella |
| | Reductase-consisting of | | typhimurium and |
| | 3 subunits | | others |
| R75 | Formate-THF-ligase | NP_939608) | C diphteriae and |
| | | | others |
| R76 | 5-formyl-tetrahydrofolate | NCgl0845 | C. glutamicum |
| | cyclo-ligase | | and others |
| R77 | O-Acetyl-homoserine- | NCgl0625 | C. glutamicum |
| | (methyl)-sulfhydrolase | | and others |
| R78 | 5,10-methyleneTHF | NCgl2091, NP_601375 | C. glutamicum |
| | reductase(NAD(P)H | | |
| | Methylenetetrahydrofolate | | |
| | dehydrogenase (NADP+) | | |
| | (EC 1.5.1.5) | | |
| R79 | formyl-tetrahydrofolate | ADD13491 | C. glutamicum |
| | deformylase | | |
| | degrades formyl-THF to | | |
| | formate and | | |
| | tetrahydrofolate | | |
| R80 | Sulfate reductase system | NP_602005, NP_602006, NP_602007, | C. glutamicum |
| | | CAF20840, CAF20841 | and others |
| R81 | Transmembrane | CAA46822 | others |
| | Transhydrogenase | | |
| R82 | Sulfate uptake transporter | YP_224929 | C. glutamicum |
| | (ABC transporter | | and others |

The above accession numbers are the official accession numbers of Genbank or are synonyms for accession numbers which have cross-references at Genbank. These numbers can be searched and found at http://www.ncbi.nlm.nih.gov/.

The present invention also envisions that the metabolic flux through other pathways and reactions may be modulated by theoretic or genetic manipulation of organisms for producing organisms with increased efficiency of methionine synthesis as long as these reactions are known e.g. in the scientific literature to participate directly or indirectly in methionine synthesis. These pathways and reactions may, of course, also be implemented in the theoretic elementary flux mode analysis. The (genetically modified) organisms obtained by these methods are also part of the invention.

As mentioned above, according to the present invention the actual metabolic flux in an organism is to be approximated to the optimal theoretical flux for an organism with increased methionine synthesis, as determined by the elementary flux mode analysis in accordance with claim 1. For the purposes of the present invention, “approximated” means that the metabolic flux of the genetically modified organism as a consequence of genetic modification resembles more the metabolic flux of the theoretical predictions than does the metabolic flux of the starting organism.

As already set out, modulation of the metabolic flux of the starting organism may be influenced by genetic alteration of the organism, e.g. by influencing the amount and/or the activity of enzymes that catalyse specific reactions of the network considered. Additionally, the metabolic flux may be influenced by the use of certain nutrients and external metabolites such as sulfate, thiosulfate, sulfite and sulfide and C1-compounds such as formate formaldehyde, methanol methanethiol and dimethyldisulfide. While the influence of external metabolites such as thiosulfate, formate or methanethiol will be explained in more detail later on, general examples are given below for the genetic modification of organisms.

In the following, it will be explained with respect to a specific reaction how the metabolic flux through a certain pathway may be channelled by genetic modification of an organism. These explanations correspondingly apply to other reactions.

If, for example, the theoretical model obtained or the model organism designed according to the method of the present invention predicts that for efficient methionine synthesis the metabolic flux should be mainly channelled into the PPP, an actual organism with increased metabolic flux through this pathway may be obtained by genetically influencing the amount and/or activity of the aforementioned reactions being part of the PPP. Thus, metabolic flux may be increased into the PPP by increasing the amount and/or activity of R3, leading to the formation of more GLC-LAC. In the same way, increasing the amount and/or activity of R4, R5, R6, R7,R8,R9 or R10 may increase the metabolic flux into the PPP. The same may be achieved by increasing the activity of R2 towards the production of G6P.

If the theoretical model obtained by the method of the present invention predicts a reduction of the metabolic flux through the TCA, this may be achieved by reducing the amount and/or activity of the following enzymes 21, R22, R23, R24, R26, R27, R28, R29 or R30. How the activity and/or amount of an enzyme may be increased or reduced is apparent to the skilled person and will also be exemplified below.

For general purposes, it should however be noted that in a metabolic pathway, such as in FIG. 1, certain reactions may be considered as being irreversible. While almost any reaction of a biological network is an equilibrium reaction being able to proceed in both directions, irreversible reactions are commonly considered to be reactions in which, by the input of e.g. energy, the reaction is predominantly driven in one direction, so that the equilibrium of the reaction lies almost exclusively on one side of the reaction.

In the case of the PPP, such irreversible reactions are e.g. the reactions catalysed by R3 and R5, both of which are favoured by the formation of NADPH. Other such irreversible reactions, as this term is used in the context of this invention, are e.g. R16 of the EMP, R24 of the TCA, etc. Irreversible reactions are indicated in FIG. 1 by arrows pointing only in one direction.

If, in the context of the present invention, it is stated that the metabolic flux through a certain reaction pathway is increased by increasing the amount and/or activity of the enzyme catalysing that direction, then this statement has to be seen in the context of how the reactions are defined above. Increasing or decreasing the amount and/or activity of an enzyme has to be understood with respect to the direction in which the reaction should be further pushed or channelled. As the reactions of the various pathways of the metabolic network in accordance with the present invention have been defined by an enzyme and the product being formed by that enzyme, increasing the amount and/or activity of an enzyme or decreasing the amount and/or activity of an enzyme are clearly understood by the person skilled in the art to influence the amount and/or activity of the enzyme in such a way that more or less product is obtained.

Thus, if e.g. it is stated that the activity of the enzyme R6 is increased, then in view of the above-mentioned description of this reaction this means that by increasing the amount and/or activity of R6, the amount of XYL-5P is increased. Similarly, if it is stated that the amount and/or activity of R23 is increased, this refers to a situation where the amount and/or activity of R23 is increased to produce more ICI.

Correspondingly, if e.g. the amount and/or activity of R29 are decreased, then this means that the amount and/or activity of R29 is reduced in order to produce less MAL.

If the theoretical model organisms with increased methionine efficiency require e.g. an increase of the metabolic flux through a certain pathway, in one embodiment of the invention it may be sufficient to modify the amount and/or activity of only one enzyme of that reaction pathway. Alternatively, the amount and/or activity of various enzymes of this metabolic pathway may be modified. If, e.g. the theoretical model obtained by elementary flux analysis suggests to e.g. increase the metabolic flux through the PPP and the TCA while the metabolic flux through the RC should be reduced, this may be achieved by increasing the amount and/or activity of only one enzyme of the PPP and the TCA cycle while the activity and/or amount of only one enzyme of the RC may be reduced. Alternatively, the amount and/or activity of various or all enzymes of these pathways may be influenced at the same time.

The person skilled in the art is also well aware that what may defined above as an enzymatic reaction being carried out by a single enzymatic activity may actually be a series of enzymatic (sub)steps by various enzymes which as a whole provide the indicated overall activity (e.g. sulfite or thiosulfate reductase). The indicated overall enzymatic activity (see above R-numbers) may also be composed of various subunits. In these case the metabolic flux thru the above identified reactions may be influenced by modifying the activity and/or amount of at least one of the enzymes carrying out one of the single (sub)steps or of at least one of the subunits. Accordingly, genes coding for (sub)steps or subunits may be considered as part of the overall respective enzymatic activity.

With respect to the Glycine cleavage system, the skilled person knows that the genes gcvT, and/or H, and/or P and/or L (lpdA) (see Tables 1 and 2) are involved in this system. The metabolic flux through this system which is defined above by the reactions R71, R72 and R71/R72 may thus be increased or introduced by e.g. over-expression of at least one of the above identified genes or their homologues. Increasing the metabolic flux may also be achieved by over-expressing all four of these genes or only two or three of these gene. The genes may be overexpressed together for example in a natural occurring operon or in an artificial operon constructed using promotors. Additionally it can be useful to also overexpress the gene lpdA together with the genes gcvH, P, T (see again Tables 1 and 2).

With respect to the methionine synthesis system, the skilled person knows that the reactions:

R47, R48, R39, R40 R46, R49, R52, R53, R54 are involved in the synthesis of methionine.

For the overexpression of the transhydrogenase (R70 and R81) at least one of the genes udh, pntA and/or pntB or their homologues (see Tables 1 and 2) may be overexpressed. The genes may also be overexpressed together e.g. in a natural occurring operon or in an artificial operon constructed using promoters. One may, of course, in addition or alternatively also overexpress a gene for a transmembrane transhydrogenase such as udhA and or pntA, B.

For the overexpression of the Thiosulfate-Reductase (R73, R45a, R49 and/or R82) the genes thiosulfate reductase cytochrome B subunit, thiosulfate reductase electron transport protein and/or thiosulfate reductase precursor may be overexpressed either alone or in combination for example in a natural occurring operon or in an artificial operon constructed using promoters. For theses purposes the phsA, B and/or C genes or their homologues may be used (see Tables 1 and 2). Similarly the genes of an ABC transporter such as YP_{—}224929 may be overexpressed.

For the overexpression of the pentose phosphate pathway the genes Glucose-6-phosphate dehydrogenase, OPCA, transketolase, transaldolase, 6-phosphoglucono lactone dehydrogenase or their homologues (see Tables 1 and 2) can be overexpressed either alone or in any combination of 2, 3, 4 or more genes for example in a natural occurring operon or in an artificial operon constructed using promotors.

For the overexpression of the sulfite reduction system (R74) the genes anaerobic sulfite reductase subunit A, B and C may be overexpressed either alone or together e.g. in a natural occurring operon or in an artificial operon constructed using promotors. The genes dsrA, dsrB and/or dsrC or their homologues (see Tables 1 and 2) may be used for these purposes.

With respect to the formate converting system (FCS), metabolic flux may be modified and in some embodiments increased or introduced by modifying the amount and/or activity of at least one of the following genes being selected from the group of Formate-THF-ligase, Formyl-THF-cycloligase, Methylene-THF-dehydrogenase, 5,10-Methylene-THF-reductase, Methylene-THF-Reductase. The homologues thereof may also be used (see Tables 1 and 2). The metabolic flux through the FCS may be also increased by overexpression of any of these genes.

The sulfate reductase system (SARS, R80) may be considered to consist of sulfate adenylate transferase subunit 1 (NP_{—}602005) and sulfate adenylate transferase subunit 2 (NP_{—}602006) constituting the ATP:sulfate adenylyltransferase, the adenosine 5′-phosphosulfate kinase (EC:2.7.1.25), the 3′-phosphoadenosine 5′-phosphosulfate (PAPS) reductase (EC: 1.8.4.8, NCgl2717) and the sulfite reductase, (EC: 1.8.1.2, CAF20840)

A preferred target for modification may be the amount and/or activity of enzymes that are considered to be irreversible in the sense of the present invention. Thus, the theoretical models obtained by the metabolic flux analysis for organisms showing an increased efficiency for methionine synthesis give the person skilled in the art a clear guidance of what genetic manipulations the skilled person should consider for obtaining a microorganism with such an optimised metabolic flux. The person skilled in the art will then single out the decisive enzymes which are all well known to him from constructing the theoretical metabolic network and will influence the amount and/or activity of these enzymes by genetic modification of the organism. How such organisms can be obtained by genetic modification belongs to the general knowledge in the art.

By genetically amending organisms in accordance with the present invention, the metabolic flux in these organisms may be amended in order to increase the efficiency of methionine synthesis such that these organisms are characterized in that methionine is produced with an efficiency of at least 10%, at least 15%, at least 20%, at least 25%, at least 30%, at least 35%, at least 40%, at least 45%, at least 50%, at least 55%, at least 60%, at least 65%, at least 70%, at least 75%, at least 80% or at least 85%.

In the theoretical part of the experimental section it is described what the optimal metabolic flux modes for C. glutamicum and E. coli with increased efficiency of methionine production look like. While it is set out there in detail how these models were calculated, what reactions were considered and what stoichiometries were used, the general conclusions from these models are listed below. The following section therefore has to be understood as an instruction to the person skilled in the art, which metabolic pathways should be genetically modified in order to approximate the metabolic flux through these pathways towards the optimal values as obtained for the theoretical model. A working schedule will then be given in the practical part of the experimental section to illustrate for certain enzymes which specific measure have to be taken for genetic manipulation.

C. glutamicum

One object of the present invention relates to a microorganism of the genus Corynebacterium which has been genetically modified in order to increase and/or introduce a metabolic flux through at least one of the following pathways compared to the starting organism:
phosphotransferase system (PTS) and/or
pentose phosphate pathway (PPP) and/or
sulfinur assimilation (SA) and/or
anaplerosis pathway (AP) and/or
methionine synthesis pathway (MS) and/or
serine/cysteine/glycine synthesis (SCGS) and/or
glycine cleavage system (GCS) and/or
transhydrogenase conversion (THGC) and/or
pathway 1 (P1) and/or
pathway 2 (P2) and/or
thiosulfate reductase system (TRS) and/or
sulfite reductase system (SRS) and/or
sulfate reductase system (SARS) and/or
Formate converting system (FCS) and/or
Methanethiol converting system (MCS) and/or

At the same time such an optimized microorganism should optionally have an at least reduced metabolic flux through at least one of the following pathways:
glycolysis (EMP) and/or
tricarboxylic acid cycle (TCA) and/or
glyoxylate shunt (GS) and/or
respiratory chain (RC) and/or
pathway 3 (P3) and/or
pathway 4 (P4) and/or
pathway 7 (P7) and/or
R19 in order to produce less pyruvate and/or
R35 in order to produce less PEP and/or
R79 in order to produce less THF.

The present invention relates to a method for producing a microorganism of the genus Corynebacterium with increased efficiency of methionine production comprising the following steps.
increasing and/or introducing the metabolic flux through at least one of the following pathways compared to the starting by genetic modification of the organism:
   phosphotransferase system (PTS) and/or
   pentose phosphate pathway (PPP) and/or
   sulfur assimilation (SA) and/or
   anaplerosis pathway (PA) and/or
   methionine synthesis pathway (MS) and/or
   serine/cysteine/glycine system (SCGS) and/or
   glycine cleavage system (GCS) and/or
   transhydrogenase conversion (THGC) and/or
   pathway 1 (P1) and/or
   pathway 2 (P2) and/or
   thiosulfate reductase system (TRS) and/or
   sulfite reductase system (SRS) and/or
   sulfate reductase system (SARS) and/or
   formate converting system (FCS) and/or
   methanethiol converting system (MCS) and/or
at least partially decreasing the metabolic flux through at least one of the following pathways compared to the starting by genetic modification of the organism:
   glycolysis (EMP) and/or
   tricarboxylic acid cycle (TCA) and/or
   glyoxylate shunt (GS) and/or
   respiratory chain (RC) and/or
   pathway 3 (P3) and/or
   pathway 4 (P4) and/or
   pathway 7 (P7) and/or
   R19 in order to produce less pyruvate and/or
   R35 in order to produce less PEP and/or
   R79 in order to produce less THF.

One embodiment of the present invention relates to a method for producing a microorganism of the genus Corynebacterium with an increased efficiency for methionine synthesis wherein
with respect to PTS, the amount and/or activity of the following enzyme is increased and/or introduced compared to the starting organism:
   a. R1 in order to produce more G6P; and/or with respect to PPP, the amount and/or activity of the following enzymes is/are increased and/or introduced compared to the starting organism:
   a. R3 in order to produce more GLC-LAC and/or
   b. R4 in order to produce more 6-P-Gluconate and/or
   c. R5 in order to produce more RIB-5P and/or
   d. R6 in order to produce more XYL-5P and/or
   e. R7 in order to produce more RIBO-5P and/or
   f. R8 in order to produce more S7P and GA3P and/or
   g. R9 in order to produce more E4p and F6P and/or
   h. R10 in order to produce more F6P and GA3P and/or
   i. R2 in order to produce more G6P; and/or
with respect to SA, the amount and/or activity of the following enzymes is/are increased and/or introduced compared to the starting organism:
   a. R55 in order to produce more H₂SO₃ and/or
   b. R58 in order to produce more H₂S and/or
with respect to AP, the amount and/or activity of the following enzyme is increased and/or introduced compared to the starting organism:
   a. R33 in order to produce more OAA and/or
with respect to MS, the amount and/or activity of the following enzyme is increased and/or introduced compared to the starting organism:
   a. R37 in order to produce more Asp and/or
   b. R39 in order to produce more HOM and/or
   c. R40 in order to produce more O-AC-HOM and/or
   d. R46 in order to produce more CYSTA and/or
   e. R47 in order to produce more ASP-P and/or
   f. R48 in order to produce more ASP-SA and/or
   g. R49 in order to produce more HOMOCYS and/or
   h. R52 in order to produce more MET and/or
   i. R53 in order to produce more METₑₓ and/or
   j. R54 in order to produce more HOMOCYS and/or
with respect to SCGS, the amount and/or activity of the following enzyme is increased and/or introduced compared to the starting organism:
   a. R38 in order to produce more M-THF and Glycineₑₓ and/or
   b. R44 in order to produce more O-AC-SER and/or
   c. R45 in order to produce more CYS and/or
with respect to CGS, the amount and/or activity of the following enzymes is/are increased and/or introduced compared to the starting organism:
   a. R71 in order to produce more M-HPL and/or
   b. R72 in order to produce more Methylene-THF
   c. R78 in order to produce more Methylene-THF and/or
with respect to THGC, the amount and/or activity of the following enzyme is increased and/or introduced compared to the starting organism:
   a. R70 in order to produce more NADPH and/or
   b. R81 in order to produce more NADPH and/or
with respect to P1, the amount and/or activity of the following enzymes is/are increased and/or introduced compared to the starting organism:
   a. R25 in order to produce more Glu; and/or
with respect to P2, the amount and/or activity of the following enzymes is/are increased and/or introduced compared to the starting organism:
   a. R33 and/or R36 in order to produce more OAA and/or
   b. R30 in order to produce more MAL and/or
   c. R57 in order to produce more Pyr; and/or
with respect to TRS, the amount and/or activity of the following enzyme is increased and/or introduced compared to the starting organism:
   a. R73 in order to metabolize thiosulfate to sulfide and sulfite and/or
   b. R82 to transport more external H₂S₂O₃ into the cell and/or
with respect to SRS, the amount and/or activity of the following enzyme is increased compared and/or introduced to the starting organism:
   a. R74 in order to metabolize sulfite into sulfide and/or
with respect to FCS, the amount and/or activity of the following enzymes is/are increased and/or introduced compared to the starting organism:
   a. R75 in order to produce 10-formyl-THF and/or
   b. R76 in order to produce Methylene-THF from 10-formyl-THF and/or
   c. R78 in order to produce more Methylene-THE and/or
with respect to MCS, the amount and/or activity of the following enzyme is increased and/or introduced compared to the starting organism:
   a. R77 in order to methyl-sulfhydrylate O-Acetyl-homoserine with methanethiol and/or
with respect to SARS, the amount and/or activity of the following enzyme(s) is/are increased and/or introduced compared to the starting organism:
   a. R80 in order to produce more sulfite and/or
with respect to EMP, the amount and/or activity of the following enzyme(s) is/are at least partially reduced compared to the starting organism:
   a. R11 in order to produce less F-1,6-BP and/or
   b. R13 in order to produce less DHAP and GA3P and/or
   c. R14 in order to produce less GA3P and/or
   d. R15 in order to produce less 1,3-PG and/or
   e. R16 in order to produce less 3-PG and/or
   f. R17 in order to produce less 2-PG and/or
   g. R18 in order to produce less PEP and/or
   h. R19 in order to produce less Pyr; and/or
with respect to TCA, the amount and/or activity of the following enzyme(s) is/are at least partially reduced compared to the starting organism:
   a. R20 in order to produce less Ac-CoA and/or
   b. R21 in order to produce less CIT and/or
   c. R22 in order to produce less Cis-ACO and/or
   d. R23 in order to produce less ICI and/or
   e. R24 in order to produce less 2-OXO and/or
   f. R26 in order to produce less SUCC-CoA and/or
   g. R27 in order to produce less SUCC and/or
   h. R28 in order to produce less FUM and/or
   i. R29 in order to produce less MAL and/or
   j. R30 in order to produce less OAA; and/or
with respect to GS, the amount and/or activity of the following enzyme(s) is/are at least partially reduced compared to the starting organism:
   a. R21 in order to produce less CIT and/or
   b. R22 in order to produce less Cis-ACO and/or
   c. R23 in order to produce less ICI and/or
   d. R31 in order to produce less GLYOXY and SUCC and/or
   e. R32 in order to produce less MAL and/or
   f. R28 in order to produce less FUM and/or
   g. R29 in order to produce less MAL and/or
   h. R30 in order to produce less OAA; and/or
with respect to RC, the amount and/or activity of the following enzyme(s) is/are at least partially reduced compared to the starting organism:
   a. R60; and/or
the amount and/or activity of the following enzyme(s) is/are at least partially reduced compared to the starting organism:
   a. R19; and/or
the amount and/or activity of the following enzyme(s) is/are at least partially reduced compared to the starting organism:
   a. R35; and/or
with respect to P3, the amount and/or activity of the following enzyme(s) is/are at least partially reduced compared to the starting organism:
   a. R56; and/or
with respect to P4, the amount and/or activity of the following enzyme(s) is/are at least partially reduced compared to the starting organism:
   a. R62; and/or
with respect to P7, the amount and/or activity of the following enzyme(s) is/are at least partially reduced compared to the starting organism:
   a. R61, and/or
with respect to P8, the amount and/or activity of the following enzyme(s) is/are at least partially reduced compared to the starting organism:
   a. R79.

A further embodiment of the present invention relates to a method for producing a microorganism of the genus Corynebacterium with an increased efficiency for methionine synthesis wherein
the amount and/or activity of the following enzyme(s) is/are increased and/or introduced compared to the starting organism:
   1. R3 in order to produce more GLC-LAC and/or
   2. R4 in order to produce more 6-P-Gluconate and/or
   3. R5 in order to produce more RIB-5P and/or
   4. R10 in order to produce more F6P and GA3P and/or
   5. R2 in order to produce more G6P and/or
   6. R55 in order to produce more H₂SO₃ and/or
   7. R58 in order to produce more H₂S and/or
   8. R71 in order to produce more M-HPL and/or
   9. R72 in order to produce more Methylene-THF and/or
   10. R78 in order to produce Methyl-THF and/or
   11. R76 in order to produce more Methylene-THF and/or
   12. R70 in order to produce more NADPH and/or
   13. R81 in order to produce more NADPH and/or
   14. R25 in order to produce more Glu and/or
   15. R33 and/or R36 in order to produce more OAA and/or
   16. R30 in order to produce more MAL and/or
   17. R57 in order to produce more Pyr and/or
   18. R80 in order to metabolize sulfate to sulfite and/or
   19. R73 in order to metabolise thiosulafte into sulfide and sulfite and/or
   20. R74 in order to metabolise sulfite into sulfide and/or
   21. R82 to transport more external H₂S₂O₃ into the cell and/or
   22. R75 in order to produce 10-formyl-THF and/or
   23. R76 in order to produce Methylene-THF from 10-formyl-THF and/or
   24. R78 in order to produce more Methylene-THF and/or
   25. R77 in order to methyl-sulfhydrylate O-Acetyl-homoserine with methanethiol and/or
the amount and/or activity of the following enzyme(s) is/are at least partially reduced compared to the starting organism:
   1. R11 in order to produce less F-1,6-BP and/or
   2. R19 in order to produce less Pyr and/or
   3. R20 in order to produce less Ac-CoA and/or
   4. R21 in order to produce less CIT and/or
   5. R24 in order to produce less 2-OXO and/or
   6. R26 in order to produce less SUCC-CoA and/or
   7. R27 in order to produce less SUCC and/or
   58. R31 in order to produce less GLYOXY and SUCC and/or
   9. R32 in order to produce less MAL and/or
   10. R35 in order to produce less PEP and/or
   10. R79 in order to produce less THF.

A further embodiment of the present invention relates to a method for producing a microorganism of the genus Corynebacterium with an increased efficiency for methionine synthesis, wherein
the amount and/or activity of the following enzyme are increased and/or introduced compared to the starting organism:
   1. R3 in order to produce more GLC-LAC and/or
   2. R4 in order to produce more 6-P-Gluconate and/or
   3. R5 in order to produce more RIB-5P and/or
   4. R10 in order to produce more F6P and GA3P and/or
   5. R2 in order to produce more G6P and
   6. R55 in order to produce more H₂SO₃ and/or
   7. R58 in order to produce more H₂S and
   8. R71 in order to produce more M-HPL and/or
   9. R72 in order to produce more Methylene-THF and/or
   10. R78 in order to produce Methyl-THF and/or
   11. R76 in order to produce more Methylene-THF and/or
   12. R70 in order to produce more NADPH and/or
   13. R81 in order to produce more NADPH and/or
   14. R25 in order to produce more Glu and/or
   15. R33 and/or R36 in order to produce more OAA
   16. R30 in order to produce more MAL
   17. R57 in order to produce more Pyr
   18. R80 in order to metabolize sulfate to sulfite and/or
   19. R75 in order to produce 10-formyl-THF and/or
   20. R76 in order to produce Methylene-THF from 10-formyl-THF and/or
   21. R77 in order to methyl-sulfhydrylate O-Acetyl-homoserine with methanethiol and/or
the amount and/or activity of the following enzymes are at least partially reduced compared to the starting organism:
   1. R11 in order to produce less F-1,6-BP and/or
   2. R19 in order to produce less Pyr and/or
   3. R20 in order to produce less Ac-CoA and/or
   4. R21 in order to produce less CIT and/or
   5. R24 in order to produce less 2-OXO and/or
   6. R26 in order to produce less SUCC-CoA and/or
   7. R27 in order to produce less SUCC and/or
   8. R31 in order to produce less GLYOXY and SUCC and/or
   9. R32 in order to produce less MAL and/or
   10. R35 in order to produce less PEP and/or
   11. R79 in order to produce less THF.

Any organism obtained by these methods is also a subject of the present invention.

Corynebacterium microorganisms used for these methods may be selected from the group consisting of
Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539, und
Corynebacterium melassecola ATCC 17965,
Corynebacterium glutamicum KFCC10065 und
Corynebacterium glutamicum ATCC21608
Corynebacterium glutamicum DSM 17322

The abbreviations KFCC means Korean Federation of Culture Collection, while the abbreviation ATCC means the American Type Strain Culture Collection and the abbreviation DSM means the German Resource Centre for Biological Material.

Particularly interesting are genetically modified organisms of the genus Corynebacterium, wherein the metabolic flux through the following pathways is introduced:
glycine cleavage system
transhydrogenase conversion
thiosulfate reductase system
sulfate reductase system
formate converting system
methanethiol converting system.

If a methanethiol converting system is introduced into Corynebacterium, the thiosulfate reductase system and formate converting system may become obsolete. These aforementioned additional pathway systems have been found to significantly contribute to the optimal metabolic flux for efficient methionine synthesis in E. coli (see below). According to the theoretical predictions, inclusion of these metabolic pathways into C. glutamicum should further increase the efficiency of C. glutamicum for methionine synthesis.

Thus, one aspect of the present invention relates to organisms which have been genetically modified in order to increase metabolic flux through any of the aforementioned pathways.

By genetically amending C. glutamicum in accordance with the present invention, the metabolic flux in these organisms may be amended in order to increase the efficiency of methionine synthesis such that these organisms are characterized in that methionine is produced with an efficiency of at least 10%, of at least 20%, of at least 30%, of at least 35%, of at least 40%, at least 45%, at least 50%, at least 55%, at least 60%, at least 65%, at least 70%, at least 75%, at least 80% or at least 85%.

The present invention does not relate, as far as C. glutamicum is concerned, to the ΔmcbR knock out strains described in Rey et al. (2003) vide supra.

E. coli

One aspect of the present invention relates to a microorganism of the genus Escherichia( ) which has been genetically modified in order to increase and/or introduce a metabolic flux through at least one of the following pathways compared to the starting:
phosphotransferase system (PTS) and/or
phosphotransferase system (PTS) and/or
glyoclysis (EMP) and/or
tricarboxylic acid cycle (TCA) and/or
glyoxylate shunt (GS) and/or
anaplerosis pathway (AP) and/or
methionine synthesis pathway (MS) and/or
serine/cysteine/glycine system (SCGS) and/or
pathway 1 (P1) and/or
sulfur assimilation (SA) and/or
glycine cleavage system (OCS) and/or
transhydrogenase conversion (THGC) and/or
thiosulfate reductase system (TRS) and/or
sulfite reductase system (SRS) and/or
sulfate reductase system (SARS) and/or
formate converting system (FCS) and/or
methanethiol converting system (MCS) and/or
serine/cysteine/glycine synthesis (SCGS).

These microorganisms with increased efficiency of methionine synthesis are optionally also characterized by an at least decreased metabolic flux through at least one of the following pathways compared to the starting which may also be achieved by genetic modification:
pentose phosphate pathway (PPP)
methionine synthesis (MS) and/or
pathway 3 (P3) and/or
pathway 4 (P4) and/or
pathway 7 (P7) and/or
pathway 8 (P8).

In some embodiments, the metabolic flux through PPP may not be decreased but increased.

Besides the above-mentioned microorganisms of the genus Escherichia, the present invention also relates to a method for producing microorganisms of the genus Escherichia with increased efficiency of methionine production comprising the following steps:
increasing and/or introducing the metabolic flux through at least one of the following pathways compared to the starting by genetic modification of the organism:
   phosphotransferase system (PTS) and/or
   glyoclysis (EMP) and/or
   tricarboxylic acid cycle (TCA) and/or
   glyoxylate shunt (GS) and/or
   anaplerosis pathway (AP) and/or
   methionine synthesis pathway (MS) and/or
   serine/cysteine/glycine system (SCGS) and/or
   pathway 1 (P1) and/or
   sulfur assimilation (SA) and/or
   glycine cleavage system (GCS) and/or
   transhydrogenase conversion (THGC) and/or
   thiosulfate reductase system (TRS) and/or
   sulfite reductase system (SRS) and/or
   sulfate reductase system (SARS) and/or
   formate converting system (FCS) and/or
   methanethiol converting system (MCS) and/or
   serine/cysteine/glycine synthesis (SCGS) and/or
at least partially decreasing the metabolic flux through at least one of the following pathways compared to the starting by genetic modification of the organism:
   pentose phosphate pathway (PPP)
   pathway 3 (P3) and/or
   pathway 4 (P4) and/or
   pathway 7 (P7) and/or
   R19 and/or
   R35 and/or
   R79.

Further aspects of the present invention are methods for producing a microorganism of the genus Escherichia with an increased efficiency for methionine synthesis wherein
with respect to PTS, the amount and/or activity of the following enzyme is increased compared to the starting organism:
   a. R1 in order to produce more G6P; and/or
with respect to EMO, the amount and/or activity of the following enzyme(s) is/are increased and/or introduced compared to the starting organism:
   a. R2 in order to produce more F6P and/or
   b. R11 in order to produce more F-1,6-BP and/or
   c. R13 in order to produce more DHAP and GA3P and/or
   d. R14 in order to produce more GA3P and/or
   e. R15 in order to produce more 1,3-PG and/or
   f. R16 in order to produce more 3-PG and/or
   g. R17 in order to produce more 2-PG and/or
   h. R18 in order to produce more PEP and/or
   i. R19 in order to produce more Pyr; and/or
with respect to TCA, the amount and/or activity of the following enzyme(s) is/are increased and/or introduced compared to the starting organism:
   a. R20 in order to produce more Ac-CoA and/or
   b. R21 in order to produce more CIT and/or
   c. R22 in order to produce more Cis-ACO and/or
   d. R23 in order to produce more ICI and/or
   e. R24 in order to produce more 2-OXO and/or
   f. R26 in order to produce more SUCC-CoA and/or
   g. R27 in order to produce more SUCC and/or
   h. R28 in order to produce more FUM and/or
   i. R29 in order to produce more MAL and/or
   j. R30 in order to produce more OAA; and/or
with respect to AP, the amount and/or activity of the following enzyme is increased and/or introduced compared to the starting organism:
   a. R33 in order to produce more OAA and/or
with respect to MS, the amount and/or activity of the following enzyme is increased and/or introduced compared to the starting organism:
   a. R37 in order to produce more Asp and/or
   b. R39 in order to produce more HOM and/or
   c. R40 in order to produce more O-AC-HOM and/or
   d. R46 in order to produce more CYSTA and/or
   e. R47 in order to produce more ASP-P and/or
   f. R48 in order to produce more ASP-SA and/or
   g. R49 in order to produce more HOMOCYS and/or
   h. R52 in order to produce more MET and/or
   i. R53 in order to produce more METₑₓ and/or
   j. R54 in order to produce more HOMOCYS and/or
with respect to SCGS, the amount and/or activity of the following enzyme is increased and/or introduced compared to the starting organism:
   a. R38 in order to produce more M-THF and Glycineₑₓ and/or
   b. R44 in order to produce more O-AC-SER and/or
   c. R45 in order to produce more CYS and/or
with respect to GS, the amount and/or activity of the following enzyme(s) is/are increased and/or introduced compared to the starting organism:
   a. R21 in order to produce more CIT and/or
   b. R22 in order to produce more Cis-ACO and/or
   c. R23 in order to produce more ICI and/or
   d. R31 in order to produce more GLYOXY and SUCC and/or
   e. R32 in order to produce more MAL and/or
   f. R28 in order to produce more FUM and/or
   g. R29 in order to produce more MAL and/or
   h. R30 in order to produce more OAA; and/or
with respect to P1, the amount and/or activity of the following enzymes is/are increased and/or introduced compared to the starting organism:
   R25 in order to produce more Glu; and/or
with respect to SA, the amount and/or activity of the following enzymes is/are increased and/or introduced compared to the starting organism:
   a. R55 in order to produce more H₂SO₃ and/or
   b. R58 in order to produce more H₂S; and/or
with respect to GCS, the amount and/or activity of the following enzymes is/are increased and/or introduced compared to the starting organism:
   a. R71 in order to produce more M-HPL and/or
   b. R72 in order to produce more Methylene-THF and/or
   c. R78 in order to produce more Methyl-THF and/or
with respect to claim THGC, the amount and/or activity of the following enzyme is increased and/or introduced compared to the starting organism:
   a. R70 in order to produce more NADPH from NADH and/or
   b. R81 in order to produce more NADPH from NADH and/or
with respect to TRS, the amount and/or activity of the following enzymes is/are increased and/or introduced compared to the starting organism:
   a. R73 in order to metabolize thiosulfate to sulfide and sulfite and/or
   b. R82 to transport more external H₂S₂O₃ into the cell and/or
with respect to SRS, the amount and/or activity of the following enzyme is increased and/or introduced compared to the starting organism:
   a. R74 in order to metabolize sulfite to sulfide and/or
with respect to SARS, the amount and/or activity of the following enzyme is increased and/or introduced compared to the starting organism:
   a. R80 in order to metabolize sulfate into sulfite and/or
with respect to FCS, the amount and/or activity of the following enzymes is/are increased and/or introduced compared to the starting organism:
   a. R75 in order to produce 10-formyl-THF and/or
   b. R76 in order to produce Methylene-THF from 10-formyl-THF and/or
   c. R78 in order to produce more Methyl-THF
with respect to MCS, the amount and/or activity of the following enzyme is increased and/or introduced compared to the starting organism:
   a. R77 in order methyl-sulfhydrylate O-Acetyl-homoserine with methanethiol and/or
with respect to SCGS, the amount and/or activity of the following enzyme is increased compared to the starting organism:
   a. R44 in order to produce more O—Ac-SER and/or
   b. R45 in order to produce more CYS; and/or
with respect to PPP, the amount and/or activity of the following enzyme(s) is/are increased compared to the starting organism:
   a. R3 in order to produce more GLC-LAC and/or
   b. R4 in order to produce more 6-P-Gluconate and/or
   c. R5 in order to produce more RIB-5P and/or
   d. R6 in order to produce more XYL-5P and/or
   e. R7 in order to produce more RIBO-5P and/or
   f. R8 in order to produce more S7P and GA3P and/or
   g. R9 in order to produce more E4p and F6P and/or
   h. R10 in order to produce less F6P and GA3P and/or
   i. R2 in order to produce 1 more G6P; and/or
with respect to PPP, in some embodiments the amount and/or activity of the following enzyme(s) may also be at least partially reduced compared to the starting organism:
   j. R3 in order to produce less GLC-LAC and/or
   k. R4 in order to produce less 6-P-Gluconate and/or
   l. R5 in order to produce less RIB-5P and/or
   m. R6 in order to produce less XYL-5P and/or
   n. R7 in order to produce less RIBO-5P and/or
   o. R8 in order to produce less S7P and GA3P and/or
   p. R9 in order to produce less E-4-p and F6P and/or
   q. R10 in order to produce less F6P and GA3P and/or
   r. R2 in order to produce less G6P; and/or
with respect to P3, the amount and/or activity of the following enzyme(s) is/are at least reduced compared to the starting organism:
   a. R56; and/or
with respect to P4, the amount and/or activity of the following enzyme(s) is/are at least reduced compared to the starting organism:
   a. R62; and/or
with respect to P7, the amount and/or activity of the following enzyme(s) is/are at least reduced compared to the starting organism:
   a. R61.
the amount and/or activity of the following enzyme(s) is/are at least reduced compared to the starting organism:
   a. R19 in order to produce less pyruvate; and/or
   b. R35 in order to produce less PEP; and/or
   c. R79 in order to produce less tetrahydrofolate.

A further embodiment of the invention with respect to the genus Escherichia relates to a method for producing Escherichia microorganisms with increased efficiency of methionine synthesis, wherein
the amount and/or activity of the following enzymes are increased and/or introduced compared to the starting organism:
   1. R1 in order to produce more G6P and/or
   2. R2 in order to produce more F6P and/or
   3. R11 in order to produce more F-1,6-BP and/or
   4. R19 in order to produce more Pyr and/or
   5. R20 in order to produce more Ac-CoA and/or
   6. R21 in order to produce more CIT and/or
   7. R24 in order to produce more 2-OXO and/or
   8. R26 in order to produce more SUCC-CoA and/or
   9. R31 in order to produce more GLYOXY and SUCC and/or
   10. R32 in order to produce more MAL and/or
   11. R25 in order to produce more Glu and/or
   12. R55 in order to produce more H₂SO₃ and/or
   13. R8 in order to produce more H₂S and/or
   14. R71 in order to produce more M-HPL and/or
   15. R72 in order to produce more M-THF and/or
   16. R78 in order to produce more Methyl-THF and/or
   17. R76 in order to produce more Methylene-THF and/or
   18. R70 in order to produce more NADPH and/or
   19. R81 in order to produce more NADPH and/or
   20. R80 in order to metabolise sulfate into sulfite and/or
   21. R73 in order to metabolize thiosulfate to sulfide and sulfite and/or
   22. R82 to transport more external H₂S₂O₃ into the cell and/or
   23. R74 in order to metabolize sulfite to sulfide and/or
   24. R75 in order to produce 10-formyl-THF and/or
   25. R76 in order to produce Methylene-THF from 10-formyl-THF and/or
   26. R77 in order methyl-sulfhydrylate O-Acetyl-homoserine with methanethiol and/or
   27. R44 in order to produce more O—Ac-SER and/or
   28. R45 in order to produce more CYS;
the amount and/or activity of the following enzyme(s) is/are at least partially reduced compared to the starting organism:
   1. R19 in order to produce less pyruvate; and/or
   2. R35 in order to produce less PEP; and/or
   3. R79 in order to produce less tetrahydrofolate.

The microorganism of the genus Escherichia which is obtainable by any of the aforementioned methods is selected from the group comprising e.g. Escherichia coli.

In some embodiments relating organisms such as to E. coli and C. glutamicum, metabolic flux is generated by overexpression of the following enzymatic activities: R37, R38, R39, R40, R44, R45, R46, R47, R48, R49, R52, R53, R54 and/or R58. E. coli and C. glutamicum organisms in which any combination of the aforementioned R numbers or any of the genes that are part of these catalytic activities are overexpressed also form an object of the invention.

Organisms such as E. coli and C. glutamicum in which any combination of R70, R81, R71/R72, R73, R82, R74, R75, R76, R77, R78, and/or R80 together with R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which one enzymatic activity of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which two enzymatic activities of the group consisting of R70, R81, R71/R72, R73, R82, R74, R75, R76, R77, R78, and R80 together with any of R37; 8, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which three enzymatic activities of the group consisting of R70, R81, R71/R72, R73, R82, R74, R75, R76, R77, R78, and R80 together with any of R37, 38, R39, R40, R44, R45, R46, R47, R48, R49, R52, R3, R54 and/or R58 or the genes that are part of these catalytic activities are overexpressed also form an object of the invention.

Organisms such as E. coli and C. glutamicum in which four enzymatic activities of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which five enzymatic activities of the group consisting of R70, R81,R71/R72, R73, R82,R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which six enzymatic activities of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which seven enzymatic activities of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which eight enzymatic activities of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which nine enzymatic activities of the group consisting of R70, R81, R71/R72, R73, R82, R74, R75, R76, R77, R78, and R80 together with any of R37, R38, R39, R40, R44, R45, R46, R47, R48, R49, R52, R53, R54 and/or R58 or the genes that are part of these catalytic activities are overexpressed also form an object of the invention.

Organisms such as E. coli and C. glutamicum in which at least one enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least two enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least three enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least four enzymatic activities of the group consisting of R70, R81, R71/R72, R73, R82, R74, R75, R76, R77, R78 and R80 together with any of 37, R38, R39, R40, R44, R45, R46, R47, R48, R49, R52, R53, R4 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least five enzymatic activities of the group consisting of R70, R81, R71/R72, R73, R82, R74, R75, R76, R77, R78 and R80 together with any of 37, R38, R39, R40, R44, R45, R46, R47, R48, R49, R52, R53, R54 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least six enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least seven enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R4 and/or R58 or the genes that are part of these catalytic activities are overexpressed and also at least one enzymatic activity of the group consisting of R19, R35 and R79 is decreased also form an object of the invention.

Organisms such as E. coli and C. glutamicum in which at least eight enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activities of the group consisting of R19, R35 and R79 is decreased also form an object of the invention.

Organisms such as E. coli and C. glutamicum in which at least nine enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least one enzymatic activities of the group consisting of R19, R35 and R79 is decreased also form an object of the invention.

Organisms such as E. coli and C. glutamicum in which at least one enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least two enzymatic activities of the group consisting of R19, R35 and R79 are decreased also form an object of the invention.

Organisms such as E. coli and C. glutamicum in which at least one enzymatic activities of the group consisting of R70, R81, R71/R72, R73, R82, R74, R75, R76, R77, R78 and R80 together with any of R37, R38, R39, R40, R44, R45, R46, R47, R48, R49, R52, R53, R54 and/or R58 or the genes that are part of these catalytic activities are overexpressed and also at least three enzymatic activities of the group consisting of R19, R35 and R79 are decreased also form an object of the invention.

In a preferred embodiment, the invention relates to a C. glutamicum organism in which metabolic flux through one of the following pathways is introduced and/or increased by e.g. genetic modification as described above: FCS or GCS or MCS or TRS or THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and GCS, FCS and MCS, FCS and TRS, or FCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and GCS and TRS, FCS and GCS and TRS, or FCS and GCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and GCS and MCS and TRS, or FCS and GCS and MCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and GCS and MCS and TRS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and MCS and TRS, or FCS and MCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and MCS and TRS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and TRS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and MCS and TRS, or FCS and MCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: FCS and MCS and TRS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: MCS and TRS, or MCS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: MCS and TRS and THGC. In another preferred embodiment of the invention, these organisms are additionally grown using Sulfid or Thiosulfate as external sulfur sources.

In another preferred embodiment, the invention relates to a C. glutamicum organism in which metabolic flux through the following pathways is introduced and/or increased: TRS and THGC. In another preferred embodiment of the invention, these organisms are additionally grown using Sulfid or Thiosulfate as external sulfinur sources.

For genetic manipulation in the case of GCS, expression of R71 and/or R72 can be increased. In the case of THGS expression of R70 and/or R81 can be increased. In the case of TRS expression of R73, R45a, R49 and/or R82 can be increased. For MCS expression of R77 can be increased. In the case of FCS, expression of R75, R76 and/or R78 can be increased.

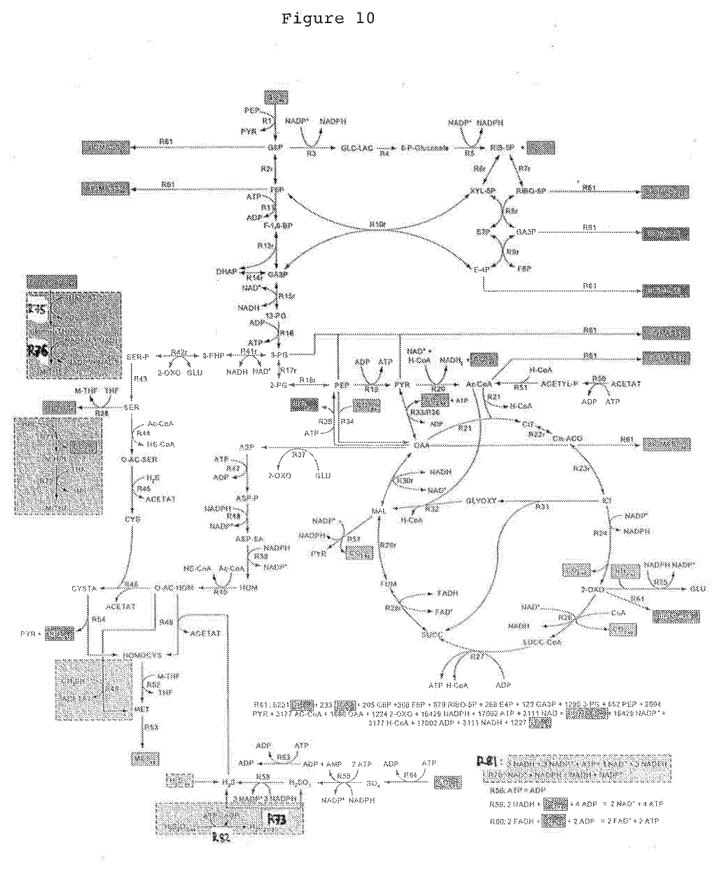

One preferred embodiment of the invention is depicted in FIG. 10. In this embodiment, an organism is depicted in which metabolic flux through the following pathways is increased and/or introduced in comparison to the starting organism e.g. by way of the already mentioned genetic manipulations: FCS and GCS and MCS and TRS and THGC. Concomitantly use of Sulfid and Thiosulfate as Sulfur-sources is considered.

The organisms of the present invention may preferably comprise a microorganism of the genus Corynebacterium, particularly Corynebacterium acetoacidophilum, C. acetoglutamicum, C. efficiens, C. jejeki, C. acetophilum, C. ammoniagenes, C. glutamicum, C. lilium, C. nitrilophilus or C. spec. The organisms in accordance with the present invention also comprise members of the genus Brevibacterium, such as Brevibacterium harmoniagenes, Brevibacterium botanicum, B. divaraticum, B. flavam, B. healil, B. ketoglutamicum, B. ketosoreductum, B. lactofermentum, B. linens, B. paraphinolyticum and B. spec. As to the genus Escherichia, the present invention concerns e.g. E. coli.

As set out above, the metabolic flux through a specific reaction or specific metabolic pathway may be modified by either increasing or decreasing the amount and/or activity of the enzymes catalyzing the respective reactions.

With respect to increasing the amount and/or activity of an enzyme, all methods that are known in the art for increasing the amount and/or activity of a protein in a host such as the above-mentioned organisms may be used.

Increasing or Introducing the Amount and/or Activity

With respect to increasing the amount, two basic scenarios can be differentiated. In the first scenario, the amount of the enzyme is increased by expression of an exogenous version of the respective protein. In the other scenario, expression of the endogenous protein is increased by influencing the activity of the promoter and/or enhancers element and/or other regulatory activities such as phosphorylation, sumoylation, ubiquitylation etc. that regulate the activities of the respective proteins either on a transcriptional, translational or post-translational level.

Besides simply increasing the amount of e.g. the enzymes of Table 1, the activity of the proteins may be increased by using enzymes can carry specific mutations that allow for an increased activity of the enzyme. Such mutations may, e.g. inactivate the regions of an enzyme that are responsible for feedback inhibition. By mutating these by e.g. introducing non-conservative mutations, the enzyme would not provide for feedback regulation anymore and thus activity of the enzyme would not be down regulated if more product was produced. The mutations may be either introduced into the endogenous copy of the enzyme, or may be provided by over-expressing a corresponding mutant form of the exogenous enzyme. Such mutations may comprise point mutations, deletions or insertions. Point mutations may be conservative or non-conservative. Furthermore, deletions may comprise only two or three amino acids up to complete domains of the respective protein.

Thus, the increase of the activity and the amount of a protein may be achieved via different routes, e.g. by switching off inhibitory regulatory mechanisms at the transcription, translation, and protein level or by increase of gene expression of a nucleic acid coding for these proteins in comparison with the starting, e.g. by inducing the endogenous R3 gene or by introducing nucleic acids coding for R3

In one embodiment, the increase of the enzymatic activity and amount, respectively, in comparison with the starting is achieved by an increase of the gene expression of a nucleic acid encoding such enzymes. Sequences may be obtained from the respective database, e.g. at NCBI (http://www.ncbi.nlm.nih.gov/), EMBL (http://www.embl.org), or Expasy (http://www.expasy.org/). Examples are given in Table 1.

In a further embodiment, the increase of the amount and/or activity of the enzymes of Table 1 is achieved by introducing nucleic acids encoding the enzymes of Table 1 into the organism, preferably C. glutamicum or E. coli.

In principle, every protein of different organisms with an enzymatic activity of the proteins listed in Table 1, can be used. With genomic nucleic acid sequences of such enzymes from eukaryotic sources containing introns, already processed nucleic acid sequences like the corresponding cDNAs are to be used in the case that the host organism is not capable or cannot be made capable of splicing the corresponding mRNAs. All nucleic acids mentioned in the description can be, e.g., an RNA, DNA or cDNA sequence.

In one method according to the present invention for producing organisms with increased efficiency of methionine synthesis, a nucleic acid sequence coding for one of the above-defined functional or non-functional, feedback-regulated or feedback-independent enzymes is transferred to a microorganism such as C. glutamicum or E. coli., respectively. This transfer leads to an increase of the expression of the enzyme, respectively, and correspondingly to more metabolic flux through the desired reaction pathway.

According to the present invention, increasing tore introducing the amount and/or the activity of a protein typically comprises the following steps:

a) production of a vector comprising the following nucleic acid sequences, preferably DNA sequences, in 5′-3′-orientation:
a promoter sequence functional in the organisms of the invention
operatively linked thereto a DNA sequence coding for a protein of Table 1 or functional equivalent parts thereof
a termination sequence functional in the organisms of the invention
   b) transfer of the vector from step a) to the organisms of the invention such as C. glutamicum or E. coli and, optionally, integration into the respective genomes.

When functionally equivalent parts of enzymes are mentioned within the scope of the present invention, fragments of nucleic acid sequences coding for enzymes of Table 1 are meant, whose expression still lead to proteins having the enzymatic activity of the respective full length protein.

According to the present invention, non-functional enzymes have the same nucleic acid sequences and amino acid sequences, respectively, as functional enzymes and functionally equivalent parts thereof, respectively, but have, at some positions, point mutations, insertions or deletions of nucleotides or amino acids, which have the effect that the non-functional enzyme are not, or only to a very limited extent, capable of catalyzing the respective reaction. These non-functional enzymes may not be intermixed with enzymes that still are capable of catalyzing the respective reaction, but which are not feedback regulated anymore. Non-functional enzymes also comprise such enzymes of Table 1 bearing point mutations, insertions, or deletions at the nucleic acid sequence level or amino acid sequence level and are not, or nevertheless, capable of interacting with physiological binding partners of the enzymes. Such physiological binding partners comprise, e.g. the respective substrates. What non-functional mutants are incapable of is to catalyse a reaction which the wild type enzyme, from which the mutant is derived, can.

According to the present invention, the term “non-functional enzyme” does not comprise such proteins having no essential sequence homology to the respective functional enzymes at the amino acid level and nucleic acid level, respectively. Proteins unable to catalyse the respective reactions and having no essential sequence homology with the respective enzyme are therefore, by definition, not meant by the term “non-functional enzyme” of the present invention. Non-functional enzymes are, within the scope of the present invention, also referred to as inactivated or inactive enzymes.

Therefore, non-functional enzymes of Table 1 according to the present invention bearing the above-mentioned point mutations, insertions, and/or deletions are characterized by an essential sequence homology to the wild type enzymes of Table 1 according to the present invention or functionally equivalent parts thereof.

According to the present invention, a substantial sequence homology is generally understood to indicate that the nucleic acid sequence or the amino acid sequence, respectively, of a DNA molecule or a protein, respectively, is at least 25%, at least 30%, at least 40%, preferably at least 50%, further preferred at least 60%, also preferably at least 70%, also preferably at least 80%, particularly preferred at least 90%, in particular preferred at least 95% and most preferably at least 98% identical with the nucleic acid sequences or the amino acid sequences, respectively, of the proteins of Table I or functionally equivalent parts thereof.

Identity of two proteins is understood to be the identity of the amino acids over the respective entire length of the protein, in particular the identity calculated by comparison with the assistance of the Lasergene software by DNA Star, Inc., Madison, Wis. (USA) applying the CLUSTAL method (Higgins et al., (1989), Comput. Appl. Biosci., 5(2), 151).

Homologies can also be calculated with the assistance of the Lasergene software by DNA Star, Inc., Madison, Wis. (USA) applying the CLUSTAL method (Higgins et al., (1989), Comput. Appl. Biosci., 5(2), 151).

Identity of DNA sequences is to be understood correspondingly.

The above-mentioned method can be used for increasing the expression of DNA sequences coding for functional or non-functional, feedback-regulated or feedback-independent enzymes of Table 1 or functionally equivalent parts thereof. The use of such vectors comprising regulatory sequences, like promoter and termination sequences are, is known to the person skilled in the art. Furthermore, the person skilled in the art knows how a vector from step a) can be transferred to organisms such as C. glutamicum or E. coli and which properties a vector must have to be able to be integrated into their genomes.

If the enzyme content in an organism such as C. glutamicum is increased by transferring a nucleic acid coding for an enzyme from another organism, like e.g. E. coli, it is advisable to transfer the amino acid sequence encoded by the nucleic acid sequence e.g. from E. coli by back-translation of the polypeptide sequence according to the genetic code into a nucleic acid sequence comprising mainly those codons, which are used more often due to the organism-specific codon usage. The codon usage can be determined by means of computer evaluations of other known genes of the relevant organisms.

According to the present invention, an increase of the gene expression and of the activity, respectively, of a nucleic acid encoding an enzyme of Table 1 is also understood to be the manipulation of the expression of the endogenous respective endogenous enzymes of an organism, in particular of C. glutamicum or E. coli. This can be achieved, e.g., by altering the promoter DNA sequence for genes encoding these enzymes. Such an alteration, which causes an altered, preferably increased, expression rate of these enzymes can be achieved by deletion or insertion of DNA sequences.

An alteration of the promoter sequence of endogenous genes usually causes an alteration of the expressed amount of the gene and therefore also an alteration of the activity detectable in the cell or in the organism.

Furthermore, an altered and increased expression, respectively, of an endogenous gene can be achieved by a regulatory protein, which does not occur in the transformed organism, and which interacts with the promoter of these genes. Such a regulator can be a chimeric protein consisting of a DNA binding domain and a transcription activator domain, as e.g. described in WO 96/06166.

A further possibility for increasing the activity and the content of endogenous genes is to up-regulate transcription factors involved in the transcription of the endogenous genes, e.g. by means of overexpression. The measures for overexpression of transcription factors are known to the person skilled in the art and are also disclosed for the enzymes of Table I within the scope of the present invention.

Furthermore, an alteration of the activity of endogenous genes can be achieved by targeted mutagenesis of the endogenous gene copies.

An alteration of the endogenous genes coding for the enzymes if Table I can also be achieved by influencing the post-translational modifications of the enzymes. This can happen e.g. by regulating the activity of enzymes like kinases or phosphatases involved in the post-translational modification of the enzymes by means of corresponding measures like overexpression or gene silencing.

In another embodiment, an enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired enzyme of Table 1 without impairing the viability of the cell. In each case, the overall yield or rate of production of one of these desired fine chemicals may be increased.

It is also possible that such alterations in the protein and nucleotide molecules of Table 1 may improve the production of other fine chemicals such as other sulfur containing compounds like cysteine or glutathione, other amino acids, vitamins, cofactors, nutraceuticals, nucleotides, nucleosides, and trehalose. Metabolism of any one compound is necessarily intertwined with other biosynthetic and degradative pathways within the cell, and necessary cofactors, intermediates, or substrates in one pathway are likely supplied or limited by another such pathway. Therefore, by modulating the activity of one or more of the proteins of Table 1, the production or efficiency of activity of another fine chemical biosynthetic or degradative pathway besides those leading to methionine may be impacted.

Enzyme expression and function may also be regulated based on the cellular levels of a compound from a different metabolic process, and the cellular levels of molecules necessary for basic growth, such as amino acids and nucleotides, may critically affect the viability of the microorganism in large-scale culture. Thus, modulation of an amino acid biosynthesis enzymes of Table 1 such that they are no longer responsive to feedback inhibition or such that they are improved in efficiency or turnover should result in higher metabolic flux through pathways of methionine production. The theoretical method of the invention will help to incorporate the effects of these nutrients, metabolites etc. into the model organisms and thus will provide valuable guidance to the metabolic pathways that should be genetically modified to increase efficiency of methionine synthesis.

These aforementioned strategies for increasing or introducing the amount and/or activity of the enzymes of Table 1 are not meant to be limiting; variations on these strategies will be readily apparent to one of ordinary skill in the art.

Reducing the Amount and/or Activity of Enzymes

For reducing the amount and/or activity of any of enzymes of Table 1, various strategies are also available.

The expression of the endogenous enzymes of Table 1 can e.g. be regulated via the expression of aptamers specifically binding to the promoter sequences of the genes. Depending on the aptamers binding to stimulating or repressing promoter regions, the amount and thus, in this case, the activity of the enzymes of Table 1 is increased or reduced.

Aptamers can also be designed in a way as to specifically bind to the enzymes themselves and to reduce the activity of the enzymes by e.g. binding to the catalytic center of the respective enzymes. The expression of aptamers is usually achieved by vector-based overexpression (see above) and is, as well as the design and the selection of aptamers, well known to the person skilled in the art (Famulok et al., (1999) Curr Top Microbiol Immunol., 243, 123-36).

Furthermore, a decrease of the amount and the activity of the endogenous enzymes of Table 1 can be achieved by means of various experimental measures, which are well known to the person skilled in the art. These measures are usually summarized under the term “gene silencing”. For example, the expression of an endogenous gene can be silenced by transferring an above-mentioned vector, which has a DNA sequence coding for the enzyme or parts thereof in antisense order, to the organisms such as C. glutamicum and E. coli. This is based on the fact that the transcription of such a vector in the cell leads to an RNA, which can hybridize with the mRNA transcribed by the endogenous gene and therefore prevents its translation.

Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews-Trends in Genetics, Vol. 1 (1) 1986.

In principle, the antisense strategy can be coupled with a ribozyme method. Ribozymes are catalytically active RNA sequences, which, if coupled to the antisense sequences, cleave the target sequences catalytically (Tanner et al., (1999) FEMS Microbiol Rev. 23 (3), 257-75). This can enhance the efficiency of an antisense strategy.

In plants, gene silencing may be achieved by RNA interference or a process that is known as co-suppression.

Further methods are the introduction of nonsense mutations into the endogenous gene by means of introducing RNA/DNA oligonucleotides into the organism (Zhu et al., (2000) Nat. Biotechnol. 18 (5), 555-558) or generating knockout mutants with the aid of homologous recombination (Hohn et al., (1999) Proc. Natl. Acad. Sci. USA. 96, 8321-8323.).

To create a homologous recombinant microorganism, a vector is prepared which contains at least a portion of gene coding for an enzyme of Table 1 into which a deletion, addition or substitution has been introduced to thereby alter, e.g., functionally disrupt, the endogenous gene.

Preferably, this endogenous gene is a C. glutamicum or E. coli gene, but it can be a homologue from a related bacterium or even from a yeast or plant source. In one embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous enzyme of Table 1). In the homologous recombination vector, the altered portion of the endogenous gene is flanked at its 5′ and 3′ ends by additional nucleic acid of the endogenous gene to allow for homologous recombination to occur between the exogenous gene carried by the vector and an endogenous gene in the (micro)organism. The additional flanking endogenous nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors).

The vector is introduced into a microorganism (e.g., by electroporation) and cells in which the introduced endogenous gene has homologously recombined with the endogenous enzymes of Table 1 are selected, using art-known techniques.

In another embodiment, an endogenous gene for the enzymes of Table 1 in a host cell is disrupted (e.g., by homologous recombination or other genetic means known in the art) such that expression of its protein product does not occur. In another embodiment, an endogenous or introduced gene of enzymes of Table 1 in a host cell has been altered by one or more point mutations, deletions, or inversions, but still encodes a functional enzyme. In still another embodiment, one or more of the regulatory regions (e.g., a promoter, repressor, or inducer) of an endogenous gene for the enzymes of table 1 in a (micro)organism has been altered (e.g., by deletion, truncation, inversion, or point mutation) such that the expression of the endogenous gene is modulated. One of ordinary skill in the art will appreciate that host cells containing more than one of the genes coding for the enzyme of Table I and protein modifications may be readily produced using the methods of the invention, and are meant to be included in the present invention.

Furthermore, a gene repression (but also gene overexpression) is also possible by means of specific DNA-binding factors, e.g. factors of the zinc finger transcription factor type. Furthermore, factors inhibiting the target protein itself can be introduced into a cell. The protein-binding factors may e.g. be the above-mentioned aptamers (Famulok et al., (1999) Curr Top Microbiol Immunol. 243, 123-36).

As further protein-binding factors, whose expression in organisms cause a reduction of the amount and/or the activity of the enzymes of table 1, enzyme-specific antibodies may be considered. The production of monoclonal, polyclonal, or recombinant enzyme-specific antibodies follows standard protocols (Guide to Protein Purification, Meth. Enzymol. 182, pp. 663-679 (1990), M. P. Deutscher, ed.). The expression of antibodies is also known from the literature (Fiedler et al., (1997) Immunotechnology 3, 205-216; Maynard and Georgiou (2000) Annu. Rev. Biomed. Eng. 2, 339-76).

The mentioned techniques are well known to the person skilled in the art. Therefore, he also knows which sizes the nucleic acid constructs used for e.g. antisense methods must have and which complementarity, homology or identity, the respective nucleic acid sequences must have. The terms complementarity, homology, and identity are known to the person skilled in the art.

Within the scope of the present invention, sequence homology and homology, respectively, are generally understood to mean that the nucleic acid sequence or the amino acid sequence, respectively, of a DNA molecule or a protein, respectively, is at least 25%, at least 30%, at least 40%, preferably at least 50%, further preferred at least 60%, also preferably at least 70%, also preferably at least 80%, particularly preferred at least 90%, in particular preferred at least 95% and most preferably at least 98% identical with the nucleic acid sequences or amino acid sequences, respectively, of a known DNA or RNA molecule or protein, respectively. Herein, the degree of homology and identity, respectively, refers to the entire length of the coding sequence.

The term complementarity describes the capability of a nucleic acid molecule of hybridizing with another nucleic acid molecule due to hydrogen bonds between two complementary bases. The person skilled in the art knows that two nucleic acid molecules do not have to have a complementarity of 100% in order to be able to hybridize with each other. A nucleic acid sequence, which is to hybridize with another nucleic acid sequence, is preferred being at least 30%, at least 40%, at least 50%, at least 60%, preferably at least 70%, particularly preferred at least 80%, also particularly preferred at least 90%, in particular preferred at least 95% and most preferably at least 98 or 100%, respectively, complementary with said other nucleic acid sequence.

Nucleic acid molecules are identical, if they have identical nucleotides in identical 5′-3′-order.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., Molecular Cloning, Cold Spring Harbor Press). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleotides). In addition, stringent conditions can comprise the addition of destabilizing agents like e.g. form amide. Typical hybridization and washing buffers are of the following composition.

| | |
|---|---|
| Pre-hybridization solution: | 0.5% SDS |
| | 5x SSC |
| | 50 mM NaPO₄, pH 6.8 |
| | 0.1% Na-pyrophosphate |
| | 5x Denhardt's reagent |
| | 100 μg/salmon sperm |
| Hybridization solution: | Pre-hybridization solution |
| | 1 × 10⁶ cpm/ml probe (5-10 min 95° C.) |
| 20x SSC: | 3 M NaCl |
| | 0.3 M sodium citrate |
| | ad pH 7 with HCl |
| 50x Denhardt's reagent: | 5 g Ficoll |
| | 5 g polyvinylpyrrolidone |
| | 5 g Bovine Serum Albumin |
| | ad 500 ml A. dest. |

A typical procedure for the hybridization is as follows:

| Optional: | wash Blot 30 min in 1× SSC/0.1% SDS at 65° C. | | |
|---|---|---|---|
| Pre-hybridization: | at least 2 h at 50-55° C. | | |
| Hybridization: | over night at 55-60° C. | | |
| Washing: 05 min | 2× SSC/0.1% SDS | | |
| | Hybridization temperature | | |
| | 30 min | 2× SSC/0.1% SDS | |
| | Hybridization temperature | | |
| | 30 min | 1× SSC/0.1% SDS | |
| | Hybridization temperature | | |
| | 45 min | 0.2× SSC/0.1% SDS | 65° C. |
| | 5 min | 0.1× SSC | room temperature |

The terms “sense” and “antisense” as well as “antisense orientation” are known to the person skilled in the art. Furthermore, the person skilled in the art knows, how long nucleic acid molecules, which are to be used for antisense methods, must be and which homology or complementarity they must have concerning their target sequences.

Accordingly, the person skilled in the art also knows, how long nucleic acid molecules, which are used for gene silencing methods, must be. For antisense purposes complementarity over sequence lengths of 100 nucleotides, 80 nucleotides, 60 nucleotides, 40 nucleotides and 20 nucleotides may suffice. Longer nucleotide lengths will certainly also suffice. A combined application of the above-mentioned methods is also conceivable.

If, according to the present invention, DNA sequences are used, which are operatively linked in 5′-3′-orientation to a promoter active in the organism, vectors can, in general, be constructed, which, after the transfer to the organism's cells, allow the overexpression of the coding sequence or cause the suppression or competition and blockage of endogenous nucleic acid sequences and the proteins expressed there from, respectively.

The activity of a particular enzyme may also be reduced by over-expressing a non-functional mutant thereof in the organism. Thus, a non-functional mutant which is not able to catalyze the reaction in question, but that is able to bind e.g. the substrate or co-factor, can, by way of over-expression out-compete the endogenous enzyme and therefore inhibit the reaction. Further methods in order to reduce the amount and/or activity of an enzyme in a host cell are well known to the person skilled in the art.

### Vectors and Host Cells

Another aspect of the invention pertains to vectors, preferably expression vectors, containing a nucleic acid encoding the enzymes of Table 1 (or portions thereof) or combinations thereof. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention may comprise a nucleic acid coding for the enzymes of Table I in a form suitable for expression of the respective nucleic acid in a host cell, which means that the recombinant expression vectors include one or more regulatory sequences, selected on the basis of the host cells to be used for expression, which is operatively linked to the nucleic acid sequence to be expressed.

Within a recombinant expression vector, “operably linked” is intended to mean that the nucleotide sequence of interest is linked to the regulatory sequence (s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-, tet-, lpp-, lac-, lpp-lac-, lacIq-, T7-, T5-, T3-, gal-, trc-, ara-, SP6-, arny, SP02, e-Pp-ore PL, which are used preferably in bacteria. Additional regulatory sequences are, for example, promoters from yeasts and fingi, such as ADC1, MFa, AC, P-60, CYC1, GAPDH, TEF, rp28, ADH, promoters from plants such asCaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids coding for the enzymes of Table 1.

The recombinant expression vectors of the invention can be designed for expression of the enzymes in Table 1 in prokaryotic or eukaryotic cells. For example, the genes for the enzymes of Table 1 can be expressed in bacterial cells such as C. glutamicum and E. coli, insect cells (using baculovirus expression vectors), yeast and other fungal cells (see Romanos, M. A. et al. (1992), Yeast 8: 423-488; van den Hondel, C. A. M. J. J. et al. (1991) in: More Gene Manipulations in Fungi, J. W. Bennet & L. L. Lasure, eds., p. 396-428: Academic Press: San Diego; and van den Hondel, C. A. M. J. J. & Punt, P. J. (1991) in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) Plant Cell Rep.: 583-586). Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 3140), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion E. coli expression vectors include pTrc (Amann et al., (1988) Gene 69: 301-315), pLG338, pACYC184, pBR322, pUC18, pUC19, pKC30, pRep4, pHS1, pHS2, pPLc236, pMBL24, pLG200, pUR290, pIN-III113-B1, egtll, pBdC1, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier New York IBSN 0 444 904018). Target gene expression from the pTrc vector relies on host RNA polymerase transcription from a hybrid trp-lac fusion promoter. Target gene expression from the pET 11d vector relies on transcription from a T7 gn1O-lac fusion promoter mediated by a coexpressed viral RNA polymerase (T7gnl). This viral polymerase is supplied by host strains BL21 (DE3) or HMS174 (DE3) from a resident X prophage harboring a T7gnl gene under the transcriptional control of the lacUV 5 promoter. For transformation of other varieties of bacteria, appropriate vectors may be selected. For example, the plasmidspIJ101, pIJ364, pIJ702 and pIJ361 are known to be useful in transforming Streptomyces, while plasmidspUB110, pC194, or pBD214 are suited for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: New York IBSN 0 444 904018).

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20: 2111-2118). Such alteration of nucleic acid sequences of the invention can be carried out by standard DNA synthesis techniques.

Examples of suitable C. glutamicum and E. coli shuttle vectors can be found in Eikmanns et al (Gene. (1991) 102, 93-8).

In another embodiment, the protein expression vector is a yeast expression vector. Examples of vectors for expression in yeast S. cerevisiae include pYepSec1 (Baldari, et al., (1987) Embo J. 6: 229-234), 21, pAG-1, Yep6, Yep13, pEMBLYe23, pMFa (Kurjan and Herskowitz, (1982) Cell 30: 933-943), pJRY88 (Schultz et al., (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

For the purposes of the present invention, an operative link is understood to be the sequential arrangement of promoter, coding sequence, terminator and, optionally, further regulatory elements in such a way that each of the regulatory elements can fulfill its function, according to its determination, when expressing the coding sequence.

In another embodiment, the proteins of Table 1 may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) Plant Mol. Biol. 20: 1195-1197; and Bevan, M. W. (1984) Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

For the purposes of the present invention, an operative link is understood to be the sequential arrangement of promoter, coding sequence, terminator and, optionally, further regulatory elements in such a way that each of the regulatory elements can fulfill its function, according to its determination, when expressing the coding sequence.

In another embodiment, the recombinant mammalian expression vector is capable of directing expression of the nucleic acid preferentially in a particular cell type, e.g. in plant cells (e.g., tissue-specific regulatory elements are used to express the nucleic acid). Tissue-specific regulatory elements are known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an enzyme of Table 1 can be expressed in bacterial cells such as C glutamicum or E. coli, insect cells, yeast or plants. Those of ordinary skill in the art know other suitable host cells.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such asG418, hygromycin and methotrexate. Nucleic acid encoding a selectable marker can be introduced into a host cell on the same vector as that encoding the enzymes of Table I or can be introduced on a separate vector. Cells stably transfected with the introduced nucleic acid can be identified by drug selection (e.g., cells that have incorporated the selectable marker gene will survive, while the other cells die).

In another embodiment, recombinant microorganisms can be produced which contain selected systems which allow for regulated expression of the introduced gene. For example, inclusion of a gene of Table 1 on a vector placing it under control of the lac operon permits expression of the gene only in the presence of IPTG. Such regulatory systems are well known in the art.

In one embodiment, the method comprises culturing the organisms of invention (into which a recombinant expression vector encoding e.g. an enzyme of table I has been introduced, or into which genome has been introduced a gene encoding a wild-type or altered enzyme) in a suitable medium for methionine production. In another embodiment, the method further comprises isolating methionine from the medium or the host cell.

It has been set out above that in order to modulate the metabolic flux of an organism, the amount and/or activity of enzymes of Table 1 catalyzing a reaction of the metabolic network may be increased or reduced. However, in order to modify the metabolic flux of an organism to produce an organism that is more efficient in methionine synthesis, changing the amount and/or activity of an enzyme is not limited to the enzymes listed in Table 1. Any enzyme that is homologous to the enzymes of Table 1 and carries out the same function in another organism may be perfectly suited to modulate the amount and/or activity in order to influence the metabolic flux by way of over-expression. The definitions for homology and identity have been given above.

In the following table, examples are given of homologues to some of the enzymes R1 to R61 of Table I which may be used for the purposes of the present invention by e.g. over-expressing them in C. glutamicum or E. coli in order to increase the amount and/or activity of the respective enzymes:

TABLE 2

| Gene product | | | Accession numbers sequences of |
|---|---|---|---|
| name | | Accession | related/alternative proteins, proteins with |
| (EC number) | Function | number(s) | conserved activity |
| glycine | recovery of | CAA52144.1 | YP_079782.1; NP_980596.1; YP_021091.1; |
| cleavage | glycine, T- | | AA11139.1; YP_148276.1; YP_175989.1; |
| system | protein | | BAB06533.1; NP_464875.1; NP_470723.1; |
| (R71/R72) | amino- | | YP_013965.1; ZP_00231385.1; ZP_00538577.1; |
| | methyl- | | NP_692823.1; NP_764775.1; YP_188676.1; |
| | transferase | | NP_372059.1; CAG43268.1; YP_253296.1; |
| | | | YP_186433.1; YP_041008.1; NP_621985.1; |
| | | | ZP_00560257.1; AAU84894.1; ZP_00574838.1; |
| | | | YP_075748.1; NP_143816.1; CAB50682.1; |
| | | | NP_662997.1; NP_972230.1; AAL82124.1; |
| | | | ZP_00590815.1; ZP_00528533.1; |
| | | | BAD85568.1; NP_228029.1; ZP_00511104.1; |
| | | | ZP_00591209.1; ZP_00532593.1; ZP_00525162.1; |
| | | | ZP_00355911.1; YP_112880.1; |
| | | | ZP_00399764.1; YP_143792.1; CAD75448.1; |
| | | | YP_004126.1; ZP_00535960.1; ZP_00334892.1; |
| | | | CAG35027.1; CAF23008.1; YP_010643.1; |
| | | | NP_951437.1; NP_840694.1; EAN28088.1; |
| | | | YP_125490.1; YP_094168.1; AAO91208.1; |
| | | | YP_122478.1; NP_110817.1; NP_214308.1; |
| | | | CAC12478.1; ZP_00602311.1; YP_256007.1; |
| | | | EAM94548.1; BAB66249.1; NP_342409.1; |
| | | | YP_023949.1; ZP_00054699.1; |
| | | | AAK25314.1; ZP_00270640.1; YP_169455.1; |
| | | | NP_148400.1; ZP_00577149.1; ZP_00375766.1; |
| | | | ZP_00303628.1; NP_280387.1; AAV46419.1; |
| | | | EAO04791.1; CAC32302.1; CAI36361.1; |
| | | | AAZ12256.1; NP_013914.1; YP_034020.1; |
| | | | BAC74698.1; Q827D7; ZP_00396527.1; |
| | | | EAK92665.1; EAK92694.1; ZP_00292858.1; |
| | | | ZP_00625542.1; NP_298674.1; |
| | | | EAO14274.1; NP_883104.1; NP_887405.1; |
| | | | ZP_00038971.2; NP_778843.1; ZP_00327636.1; |
| | | | EAA72140.1; NP_879086.1; NP_102591.1; |
| | | | CAJ04455.1; CAG61762.1; NP_716412.1; |
| | | | CAG88846.1; ZP_00582521.1; CAD17083.1; |
| | | | AAM36086.1; YP_191522.1; EAM75690.1; |
| | | | CAH00726.1; CAF26479.1; AAF11360.1; |
| | | | YP_202186.1; ZP_00308932.1; EAA47849.1; |
| | | | ZP_00278041.1; CAA81076.1; CAA91000.1; |
| | | | CAA85353.1; EAM85167.1; YP_071681.1; |
| | | | ZP_00638529.1; CAB16911.1; |
| | | | EAN06353.1; BAD35509.1; EAL84525.1; |
| | | | YP_118701.1; AAQ24377.1; YP_223286.1; |
| | | | NP_541539.1; ZP_00634544.1; CAB16916.1; |
| | | | BAD82264.1; AAN47559.1; NP_772393.1; |
| | | | YP_000299.1; CAB16918.1; CAD52982.1; |
| | | | XP_314216.2; NP_930808.1; AAD56281.1; |
| | | | AAQ66378.1; AAO76254.1; AAN33907.1; |
| | | | CAG73659.1; XP_219785.3; ZP_00111607.1; |
| | | | ZP_00585062.1; AAL21928.1; AAH42245.1; |
| | | | AAX66900.1; AAS46734.1; NP_949187.1; |
| | | | NP_989653.1; AAC31228.1;; AAA63798.1; |
| | | | ZP_00004510.1; CAA42443.1; ZP_00555139.1; |
| | | | YP_132995.1; NP_251135.1; |
| | | | NP_636487.1; ZP_00213263.1; ZP_00140178.2; |
| | | | ZP_00516950.1; AAB82711.1; AAZ27773.1; |
| | | | EAA61388.1; ZP_00167208.2; EAN04068.1; |
| | | | AAO07159.1; NP_936747.1; ZP_00499479.1; |
| | | | NP_806663.1; AAW42121.1; |
| | | | CAG83849.1; NP_923192.1; NP_441838.1; |
| | | | ZP_00593912.1; CAA52146.1; BAA14286.1; |
| | | | Q8XD33; AAA69071.1; EAM28058.1; ZP_00494650.1; |
| | | | ZP_00489316.1; ZP_00470629.1; |
| | | | YP_152074.1; AAM14125.1; AAM91322.1; |
| | | | ZP_00569907.1; ZP_00459442.1; NP_755358.1; |
| | | | ZP_00436271.1; NP_613061.1; |
| | | | CAE59244.1; BAC38022.1; CAA91099.1; |
| | | | ZP_00264533.1; AAC46780.1; AAG58030.1; |
| | | | YP_261725.1; AAL24244.1; NP_708666.1; |
| | | | CAH07776.1; Q8YNF9; YP_099306.1; ZP_00474391.1; |
| | | | AAL57651.1; YP_055456.1; |
| | | | NP_682393.1; NP_893785.1; YP_172756.1; |
| | | | CAH74116.1; ZP_00622638.1; YP_234188.1; |
| | | | NP_791106.1; NP_967658.1; EAN85656.1; |
| | | | ZP_00379711.1; XP_520482.1; AAN66613.1; |
| | | | ZP_00162707.1; ZP_00417692.1; YP_264083.1; |
| | | | AAQ61092.1; NP_216348.1; NP_855515.1; |
| | | | CAE76410.1; AAO39460.1; CAE22343.1; |
| | | | NP_800311.1; YP_206661.1; ZP_00506636.1; |
| | | | ZP_00417032.1; YP_164890.1; |
| | | | XP_760322.1; YP_266091.1; YP_156473.1; |
| | | | CAC46126.1; ZP_00244924.1; EAN91112.1; |
| | | | XP_637330.1; NP_960479.1; YP_160522.1; |
| | | | ZP_00317484.1; ZP_00412767.1; |
| | | | NP_253900.1; AAQ00873.1; NP_532152.1; |
| | | | ZP_00151464.1; EAO09970.1; ZP_00631036.1; |
| | | | ZP_00141690.2; NP_302381.1; CAG08109.1; |
| | | | CAE08889.1; YP_263017.1; AAN70757.1; |
| | | | ZP_00264788.1; XP_538655.1; AAZ27305.1; |
| | | | AAN17423.1; AAO38610.1; CAB85154.1; |
| | | | AAW89976.1; AAO44232.1; NP_789087.1; |
| | | | ZP_00496567.1; AAH57478.1; |
| | | | AAS16361.1; AAL33595.1; ZP_00048418.1; |
| | | | CAA72255.1; XP_598207.1; BAD82265.1; |
| | | | AAK26613.1; AAM93931.1; XP_517277.1; |
| | | | CAB50683.1; NP_143817.1; AAL82123.1; |
| | | | BAD82266.1; EAN80863.1; BAD85569.1; |
| | | | ZP_00574837.1; BAB26854.1; ZP_00560256.1; |
| | | | AAU84893.1; AAD33990.1; YP_175990.1; |
| | | | ZP_00641578.1; NP_390336.1; YP_023948.1; |
| | | | AAK25315.1; CAA38252.1; EAM94547.1; |
| | | | ZP_00530977.1; YP_013964.1; NP_470722.1; |
| | | | NP_464874.1; ZP_00525161.1; |
| | | | NP_148401.1; ZP_00589915.1; YP_079783.1; |
| | | | ZP_00233535.1; ZP_00577150.1; NP_228028.1; |
| | | | ZP_00238490.1; NP_980597.1; YP_021092.1; |
| | | | AAP11140.1; YP_038289.1; BAB06534.1; |
| | | | ZP_00399763.1; YP_148277.1; |
| | | | XP_606427.1; AAL04442.1; YP_075749.1; |
| | | | ZP_00527784.1; NP_692824.1; ZP_00334895.1; |
| | | | CAD75449.1; NP_840693.1; ZP_00591425.1; |
| | | | CAF23007.1; ZP_00054162.1; NP_621986.1; |
| | | | CAC12479.1; NP_662508.1; ZP_00512041.1; |
| | | | AAV46420.1; ZP_00375765.1; |
| | | | NP_110816.1; NP_280388.1; BAB66248.1; |
| | | | ZP_00538578.1; NP_951436.1; ZP_00367500.1; |
| | | | YP_169454.1; AAX77943.1; YP_094170.1; |
| | | | YP_122480.1; YP_125492.1;; ZP_00270641.1; |
| | | | YP_178313.1; XP_473181.1; ZP_00535961.1; |
| | | | NP_372060.1; ZP_00602310.1; |
| | | | YP_255986.1; CAE05443.2; CAB72709.1; |
| | | | CAG43269.1; NP_342410.1; BAB95353.1; |
| | | | AAA92036.1; YP_186434.1; YP_041009.1; |
| | | | AAM63785.1; XP_584346.1; AAM51302.1; |
| | | | AAF99780.1; ZP_00563258.1; EAN09988.1; |
| | | | NP_148290.1; YP_253295.1; ZP_00627278.1; |
| | | | YP_256008.1; AAO91209.1; XP_655257.1; |
| | | | ZP_00303629.1; CAC11669.1; NP_394004.1; |
| | | | EAM93962.1; CAB57769.1; CAB50124.1; |
| | | | ZP_00520055.1; ZP_00369709.1; EAN28089.1; |
| | | | CAC12627.1; AAB85605.1; Q9HI38; |
| | | | AAH26135.1; EAM24571.1; AAC03768.1; |
| | | | AAP98645.1; CAJ18422.1; NP_213757.1; |
| | | | CAA05909.1; NP_142859.1; BAD32415.1; |
| | | | BAC31437.1; NP_947805.1; YP_080623.1; |
| | | | CAH93356.1; ZP_00600159.1; NP_775139.1; |
| | | | ZP_00400734.1; CAA09590.2; XP_321361.2; |
| | | | AAL81283.1; BAB81491.1; ZP_00506445.1; |
| | | | ZP_00533640.1; YP_160688.1; |
| | | | NP_764776.1; NP_967201.1; AAH52991.1; |
| | | | AAO38289.1; NP_837365.1; XP_521504.1; |
| | | | NP_371248.1; NP_753970.1; CAG42465.1; |
| | | | AAC74750.1; AAU93791.1; Q7ADI4; 1KMK; |
| | | | 1JF9; BAA86566.1; YP_170184.1; YP_165179.1; |
| | | | NP_624289.1; AAX77932.1; CAD14721.1; |
| | | | BAD84717.1; ZP_00048472.2; AAF73526.1; |
| | | | XP_641773.1; ZP_00570672.1; |
| | | | ZP_00400089.1; NP_391148.1; NP_001007947.1; |
| | | | CAE08806.1; CAB50330.1; YP_192068.1; |
| | | | ZP_00152262.1; ZP_00502920.1; YP_096188.1; |
| | | | NP_796975.1; YP_254084.1; YP_124440.1; |
| | | | BAD86003.1; CAH04407.1; BAB07188.1; |
| | | | Q9K7A0; XP_313472.2; NP_103175.1; |
| | | | AAF63783.1; AAN58019.1; YP_127437.1; |
| | | | AAM62682.1; CAG90500.1; AAB85857.1; |
| | | | ZP_00387883.1; XP_562557.1; O27433; |
| | | | ZP_00170920.2; CAA63066.1; EAN08419.1; |
| | | | CAB73027.1; ZP_00335679.1; AAB85866.1; |
| | | | ZP_00129032.1; YP_178855.1; |
| | | | YP_145876.1; AAV94639.1; NP_623991.1; |
| | | | YP_040300.1; YP_040204.1; YP_080545.1; |
| | | | YP_253952.1; ZP_00638523.1; NP_371368.1; |
| | | | YP_255707.1; AAU38404.1; NP_085678.1; |
| | | | AAK68403.2; BAB66416.1; XP_624944.1; |
| | | | ZP_00135005.2; ZP_00595047.1; NP_950100.1; |
| | | | ZP_00458730.1; AAQ75173.1; AAP99129.1; |
| | | | YP_004062.1; XP_601722.1; CAC45089.1; |
| | | | CAA07007.1; EAL01528.1; XP_694140.1; |
| | | | NP_770978.1; AAC49935.1; EAA21518.1 |
| glycine | recovery of | Q8FE66. | NP_391159; |
| cleavage | glycine: H- | | YP_152075.1; NP_806664.1; NP_755359.1; |
| system | protein | | Q8FE66; AAX66901.1; ZP_00585063.1; NP_716411.1; |
| (R71/R72) | | | ZP_00582520.1; NP_930809.1; |
| | | | YP_071682.1; ZP_00638530.1; CAG73658.1; |
| | | | YP_156474.1; ZP_00634545.1; YP_131233.1; |
| | | | YP_268018.1; ZP_00417033.1; AAN70758.1; |
| | | | ZP_00141691.2; NP_253901.1; AAZ18651.1; |
| | | | EAO18275.1; ZP_00264789.1; |
| | | | ZP_00474390.1; ZP_00499480.1; ZP_00459441.1; |
| | | | EAM28059.1; ZP_00318113.1; AAQ61093.1; |
| | | | NP_790167.1; AAO91210.1; CAH37374.1; |
| | | | YP_169453.1; AAW49868.1; NP_840692.1; |
| | | | AAO07160.1; YP_263019.1; Q9K0L7; |
| | | | YP_160523.1; NP_800312.1; ZP_00334896.1; |
| | | | ZP_00278042.1; YP_233358.1; AAW90049.1; |
| | | | ZP_00213264.1; AAF96187.1; |
| | | | CAB84042.1; AAM37905.1; ZP_00658478.1; |
| | | | YP_112591.1; YP_132994.1; YP_200434.1; |
| | | | ZP_00593911.1; YP_206660.1; ZP_00167207.1; |
| | | | XP_464281.1; NP_638224.1; EAM75684.1; |
| | | | ZP_00574836.1; ZP_00151463.1; |
| | | | YP_270507.1; CAD17082.1; BAD45416.1; |
| | | | YP_004124.1; YP_143790.1; YP_075750.1; |
| | | | AAF11361.1; NP_465948.1; CAG35029.1; |
| | | | NP_960473.1; ZP_00568923.1; EAN06352.1; |
| | | | ZP_00506635.1; NP_532153.1; YP_223285.1; |
| | | | NP_541538.1; YP_014985.1; AAO63775.1; |
| | | | CAA20174.1; ZP_00399762.1; YP_010645.1; |
| | | | NP_471849.1; AAO77626.1; YP_094171.1; |
| | | | NP_693309.1; BAC70485.1; NP_228027.1; |
| | | | YP_253966.1; 1ZKO; YP_148857.1; |
| | | | YP_046528.1; YP_040289.1; ZP_00131107.1; |
| | | | YP_176481.1; NP_297474.1; Q9PGW7; |
| | | | ZP_00355907.1; ZP_00651773.1; NP_778393.1; |
| | | | YP_021882.1; NP_855509.1; CAF26480.1; |
| | | | CAG42548.1; ZP_00389942.1; NP_981423.1; |
| | | | AAP11863.1; NP_302386.1; NP_764151.1; |
| | | | YP_266090.1; ZP_00237746.1; ZP_00244923.1; |
| | | | ZP_00396528.1; YP_188077.1; |
| | | | CAC46127.1; ZP_00375764.1; ZP_00560255.1; |
| | | | ZP_00270642.1; BAB07203.1; EAN28788.1; |
| | | | NP_216342.1; CAH09835.1; NP_251136.1; |
| | | | YP_185749.1; AAT49691.1; ZP_00264532.1; |
| | | | NP_102590.1; YP_034021.1; |
| | | | NP_879085.1; AAP54618.1; ZP_00625541.1; |
| | | | ZP_00525160.1; AAV46421.1; ZP_00412765.1; |
| | | | CAH02746.1; CAA81075.1; CAB16912.1; |
| | | | CAB16710.1; CAA85761.1; ZP_00622637.1; |
| | | | CAB16914.1; CAA85759.1; NP_143205.1; |
| | | | YP_101635.1; ZP_00380048.1; ZP_00004511.1; |
| | | | ZP_00414055.1; AAC61829.1; |
| | | | AAL81616.1; YP_261724.1; CAB49742.1; |
| | | | AAN66614.1; Q9V0G1; YP_080558.1; BAD84339.1; |
| | | | AAK25316.1; NP_949186.1; CAJ13836.1; |
| | | | AAQ67414.1; 1DXM; CAA85768.1; |
| | | | CAA85757.1; P93255; CAJ13723.1; CAA85755.1; |
| | | | ZP_00292299.1; ZP_00308328.1; |
| | | | NP_772392.1; CAI36363.1; NP_147622.1; |
| | | | NP_391159.1; ZP_00577152.1; CAA85756.1; |
| | | | AAQ66080.1; AAV94183.1; CAA88734.1; |
| | | | ZP_00547429.1; NP_621789.1; CAA85760.1; |
| | | | AAR37471.1; ZP_00555140.1; AAW47059.1; |
| | | | ZP_00303630.1; NP_621987.1; |
| | | | AAG48828.1; CAG86839.1; YP_191521.1; |
| | | | YP_055457.1; ZP_00631037.1; YP_118696.1; |
| | | | CAA85767.1; XP_637044.1; AAM64413.1; |
| | | | CAC19751.1; ZP_00379709.1; NP_280389.1; |
| | | | CAF23006.1; EAO25275.1; AAU84892.1; |
| | | | CAF92157.1; EAN04067.1; AAH91548.1; |
| | | | YP_164889.1; NP_213756.1; CAG62852.1; |
| | | | AAW49010.1; CAA94317.1; ZP_00527786.1; |
| | | | YP_234189.1; CAD52976.1; NP_967650.1; |
| | | | CAE66592.1; EAA66192.1; AAW27708.1; |
| | | | ZP_00565058.1; XP_536768.1; BAB66246.1; |
| | | | CAA95820.1; AAL68248.1; AAH14745.1; |
| | | | NP_791107.1; EAL90537.1; XP_579628.1; |
| | | | XP_316586.2; AAP88829.1; NP_004474.2; |
| | | | NP_080848.1; NP_951435.1; XP_523434.1; |
| | | | CAA85754.1; Q9N121; AAS59848.1; |
| | | | YP_172757.1; ZP_00534758.1; ZP_00591423.1; |
| | | | ZP_00512042.1; ZP_00661686.1; |
| | | | NP_883103.1; ZP_00054163.1; NP_598282.1; |
| | | | AAW31875.1; XP_756407.1; AAH76212.1; |
| | | | CAF99616.1; CAA94316.1; NP_953067.1; |
| | | | EAA72139.1; NP_110807.1; AAS52315.1; |
| | | | ZP_00575020.1; P20821; NP_662509.1; |
| | | | CAG33353.1; CAE63163.1; ZP_00589916.1; |
| | | | XP_217678.1; YP_256010.1; XP_582835.1; |
| | | | CAC12487.1; NP_394822.1; ZP_00535962.1; |
| | | | NP_001004372.1; EAL29812.1; CAB05472.1; |
| | | | XP_615385.1; EAL03567.1; O22535; |
| | | | AAH82740.1; AAX07637.1; NP_926477.1; |
| | | | EAM93557.1; XP_584988.1; AAM92707.1 |
| | | | ZP_00515529.1; ZP_00530979.1; AAH81062.1; |
| | | | ZP_00111606.1; NP_682468.1; |
| | | | Q8DIB2; EAA77334.1; AAN47560.1; AAL33596.1; |
| | | | ZP_00162706.2; NP_342412.1; Q8G4Z7; |
| | | | ZP_00120558.2; ZP_00136571.1; NP_972232.1; |
| | | | CAE18120.1; Q8YNF8; XP_604979.1; |
| | | | CAE08890.1; CAE76092.1; YP_023402.1; |
| | | | NP_009355.2; P39726; CAD75450.1; |
| | | | NP_440920.1; CAE47935.1; XP_498178.1; |
| | | | NP_893786.1; ZP_00327635.1; EAN76953.1; |
| | | | ZP_00140179.2; EAN99694.1; AAZ14696.1; |
| | | | CAE22344.1; EAN83079.1; BAB26349.1; |
| | | | YP_169819.1; AAX78078.1; AAQ00874.1; |
| | | | AAC36844.1; CAG78944.1; XP_694123.1; |
| | | | ZP_00050263.1; AAO44734.1; XP_414165.1; |
| | | | ZP_00654389.1; ZP_00575538.1; XP_518701.1; |
| | | | BAB66989.1; NP_342534.1; XP_583383.1; |
| | | | NP_213643.1; YP_255059.1; NP_214139.1; |
| | | | NP_213280.1; ZP_00540302.1; |
| | | | YP_055788.1; AAP05107.1; AAF39393.1; |
| | | | ZP_00399135.1; NP_701199.1; CAD75020.1; |
| | | | XP_343995.2; AAP98380.1; NP_300490.1; |
| | | | XP_635521.1; YP_219766.1; XP_637059.1; |
| | | | AAV71155.1; XP_739104.1; EAA18076.1; |
| | | | NP_219787.1; ZP_00400682.1; NP_967270.1; |
| | | | ZP_00384691.1; ZP_00401280.1; NP_816146.1; |
| | | | ZP_00526092.1; NP_213714.1; |
| | | | YP_255058.1; NP_785822.1; BAB66988.1; |
| | | | NP_253484.1; ZP_00141248.1; ZP_00399137.1; |
| | | | XP_676766.1; EAO22790.1; NP_342572.1; |
| | | | AAF07900.1; ZP_00152140.2; XP_518003.1; |
| | | | AAA23866.1; AAP06383.1; AAM99940.1; |
| | | | ZP_00523572.1; AAH09065.1; YP_039780.1; |
| | | | NP_735539.1; NP_326272.1; YP_252181.1; |
| | | | NP_802319.1; XP_637062.1; AAK70873.1; |
| | | | ZP_00523574.1; NP_784119.1; |
| | | | XP_356748.3; YP_115837.1; XP_520481.1; |
| | | | BAB94166.1; XP_527720.1; NP_975513.1; |
| | | | ZP_00511802.1; ZP_00660576.1; XP_598843.1; |
| | | | ZP_00335312.1; ZP_00501149.1; ZP_00496208.1; |
| | | | ZP_00488962.1; ZP_00481092.1; |
| | | | ZP_00470769.1; YP_112037.1; ZP_00315286.1; |
| | | | ZP_00591859.1; NP_622848.1; |
| | | | CAB59889.1; ZP_00566435.1; ZP_00633779.1; |
| | | | AAG13505.2; CAB16915.1; ZP_00585787.1; |
| | | | ZP_00531610.1; AAW51218.1; YP_263215.1; |
| | | | AAK22373.1; AAT58044.1; EAA20319.1; |
| | | | NP_842410.1; EAN32517.1; NP_532021.1; |
| | | | AAH75478.1; NP_533976.1; ZP_00385854.1; |
| | | | AAL04441.1; AAK89915.1; |
| | | | BAD16654.1; NP_216731.1; NP_770578.1; |
| | | | ZP_00547768.1; NP_522769.1; ZP_00383064.1; |
| | | | ZP_00322497.1; YP_022846.1; NP_961247.1; |
| | | | YP_238125.1; ZP_00412124.1; XP_475165.1; |
| | | | XP_470945.1; NP_910410.1; NP_635909.1; |
| | | | CAE01575.2; XP_419793.1; ZP_00170555.1; |
| | | | NP_217017.1;; ZP_00556997.1; |
| | | | AAP99073.1; NP_297341.1; AAX73221.1; |
| | | | NP_214109.1; NP_440434.1; CAG87711.1; |
| | | | ZP_00051435.1; BAB80778.1; XP_393389.2; |
| | | | ZP_00651791.1; ZP_00269419.1; ZP_00423019.1; |
| | | | YP_170419.1; NP_001006383.1; |
| | | | XP_482561.1; NP_778293.1; CAB03400.1; |
| | | | CAH04871.1; XP_655127.1; AAF61288.1; |
| | | | ZP_00356683.1; ZP_00565878.1; CAJ01708.1; |
| | | | XP_758384.1; AAU38115.1; AAK23858.1; |
| | | | XP_688912.1; BAB06344. |
| glycine | recovery of | CAA52144 | NP_390336; NP_708668.1; Q8XD32; NP_755360.1; |
| cleavage | glycine: | | 1VLO; YP_152076.1; AAX66902.1; |
| system | P-protein | | YP_071683.1; NP_670593.1; NP_930810.1; |
| (R71/R72) | glycine | | CAG73657.1; ZP_00585064.1; ZP_00634546.1; |
| | dehydrogenase | | NP_716410.1; ZP_00638531.1; ZP_00582519.1; |
| | (decarboxylating) | | YP_156475.1; YP_268017.1; ZP_00141692.2; |
| | | | ZP_00417034.1; NP_253902.1; |
| | | | AAT51348.1; ZP_00318114.1; YP_233357.1; |
| | | | AAO91211.1; AAM37906.1; ZP_00264790.1; |
| | | | NP_790166.1; YP_125494.1; YP_263020.1; |
| | | | YP_122482.1; YP_094172.1; AAN70759.1; |
| | | | Q5ZZ93; YP_200433.1; NP_638225.1; |
| | | | YP_112882.1; NP_778394.1; NP_840691.1; |
| | | | EAO18276.1; NP_297476.1; ZP_00651772.1; |
| | | | YP_160524.1; AAQ61094.1; ZP_00334897.1; |
| | | | YP_104498.1; ZP_00213265.1; |
| | | | EAM28060.1; NP_887403.1; NP_883102.1; |
| | | | ZP_00151462.1; ZP_00654390.1; NP_879084.1; |
| | | | ZP_00459440.1; ZP_00454903.1; ZP_00278043.1; |
| | | | ZP_00499481.1; EAN28090.1; |
| | | | CAD17081.1; Q9K0L8; AAZ18652.1; CAB84041.1; |
| | | | Q9JVP2; ZP_00167206.1; YP_169452.1; |
| | | | AAW90051.1; AAW49997.1; ZP_00593910.1; |
| | | | ZP_00419736.1; ZP_00560254.1; |
| | | | YP_041010.1; YP_186435.1; YP_253294.1; |
| | | | NP_764777.1; YP_079784.1; ZP_00325013.1; |
| | | | YP_172504.1; BAB06535.1; NP_621988.1; |
| | | | NP_390337.1; NP_464873.1; ZP_00233534.1; |
| | | | ZP_00165293.2; YP_013963.1; |
| | | | NP_470721.1; NP_681534.1; BAB76308.1; |
| | | | ZP_00399761.1; ZP_00517920.1; NP_692825.1; |
| | | | Q8CXD9; NP_926476.1; Q7NFJ5; YP_075751.1; |
| | | | NP_228026.1; ZP_00162705.1; |
| | | | YP_148278.1; AAQ00895.1; 1YX2; ZP_00355906.1; |
| | | | ZP_00111605.1; AAU84891.1; NP_441988.1; |
| | | | YP_085561.1; ZP_00238491.1; |
| | | | NP_980598.1; ZP_00530333.1; NP_662667.1; |
| | | | AAP11141.1; CAE22389.1; CAE08940.1; |
| | | | EAO25274.1; ZP_00574835.1; ZP_00511544.1; |
| | | | NP_967651.1; YP_004123.1; ZP_00590889.1; |
| | | | YP_175991.1; ZP_00538579.1; YP_143789.1; |
| | | | ZP_00533333.1; ZP_00307846.1; |
| | | | ZP_00396529.1; ZP_00660916.1; ZP_00588126.1; |
| | | | ZP_00525159.1; ZP_00531283.1; |
| | | | NP_893804.1; CAI36362.1; AAO79689.1; |
| | | | AAX16385.1; NP_393488.1; CAH07007.1; |
| | | | CAA20175.1; NP_110577.1; ZP_00292300.1; |
| | | | YP_055458.1; BAB59199.1; BAC70484.1; |
| | | | EAM94419.1; NP_960885.1; ZP_00412766.1; |
| | | | YP_117906.1; NP_301653.1; NP_972233.1; |
| | | | NP_295535.1; AAQ66593.1; NP_216727.1; |
| | | | P64220; ZP_00646130.1; YP_023281.1; |
| | | | CAC11159.1; O67441; EAM73669.1; CAF23005.1; |
| | | | ZP_00656447.1; AAN47561.1; YP_000301.1; |
| | | | CAD75451.1; Q8F935; AAV46422.1; |
| | | | Q5V230; ZP_00631038.1; AAO07163.1; |
| | | | ZP_00379710.1; AAK25317.1; ZP_00574284.1; |
| | | | ZP_00549460.1; AAL33597.1; NP_936752.1; |
| | | | XP_473945.1; CAA81081.1; ZP_00054164.1; |
| | | | ZP_00577153.1; CAB16917.1; |
| | | | NP_143049.1; O58888; CAB50008.1; BAD86224.1; |
| | | | AAB38502.1; YP_164888.1; EAL00308.1; |
| | | | NP_280390.1; YP_206658.1; Q9HPJ7; |
| | | | CAA52800.1; EAL00186.1; ZP_00130510.2; |
| | | | AAP21169.1; NP_800315.1; CAA81077.1; |
| | | | CAA94902.1; NP_789581.1; CAA10976.1; |
| | | | AAO44735.1; XP_756577.1; YP_266089.1; |
| | | | CAB11698.1; ZP_00535963.1; NP_949185.1; |
| | | | XP_629708.1; ZP_00270643.1; AAK87256.1; |
| | | | AAL81465.1; YP_261727.1; NP_010302.1; |
| | | | EAN06351.1; YP_010902.1; CAG77727.1; |
| | | | AAB05000.1; ZP_00303631.1; |
| | | | NP_001006021.1; ZP_00625540.1; CAG85941.1; |
| | | | NP_951434.1; NP_772391.1; CAF93361.1; |
| | | | CAH02226.1; YP_270503.1; CAF26481.1; |
| | | | ZP_00601921.1; XP_394029.2; ZP_00474388.1; |
| | | | CAC46128.1; AAN66611.1; ZP_00264535.1; NP_251132.1; ZP_00375763.1; |
| | | | ZP_00140175.2; ZP_00555141.1; YP_234187.1; |
| | | | AAT51611.1; ZP_00622636.1; ZP_00506634.1; |
| | | | NP_791105.1; AAR21108.1; ZP_00417689.1; |
| | | | EAN86200.1; AAW42395.1; |
| | | | EAL33114.1; BAB66247.1; NP_532154.1; |
| | | | YP_034022.1; NP_102589.1; XP_331926.1; |
| | | | EAN85387.1; CAG58515.1;; AAB37080.1; |
| | | | ZP_00244922.1; YP_191520.1; CAJ09347.1; |
| | | | CAJ09346.1; CAE64583.1; AAN33909.1; |
| | | | YP_223284.1; NP_214006.1; BAA02967.1; |
| | | | BAA03512.1; AAF52996.1; AAX33383.1; |
| | | | XP_322034.2; NP_541537.1; NP_001013836.1; |
| | | | EAA68431.1; NP_001014026.1; XP_620786.1; |
| | | | EAA51066.1; EAN04066.1; XP_541886.1; |
| | | | YP_256009.1; Q9TSZ7; YP_132990.1; |
| | | | NP_990119.1; AAH07546.2; EAA65791.1; |
| | | | Q9YBA2; EAL90405.1; CAC41491.1; |
| | | | NP_342411.1; EAN79919.1; XP_517018.1; |
| | | | NP_107651.1; ZP_00004512.2; BAA12709.1; |
| | | | NP_148104.1; AAV94849.1; YP_266710.1; |
| | | | XP_516459.1; NP_105581.1; ZP_00620069.1; |
| | | | ZP_00631895.1; AAL13520.1; YP_047137.1; |
| | | | AAF68432.3; CAC46853.1; XP_542460.1; |
| | | | ZP_00460296.1; ZP_00554633.1; ZP_00282393.1; |
| | | | NP_102909.1; YP_235303.1; |
| | | | AAQ87218.1; ZP_00213445.1; NP_106044.1; |
| | | | ZP_00169723.2; ZP_00500952.1; ZP_00489849.1; |
| | | | ZP_00480439.1; ZP_00450235.1; |
| | | | ZP_00436058.1; AAY59105.1; EAM32228.1; |
| | | | AAW21506.1; CAD14805.1; ZP_00410721.1; |
| | | | NP_792264.1; Q46337; NP_521609.1; |
| | | | BAD97818.1; 1X31; NP_534554.1; ZP_00602139.1; |
| | | | AAK16489.1; YP_266475.1; EAA22341.1; |
| | | | XP_318114.2; CAC47432.1; ZP_00004192.1; |
| | | | NP_534790.1; AAL52901.1; |
| | | | ZP_00565350.1; ZP_00657316.1; YP_134767.1; |
| | | | ZP_00379371.1; YP_134764.1; XP_742683.1; |
| | | | AAN29180.1; ZP_00602144.1; CAG35030.1; |
| | | | EAM31940.1; ZP_00556129.1; |
| | | | CAC49374.1; ZP_00050264.2; ZP_00379741.1; |
| | | | NP_705537.1; ZP_00471825.1; NP_104736.1; |
| | | | ZP_00600293.1; AAY87206.1; YP_266690.1; |
| | | | AAL76414.1; AAC31611.1; AAR38319.1; |
| | | | ZP_00658996.1; YP_266673.1; |
| | | | NP_105928.1; ZP_00630198.1; YP_266631.1; |
| | | | NP_254105.1; NP_107666.1; NP_104289.1; |
| | | | NP_102901.1; AAV96623.1; BAC74662.1; |
| | | | AAV95607.1; ZP_00620942.1; ZP_00645790.1; |
| | | | CAC41486.1; EAN05741.1; AAN65213.1; |
| | | | YP_269196.1; ZP_00620654.1; ZP_00660136.1; |
| | | | NP_885663.1; NP_436414.1; |
| | | | NP_881143.1; AAM75070.1; NP_572162.2; |
| | | | ZP_00379745.1; CAA39468.1; ZP_00264573.1; |
| | | | YP_262784.1; ZP_00380782.1; ZP_00278582.1; |
| | | | AAV94866.1; XP_414684.1; YP_237780.1; |
| | | | ZP_00602141.1; YP_165138.1; NP_790307.1; |
| | | | NP_620802.2; NP_534700.1; |
| | | | AAV95690.1; EAL32452.1; AAF21941.1; |
| | | | AAN65956.1; NP_083048.1; AAV94935.1; |
| | | | CAC46854.1; CAC41470.1; AAH24126.1; |
| | | | NP_037523.2; ZP_00565365.1; NP_102887.1; |
| | | | YP_134760.1; AAQ87217.1; AAH89599.1; |
| | | | ZP_00554816.1; AAK16482.1; NP_103085.1; |
| | | | ZP_00620147.1; NP_102906.1; ZP_00556188.1; |
| | | | XP_307967.2; ZP_00622120.1; CAC47100.1; |
| | | | NP_106776.1; CAH90377.1; ZP_00554918.1; |
| | | | CAD31640.1; XP_672544.1; |
| | | | AAT81177.1; AAK27867.2; AAD33412.1; |
| | | | NP_107653.1; CAI12276.1; BAD97122.1; ZP_00410737.1; |
| | | | AAD43585.1; NP_103793.1; |
| | | | XP_526883.1; XP_548398.1; CAB63337.2; |
| | | | YP_266661.1; ZP_00521798.1; EAA60688.1; |
| | | | AAL51865.1; AAV95026.1; EAL26357.1; |
| | | | ZP_00619907.1; CAE74368.1; NP_103190.1; |
| | | | AAV94915.1; AAG55663.1; XP_395831.2; |
| | | | AAK92969.1; NP_446116.1; AAF57796.1; |
| | | | AAN71380.1; ZP_00005411.1; CAE58942.1; |
| | | | EAA70894.1; AAV94873.1; ZP_00631887.1; |
| | | | ZP_00629673.1; BAB34711.1; ZP_00619923.1; |
| | | | ZP_00622863.1; EAL85410.1; |
| | | | AAL76413.1; AAR38318.1; CAD47921.1; |
| | | | AAH03456.1; NP_102854.1; AAH76859.1; |
| | | | AAL04443.1; AAH68953.1; YP_165137.1; |
| | | | XP_580581.1; ZP_00050273.2; ZP_00556400.1; |
| | | | ZP_00555517.1; AAV96627.1; AAV93943.1; |
| | | | AAH81271.1; AAK87410.1; ZP_00556086.1; |
| | | | AAV93879.1; AAH44792.1; XP_527208.1; |
| | | | ZP_00327983.1; ZP_00554213.1; |
| | | | NP_532318.1; CAI12274.1; AAH22388.1; |
| | | | XP_546052.1; XP_676622.1; AAV93533.1; |
| | | | YP_265671.1; AAV47350.1; AAV95190.1; |
| | | | CAD31286.1; ZP_00516133.1; ZP_00620892.1; |
| | | | AAH55193.1; AAY82706.1; ZP_00052785.2; |
| | | | AAR38102.1 |
| glycine | LpdA- | P0A9P0 | NP_706070.2; NP_752095.1; |
| cleavage | protein | | CAA24742.1; AAX64059.1; |
| system | | | AAL19118.1; NP_804043.1; |
| (R71/R72) | | | YP_149503.1; CAG76686.1; |
| | | | YP_069256.1; NP_930833.1; |
| | | | NP_935564.1; AAO10051.1; |
| | | | AAF95555.1; ZP_00585786.1; |
| | | | AAK02977.1; NP_798896.1; |
| | | | AAC46405.1; ZP_00122566.1; |
| | | | ZP_00132373.2; YP_131302.1; |
| | | | YP_205561.1; NP_716063.1; |
| | | | ZP_00637900.1; ZP_00633839.1; |
| | | | ZP_00582828.1; AAU37941.1; |
| | | | ZP_00134358.2; ZP_00157402.1; |
| | | | AAX88688.1; NP_439387.1; |
| | | | ZP_00154973.1; AAP96400.1; |
| | | | YP_154852.1; YP_271444.1; |
| | | | ZP_00464633.1; ZP_00451158.1; |
| | | | ZP_00212747.1; ZP_00500723.1; |
| | | | ZP_00486500.1; ZP_00463487.1; |
| | | | ZP_00467577.1; ZP_00423839.1; |
| | | | ZP_00423458.1; ZP_00283805.1; |
| | | | AAO90013.1; ZP_00595215.1; |
| | | | ZP_00170705.2; NP_879789.1; |
| | | | YP_123783.1; NP_889077.1; |
| | | | YP_126870.1; NP_240038.1; |
| | | | YP_095531.1; CAD15305.1; |
| | | | AAQ58205.1; NP_883762.1; |
| | | | NP_770362.1; YP_170418.1; |
| | | | CAA61894.1; AAV29309.1; |
| | | | CAB84783.1; AAF41719.1; |
| | | | ZP_00565931.1; CAA59171.1; |
| | | | CAA54878.1; AAW89295.1; |
| | | | CAA62435.1; ZP_00150164.2; 1BHY; |
| | | | 1OJT; CAA61895.1; YP_157096.1; |
| | | | CAA57206.1; AAM38502.1; |
| | | | NP_635936.1; YP_199361.1; |
| | | | NP_660554.1; NP_842161.1; |
| | | | ZP_00507350.1 NP_779995.1; |
| | | | YP_115390.1; ZP_00651360.1; |
| | | | NP_298158.1; AAR38073.1; |
| | | | EAO17659.1; ZP_00245305.1; |
| | | | AAR38213.1; AAR38090.1; |
| | | | NP_777818.1; BAC24467.1; |
| | | | NP_891227.1; NP_879460.1; |
| | | | NP_878457.1; CAD71978.1; |
| | | | YP_078853.1; AAK50273.1; |
| | | | AAK50266.1; AAF11916.1; |
| | | | NP_389344.1; ZP_00396676.1; |
| | | | EAO21015.1; CAA37631.1; 1EBD; |
| | | | YP_146914.1; BAB06371.1; |
| | | | YP_085309.1; AAP10890.1; |
| | | | YP_020826.1; YP_175913.1; Q04829; |
| | | | AAN03817.1; NP_692336.1; |
| | | | AAG17888.1; ZP_00474314.1; |
| | | | NP_764349.1; AAA99234.1; 1LPF; |
| | | | NP_250278.1; XP_475628.1; |
| | | | YP_253771.1; YP_143499.1; |
| | | | YP_257414.1; AAF34795.3; |
| | | | AAF79529.1; YP_040483.1; |
| | | | YP_005722.1; YP_074243.1; |
| | | | AAN23154.1; AAK50305.1; |
| | | | AAS20045.1; ZP_00540244.1; |
| | | | EAN07674.1; AAC26053.1; |
| | | | NP_815077.1; NP_908725.1; |
| | | | ZP_00307577.1; AAS47493.1; |
| | | | AAF34796.1; CAA11554.1; |
| | | | YP_013676.1; ZP_00317120.1; |
| | | | AAV48381.1; CAB84413.1; |
| | | | AAB30526.1; NP_969527.1; |
| | | | BAB44156.1; NP_464580.1; |
| | | | XP_635122.1; AAF41363.1; |
| | | | AAK50280.1; ZP_00397330.1; |
| | | | YP_265659.1; NP_470384.1; |
| | | | CAA44729.1; AAW89611.1; 1DXL; |
| | | | ZP_00401182.1 ZP_00418304.1; |
| | | | NP_792022.1; ZP_00625011.1; |
| | | | AAD53185.1; 3LAD; EAN08634.1; |
| | | | AAH18696.1; CAH93405.1; |
| | | | YP_235092.1; NP_967737.1;; |
| | | | CAJ08862.1; NP_945538.1; |
| | | | NP_763632.1; BAE00452.1; |
| | | | BAD92940.1; NP_000099.1; IZMD; |
| | | | AAB01381.1; NP_999227.1; |
| | | | NP_767089.1; AAS47708.1; |
| | | | AAR21288.1; AAA35764.1; |
| | | | YP_034342.1; EAN90443.1; |
| | | | EAN96941.1; CAA61483.1; |
| | | | AAN69768.1; AAF12067.1; P31052; |
| | | | NP_105199.1; ZP_00263252.1; |
| | | | AAH62069.1; CAA72132.1; |
| | | | NP_031887.2; CAG58981.1; |
| | | | CAF26798.1; EAN80618.1; AAN15202.1; |
| | | | CAA72131.1; ZP_00269527.1; |
| | | | CAD72797.1; ZP_00554136.1; |
| | | | CAD61860.1; AAC53170.1; CAF05589.1; |
| | | | ZP_00622437.1; CAG81278.1; |
| | | | ZP_00284261.1; ZP_00497224.1; |
| | | | EAK93183.1; ZP_00492121.1; |
| | | | NP_533297.1; AAS53883.1; |
| | | | YP_160845.1; AAV93660.1; |
| | | | CAG31211.1; CAA49991.1; |
| | | | AAM93255.1; AAK11679.1; |
| | | | ZP_00427535.1; YP_258846.1; |
| | | | AAN30810.1; ZP_00449174.1; |
| | | | CAF92514.1; AAQ91233.1; |
| | | | AAH44432.1; XP_320877.2; |
| | | | AAH56016.1; YP_222565.1; |
| | | | CAC47627.1; AAA96487.1; |
| | | | ZP_00464142.1; NP_280867.1; |
| | | | YP_067405.1; CAB65609.1; |
| | | | AAL51327.1; AAK22329.1; |
| | | | YP_246823.1; NP_105334.1; |
| | | | XP_758608.1; CAH00655.1; |
| | | | NP_772974.1; AAB88282.1; |
| | | | ZP_00211386.1; EAN27796.1; |
| | | | AAN70931.1; EAL29693.1; |
| | | | ZP_00340462.1; ZP_00153792.2; |
| | | | ZP_00579524.1; AAZ17978.1; |
| | | | NP_266215.1; AAN33719.1; |
| | | | AAD30450.1; ZP_00383074.1; |
| | | | ZP_00597315.1; CAC47514.1; |
| | | | AAF49294.1; YP_223465.1; |
| | | | NP_220840.1; NP_360330.1; |
| | | | EAA26462.1; CAA39235.1; |
| | | | ZP_00578463.1; YP_047424.1; |
| | | | AAM36402.1; BAB03935.1; |
| | | | AAN69982.1; NP_116635.1; |
| | | | ZP_00654346.1; CAG85768.1; 1V59; |
| | | | NP_623271.1; AAA65618.1; |
| | | | ZP_00305550.1; XP_623438.1; |
| | | | ZP_00007570.1; ZP_00320049.1; |
| | | | AAN75183.1; ZP_00323583.1; |
| | | | YP_200681.1; 1LVL; ZP_00151187.2; |
| | | | AAP03132.1; CAD14973.1; |
| | | | ZP_00630163.1; ZP_00139957.1; |
| | | | NP_250940.1; NP_636857.1; |
| | | | CAB05249.2; ZP_00166998.2; |
| | | | AAN75720.1; CAA62982.1; |
| | | | ZP_00265019.1; ZP_00384289.1; |
| | | | AAV47687.1; ZP_00303079.1; |
| | | | NP_842316.1; XP_331183.1; |
| | | | AAV28779.1; AAN48422.1; |
| | | | ZP_00597992.1; AAN75618.1; |
| | | | AAV28746.1; ZP_00267415.1; |
| | | | ZP_00650982.1; AAQ58749.1; |
| | | | NP_298837.1; AAN75159.1; |
| | | | NP_778978.1; ZP_00575798.1; |
| | | | YP_002403.1; AAB97089.1; |
| | | | ZP_00511405.1; YP_005669.1; |
| | | | EAO21998.1; XP_613473.1; |
| | | | ZP_00245417.1; ZP_00210841.1; |
| | | | ZP_00561492.1; YP_259638.1; |
| | | | EAO16949.1; NP_785656.1; |
| | | | CAF23812.1; ZP_00055963.2; |
| | | | YP_143553.1; NP_953492.1; |
| | | | CAA63810.1; CAF22875.1; AAV89136.1; |
| | | | ZP_00536790.1; AAF39644.1; |
| | | | ZP_00621355.1; ZP_00486105.1; |
| | | | ZP_00020745.2; ZP_00589771.1; |
| | | | YP_180376.1; NP_220072.1; |
| | | | CAI27032.1; EAL87307.1; YP_112273.1; |
| | | | ZP_00376179.1; ZP_00498294.1; |
| | | | ZP_00492099.1; ZP_00463379.1; |
| | | | ZP_00217095.1; CAI27980.1; |
| | | | AAK23707.1; CAD60736.1; |
| | | | ZP_00268854.1; EAA77706.1; |
| | | | ZP_00629856.1; NP_879905.1; |
| | | | EAA51976.1; NP_885384.1; CAI29613.1; |
| | | | AAA91879.1; ZP_00376555.1; |
| | | | ZP_00141283.2; NP_253516.1; |
| | | | NP_300890.1; AAB40885.1; |
| | | | AAN03814.1; ZP_00644737.1; |
| | | | AAO36548.1; AAP98791.1; |
| | | | YP_079735.1; NP_388690.1; |
| | | | AAP05672.1; NP_966507.1; P95596; |
| | | | EAN04065.1; NP_532124.1; |
| | | | ZP_00507305.1; NP_948204.1; |
| | | | ZP_00557093.1; YP_220287.1; |
| | | | ZP_00642506.1; ZP_00591535.1; |
| | | | NP_102193.1; NP_771418.1; |
| | | | AAA19188.1; AAK72471.1; |
| | | | AAK72470.1; NP_345630.1; |
| | | | NP_756887.1; ZP_00404212.1; |
| | | | AAK72472.1; AAN50085.1; |
| | | | CAC46029.1; YP_001129.1; |
| | | | AAL64341.1; ZP_00526430.1; |
| | | | ZP_00308867.1; YP_053282.1; |
| | | | YP_036862.1; ZP_00210426.1; |
| | | | ZP_00625423.1; ZP_00601791.1; |
| | | | YP_045732.1; YP_016277.1; P54533; |
| | | | AAV95488.1; AAW71149.1; |
| | | | YP_021029.1; YP_153983.1; |
| | | | ZP_00240355.1; XP_395801.2; |
| | | | EAN08156.1; AAP94898.1; |
| | | | NP_326592.1; AAP11076.1; |
| | | | ZP_00239726.1; NP_980528.1; |
| | | | ZP_00620223.1; ZP_00512893.1; |
| | | | YP_019413.1; NP_148088.1; |
| | | | XP_678378.1; YP_180009.1; |
| | | | NP_735347.1; CAI26632.1; AAN30046.1; |
| | | | BAB04498.1; AAN57909.1; |
| | | | ZP_00373647.1; NP_692788.1; |
| | | | ZP_00053288.1; EAM72947.1; |
| | | | YP_078075.1; ZP_00006401.1; |
| | | | EAA16706.1; YP_221832.1; |
| | | | XP_742153.1; AAL52038.1; |
| | | | NP_966125.1; YP_247286.1; |
| | | | AAA74473.1; ZP_00589476.1; |
| | | | CAI38117.1; AAO78292.1; EAA26057.1; |
| | | | BAD11090.1; ZP_00545191.1; |
| | | | CAE73952.1; YP_060098.1; |
| | | | BAB05544.1; NP_802451.1; |
| | | | NP_360876.1; AAL97648.1; |
| | | | ZP_00154188.2; ZP_00331725.1; |
| | | | AAV62625.1; CAG35032.1; |
| | | | YP_084091.1; ZP_00366080.1; |
| | | | YP_139515.1; YP_121481.1; |
| | | | ZP_00340821.1; ZP_00531539.1; |
| | | | ZP_00630106.1; CAB06298.1; |
| | | | NP_979105.1;; ZP_00162168.1; |
| | | | AAP09729.1; YP_175948.1; |
| | | | NP_938751.1; ZP_00277446.1; |
| | | | BAB76444.1; YP_148232.1; |
| | | | AAK33923.1; AAO75416.1; |
| | | | AAT58044.1; YP_116014.1; |
| | | | BAD11095.1; ZP_00571989.1; |
| | | | YP_033412.1; AAT47753.1; |
| | | | NP_221155.1; ZP_00111840.1; |
| | | | ZP_00293744.1; CAF34426.1; |
| | | | YP_055934.1; EAM93810.1; |
| | | | NP_214976.1; NP_975267.1; |
| | | | NP_701672.1; NP_662186.1; |
| | | | AAG12404.1; NP_390286.1; |
| | | | AAB96096.1; BAB64316.1; |
| | | | YP_075992.1; ZP_00656450.1; |
| | | | ZP_00413985.1 |
| thiosulfate | reduction of | NP_461008 | AAL20967.1 NP_804652.1 AAX65978.1 |
| reductase | thiosulfate | | YP_150111.1 AAA68433.1 NP_753959.1 |
| (R73) | to sulfide | | AAG56657.1 NP_707568.1 AAC74740.1 |
| phsA | | | AAA68434.1 NP_951651.1 YP_012355.1 |
| | | | ZP_00532396.1 ZP_00591595.1 |
| | | | ZP_00588551.1 ZP_00535768.1 |
| | | | ZP_00552446.1 ZP_00511962.1 |
| | | | ZP_00528880.1 EAN28924.1 |
| | | | ZP_00303670.1 |
| thiosulfate | reduction of | NP_461009 | YP_150110.1; AAX65979.1 |
| reductase | thiosulfate | | AAA68432.1 ZP_00585996.1; NP_719591.1 |
| (R73) | to sulfide | | AAC74741.1; P77375; ZP_00639669.1 |
| PhsB | electron | | NP_753960.1; NP_837355.1; Q8X616 |
| | transport | | AAG56658.1; NP_707569.1 |
| | protein | | ZP_00583250.1 |
| | | | YP_129473.1; AAO11357.1; NP_934045.1 |
| | | | CAG73826.1; CAA46177.1; NP_709848.1 |
| | | | NP_756920.1; YP_204935.1 |
| | | | ZP_00585235.1 |
| thiosulfate | thiosulfate | NP_461010 | AAL20969.1; AAX65980.1; NP_804650.1; |
| reductase | reductase | | NP_719592.1; ZP_00639668.1; ZP_00585997.1; |
| (R73) | precursor | | CAA46176.1; CAE09281.1; ZP_00575008.1; |
| PhsC | | | ZP_00401980.1; YP_009398.1; ZP_00130114.2; |
| | | | YP_004130.1; ZP_00130395.1; |
| | | | CAE09834.1; ZP_00583252.1; ZP_00583251.1; |
| | | | ZP_00550659.1; ZP_00534062.1; ZP_00529653.1; |
| | | | NP_070031.1; ZP_00531012.1; |
| | | | ZP_00511630.1; ZP_00557320.1; ZP_00583253.1; |
| | | | ZP_00589338.1; ZP_00592921.1; YP_073938.1; |
| | | | ZP_00550240.1; ZP_00558485.1; |
| | | | ZP_00537210.1; YP_157558.1; YP_160703.1; |
| | | | AAL64489.1; EAN27258.1; ZP_00550224.1; |
| | | | AAQ08379.1; CAB71267.1; ZP_00557316.1; |
| | | | ZP_00575750.1; ZP_00150980.1; |
| | | | ZP_00559031.1; ZP_00558228.1; ZP_00557853.1; |
| | | | YP_132003.1; CAE10491.1; YP_206040.1; |
| | | | NP_668198.1; CAC92557.1; YP_069346.1; |
| | | | AAC74659.1; AAK03838.1; ZP_00557324.1; |
| | | | CAE11227.1; AAX88061.1; ZP_00155680.2; |
| | | | NP_439206.1; ZP_00557867.1; |
| | | | AAG56574.1; ZP_00156893.2; NP_753872.1; |
| | | | AAB06233.1; ZP_00557301.1; NP_805734.1; |
| | | | ZP_00053862.2; AAL21424.1; NP_752960.1; |
| | | | AAX64824.1; AAL19899.1; NP_309006.2; |
| | | | CAB50383.1; P18775; EAM94677.1; |
| | | | ZP_00550524.1; AAX66433.1; AAL19562.1; |
| | | | ZP_00600783.1; BAB34402.1; AAC73980.1; |
| | | | YP_074347.1; AAG55381.1; NP_836552.1; |
| | | | NP_753873.1; ZP_00509629.1; EAM95020.1; |
| | | | AAG56575.1; ZP_00557953.1; |
| | | | BAB35717.1; AAX64548.1; CAE09880.1; |
| | | | AAX65422.1; AAC74660.1; P77783; YP_150613.1; |
| | | | ZP_00550244.1; AAL20417.1; NP_805214.1; |
| | | | YP_075885.1; NP_805215.1; AAX65421.1; |
| | | | NP_106243.1; YP_151327.1; AAX68089.1; |
| | | | BAB36807.1; ZP_00559319.1; |
| | | | AAG57632.1; AAL23129.1; CAB49710.1; |
| | | | NP_463170.2; CAG74160.1; BAB65038.1; |
| | | | YP_160885.1; YP_079335.1; NP_805998.1; |
| | | | YP_119157.1;; |
| anaerobic | converts | AAL21442 | YP_149649.1; NP_804183.1; AAX66448.1; |
| sulfite | sulfite to | | AAK79480.1; BAB81146.1; AAO36949.1; |
| reductase | sulfide | | ZP_00576801.1; BAB81244.1; ZP_00145179.1; |
| subunit A | | | ZP_00662254.1; ZP_00575186.1; ZP_00536228.1; |
| R(74) | | | BAD86261.1; AAL81453.1; NP_143176.1; |
| DsrA | | | ZP_00667024.1; NP_951149.1; |
| | | | CAA53034.1; AAL81015.1; YP_011613.1; |
| | | | ZP_00588237.1; NP_662768.1; ZP_00511990.1; |
| | | | CAB49782.1; NP_110522.1; CAC11194.1; |
| | | | ZP_00130780.1; ZP_00528291.1; ZP_00590992.1; |
| | | | AAU82616.1; ZP_00532275.1; |
| | | | ZP_00347064.1; EAN28921.1; ZP_00547737.1; |
| | | | AAB94933.1; YP_096477.1; ZP_00335593.1; |
| | | | YP_124840.1; CAB49860.1; YP_127720.1; |
| | | | ZP_00416523.1; NP_662137.1; ZP_00661877.1; |
| | | | ZP_00588474.1; ZP_00511238.1; |
| | | | EAO34975.1; ZP_00528312.1; NP_147088.1; |
| | | | ZP_00346179.1; NP_246968.1; AAG53710.1; |
| | | | NP_632708.1; AAC37042.1; ZP_00582131.1; |
| | | | NP_937321.1; AAO07727.1; XP_652137.1; |
| | | | AAM94492.1; ZP_00536224.1; |
| | | | ZP_00557435.1; NP_701624.1; AAU06262.1; |
| | | | NP_880822.1; NP_889860.1; AAU83862.1 |
| anaerobic | converts | AAL21443 | YP_149648.1; NP_804182.1; AAX66449.1; |
| sulfite | sulfite to | | AAA99276.1; ZP_00576802.1; BAB81243.1; |
| reductase | sulfide | | BAB81145.1; AAK79481.1; AAO36948.1; |
| subunit B (r74) | | | ZP_00575185.1; YP_011612.1; CAC11195.1; |
| DsrB | | | ZP_00145178.1; ZP_00662255.1; NP_110523.1; |
| | | | EAN28920.1; ZP_00588238.1; ZP_00130781.1; |
| | | | NP_951147.1; ZP_00667025.1; |
| | | | AAB94934.1; ZP_00347063.1; EAO34974.1; |
| | | | CAA53035.1; AAL81016.1; BAD86260.1; |
| | | | ZP_00536229.1; ZP_00590993.1; CAB49781.1; |
| | | | CAB49861.1; ZP_00511991.1; NP_143177.1; |
| | | | ZP_00416521.1; ZP_00528292.1; |
| | | | AAL81454.1; NP_662769.1; YP_124839.1; |
| | | | YP_127719.1; ZP_00532276.1; YP_096476.1; |
| | | | ZP_00511237.1; ZP_00528313.1; ZP_00661878.1; |
| | | | AAU82615.1; ZP_00588475.1; |
| | | | NP_662138.1; ZP_00335592.1; ZP_00547738.1; |
| | | | ZP_00417715.1; CAB50658.1; BAD85995.1; |
| | | | AAL80312.1; ZP_00562249.1; O57738; |
| | | | ZP_00541622.1; NP_143791.1; AAM04028.1; |
| | | | YP_022952.1; AAA23200.2; NP_633770.1; |
| | | | NP_988039.1; NP_971592.1; NP_111690.1; |
| | | | CAC11546.1; NP_069364.1; NP_248450.1; |
| | | | NP_632687.1; AAT38120.1; AAQ66178.1; |
| | | | AAM18706.1; AAC65704.1; BAB80961.1; |
| | | | NP_971022.1; NP_613849.1; NP_622237.1; |
| | | | AAK80598.1; NP_622352.1; AAB85701.1; |
| | | | YP_147006.1; CAA51740.1; AAM07137.1; |
| | | | AAN58909.1; NP_229439.1; EAM94068.1; |
| | | | NP_267503.1; CAG37745.1; NP_623138.1; |
| | | | AAO36853.1; ZP_00541900.1; |
| | | | AAL94626.1; ZP_00563837.1; ZP_00539594.1; |
| | | | AAO36887.1; CAB49859.1; Q8XL63; |
| | | | AAP10806.1; NP_389436.1; YP_020666.1; |
| | | | YP_078946.1; ZP_00143318.1; AAK99669.1; |
| | | | Q8DQ38; NP_816202.1; NP_345444.1; NP_692413.1; |
| | | | ZP_00240193.1; YP_011687.1; |
| | | | ZP_00504646.1; ZP_00575869.1; AAO35521.1; |
| | | | YP_055711.1; NP_758176.1; P56968; |
| | | | 1EP2; ZP_00520109.1; NP_815421.1; YP_181916.1; |
| | | | ZP_00575375.1; YP_180792.1; YP_175828.1; |
| | | | ZP_00382098.1; ZP_00130793.1; |
| | | | ZP_00401561.1; ZP_00561001.1; ZP_00655811.1; |
| | | | NP_012221.1; AAO75998.1; NP_952806.1; |
| | | | AAQ97765.1; AAL81452.1; AAS51833.1; |
| | | | ZP_00128609.1; NP_531891.1; CAE71880.1; |
| | | | NP_946031.1; CAH00358.1; CAA37672.1; |
| | | | CAG35490.1; AAN68771.1; AAV52085.1; |
| | | | AAV62537.1; ZP_00389517.1; ZP_00535043.1; |
| | | | XP_550297.1; ZP_00664777.1; |
| | | | NP_465359.1; CAD14793.1; XP_330548.1; |
| | | | AAN15927.1; ZP_00234145.1; P23312; |
| | | | AAA18377.1; ZP_00591027.1; CAI47849.1; |
| | | | AAA67175.1; BAA13047.1; AAA72422.1; |
| | | | ZP_00503086.1; NP_057313.2; ZP_00401594.1; |
| | | | P39866; XP_396639.1; XP_623086.1; |
| | | | BAB55002.1; P39870; AAB66010.1; NP_471282.1; |
| | | | EAA51298.1;; EAN09251.1; CAA56696.1; |
| | | | AAC69483.1; AAC49460.1; AAF63450.1; |
| | | | ZP_00505244.1; CAA32217.1; AAU84695.1; |
| | | | AAF04811.1; AAB93308.1; NP_796190.1; |
| | | | CAH08182.1; AAG30576.1; AAB39554.1 |
| anaerobic | converts | NP_804181 | YP_149647.1; AAA99277.1; BAB81242.1; |
| sulfite | sulfite to | | AAO36947.1; BAB81144.1; AAK79482.1; |
| reductase | sulfide | | ZP_00576803.1; CAA60228.1; NP_952404.1; |
| subunit C | | | AAM06806.1; ZP_00542975.1; ZP_00503774.1; |
| (R74) | | | ZP_00561650.1; ZP_00560787.1; ZP_00535294.1; |
| DsrC | | | NP_635288.1; ZP_00145130.1; |
| | | | ZP_00576896.1; ZP_00563866.1; NP_247865.1; |
| | | | AAU83232.1; AAO35782.1; NP_987198.1; |
| | | | NP_614083.1; AAB84786.1; AAM06538.1; |
| | | | ZP_00540799.1; NP_632386.1; AAM04125.1; |
| | | | ZP_00558670.1; NP_614085.1; |
| | | | ZP_00130988.2; AAM06540.1; NP_632384.1; |
| | | | NP_633866.1; ZP_00631188.1; ZP_00541754.1; |
| | | | EAN05933.1; ZP_00056315.1; ZP_00562004.1; |
| | | | ZP_00667805.1; YP_147721.1; |
| | | | AAK49018.1; AAC17127.1; NP_534394.1; |
| | | | AAK89517.1; BAB92078.1; NP_104101.1; |
| | | | AAQ18184.1; AAM73544.1; P17847; AAA60450.1; |
| | | | NP_247530.1; ZP_00623963.1; |
| | | | AAK22600.1; ZP_00303419.1; ZP_00536025.1; |
| | | | AAP46170.1; NP_442378.1; NP_954306.1; |
| | | | CAC49509.1; AAP79144.1; BAD15364.1; |
| | | | ZP_00576654.1; ZP_00575700.1; CAG36393.1; |
| | | | ZP_00579652.1; NP_918873.1;; ZP_00556586.1; |
| | | | ZP_00333573.1; CAA46940.1; |
| | | | BAD53072.1; CAA46942.1; BAD15363.1; |
| | | | CAA34893.1; ZP_00558945.1; NP_952142.1; |
| | | | BAD15365.1; ZP_00535147.1; NP_924503.1; |
| | | | BAE06055.1; BAB55003.1; ZP_00415177.1; |
| | | | YP_036299.1; AAM06247.1; ZP_00392410.1; |
| | | | ZP_00674499.1; EAM75787.1; |
| | | | AAO38372.1; YP_018789.1; AAN31831.1; |
| | | | AAN31830.1; AAN13223.1; BAD93723.1; |
| | | | YP_083541.1; ZP_00575701.1; BAA06530.1; |
| | | | ZP_00571935.1; ZP_00534536.1; AAP09103.1; |
| | | | YP_010301.1; ZP_00500892.1; ZP_00496099.1; |
| | | | ZP_00486991.1; ZP_00482951.1; |
| | | | ZP_00467711.1; ZP_00452175.1; YP_104614.1; |
| | | | ZP_00645283.1; CAH34502.1; |
| | | | ZP_00237633.1; ZP_00516217.1; BAB04332.1; |
| | | | CAA42690.1; NP_632080.1; CAG35527.1; |
| | | | AAU83223.1; ZP_00544005.1; EAO35589.1; |
| | | | EAL90616.1; AAB09032.1; ZP_00575732.1; |
| | | | ZP_00558782.1; NP_250472.1; NP_637372.1; |
| | | | ZP_00139438.2; ZP_00107422.2; |
| | | | ZP_00294167.1; ZP_00265695.1; CAG36396.1; |
| | | | EAA65575.1; ZP_00149626.2; ZP_00549111.1; |
| | | | ZP_00413634.1; ZP_00525511.1; |
| | | | YP_157671.1; YP_077721.1; NP_229093.1; |
| | | | CAF19045.1; YP_120776.1; ZP_00242120.1; |
| | | | ZP_00507972.1; ZP_00667812.1; NP_388212.1;; |
| | | | ZP_00653668.1; YP_257799.1; |
| | | | CAE22413.1; P22944; ZP_00537258.1; YP_041840.1; |
| | | | YP_187201.1; NP_372924.1; CAG44104.1; |
| | | | BAC79016.1; NP_771211.1; YP_175117.1; |
| | | | CAG75891.1; CAD29755.1; ZP_00379608.1; |
| | | | AAL64294.1; AAC17122.1; |
| | | | ZP_00162550.1; EAO35459.1; AAD20825.1; |
| | | | CAC06095.1; AAC46074.1; ZP_00563605.1; |
| | | | ZP_00268907.1; NP_522783.1; NP_882747.1; |
| | | | NP_886946.1; ZP_00281075.1; NP_765533.1; |
| | | | YP_189546.1; CAF32236.1; ZP_00169751.1; |
| | | | ZP_00535570.1; YP_171020.1; |
| | | | ZP_00563990.1; ZP_00402377.1; NP_252819.1; |
| | | | AAV68379.1; NP_883424.1; YP_252566.1; |
| | | | NP_887867.1; AAC46135.1; ZP_00205156.1; |
| | | | ZP_00541555.1; ZP_00592076.1; YP_009627.1; |
| | | | ZP_00493035.1; ZP_00467311.1; |
| | | | ZP_00450531.1; ZP_00411899.1; NP_613536.1; |
| | | | YP_111251.1; NP_285337.1; YP_011484.1; |
| | | | YP_105749.1; NP_987944.1; AAB84911.1; |
| | | | CAC09931.1; NP_768957.1; Q8TYP4; |
| | | | CAA79655.1; AAB28156.1; ZP_00529166.1; |
| | | | NP_069756.1; YP_010816.1; AAM18137.1; |
| | | | NP_961142.1; AAA23383.1; ZP_00567851.1; |
| | | | ZP_00397695.1; AAP08405.1; |
| | | | NP_216907.1; YP_113108.1; NP_613552.1; |
| | | | NP_070472.1; NP_635324.1; CAE08992.1; |
| | | | AAO36006.1; AAK46756.1; 1ZJ9; AAB50233.1; |
| | | | ZP_00532427.1; ZP_00521920.1; AAO07346.1; |
| | | | NP_937004.1; NP_952492.1; AAO61105.1; |
| | | | YP_011615.1; NP_881958.1; CAB69775.1; |
| | | | ZP_00215253.1; ZP_00130392.2; |
| | | | ZP_00130682.2; ZP_00131127.1; ZP_00592685.1; |
| | | | ZP_00558070.1; ZP_00546885.1; |
| | | | ZP_00465281.1; ZP_00454984.1; ZP_00668317.1; |
| | | | ZP_00665867.1; NP_614767.1; CAA43512.1; |
| | | | AAU83053.1; NP_248188.1; NP_682139.1; |
| | | | CAA40717.1; ZP_00510136.1; |
| | | | YP_018067.1;; ZP_00563478.1; ZP_00661349.1; |
| | | | YP_045466.1; AAV68654.1; YP_035640.1; |
| | | | NP_971884.1; CAC33947.1; ZP_00048074.2; |
| | | | ZP_00050155.1; AAM03878.1; AAK81453.1; |
| | | | AAC78310.1; CAA86992.1; ZP_00497750.1; |
| | | | EAO36822.1; AAO38151.1; |
| | | | NP_709140.2; NP_633643.1; NP_953151.1; |
| | | | CAA32416.1; AAT47760.1; AAU83339.1; |
| | | | AAT99257.1; BAC73373.1; NP_247490.1; |
| | | | AAC76390.1; AAK78079.1; AAG58473.1; |
| | | | BAB37639.1; ZP_00541003.1; ZP_00503951.1; |
| | | | ZP_00417521.1; ZP_00265988.1; ZP_00621912.1; |
| | | | P00202; NP_614213.1; NP_068995.1; |
| | | | NP_800565.1; AAB85622.1; AAM07522.1; |
| | | | AAF11421.1; AAB02352.1; YP_082906.1; |
| | | | ZP_00166363.1; ZP_00560684.1; |
| | | | ZP_00510774.1; EAM24821.1; ZP_00551813.1; |
| | | | ZP_00667348.1; NP_621874.1; YP_266101.1; |
| | | | NP_794611.1; CAA76373.1; CAA76342.1; |
| | | | CAA08858.1; AAK78015.1; AAC47160.1; |
| | | | BAB80366.1; ZP_00562782.1; ZP_00544290.1; |
| | | | AAZ18451.1;; ZP_00511163.1; |
| | | | ZP_00462654.1; ZP_00653977.1; NP_214766.1; |
| | | | NP_716642.1; YP_259653.1; YP_012009.1; |
| | | | YP_117628.1; CAG37108.1; AAU95491.1; |
| | | | CAC39231.1; CAB95043.1; AAM92180.1; |
| | | | ZP_00237342.1; AAK23103.1; NP_739254.1; |
| | | | CAC19472.1; NP_977868.1; AAS07951.1; |
| | | | ZP_00217308.1; ZP_00565232.1; |
| | | | ZP_00541377.1; NP_949048.1; ZP_00267392.1; |
| | | | ZP_00419332.1; ZP_00513264.1; ZP_00486799.1; |
| | | | EAO38190.1; ZP_00397962.1; |
| | | | YP_276576.1; AAV94843.1; AAV94139.1; |
| | | | NP_622539.1; NP_799995.1; NP_440189.1; |
| | | | CAA74092.1; ZP_00054379.1; AAK81291.1; |
| | | | BAB96809.1; AAQ21342.1; AAY20996.1; |
| | | | ZP_00130766.1; ZP_00108506.2; ZP_00541311.1; |
| | | | ZP_00542829.1; EAM28721.1; |
| | | | ZP_00658370.1; AAP99874.1; YP_132435.1; |
| | | | NP_808180.1; NP_777779.1; NP_229096.1; |
| | | | AAO61114.1; AAL23380.1; CAG36553.1; |
| | | | AAU95489.1; CAD16132.1; ZP_00053119.1; |
| | | | AAM21749.1; ZP_00128864.2; ZP_00593542.1; |
| | | | ZP_00574875.1; ZP_00575487.1; |
| | | | ZP_00543494.1; CAA48368.1; ZP_00595533.1; |
| | | | ZP_00557356.1; ZP_00435740.1; AAO08247.1; |
| | | | EAA71154.1; EAO35986.1; YP_153414.1; |
| | | | YP_149857.1; NP_708163.1; |
| | | | NP_936256.1; NP_614526.1; YP_071090.1; |
| | | | YP_046560.1; AAO61117.1; AAO61110.1; |
| | | | CAG75920.1; CAA11230.1; CAA46941.1; |
| | | | AAV47410.1; CAA92206.1; AAC47159.1; |
| | | | BAA16109.1; ZP_00356596.1; ZP_00345938.1; |
| | | | ZP_00576139.1; ZP_00426552.1; ZP_00263533.1; |
| | | | EAN06682.1; AAY38236.1; EAM23497.1; |
| | | | ZP_00504882.1; ZP_00498390.1; |
| | | | ZP_00493060.1; ZP_00469245.1; ZP_00450851.1; |
| | | | ZP_00668314.1; YP_275286.1; |
| | | | YP_146312.1; NP_613608.1; AAZ15776.1; |
| | | | NP_251334.1; NP_632784.1; YP_102259.1; |
| | | | NP_793155.1; XP_759995.1; YP_261004.1; |
| | | | CAH09344.1; CAC41649.1; ZP_00056531.1; |
| | | | AAO75724.1; AAM22202.1; AAL89571.1; |
| | | | AAN69709.1; AAT50267.1; AAU14235.1; |
| | | | YP_101168.1; ZP_00576374.1; ZP_00564969.1; |
| | | | ZP_00540769.1; ZP_00504835.1; |
| | | | P38681; Q01700; ZP_00397512.1; ZP_00397295.1; |
| | | | YP_174119.1; AAO38143.1; NP_707572.1; |
| | | | NP_623466.1; YP_221069.1; NP_069003.1; |
| | | | NP_251596.1; NP_800497.1; NP_988812.1; |
| | | | NP_753963.1; AAN29231.1; CAD71547.1; |
| | | | BAD84891.1; AAL52820.1; AAB85352.1; |
| | | | NP_962636.1; XP_324077.1; AAG23566.1; |
| | | | AAF87215.1; BAB55574.1; ZP_00562084.1; |
| | | | ZP_00563739.1; ZP_00540990.1; |
| | | | ZP_00543828.1; ZP_00537084.1; ZP_00537423.1; |
| | | | CAA42917.1; Q59110; AAQ79821.1; |
| | | | AAD54888.1; YP_074057.1; NP_069260.1; |
| | | | NP_249130.1; NP_634587.1; NP_954288.1; |
| | | | NP_952717.1; AAO61104.1; AAV68690.1; |
| | | | AAU95493.1; CAB95039.1 |
| glucose-6- | supply of | BAB98969 | NP_738306.1; NP_939657.1; CAI37158.1; |
| phopshate | reduction | | YP_119787.1; NP_215963.1; |
| dehydrogenase | equivalents | | NP_960110.1; BAC74024.1; |
| (R3 | for | | EAM76742.1; CAB50762.1; |
| | biochemical | | BAC69479.1;; CAE53636.1; |
| | reductions | | YP_062112.1;; ZP_00294054.1; |
| | such as | | ZP_00658920.1; ZP_00413483.1; |
| | Sulfate to | | CAA19940.1;; NP_695641.1;; |
| | sulfide | | ZP_00120909.1; YP_056264.1;; |
| | reductions | | ZP_00569429.1; ZP_00548313.1; |
| | or | | ZP_00600254.1; ZP_00357971.1; |
| | methylene | | CAD28141.1; NP_926124.1;; |
| | tetrathydrofolate | | AAO44440.1;; ZP_00395318.1; |
| | to | | YP_172478.1;; ZP_00121962.1; |
| | methyl THF | | NP_681330.1;; ZP_00326212.1; |
| | | | NP_440771.1;; P29686; AAF11158.1;; |
| | | | ZP_00516458.1; ZP_00519749.1; |
| | | | ZP_00536369.1; P48848; CAF23545.1;; |
| | | | AAP98177.1;; P48992;; ZP_00160727.1; |
| | | | AAA98853.1; AAF73556.1;; |
| | | | NP_924116.1;; AAB41225.1; |
| | | | NP_893191.1;; NP_622656.1;; |
| | | | ZP_00623650.1; CAE21277.1;; |
| | | | AAQ00169.1;; CAE07265.1;; |
| | | | AAU36623.1;; YP_219937.1;; |
| | | | ZP_00399101.1; NP_639332.1;; |
| | | | EAN05140.1; NP_228961.1;; P77809; |
| | | | ZP_00135250.2; AAN03818.1; |
| | | | AAP05283.1;; AAM38916.1;; |
| | | | ZP_00574687.1; NP_948974.1;; |
| | | | YP_199106.1;; ZP_00131780.2; |
| | | | ZP_00123638.1; NP_773400.1;; |
| | | | YP_129655.1;; YP_236060.1;; |
| | | | AAK03633.1;; AAP95731.1;; |
| | | | CAD72806.1;; NP_792913.1;; |
| | | | YP_223238.1;; NP_541491.1;; |
| | | | AAX87606.1;; NP_531301.1;; |
| | | | AAN33959.1;; AAK86411.1;; |
| | | | NP_438715.1;; ZP_00156377.2; |
| | | | ZP_00265291.1; ZP_00416738.1; |
| | | | CAC45276.1;; AAN69632.1;; |
| | | | AAD12043.1; AAC65465.1;; |
| | | | ZP_00128741.1; YP_260249.1;; |
| | | | AAO76328.1;; AAN70916.1;; |
| | | | YP_014595.1;; NP_471419.1;; |
| | | | CAG75379.1;; YP_115360.1;; |
| | | | NP_840485.1;; CAH07618.1;; |
| | | | NP_219689.1;; YP_070566.1;; |
| | | | NP_465502.1;; NP_798089.1;; |
| | | | AAQ57824.1;; NP_107009.1;; |
| | | | ZP_00596515.1; ZP_00278393.1; |
| | | | NP_669553.1;; CAA52858.1; |
| | | | CAC90878.1;; NP_754157.1;; |
| | | | AAG56842.1;; YP_150270.1;; |
| | | | NP_929382.1;; AAX65797.1;; |
| | | | NP_934398.1;; NP_837434.1;; |
| | | | YP_206426.1;; AAO11031.1;; |
| | | | EAO17699.1; ZP_00637092.1; |
| | | | NP_804814.1;; AAW29927.1; |
| | | | ZP_00005413.2; YP_112566.1;; |
| | | | AAW29929.1; AAV96269.1;; |
| | | | YP_079709.1;; AAL14620.1; |
| | | | ZP_00498362.1; ZP_00472038.1; |
| | | | AAW29926.1; ZP_00633694.1; |
| | | | YP_148187.1;; AAA24775.1; |
| | | | ZP_00587566.1; ZP_00446786.1; |
| | | | NP_718076.1;; NP_814740.1;; |
| | | | ZP_00350648.1; NP_254126.1;; |
| | | | AAF96793.1;; NP_390266.1;; |
| | | | ZP_00423718.1; ZP_00583219.1; |
| | | | ZP_00303587.1; EAN10723.1; |
| | | | ZP_00217377.1; ZP_00568967.1; |
| | | | ZP_00455268.1; YP_269001.1; |
| | | | YP_175420.1;; NP_786078.1;; |
| | | | CAF25793.1;; NP_251873.1;; |
| | | | ZP_00334099.1; ZP_00280824.1; |
| | | | ZP_00218486.1; ZP_00166002.1; |
| | | | ZP_00579866.1; ZP_00316750.1; |
| | | | AAV95319.1;; ZP_00264504.1; |
| | | | ZP_00555465.1; ZP_00462564.1; |
| | | | NP_660655.1;; ZP_00152367.1; |
| | | | BAA90547.1; ZP_00629685.1; |
| | | | ZP_00384027.1; ZP_00285042.1; |
| | | | NP_693860.1;; EAM33140.1; |
| | | | YP_033231.1;; NP_662750.1;; |
| | | | AAN66647.1;; YP_234212.1;; |
| | | | NP_878735.1;; YP_261694.1;; |
| | | | AAB91531.1;; NP_791129.1;; |
| | | | CAC14908.1; AAU07486.1;; |
| | | | ZP_00509653.1; ZP_00136528.2; |
| | | | ZP_00620998.1; NP_268377.1;; |
| | | | ZP_00643038.1; ZP_00564303.1; |
| | | | CAJ07708.1; AAK24030.1;; |
| | | | CAB84837.1;; EAO18485.1; |
| | | | YP_188644.1;; ZP_00415411.1; |
| | | | AAF41756.1;; ZP_00416202.1; |
| | | | NP_764743.1;; NP_523118.1;; |
| | | | YP_040979.1;; ZP_00588223.1; |
| | | | ZP_00590972.1; YP_037489.1;; |
| | | | AAM64291.1;; YP_084670.1;; |
| | | | ZP_00235566.1; AAL76389.1; |
| | | | YP_029439.1;; ZP_00530891.1; |
| | | | YP_020068.2;; ZP_00377677.1; |
| | | | ZP_00419457.1; YP_125825.1;; |
| | | | YP_094460.1;; YP_122821.1;; |
| | | | ZP_00528275.1; ZP_00660845.1; |
| | | | AAM64228.1; AAW89435.1;; |
| | | | CAA59012.1; ZP_00051756.1; |
| | | | NP_979712.1;; P21907; NP_240142.1;; |
| | | | CAB52708.1; AAO37825.1; |
| | | | CAA54841.1; Q42919; EAL92729.1;; |
| | | | YP_253326.1;; CAB52675.1; |
| | | | CAA04696.1; AAO42879.1;; |
| | | | ZP_00385429.1; BAC23041.1; |
| | | | AAD11426.1; AAO36382.1;; |
| | | | YP_190594.1;; BAA97662.1; |
| | | | CAA03939.1; BAA97664.1; AAF87216.1; |
| | | | AAM64230.1; BAB96757.1; |
| | | | CAA67782.1; XP_468660.1;; |
| | | | BAA97663.1; CAA61194.1; |
| | | | AAQ02671.1; XP_477654.1;; |
| | | | CAA58825.1; CAA54840.1; |
| | | | CAA59011.1; CAA97412.1;; |
| | | | CAC05439.1; NP_196815.2;; |
| | | | CAE62054.1; XP_466575.1;; |
| | | | AAB69317.1; CAA52442.1; |
| | | | CAA04994.1; AAL57678.1; CAB52674.1; |
| | | | CAA04993.1; BAB02125.1;; |
| | | | ZP_00323827.1; CAA58775.1; |
| | | | AAM98087.1; BAD08586.1; |
| | | | NP_173838.1;; AAK99925.1;; |
| | | | EAN98209.1; NP_345708.1;; |
| | | | EAN77674.1; AAL57688.1; AAB25541.1; |
| | | | ZP_00511972.1; XP_644814.1;; |
| | | | XP_472942.1;; CAC07816.1; |
| | | | CAA04992.1; AAB69319.1; |
| | | | CAB52681.1; NP_777918.1;; |
| | | | AAW44738.1;; EAL04742.1; |
| | | | EAL04547.1; EAA46705.1;; |
| | | | ZP_00659395.1; EAA70588.1;; |
| | | | ZP_00332697.1; CAB52685.1; |
| | | | AAS50565.1; AAB69318.1; AAZ23850.1; |
| | | | AAL79959.1; AAW24823.1; |
| | | | CAA49834.1; CAG86200.1;; |
| | | | XP_761077.1; AAA34619.1; |
| | | | ZP_00110439.1; CAG07451.1; |
| | | | NP_535313.1;; NP_014158.1;; |
| | | | AAT93017.1; O55044; NP_001017312.1;; |
| | | | CAG79872.1;; NP_032088.1;; Q00612;; |
| | | | AAH59324.1;; BAD17912.1; |
| | | | XP_331503.1;; ZP_00110078.1; |
| | | | NP_058702.1;; XP_311452.2;; Q29492; |
| | | | NP_105132.1;; ZP_00063705.1; |
| | | | BAD17947.1; CAG60989.1;; P11413;; |
| | | | AAP36661.1; AAL27011.1;; 2BHL;; |
| | | | ZP_00161394.2; NP_000393.2;; 1QKI;; |
| | | | AAA92653.1;; AAA52500.1;; |
| | | | XP_538209.1;; ZP_00644450.1; |
| | | | AAA41179.1; AAA63175.1;; |
| | | | XP_307095.2;; CAA03941.1; |
| | | | XP_699168.1;; AAN76409.1;; |
| | | | ZP_00319846.1; AAN76408.1;; |
| | | | AAW82643.1; AAN76413.1;; |
| | | | AAB29395.1; BAD17951.1; Q27638; |
| | | | EAL31619.1; CAB57419.1; BAD94743.1; |
| | | | P11411; NP_062341.1;; BAD17934.1; |
| | | | 1H9B;; 1H94;; 1DPG;; AAB96363.1; |
| | | | BAD17891.1; BAD17877.1; |
| | | | BAD17941.1; BAD17905.1; 1E7Y;; |
| | | | CAA58590.2; AAF48999.1;; |
| | | | AAB02812.1; AAB02811.1; AAF49000.2;; |
| | | | BAD17954.1; 2DPG;; NP_961605.1;; |
| | | | AAB02809.1; AAA99073.1; |
| | | | AAK45410.1;; AAK93503.1; |
| | | | BAD17927.1; BAD17920.1; |
| | | | YP_177789.1;; BAD17898.1; |
| | | | NP_637497.1;; YP_193335.1;; |
| | | | BAD17884.1; YP_200953.1;; |
| | | | AAM36928.1;; AAF19030.2; |
| | | | ZP_00464292.1; CAB08746.1;; |
| | | | AAM51346.1;; XP_579385.1; |
| | | | AAA51463.1; AAM64231.1; |
| | | | ZP_00404158.1; NP_778577.1;; |
| | | | AAC33202.1; NP_298355.1;; |
| | | | ZP_00651890.1; CAD28863.1; |
| | | | AAM64229.1; CAD28862.1; |
| | | | CAD43148.1; ZP_00046060.1; |
| | | | NP_964496.1;; ZP_00387215.1; |
| | | | XP_583628.1;; XP_559252.1;; |
| | | | AAA52499.1; CAD97761.1; |
| | | | AAR12945.1; AAR12953.1; |
| | | | AAR12952.1; AAR12943.1; |
| | | | AAR12946.1; CAG04059.1; |
| | | | ZP_00413080.1; CAA19129.1;; |
| | | | CAA03940.1; NP_960621.1;; |
| | | | EAM75831.1; CAB16743.1;; |
| | | | CAE51228.1; CAE51222.1; AAG28730.1; |
| | | | AAG28728.1; AAA57029.1; |
| | | | AAA57025.1; CAE51229.1; |
| | | | ZP_00380754.1; EAM75226.1; |
| | | | AAO19918.1; AAO19916.1; |
| | | | AAO19914.1; AAO19917.1; |
| | | | XP_769603.1; CAC24715.1; |
| | | | EAA18517.1; NP_213347.1;; |
| | | | AAR26303.1; XP_680237.1;; |
| | | | AAA65930.1;; NP_702400.1;; |
| | | | NP_223744.1;; AAD08144.1;; |
| | | | EAN31303.1;; NP_649376.2;; |
| | | | EAN81514.1; ZP_00048966.1; |
| | | | AAG23802.1; CAI75777.1; AAV37033.1; |
| | | | NP_737152.1;; AAF24764.1; |
| | | | XP_233688.3;; AAS87299.1; |
| | | | NP_775547.2;; AAH42677.1;; |
| | | | CAH18137.1;; XP_425746.1;; |
| | | | CAC27532.1; BAA82155.1; |
| | | | AAH81559.1;; NP_004276.2;; |
| | | | CAA10071.1;; ZP_00131371.2; |
| | | | AAN06152.1; P56201; XP_697820.1;; |
| | | | AAN06169.1; AAU95204.1; |
| | | | AAC08804.1; AAC08813.1; |
| | | | CAG06984.1; AAC08802.1; |
| | | | AAD35084.1; AAW81980.1;; |
| | | | ZP_00572395.1; CAA45220.1; |
| | | | AAP44069.1; AAP44068.1; AAP44065.1; |
| | | | AAP44063.1; AAN06140.1; AAP44061.1; |
| | | | AAP44072.1; AAP44078.1; EAN85689.1; |
| | | | AAP44081.1; AAP44076.1; AAP44066.1; |
| | | | AAP44070.1; AAP44079.1; AAP44077.1; |
| | | | AAP44071.1; AAP44080.1; AAP44062.1; |
| | | | XP_744368.1;; AAP44073.11 |
| transketolase | pentose- | YP_225858 | NP_738304.1; NP_939655.1; CAI37156.1; |
| (R8) | phosphate | | YP_119785.1; NP_9601121; NP_301494.1; |
| | pathway, | | NP_855136.1; AAK45759.1; NP_215965.1; |
| | supply of | | ZP_00381421.1; ZP_00413480.1; YP_062115.1; |
| | reduction | | EAM76740.1; ZP_00294057.1; CAB50760.1; |
| | equivalents | | BAC74026.1; BAC69477.1; CAA19942.1; |
| | for | | AAG12171.2; YP_056980.1; NP_789362.1; |
| | biochemical | | AAO44437.1; NP_695899.1; YP_147185.1; |
| | reductions | | AAP10611.1; ZP_00239892.1; NP_980015.1; |
| | such as | | YP_013921.1; NP_464830.1; NP_470679.1; |
| | sulfate to | | YP_037757.1; ZP_00234507.1; |
| | sulfide | | YP_020383.1; YP_084972.1; Q9KAD7; NP_692593.1; |
| | reductions | | NP_621887.1; ZP_00106110.1; |
| | or | | YP_171693.1; ZP_00539439.1; ZP_00396639.1; |
| | methylene | | ZP_00393910.1; AAR39402.1; YP_143374.1; |
| | tetrathydrofolate | | YP_079202.1; YP_005865.1; NP_371866.1; |
| | to | | YP_175660.1; YP_040758.1; NP_440630.1; |
| | methyl THF | | CAA75777.1; ZP_00163127.2; YP_192099.1; |
| | | | NP_389672.1; ZP_00464976.1; |
| | | | BAD08582.1 |
| | | | BAB75043.1; NP_979711.1; EAN28966.1; |
| | | | ZP_00590971.1; YP_253480.1;; |
| | | | CAD77853.1; CAA90427.1; YP_037488.1; |
| | | | YP_181386.1; ZP_00235565.1; |
| | | | YP_084669.1; XP_476303.1; |
| | | | ZP_00282112.1; AAN65341.1; |
| | | | 1ITZ; ZP_00231883.1; |
| | | | AAK78920.1; YP_020067.1; CAA86609.1; |
| | | | CAA86608.1; |
| | | | NP_662747.1; NP_925243.1; NP_734737.1; |
| | | | YP_188491.1; AAO35896.1; NP_764580.1; |
| | | | NP_937158.1; AAM91794.1; AAK79316.1; |
| | | | XP_471447.1; ZP_00328100.1; |
| | | | AAO29950.1; AAO07501.1; NP_687313.1; |
| | | | AAM62766.1; |
| | | | NP_935655.1; NP_682660.1; AAD10219.1; |
| | | | NP_670609.1; CAE06656.1; NP_801666.1; |
| | | | AAK34434.1; YP_203823.1; NP_566041.2; |
| | | | AAL98225.1; YP_071699.1; NP_954463.1; |
| | | | NP_800691.1; YP_075950.1; YP_060741.1; |
| | | | CAE22131.1; NP_214208.1; AAF11802.1; |
| | | | NP_893727.1; ZP_00527389.1; |
| | | | AAO09963.1; CAB82679.1; |
| | | | AAF93646.1; AAF96525.1; YP_015228.1; |
| | | | ZP_00233073.1; ZP_00155697.1; |
| | | | NP_928282.1; ZP_00416485.1; |
| | | | NP_466182.1; ZP_00156879.2; |
| | | | ZP_00528273.1; |
| | | | NP_472138.1; NP_798983.1; NP_267781.1; |
| | | | YP_131731.1; NP_439183.1; YP_131250.1; |
| | | | AAX88048.1; ZP_00585102.1; |
| | | | ZP_00381829.1; |
| | | | YP_206644.1; CAB58135.1; |
| | | | XP_550612.1; ZP_00129328.1; |
| | | | ZP_00530889.1; ZP_00132914.1; |
| | | | ZP_00537410.1; |
| | | | AAQ00814.1; ZP_00123444.1; |
| | | | AAO17218.1; |
| | | | CAG73773.1; AAT48155.1; BAB37233.1; |
| | | | NP_708699.2; NP_786741.1; 1QGD; NP_755395.1; |
| | | | CAG76812.1; ZP_00473037.1; |
| | | | AAK03722.1; AAG58065.1; ZP_00134256.2; |
| | | | AAV61915.1; ZP_00389210.1; |
| | | | NP_949977.1; NP_790234.1; NP_249239.1; |
| | | | XP_651488.1; XP_650850.1; YP_174605.1; |
| | | | AAU36664.1; AAK03326.1; XP_650836.1; |
| | | | AAQ57870.1; YP_237857.1; CAC18218.1; |
| | | | YP_152097.1; CAC47341.1; CAA48166.1; |
| | | | AAX66924.1; ZP_00347807.1; |
| | | | ZP_00473067.1; |
| | | | AAL21951.1; NP_346455.1; NP_359433.1; |
| | | | ZP_00418725.1; |
| | | | AAN58055.1; EAK85797.1; |
| | | | ZP_00398675.1; |
| | | | NP_840415.1; ZP_00267930.1; |
| | | | AAB82634.2; XP_326821.1; BAB62078.1; |
| | | | YP_011742.1; NP_756618.1; ZP_00631655.1; |
| | | | YP_222392.1; AAL51492.1; AAN30626.1; |
| | | | EAA69343.1; ZP_00315920.1; |
| | | | EAA54486.1; ZP_00584101.1; |
| | | | ZP_00334879.1; |
| | | | AAP96482.1; NP_299218.1; YP_115427.1; |
| | | | YP_115433.1; YP_156595.1; YP_157602.1; |
| | | | CAF26661.1; EAL90682.1; |
| | | | NP_534230.1; NP_779080.1; ZP_00040463.1; |
| | | | YP_046678.1; NP_463872.1; ZP_00230073.1; |
| | | | YP_012971.1; CAD80256.1; |
| | | | ZP_00234258.1; |
| | | | NP_769223.1; ZP_00303056.1; |
| | | | AAN70532.1; YP_199815.1; NP_469705.1; |
| | | | NP_784768.1; ZP_00038813.1; |
| | | | CAB82464.1; |
| | | | AAM38215.1; ZP_00145579.2; |
| | | | EAA65464.1; NP_716559.1; AAP86169.1; |
| | | | ZP_00283416.1; |
| | | | NP_638566.1; ZP_00264631.1; P21725; |
| | | | YP_262844.1; EAN07635.1; |
| | | | ZP_00281448.1; ZP_00566165.1; |
| | | | YP_034207.1; YP_125516.1; YP_094193.1; |
| | | | ZP_00151666.2; ZP_00459681.1; |
| | | | ZP_00640383.1; ZP_00004561.2; |
| | | | ZP_00453223.1; |
| | | | CAG88854.1; NP_879793.1; NP_887926.1; |
| | | | AAO91278.1; NP_883480.1; YP_122504.1; |
| | | | CAF32073.1; |
| | | | CAD16457.1; ZP_00635534.1; |
| | | | ZP_00629444.1; |
| | | | AAL21368.1; 1AY0; AAB68125.1; ZP_00376744.1; |
| | | | 1TKC; ZP_00243671.1; |
| | | | AAX66376.1; P29277; YP_149718.1; ZP_00270019.1; |
| | | | YP_174448.1; NP_804254.1; NP_971914.1; |
| | | | NP_708304.2; AAC75518.1; Q52723; |
| | | | CAH36963.1; ZP_00216610.1; |
| | | | ZP_00168684.2; |
| | | | NP_786435.1; NP_754872.1; CAA81260.1; |
| | | | CAA21881.1; AAK25582.1; BAB36750.1; |
| | | | ZP_00494674.1; |
| | | | YP_104015.1; ZP_00488354.1; |
| | | | CAG79209.1; ZP_00451919.1; |
| | | | AAF41816.1; NP_104787.1; CAH02329.1; |
| | | | EAL21160.1; ZP_00500476.1; |
| | | | ZP_00424086.1; ZP_00598759.1; |
| | | | ZP_00579602.1; AAA96746.2; |
| | | | ZP_00599356.1; |
| | | | AAG57574.1; CAA85074.1; ZP_00502496.1; |
| | | | ZP_00464206.1; |
| | | | AAW89704.1; NP_220269.1; AAS51554.1; |
| | | | CAB84897.1; CAA21989.1; EAK98686.1; |
| | | | CAF24238.1; AAW79357.1; |
| | | | ZP_00243955.1; AAX69269.1; |
| | | | ZP_00151635.1; |
| | | | NP_768164.1; CAJ05341.1; |
| | | | NP_946298.1; CAG58382.1; ZP_00556292.1; |
| | | | ZP_00006414.1; |
| | | | AAV95144.1; NP_878796.1; ZP_00229277.1; |
| | | | AAB06805.1; ZP_00319310.1; |
| | | | AAF39009.1; NP_660445.1; CAD20572.1; |
| | | | 1R9J; YP_170318.1; ZP_00626725.1; |
| | | | AAC26564.1; AAO51318.1; CAE09695.1; |
| | | | NP_239927.1; NP_777718.1; CAC48593.1; |
| | | | ZP_00210881.1; ZP_00620206.1; |
| | | | EAN96078.1; |
| | | | AAP76623.1; AAV88800.1; AAA96741.1; |
| | | | YP_220229.1; AAP98853.1; AAP05616.1; |
| | | | NP_300950.1; AAF38753.1; NP_225088.1; |
| | | | ZP_00374031.1; |
| | | | AAW71252.1; CAC47149.1; ZP_00507473.1; |
| | | | YP_180423.1; CAI28030.1; NP_966179.1; |
| | | | YP_154024.1; AAF25377.1; |
| | | | BAC24728.1; AAD08131.1; CAG28449.1; |
| | | | EAA22643.1; CAA86607.1; |
| | | | NP_223056.1; XP_743287.1; |
| | | | NP_703770.1; ZP_00062992.2; |
| | | | YP_053590.1; AAA35168.1; |
| | | | ZP_00545078.1; ZP_00371544.1; |
| | | | NP_975365.1; XP_674383.1; YP_179787.1; |
| | | | ZP_00419453.1; |
| | | | CAB73633.1; ZP_00367926.1; |
| | | | XP_651838.1; CAH07360.1; AAO75454.1; |
| | | | EAL17469.1; EAL20938.1; |
| | | | AAQ66751.1; EAA66976.1; ZP_00368598.1; |
| | | | EAA53893.1; XP_648840.1; ZP_00120373.1; |
| | | | AAA26967.1; ZP_00050045.2; |
| | | | EAA64069.1; EAL17468.1; |
| | | | CAD25372.1; CAG88707.1; YP_115941.1; |
| | | | EAA70882.1; EAL86575.1; AAC83349.1; |
| | | | AAG59818.1; ZP_00514509.1; |
| | | | ZP_00645002.1; |
| | | | CAG84976.1; NP_326342.1; YP_016250.1; |
| | | | ZP_00020488.2; |
| | | | NP_757837.1; EAA37632.1; EAN84471.1; |
| | | | NP_078425.1; NP_757463.1; ZP_00053756.1; |
| | | | CAA26276.1; |
| | | | NP_072728.1; AAB95721.1; AAP56380.1; |
| | | | BAA13834.1; AAN18173.1; |
| | | | ZP_00404457.1; CAH25336.1; |
| | | | ZP_00403049.1; ZP_00403048.1; |
| | | | XP_648680.1; ZP_00234017.1; |
| | | | ZP_00404458.1; ZP_00332380.1; |
| | | | ZP_00514508.1; ZP_00357197.1; |
| | | | ZP_00642210.1; ZP_00372877.1; |
| | | | AAM94004.1; BAA95691.1; |
| | | | AAG43112.1; ZP_00405278.1; |
| | | | ZP_00053393.1; ZP_00374249.1; |
| | | | ZP_00120372.1; AAA50394.1; |
| | | | AAS93346.1; ZP_00642816.1; |
| | | | CAC21145.1; CAC21168.1; CAC21141.1; |
| | | | CAC21161.1; CAC21159.1; CAC21148.1; |
| | | | CAC21142.1; CAC21140.1; AAS93351.1; |
| | | | AAK63242.1; AAK63239.1; |
| | | | AAS93349.1; AAS93347.1; CAC21156.1; |
| | | | AAS93356.1; CAC21157.1; CAC21162.1; |
| | | | CAC21137.1; CAE75672.1; CAA13584.1; |
| | | | CAA13374.1; AAK63244.1; |
| | | | AAQ20076.1; AAU95206.1; |
| | | | CAB60654.1; CAA13585.1; |
| | | | ZP_00405277.1; ZP_00510627.1; |
| | | | ZP_00374310.1; AAK63246.1; |
| | | | ZP_00574965.1; |
| | | | AAL94500.1; CAC11757.1; YP_076140.1; |
| | | | ZP_00534343.1; |
| | | | NP_228762.1; EAN81253.1; |
| | | | NP_953961.1; AAK17116.1; |
| | | | XP_651839.1; AAN50417.1; YP_077731.1; |
| | | | YP_090144.1; ZP_00207814.1; |
| | | | NP_247665.1; ZP_00559948.1; |
| | | | NP_346546.1; XP_734906.1; |
| | | | BAB80002.1; NP_111187.1; ZP_00403604.1; |
| | | | NP_687234.1; CAB71601.1; |
| | | | YP_023469.1; NP_532581.1; NP_988235.1; |
| | | | AAX66248.1; CAB71607.1; |
| | | | CAB71595.1; CAB71613.1; CAB39235.1 |
| subunit of | supply of | YP_225861 | CAF21585.1; NP_738307.1; NP_939658.1; |
| glucose-6-P | reduction | | CAI37159.1; YP_119788.1; EAM76743.1; |
| dehydrogenase | equivalents | | NP_855133.1;; NP_215962.1;; |
| opcA (R3) | for | | ZP_00413484.1; YP_062111.1;; |
| | biochemical | | NP_960109.1;; ZP_00294053.1; |
| | reductions | | BAC74023.1;; BAC69480.1;; |
| | such as | | CAA19939.1;; CAB50763.1;; |
| | sulfate to | | ZP_00120910.2; NP_695642.1;; |
| | sulfide | | ZP_00658921.1; YP_056265.1;; |
| | reductions | | ZP_00548312.1; ZP_00569428.1; |
| | or | | NP_301492.1;; ZP_00357972.1; |
| | methylene | | CAF23544.1;; ZP_00519748.1; |
| | tetrathydrofolate | | NP_926125.1;; ZP_00326213.1; |
| | to | | ZP_00395319.1; P48971; ZP_00112207.2; |
| | methyl THF | | AAF11159.1;; YP_172479.1;; Q54709; |
| | | | ZP_00328805.1; ZP_00160728.2; |
| | | | NP_441088.1;; BAB75717.1;; |
| | | | CAE07264.1;; CAE21278.1;; |
| | | | AAQ00170.1; ZP_00516459.1 |
| 6- | supply of | BAB98845 | NP_939570.1; NP_738198.1; CAI37076.1; |
| Phosphoglucono | reduction | | YP_117384.1; AAK46163.1; |
| lactone | equivalents | | YP_177848.1; |
| dehydrogenase | for | | NP_960491.1; NP_302377.1; CAA15451.1; |
| (R5) | biochemical | | BAC74960.1; CAC44325.1; NP_695644.1; |
| | reductions | | YP_062600.1; ZP_00120912.2; ZP_00412831.1; |
| | such as | | EAM73768.1; NP_855527.1 |
| | sulfate to | | NP_691106.1; |
| | sulfide | | YP_148197.1; ZP_00283191.1; NP_470749.1; |
| | reductions | | YP_079721.1; NP_464901.1; CAG74354.1; |
| | or | | NP_928851.1; YP_056324.1; NP_390267.2; |
| | methylene | | YP_070081.1; NP_669932.1; ZP_00212780.1; |
| | tetrathydrofolate | | ZP_00424035.1; ZP_00462313.1; YP_016771.1; |
| | to | | YP_111755.1; AAO44589.1; NP_992794.1; |
| | methyl THF | | YP_034514.1; ZP_00390566.1; |
| | | | ZP_00236407.1; ZP_00232091.1; YP_081773.1; |
| | | | ZP_00494517.1; EAN09346.1; ZP_00502057.1; |
| | | | ZP_00403921.1; NP_344902.1; ZP_00111860.1; |
| | | | NP_814782.1; AAL67561.1; |
| | | | AAA24492.1; AAG35235.1; AAG35224.1; |
| | | | AAG57088.1; BAB76974.1; AAG35219.1; |
| | | | AAA24490.1; ZP_00163835.2; ZP_00158100.1; |
| | | | AAA24208.1; AAO37703.1; YP_175422.1; |
| | | | AAV74553.1; AAV27335.1; AAA24494.1; |
| | | | AAA23918.1; AAC75090.1; AAG35218.1;; |
| | | | AAA24207.1; YP_172170.1; AAA24495.1; |
| | | | AAA24493.1; AAG35221.1; AAA24209.1; |
| | | | NP_707923.1; AAG35223.1; AAA24489.1; |
| | | | P41576; AAD50492.1; AAV34527.1; |
| | | | AAL20985.1; AAA24206.1; BAA28321.1; |
| | | | AAV74381.1; AAX65997.1; NP_804634.1; |
| | | | AAA24488.1; YP_150095.1; P37754; AAD46733.1; |
| | | | BAA77736.1; NP_372035.1; P21577; |
| | | | YP_040985.1; ZP_00384155.1; ZP_00379330.1; |
| | | | YP_253321.1; NP_924063.1; NP_764747.1; |
| | | | NP_266778.1; ZP_00323177.1; NP_681366.1; |
| | | | P96789; ZP_00315559.1; AAC43775.1; |
| | | | P41582; AAC43777.1; AAC43778.1; |
| | | | AAC43807.1; AAC43817.1; P41580; AAC43818.1; |
| | | | AAC43805.1; AAC43793.1; ZP_00326299.1; |
| | | | P41579; AAC43782.1; AAC43811.1; |
| | | | AAC43808.1; AAC43800.1; AAC43798.1; |
| | | | AAC43795.1; NP_785144.1; AAC43806.1; |
| | | | AAC43804.1; AAC43781.1; CAD72844.1; |
| | | | AAC43813.1; AAC43809.1; AAC43803.1; |
| | | | AAC43787.1; P41578; P41574; AAC43786.1; |
| | | | P41581; AAC43797.1; AAC43788.1; |
| | | | AAC43834.1; AAC43810.1; AAC43785.1; |
| | | | AAC43784.1; AAC43812.1; P41577; AAS99175.1; |
| | | | P41575; AAC43799.1; AAC43913.1; |
| | | | AAC43906.1; AAC43828.1; P41583; AAC43794.1; |
| | | | AAC43825.1; AAL76323.1; AAC43902.1; |
| | | | AAC43832.1; AAC43923.1; AAC43916.1; |
| | | | AAC43914.1; AAC43912.1; AAC43911.1; |
| | | | AAC43907.1; AAC43905.1; AAC43824.1; |
| | | | CAA41555.1; AAC43918.1; AAL27335.1; |
| | | | AAC43920.1; AAC43901.1; AAC43831.1; |
| | | | AAC43904.1; AAW29822.1; AAC43908.1; |
| | | | AAC43829.1; AAC43919.1; AAC43910.1; |
| | | | XP_342980.2; YP_114383.1; AAC43830.1; |
| | | | ZP_00319235.1; Q9DCD0; AAH59958.1; |
| | | | AAA74174.1; AAH11329.1; AAA74166.1; |
| | | | AAC43921.1; AAC43915.1; YP_081362.1; |
| | | | CAG32303.1; ZP_00564543.1; NP_694109.1; |
| | | | AAA74172.1; AAA74152.1; AAA74154.1; |
| | | | AAA74149.1; AAA74163.1; AAA74146.1; |
| | | | AAR24280.1; AAA74159.1; CAI95751.1; |
| | | | AAP88742.1; AAL27345.1; AAA74162.1; |
| | | | AAA74157.1; AAA74147.1; AAA27330.1; |
| | | | AAA74173.1; AAA74143.1; AAA74169.1; |
| | | | NP_391888.1; AAA74144.1; P52207; |
| | | | AAA74167.1; AAA74164.1; AAA74158.1; |
| | | | NP_442035.1; AAL76320.1; AAA75302.1; |
| | | | AAA74171.1; ZP_00516323.1; AAL27356.1; |
| | | | AAR97968.1; AAA74145.1; CAF23041.1; |
| | | | CAA42751.1; AAA74151.1; 2PGD; ZP_00062611.2; |
| | | | AAA74156.1; YP_129657.1; |
| | | | ZP_00539485.1; AAK51690.1; AAA74161.1; |
| | | | AAA74150.1; AAK64376.1; AAA74168.1; |
| | | | AAA74175.1; AAA74165.1; XP_758724.1; |
| | | | AAA74170.1; AAA74155.1; AAA74160.1; |
| | | | CAG07546.1; AAA74153.1; AAQ13889.1; |
| | | | NP_910282.1; AAO32456.1; AAH44196.1; |
| | | | CAA76734.1; AAF40494.1; AAP92648.1; |
| | | | AAQ13881.1; CAE46650.1; NP_998717.1; |
| | | | AAO42814.1; AAM64891.1; EAL03585.1; |
| | | | O13287; CAA94380.1; AAK49897.1; AAQ13887.1; |
| | | | AAP33506.2; CAH02996.1; XP_625090.1; |
| | | | AAA74142.1; NP_798087.1; CAG62903.1; |
| | | | NP_012053.1; AAO11029.1; AAQ13885.1; |
| | | | NP_934400.1; AAO19944.1; AAO19943.1; |
| | | | AAM78095.1; AAL76326.1; AAQ13883.1; |
| | | | AAH95571.1; ZP_00524274.1; AAQ13879.1; |
| | | | CAG86870.1; CAG83189.1; AAC27703.1; |
| | | | NP_239940.1;; CAB61332.1; AAO19942.1; |
| | | | AAO19941.1; ZP_00123635.1; |
| | | | CAB83570.1; AAO19934.1; AAQ13880.1; |
| | | | ZP_00131777.2; ZP_00135245.2; AAQ13888.1; |
| | | | AAQ13882.1; EAL18227.1; NP_011772.1; |
| | | | BAD98151.1; NP_438711.1; AAO32396.1; |
| | | | EAA48517.1; NP_777731.1; AAO32497.1; |
| | | | AAU36620.1; AAM61057.1; AAX87602.1; |
| | | | AAQ13878.1; CAE70848.1; BAD36766.1; |
| | | | EAL88658.1; AAS53500.1; XP_330536.1; |
| | | | ZP_00156373.2; XP_313091.2; AAF96795.1; |
| | | | CAE53864.1; AAB41553.1; CAA22536.1; |
| | | | AAC27702.1; YP_206428.1; BAC06328.1; |
| | | | EAA59263.1; EAL31500.1; AAP95728.1; |
| | | | CAB10974.1; AAQ13886.1; P70718; AAF45732.1;; |
| | | | AAK03638.1; AAC65319.1; EAA67653.1; |
| | | | AAR25841.1; ZP_00152366.1; |
| | | | AAB29396.1; EAN05374.1; P41573; YP_219832.1; |
| | | | CAD80254.1; CAD56883.1; CAC46511.1; |
| | | | AAP05178.1; NP_532215.1; AAQ13884.1; |
| | | | NP_104453.1; AAL27320.1; NP_660459.1; |
| | | | AAO36383.1; AAO76329.1; CAH07617.1; |
| | | | YP_099135.1; NP_542102.1; YP_222920.1; |
| | | | AAF39196.1; AAB20377.1; AAC97362.1; |
| | | | NP_878753.1; CAE07634.1; AAP98300.1; |
| | | | NP_892888.1; NP_228248.1; NP_623516.1; |
| | | | NP_219566.1; CAE20740.1; ZP_00580432.1; |
| | | | AAZ25595.1; NP_717509.1; EAA18974.1; |
| | | | YP_194797.1; BAD36765.1; AAP99887.1; |
| | | | XP_550483.1; AAO37720.1; AAL76318.1; |
| | | | AAV34504.1; NP_965830.1; ZP_00586799.1; |
| | | | AAW29924.1; AAW29923.1; |
| | | | ZP_00383992.1 |
| transaldolase | supply of | CAF21583.1 | ; NP_738305.1; NP_939656.1; |
| (R9) | reduction | | CAI37157.1; NP_960111.1; |
| | equivalents | | YP_119786.1; NP_215964.1; |
| | for | | NP_855135.1; NP_301493.1; |
| | biochemical | | ZP_00413481.1; YP_062114.1;; |
| | reductions | | ZP_00294056.1; BAC69478.1;; |
| | such as | | ZP_00658918.1; BAC74025.1;; |
| | sulfate to | | CAB50761.1;; CAA19941.1;; |
| | sulfide | | EAM76741.1; ZP_00548314.1; |
| | reductions | | ZP_00569430.1; ZP_00120374.1; |
| | or | | NP_695898.1;; ZP_00381422.1; |
| | methylene | | AAL15881.1; AAO44438.1;; |
| | tetrathydrofolate | | NP_789361.1;; ZP_00600251.1; |
| | to | | BAD88191.1; ZP_00655611.1; |
| | methyl THF | | AAP83926.1; AAG16981.1;; |
| | | | AAM64693.1;; ZP_00326211.1; |
| | | | YP_192100.1;; XP_463680.1;; |
| | | | BAB75719.1;; ZP_00333934.1; |
| | | | AAA98852.1; P48983; BAD08583.1; |
| | | | ZP_00160726.2; NP_773398.1;; |
| | | | NP_926323.1;; ZP_00623652.1; |
| | | | YP_159671.1;; NP_948972.1;; |
| | | | EAO16453.1; AAW90238.1;; |
| | | | CAB85348.1;; AAQ58240.2;; Q9K139; |
| | | | ZP_00203717.1; ZP_00522015.1; |
| | | | NP_842140.1;; AAT08720.1; |
| | | | AAA17145.1; CAE11079.1;; |
| | | | ZP_00648773.1; AAA17140.1; |
| | | | AAD08536.1;; CAD73047.1;; |
| | | | NP_224106.1;; ZP_00369384.1; |
| | | | ZP_00367543.1; YP_178349.1;; |
| | | | AAL15878.1; AAP77737.1;; |
| | | | AAL15879.1; AAL15872.1; AAL15875.1; |
| | | | AAL15876.1; AAL15874.1; |
| | | | ZP_00369996.1; AAL15880.1; |
| | | | ZP_00657668.1; YP_055164.1;; |
| | | | ZP_00328326.1; ZP_00416486.1; |
| | | | CAD14933.1;; ZP_00280696.1; |
| | | | ZP_00107110.1; AAP05431.1;; |
| | | | NP_719093.1;; AAH09680.1;; |
| | | | AAH10103.1;; AAH18847.2;; |
| | | | ZP_00587113.1; YP_172513.1;; |
| | | | ZP_00263754.1; ZP_00165284.2; |
| | | | AAO32594.1; AAL55523.1; |
| | | | YP_115432.1;; ZP_00516112.1; P51778; |
| | | | NP_251486.1;; NP_791941.1;; |
| | | | AAX66375.1;; NP_754871.1;; |
| | | | AAO32544.1; AAT51194.1; AAS51032.1; |
| | | | NP_035658.1;; NP_804255.1;; |
| | | | NP_440132.1;; NP_439282.1;; |
| | | | ZP_00495461.1; NP_113999.2;; |
| | | | YP_046627.1;; AAL21367.1;; |
| | | | ZP_00160100.2; AAF96524.1;; |
| | | | YP_149719.1;; XP_533146.1;; |
| | | | NP_924543.1;; XP_420949.1;; |
| | | | BAB74262.1;; XP_397306.2;; |
| | | | ZP_00242010.1; ZP_00415676.1; |
| | | | AAP98016.1;; CAG73774.1;; |
| | | | CAA18994.1;; ZP_00269391.1; |
| | | | AAF38500.1;; AAF39419.1;; |
| | | | NP_892637.1;; ZP_00653780.1; |
| | | | NP_800690.1;; YP_220057.1;; |
| | | | NP_219818.1;; AAX46381.1; |
| | | | AAG43169.1;; ZP_00133180.1; |
| | | | ZP_00498695.1; ZP_00451399.1; |
| | | | ZP_00635839.1; AAK03686.1;;; |
| | | | ZP_00212331.1; CAA78965.1; |
| | | | ZP_00583402.1; ZP_00473621.1; |
| | | | CAG31705.1; AAZ19038.1;; |
| | | | YP_234996.1;; NP_671009.1;; |
| | | | AAH61957.1;; CAC89319.1;; |
| | | | NP_681257.1;; AAH68191.1;; |
| | | | AAH84118.1;; ZP_00156967.1; |
| | | | ZP_00509652.1; XP_306040.2;; |
| | | | NP_705968.2;; AAO07500.1;; |
| | | | YP_149357.1;; NP_937157.1;; |
| | | | AAX63913.1;; NP_751968.1;; |
| | | | YP_202288.1;; Q8FLD1; 1I2R;; 1I2P;; |
| | | | 1ONR;; CAE08274.1;; ZP_00444183.1; |
| | | | ZP_00642538.1; CAG76785.1;; |
| | | | EAK96114.1; 1I2N;; XP_760285.1; |
| | | | AAN67781.1;; ZP_00566285.1; |
| | | | ZP_00534289.1; ZP_00459221.1; |
| | | | YP_270759.1; YP_069148.1;; |
| | | | NP_838015.1;; NP_228107.1;; |
| | | | ZP_00640073.1; NP_534942.1;; |
| | | | EAL18010.1; AAW46393.1;; |
| | | | NP_001017131.1;; 1VPX;; |
| | | | ZP_00167021.1; ZP_00135489.1; |
| | | | ZP_00561481.1; ZP_00314890.1; |
| | | | YP_017300.1;; ZP_00507474.1; |
| | | | EAA72420.1;; NP_463873.1;; |
| | | | NP_883543.1;; NP_880193.1;; |
| | | | NP_887991.1;; NP_636221.1;; |
| | | | YP_206643.1;; XP_640977.1;; |
| | | | AAX88128.1;; ZP_00551628.1; |
| | | | AAP95288.1;; YP_131732.1;; 1I2Q;; |
| | | | ZP_00316642.1; AAP07679.1;; |
| | | | AAL60146.1; YP_259093.1;; |
| | | | AAU38962.1;; AAM35790.1;; |
| | | | AAK03723.1;; 1I2O;; 1UCW;; |
| | | | YP_170072.1;; NP_927918.1;; |
| | | | NP_736284.1;; AAO32444.1; Q9S0X4; |
| | | | CAF99897.1; AAW50031.1; |
| | | | ZP_00597354.1; NP_708303.1;; |
| | | | NP_623494.1;; NP_247955.1;; |
| | | | ZP_00422910.1; CAE21423.1;; |
| | | | AAZ22459.1; NP_777717.1;; |
| | | | CAG78269.1;; CAG35157.1;; |
| | | | AAP99564.1;; AAN31490.1; |
| | | | CAA34078.1; NP_013458.1;; |
| | | | NP_239926.1;; YP_144332.1;; |
| | | | AAX15925.1; ZP_00473066.1; |
| | | | YP_149238.1;; NP_011557.1;; |
| | | | AAO32543.1; AAS56158.1; |
| | | | XP_320715.2;; NP_988428.1;; |
| | | | CAG61370.1;; CAF24415.1;; |
| | | | AAM75991.1; YP_081036.1;; |
| | | | XP_508205.1;; XP_591134.1;; |
| | | | CAG58006.1;; ZP_00346593.1; |
| | | | EAA66113.1;; BAB07504.1;; |
| | | | BAC24729.1;; CAE58522.1; |
| | | | NP_954019.1;; NP_466265.1;; |
| | | | AAX69845.1; YP_015318.1;; |
| | | | NP_660444.1;; AAL65638.1; |
| | | | ZP_00230153.1; AAW69342.1; |
| | | | ZP_00538489.1; ZP_00530466.1; |
| | | | AAP37846.1;; NP_878795.1;; |
| | | | AAL65636.1; AAL65632.1; AAL65631.1; |
| | | | AAL65625.1; AAL65622.1; AAL65620.1; |
| | | | AAL65619.1; AAL65613.1; BAD94458.1; |
| | | | AAK79315.1;; AAO34876.1;; Q899F3; |
| | | | YP_182117.1;; NP_213080.1;; |
| | | | NP_660916.1;; XP_480152.1;; |
| | | | AAO32445.1; AAN87407.1; |
| | | | ZP_00576424.1; AAK93861.1;; |
| | | | EAO20247.1; NP_786742.1;; |
| | | | AAM66063.1; CAG89600.1;; |
| | | | ZP_00053003.1; ZP_00357963.1; |
| | | | ZP_00523938.1; NP_801665.1;; |
| | | | EAL91678.1;; YP_004676.1;; |
| | | | BAB80398.1;; AAF10909.1;; Q9RUP6; |
| | | | CAA89874.1; EAO23438.1; |
| | | | NP_111185.1;; CAJ03645.1; |
| | | | NP_967077.1;; NP_391592.2;; |
| | | | AAG34725.1; AAR10031.1; |
| | | | AAQ65460.1;; AAP10311.1;; |
| | | | YP_177374.1;; NP_472214.1;; |
| | | | AAM50780.1; AAF47106.2;; |
| | | | ZP_00513183.1; AAQ17460.1; |
| | | | CAH08777.1;; EAN83889.1; |
| | | | YP_214732.1;; YP_100521.1;; |
| | | | EAN81646.1; XP_329326.1;; |
| | | | ZP_00622649.1; ZP_00661024.1; |
| | | | AAO76765.1;; EAA58088.1;; |
| | | | EAA49912.1;; YP_020065.1;; |
| | | | AAQ59919.1;; ZP_00402027.1; |
| | | | YP_037486.1;; NP_693926.1;; |
| | | | YP_073895.1;; YP_010876.1;; |
| | | | AAK15382.1; AAK15373.1; |
| | | | ZP_00302582.1; ZP_00554321.1; |
| | | | ZP_00397276.1; ZP_00332965.1; |
| | | | AAK15379.1; ZP_00577093.1; |
| | | | EAL86159.1;; CAC11755.1;; |
| | | | ZP_00207691.1; ZP_00386434.1; |
| | | | YP_023467.1;; AAK25576.1;; |
| | | | ZP_00266165.1; ZP_00311094.1; |
| | | | ZP_00592141.1; ZP_00588840.1; |
| | | | AAK15385.1; NP_816912.1;; |
| | | | AAN49485.1;; AAK34644.1;; |
| | | | AAN58723.1;; NP_802941.1;; |
| | | | YP_253060.1;; AAM80284.1;; |
| | | | ZP_00376041.1; YP_222463.1;; |
| | | | ZP_00207851.1; XP_417183.1;; |
| | | | EAA76445.1;; YP_041250.1;; |
| | | | NP_372305.1;; YP_060987.1;; |
| | | | AAL98495.1;; XP_417182.1;; |
| | | | AAL80779.1;; ZP_00579645.1; |
| | | | EAM28893.1; YP_080940.1;; |
| | | | YP_093370.1;; AAU21341.1; |
| | | | AAF39749.1;; AAL51426.1;; |
| | | | NP_772111.1;; Q8WKN0; Q8SEL8; |
| | | | CAD27636.1; CAD27635.1; |
| | | | CAD27630.1; CAD27629.1; |
| | | | CAD27627.1; CAD27625.1; |
| | | | CAD27624.1; ZP_00215873.1; |
| | | | AAN02022.1;; ZP_00267913.1; |
| | | | AAS53358.1 |
| transhydrogenase | redox- | AAC76944 | P27306; AAC43068.1; NP_756777.1; NP_709766.2; |
| udh (R70) | conversion | | Q83MI1; Q8X727; AAG59164.1; |
| | of pyrimidin | | AAX67921.1; YP_153038.1; CAG77139.1; |
| | nucleotides | | YP_068670.1; NP_667661.1; NP_931901.1; |
| | | | NP_799321.1; AAF93328.1; AAO09639.1; |
| | | | YP_205822.1; YP_131541.1; CAA46822.1; |
| | | | AAZ24633.1; YP_154714.1; ZP_00416263.1; |
| | | | NP_251681.1; EAM24138.1; AAY36945.1; |
| | | | NP_791929.1; AAN67764.1; YP_274087.1; |
| | | | YP_259077.1; O05139; ZP_00263769.1; |
| | | | CAD74394.1; ZP_00315937.1; YP_046885.1; |
| | | | ZP_00653465.1; AAZ19183.1; YP_169700.1; |
| | | | AAW50006.1; NP_961763.1; ZP_00574185.1; |
| | | | ZP_00546586.1; ZP_00523033.1; |
| | | | NP_217229.1; NP_532346.1; AAV97042.1; |
| | | | CAF23449.1; CAC46308.1; ZP_00622185.1; |
| | | | NP_108478.1; ZP_00400087.1; P71317; ZP_00317524.1; |
| | | | ZP_00524635.1; YP_143553.1; |
| | | | YP_005669.1; EAN07674.1; ZP_00625011.1; |
| | | | NP_105199.1; AAN30810.1;; AAL51327.1; |
| | | | EAO37648.1; YP_222565.1; ZP_00307577.1; |
| | | | ZP_00265019.1; AAN70931.1; CAF22875.1; |
| | | | CAC47627.1; CAA39235.1; NP_213506.1; |
| | | | ZP_00284261.1; NP_945538.1; |
| | | | EAN27796.1; AAF34795.3; AAF79529.1; ZP_00141283.2; |
| | | | ZP_00492121.1; AAR21288.1; |
| | | | NP_253516.1; AAN03817.1; AAG17888.1; |
| | | | ZP_00449174.1; YP_180009.1; YP_034342.1; |
| | | | NP_533297.1; CAI26632.1; NP_767089.1; |
| | | | AAC26053.1; YP_246823.1; ZP_00497224.1; |
| | | | YP_067405.1; XP_331183.1; AAD53185.1; |
| | | | EAA51976.1; NP_220840.1; ZP_00269527.1; |
| | | | AAF34796.1; CAA11554.1; CAA44729.1; |
| | | | 1DXL; EAM25883.1; AAF95555.1; |
| | | | YP_115390.1; AAX88688.1; NP_439387.1; |
| | | | YP_160845.1; EAK93183.1; ZP_00157402.1; |
| | | | ZP_00464142.1; CAA70224.1; ZP_00055963.2; |
| | | | EAO33154.1; ZP_00575798.1; EAL87307.1; |
| | | | ZP_00211386.1; ZP_00340462.1; |
| | | | AAR38073.1; NP_240038.1; AAS47493.1; |
| | | | ZP_00317120.1; NP_298158.1; ZP_00210426.1; |
| | | | ZP_00637900.1; AAN23154.1; CAF26798.1; |
| | | | ZP_00154973.1; YP_170418.1; NP_779995.1; |
| | | | ZP_00151187.2; AAB30526.1; |
| | | | ZP_00526430.1; EAA77706.1; CAD60736.1; |
| | | | BAB05544.1; ZP_00153792.2; AAU37941.1; |
| | | | AAV93660.1; NP_969527.1; CAD14973.1; |
| | | | ZP_00511405.1; NP_798896.1; EAO30592.1; |
| | | | ZP_00665518.1; YP_265659.1; NP_925975.1; |
| | | | NP_388690.1; ZP_00633839.1; AAV29309.1; |
| | | | CAG35032.1; AAK02977.1; CAF92514.1; |
| | | | ZP_00597315.1; CAA37631.1; |
| | | | ZP_00582828.1; AAK22329.1; 1EBD; ZP_00545191.1; |
| | | | NP_662186.1; CAD72797.1; AAG12404.1; |
| | | | ZP_00601791.1; BAB44156.1; |
| | | | ZP_00644737.1; XP_475628.1; NP_360330.1; |
| | | | AAA91879.1; AAC46405.1; ZP_00139702.1; |
| | | | YP_131302.1; CAF23812.1; ZP_00340821.1; |
| | | | EAA26462.1; ZP_00122566.1; ZP_00150164.2; |
| | | | ZP_00132373.2; YP_021029.1; |
| | | | NP_908725.1; CAG76686.1; AAO10051.1; |
| | | | NP_935564.1; NP_953492.1; CAB05249.2; |
| | | | ZP_00240355.1; ZP_00589771.1; ZP_00585786.1; |
| | | | NP_794013.1; XP_635122.1; YP_274206.1; |
| | | | NP_757897.1; NP_692788.1; NP_892685.1; |
| | | | AAP11076.1; CAG85768.1; NP_980528.1; |
| | | | NP_250715.1; YP_146914.1; CAC14663.1; |
| | | | AAP96400.1; XP_758608.1; YP_001129.1; |
| | | | NP_767536.1; ZP_00538550.1; YP_069256.1; |
| | | | CAA71040.1; ZP_00597992.1; |
| | | | AAY37054.1; NP_706070.2; NP_752095.1; |
| | | | CAA24742.1; AAF49294.1; YP_205561.1; |
| | | | NP_999227.1; CAJ08862.1; CAA71038.2; |
| | | | ZP_00283805.1; XP_320877.2; ZP_00528740.1; |
| | | | YP_148949.1; YP_040992.1; NP_792022.1; |
| | | | YP_002403.1; CAG81278.1; YP_085309.1; |
| | | | AAS47708.1; ZP_00662383.1; EAN80618.1; |
| | | | ZP_00411894.1; YP_149503.1; YP_078853.1; |
| | | | NP_470744.1; YP_013986.1; XP_623438.1; |
| | | | ZP_00020745.2; ZP_00134358.2; |
| | | | ZP_00536790.1; AAP10890.1; NP_804043.1; |
| | | | AAX64059.1; YP_186404.1; NP_372042.1; |
| | | | AAL19118.1; AAR38213.1; ZP_00507305.1; |
| | | | EAO31379.1; ZP_00415841.1; CAA49991.1; |
| | | | ZP_00589476.1; EAN90443.1; NP_716063.1; |
| | | | NP_777818.1; YP_134400.1; NP_464896.1; |
| | | | AAQ91233.1; AAN50085.1; AAH44432.1; |
| | | | CAB06298.1; ZP_00233557.1; |
| | | | AAN69413.1; EAN96941.1; NP_930833.1; |
| | | | NP_266215.1; AAN48422.1; CAA72131.1; |
| | | | CAA61483.1; ZP_00383074.1; AAD55376.1; |
| | | | YP_020826.1; NP_885384.1; NP_879905.1; |
| | | | CAA62982.1; CAE46806.1; YP_247286.1; |
| | | | AAR10425.1; AAV48381.1; CAA67822.1; |
| | | | NP_635936.1; EAA26057.1; AAM38502.1; |
| | | | CAE46804.1; NP_389344.1; AAH56016.1; |
| | | | YP_253312.1; YP_257414.1; YP_260503.1; |
| | | | CAA72132.1; CAG31211.1; YP_199361.1; |
| | | | ZP_00166998.2; ZP_00565931.1; NP_842161.1; |
| | | | ZP_00591535.1; ZP_00499160.1; NP_221155.1; |
| | | | NP_660554.1; AAS53883.1; NP_815369.1; |
| | | | CAG58981.1; ZP_00154188.2; |
| | | | ZP_00554136.1; AAP98791.1; AAM93255.1; |
| | | | EAN04065.1; YP_180376.1; NP_953634.1; |
| | | | NP_300890.1; CAI27032.1; AAR38090.1; |
| | | | EAO31664.1; ZP_00595215.1; ZP_00661894.1; |
| | | | YP_078075.1; CAI27980.1;; ZP_00396676.1; |
| | | | AAN75618.1; YP_253771.1; NP_220072.1; |
| | | | CAB84783.1; EAM31433.1; ZP_00263252.1; |
| | | | YP_019413.1; AAN75159.1; CAA62435.1; |
| | | | NP_778978.1; AAV28779.1; NP_360876.1; |
| | | | NP_031887.2; NP_878457.1; |
| | | | NP_966125.1; AAC53170.1; AAH18696.1; |
| | | | AAH62069.1; AAW71149.1; CAA59171.1; |
| | | | AAN69768.1; AAF39644.1; ZP_00373647.1; |
| | | | EAL29693.1; NP_763640.1; AAA35764.1; |
| | | | NP_116635.1; YP_148232.1; AAV28746.1; |
| | | | AAW89295.1; AAN05019.1; YP_084091.1; |
| | | | 1V59; P31052; ZP_00245417.1; ZP_00212990.1; |
| | | | ZP_00266952.1; AAF41719.1; NP_298837.1; |
| | | | NP_623271.1; AAN75183.1; NP_883660.1; |
| | | | NP_888964.1; AAN15202.1; AAU45403.1; |
| | | | ZP_00308867.1; BAD92940.1; NP_000099.1; |
| | | | 1ZMD; ZP_00418304.1; ZP_00399987.1; |
| | | | AAO90013.1; NP_764754.1; YP_258846.1; |
| | | | BAE00452.1; ZP_00305550.1; |
| | | | ZP_00210841.1; 3LAD; YP_188656.1; NP_880776.1; |
| | | | ZP_00007570.1; BAD11090.1; AAW89611.1; |
| | | | EAN09173.1; AAY38013.1; AAF41363.1; |
| | | | ZP_00670517.1; AAN75720.1; |
| | | | CAB84413.1; AAM36402.1; NP_966507.1; |
| | | | AAC44345.1; ZP_00107537.1; CAA61894.1; |
| | | | CAA57206.1; CAB65609.1; ZP_00578463.1; |
| | | | ZP_00550077.1; YP_156710.1; CAD61860.1; |
| | | | BAD11095.1; ZP_00537692.1; NP_764349.1; |
| | | | NP_792904.1; AAN00129.1; AAB88282.1; |
| | | | 1BHY; 1OJT; ZP_00245307.1; AAA83977.1; |
| | | | ZP_00592008.1; ZP_00557093.1; |
| | | | AAQ58205.1; ZP_00669696.1; AAN00882.1; |
| | | | CAA54878.1; YP_200681.1; AAQ58749.1; |
| | | | YP_274470.1; YP_154852.1; NP_763920.1; |
| | | | YP_079735.1; NP_879789.1; NP_889077.1; |
| | | | YP_067730.1; NP_636857.1; AAO78292.1; |
| | | | BAB33285.1; CAH93405.1; NP_842316.1; |
| | | | AAD30450.1; ZP_00170705.2; AAF99445.1; |
| | | | AAB01381.1; ZP_00245305.1; AAW71147.1; |
| | | | NP_250278.1; CAD15305.1; AAK23707.1; |
| | | | ZP_00160593.1; AAO44599.1; |
| | | | NP_975267.1; CAH00655.1; ZP_00212747.1; |
| | | | CAE20510.1; YP_040483.1; ZP_00474314.1; |
| | | | ZP_00464633.1; NP_789199.1; AAP05672.1; |
| | | | AAV47687.1; ZP_00108447.1; ZP_00516811.1; |
| | | | ZP_00467577.1; ZP_00451158.1; |
| | | | NP_883762.1; YP_187838.1; ZP_00376179.1; |
| | | | BAB06371.1; BAC24467.1; NP_763632.1; |
| | | | YP_036862.1; AAA99234.1; 1LPF; |
| | | | ZP_00579524.1; ZP_00561492.1; ZP_00500723.1; |
| | | | ZP_00486500.1; YP_220287.1; ZP_00239726.1; |
| | | | NP_681658.1; NP_893415.1; EAN08634.1; |
| | | | ZP_00578482.1; ZP_00531539.1; |
| | | | ZP_00463093.1; ZP_00397330.1; ZP_00642506.1; |
| | | | ZP_00620223.1; YP_126870.1; CAC46029.1; |
| | | | AAP94898.1; CAE08145.1; ZP_00512893.1; |
| | | | YP_123783.1; ZP_00401182.1; |
| | | | NP_148088.1; ZP_00207996.1; AAF64138.1; |
| | | | AAA96487.1; EAM93501.1; ZP_00463487.1; |
| | | | YP_074243.1; YP_095531.1; NP_734581.1; |
| | | | NP_692336.1; NP_979105.1; YP_116095.1; |
| | | | ZP_00630106.1; AAP95326.1; CAA61895.1; |
| | | | AAL00246.1 |
| transhydrogenase | redox- | NP_334574 | NP_962508.1; NP_302686.1; YP_117224.1; |
| pntB (R70) | conversion | | ZP_00517957.1; ZP_00112135.1; NP_681485.1; |
| | of pyrimidin | | ZP_00203498.1; ZP_00411543.1; BAB75107.1; |
| | nucleotides | | ZP_00673742.1; ZP_00315150.1; |
| | | | AAZ25627.1; EAN07189.1; ZP_00549520.1; |
| | | | NP_105891.1; CAC47439.1; ZP_00318702.1; |
| | | | ZP_00400206.1; CAD77499.1; ZP_00164663.2; |
| | | | YP_170777.1; YP_005747.1; YP_143474.1; |
| | | | ZP_00626042.1; YP_190751.1; |
| | | | AAQ87239.1; NP_773764.1; Q59765; NP_949516.1; |
| | | | YP_223058.1; AAK25265.1; AAQ57778.1; |
| | | | ZP_00348709.1; YP_266000.1; ZP_00267648.1; |
| | | | AAN47534.1; AAM35812.1; |
| | | | ZP_00241933.1; ZP_00523138.1; CAE21339.1; |
| | | | ZP_00577769.1; YP_159578.1; ZP_00417258.1; |
| | | | ZP_00599375.1; ZP_00377317.1; |
| | | | ZP_00314523.1; NP_719280.1; NP_795188.1; |
| | | | NP_888489.1; NP_884728.1; YP_257265.1; |
| | | | YP_202268.1; ZP_00215077.1; ZP_00051959.2; |
| | | | ZP_00420704.1; ZP_00262288.1; |
| | | | NP_840935.1; CAB84437.1; ZP_00600539.1; |
| | | | ZP_00507651.1; NP_248887.1; NP_696033.1; |
| | | | EAN27201.1; AAY40045.1; AAN65789.1; |
| | | | YP_126266.1; ZP_00303636.1; AAN62246.1; |
| | | | NP_893262.1; YP_094909.1; YP_123265.1; |
| | | | CAD16440.1; YP_115164.1; CAE07209.1; |
| | | | AAW90112.1; AAF41382.1; YP_277177.1; |
| | | | CAG75106.1; ZP_00497831.1; NP_929428.1; |
| | | | NP_440860.1; ZP_00339822.1; |
| | | | YP_246075.1; ZP_00452010.1; ZP_00216676.1; |
| | | | ZP_00350599.1; AAQ00284.1; AAO91446.1; |
| | | | ZP_00655137.1; AAZ18418.1; ZP_00464938.1; |
| | | | ZP_00457884.1; ZP_00669207.1; |
| | | | YP_047599.1; YP_070739.1; NP_800431.1; |
| | | | AAF96466.1; NP_359741.1; YP_067026.1; |
| | | | ZP_00598774.1; ZP_00168669.2; YP_206543.1; |
| | | | YP_132575.1; ZP_00170342.1; YP_063030.1; |
| | | | ZP_00282750.1; EAA25826.1; |
| | | | NP_936867.1; ZP_00634798.1; ZP_00153170.2; |
| | | | ZP_00585179.1; NP_220468.1; AAX65404.1; |
| | | | NP_707499.1; YP_150630.1; AAZ27182.1; |
| | | | ZP_00581426.1; ZP_00170332.1; |
| | | | ZP_00278457.1; ZP_00170182.2; ZP_00167836.2; |
| | | | NP_533165.1; AAK02836.1; ZP_00135326.1; |
| | | | NP_439514.1; ZP_00157199.1; ZP_00638575.1; |
| | | | AAX88574.1; BAC68112.1; |
| | | | YP_055340.1; CAC16724.1; ZP_00169560.2; |
| | | | AAU37830.1; ZP_00006258.1; ZP_00628636.1; |
| | | | AAQ87370.1; ZP_00133010.1; AAQ66400.1; |
| | | | ZP_00657269.1; AAP96434.1; ZP_00166114.2; |
| | | | ZP_00414377.1; EAM24331.1; |
| | | | AAV96060.1; CAE68875.1; ZP_00620416.1; |
| | | | ZP_00554983.1; AAB52670.1; EAA53262.1; |
| | | | EAL86026.1; ZP_00644761.1; ZP_00149697.1; |
| | | | XP_312859.2; CAB88572.2; |
| | | | XP_326633.1;; CAF99856.1; CAF99322.1; |
| | | | XP_424784.1; NP_032736.2; AAH08518.1; |
| | | | BAC39226.1; AAH91271.1; CAA89065.1; |
| | | | ZP_00048453.1; AAF72982.2; XP_536481.1; |
| | | | AAH66499.1; BAC39564.1; BAC30596.1;; |
| | | | ZP_00533817.1; AAH81117.1; NP_776368.1; |
| | | | AAA21440.1; P11024; AAC43725.1; |
| | | | Q13423; CAD38536.1; XP_679831.1; CAH90079.1; |
| | | | CAA90428.1; ZP_00054747.1; AAC41577.2; |
| | | | EAA18012.1; NP_702397.1; EAK89427.1; |
| | | | CAA10358.1; AAC51914.1; AAK18179.1; |
| | | | NP_971796.1; 1NM5; NP_522515.1; |
| | | | 1PTJ; AAG02246.2; 1XLT; 1PNQ; EAK88482.1; |
| | | | XP_666495.1; XP_646840.1; EAA77364.1; |
| | | | XP_648285.1; XP_666155.1; AAH32370.1; |
| | | | 1D4O; XP_669801.1; XP_694555.1; |
| | | | XP_517776.1; AAO07275.1; EAA58767.1; |
| | | | AAO07276.1; AAP50917.1; AAP50916.1; |
| | | | AAP15452.1; AAP50915.1; AAP50914.1; |
| | | | XP_695634.1; AAA29081.1; NP_522512.1; |
| | | | ZP_00202835.1; ZP_00208470.1; AAD09942.1; |
| | | | XP_738089.1; XP_653216.1; CAI37445.1; |
| | | | XP_582741.1; ZP_00655886.1; ZP_00166345.1; |
| | | | ZP_00048483.2; ZP_00675280.1; |
| | | | ZP_00412076.1; ZP_00170534.2; BAB05258.1; |
| | | | XP_422112.1; AAB23106.1; ZP_00665383.1; |
| | | | ZP_00544728.1; ZP_00385815.1; ZP_00674068.1; |
| | | | ZP_00634699.1; NP_990622.1 |
| transhydrogenase | redox- | NP_214669 | NP_962506.1; NP_302688.1; YP_117222.1; |
| pntA (R70) | conversion | | ZP_00673740.1; ZP_00400208.1; YP_202272.1; |
| | of pyrimidin | | YP_005749.1; ZP_00162920.2; ZP_00315152.1; |
| | nucleotides | | BAB75109.1; NP_681483.1; ZP_00150890.1; |
| | | | ZP_00507653.1; ZP_00549518.1; |
| | | | ZP_00203569.1; YP_159576.1; ZP_00112137.1; |
| | | | YP_190749.1; ZP_00417256.1; CAD16437.1; |
| | | | ZP_00523136.1; AAN62244.1; |
| | | | YP_103924.1; ZP_00282748.1; NP_248885.1; |
| | | | ZP_00140616.1; ZP_00669209.1; AAY40043.1; |
| | | | ZP_00485169.1; AAO91444.1; ZP_00517955.1; |
| | | | ZP_00241930.1; ZP_00170340.1; |
| | | | CAC16725.1; YP_277175.1; NP_522510.1; |
| | | | YP_257267.1; NP_881481.1; EAA53262.1; |
| | | | ZP_00166078.2; NP_884730.1; ZP_00167837.2; |
| | | | NP_840933.1; ZP_00262286.1; ZP_00464936.1; |
| | | | ZP_00497829.1; YP_170779.1; |
| | | | ZP_00411541.1; ZP_00216674.1; CAE21341.1; |
| | | | ZP_00492956.1; ZP_00170330.2; |
| | | | ZP_00168666.1; AAN47251.1; AAM35806.1; |
| | | | BAC68113.1; CAE07207.1; ZP_00278459.1; |
| | | | AAQ57780.1; YP_115165.1; XP_326633.1; |
| | | | ZP_00598776.1; ZP_00170183.2; AAQ87237.1; |
| | | | CAB88572.2; ZP_00166343.1; AAQ00286.1; |
| | | | CAD77501.1; ZP_00314525.1; EAL86026.1; |
| | | | NP_105889.1; YP_055339.1; NP_773766.1; |
| | | | EAN07187.1; EAN27202.1; CAB84439.1; |
| | | | ZP_00318700.1; ZP_00414378.1; |
| | | | CAG75105.1; AAB52670.1; ZP_00267650.1; |
| | | | 1PTJ; 1L7D; EAA77364.1; XP_562937.1; |
| | | | ZP_00600541.1; AAK25267.1; CAE68875.1; |
| | | | ZP_00420705.1; NP_949518.1; AAW90110.1; |
| | | | 1NM5; ZP_00377320.1; NP_893264.1; |
| | | | ZP_00169561.2; NP_696034.1; NP_929427.1; |
| | | | YP_247378.1; AAF41384.1; NP_541301.1; |
| | | | YP_070740.1; NP_669446.1; CAC47441.1; |
| | | | AAN34144.1; ZP_00133009.1; ZP_00122250.1; |
| | | | ZP_00638574.1; YP_223056.1; |
| | | | ZP_00208471.1; ZP_00626044.1; AAK02837.1; |
| | | | AAZ18416.1; CAA46884.1; NP_753890.1; |
| | | | NP_360971.1; ZP_00154275.2; AAP96435.1; |
| | | | AAX88575.1; XP_646840.1; ZP_00655135.1; |
| | | | AAU37831.1; 1F8G; ZP_00585178.1; |
| | | | YP_150631.1; NP_805194.1; AAL20398.1; |
| | | | NP_439513.1; NP_707500.2; YP_047601.1; |
| | | | ZP_00628637.1; EAM24330.1; AAF96465.1; |
| | | | NP_800432.1; NP_440856.1; AAK00588.1; |
| | | | NP_719279.1; AAG56590.1; NP_221211.1; |
| | | | ZP_00577994.1; ZP_00340908.1; |
| | | | ZP_00581427.1; AAZ27926.1; YP_063028.1; |
| | | | AAH66499.1; YP_206544.1; YP_132574.1; |
| | | | AAV96061.1; NP_533164.1; ZP_00006259.2; |
| | | | AAH91271.1; AAQ87369.1; ZP_00215076.1; |
| | | | NP_032736.2; AAH08518.1; CAA89065.1; |
| | | | AAF72982.2; YP_067788.1; ZP_00303638.1; |
| | | | AAK18179.1; ZP_00634797.1; ZP_00135325.1; |
| | | | CAF99322.1; AAO07277.1; |
| | | | NP_936868.1; ZP_00657268.1; ZP_00554982.1; |
| | | | AAG02246.2; XP_536481.1; NP_776368.1; |
| | | | CAD38536.1; P11024; AAA21440.1; |
| | | | Q13423; XP_424784.1; AAH81117.1; AAC51914.1; |
| | | | CAA90428.1; YP_126268.1; ZP_00620417.1; |
| | | | YP_094911.1; CAF99856.1; CAH90079.1; |
| | | | YP_265998.1; XP_666495.1; EAK89427.1;; |
| | | | AAC41577.2; ZP_00120256.2; |
| | | | AAA29081.1; AAA61928.1; ZP_00659346.1; |
| | | | ZP_00599372.1; XP_666155.1; EAK88482.1; |
| | | | BAC39226.1; BAC30596.1; BAC39564.1; |
| | | | AAB81400.1; ZP_00599373.1; NP_702397.1; |
| | | | EAA18012.1; XP_603436.1; XP_679831.1; |
| | | | XP_517777.1; AAM44187.1; NP_217296.1; |
| | | | AAK47169.1; YP_080482.1;; CAA44791.1; |
| | | | NP_302068.1; CAE07016.1; NP_694147.1; |
| | | | ZP_00539848.1; ZP_00386339.1; AAM44190.1; |
| | | | XP_598602.1; ZP_00293711.1; |
| | | | XP_740533.1; EAM73707.1; YP_117835.1; |
| | | | YP_126314.1; ZP_00526155.1; BAB06048.1; |
| | | | YP_094958.1; YP_123314.1; YP_062161.1; |
| | | | NP_693109.1; CAB52837.1; ZP_00333957.1; |
| | | | AAP97897.1; AAT40119.1; YP_149301.1; |
| | | | NP_840123.1; YP_082111.1; 1PJC; |
| | | | AAP07610.1; AAP11530.1; AAM12899.1; |
| | | | YP_021515.1; NP_391071.1; NP_661601.1; |
| | | | YP_081348.1; ZP_00170826.2; ZP_00601871.1; |
| | | | BAC74218.1; ZP_00241359.1; NP_769819.1; |
| | | | ZP_00551096.1; ZP_00462872.1; |
| | | | ZP_00456225.1; YP_174267.1; NP_975075.1; |
| | | | XP_517776.1; ZP_00375402.1; ZP_00167698.2; |
| | | | YP_075651.1; YP_253131.1; YP_056938.1; |
| | | | CAH07118.1; NP_682897.1; BAB06899.1; |
| | | | AAC98487.1; ZP_00659771.1; ZP_00411612.1; |
| | | | AAF11449.1; AAC23577.1; |
| | | | BAC39793.1; ZP_00151223.1; YP_148605.1; |
| | | | P17556; YP_041174.1; YP_005051.1; NP_856449.1; |
| | | | YP_186592.1; NP_372233.1; NP_374819.1; |
| | | | YP_144713.1; ZP_00215625.1; |
| | | | ZP_00378064.1; CAE21637.1; ZP_00323350.1; |
| | | | EAM27747.1; ZP_00497694.1; ZP_00467473.1; |
| | | | NP_764939.1; AAK25536.1; ZP_00303801.1; |
| | | | YP_159073.1; ZP_00517716.1; |
| | | | ZP_00553800.1; ZP_00629472.1; CAF24210.1; |
| | | | ZP_00208007.1; ZP_00671129.1; ZP_00008120.2; |
| | | | YP_129373.1; NP_621858.1; |
| | | | NP_470950.1; YP_130399.1; YP_111103.1; |
| | | | ZP_00310735.1; AAQ59694.1; AAF95053.1; |
| | | | NP_465104.1; ZP_00400392.1; YP_014199.1; |
| | | | AAO76661.1; ZP_00231205.1; AAP44334.1; |
| | | | YP_005739.1; YP_143482.1; NP_797482.1; |
| | | | BAB74054.1; ZP_00120255.1; AAO90629.1; |
| | | | ZP_00636037.1; NP_988633.1; |
| | | | CAB60094.1; CAB59281.2; YP_204286.1; |
| | | | EAM23962.1; YP_040853.1; NP_926915.1; |
| | | | YP_186323.1; CAG43158.1; ZP_00673302.1; |
| | | | ZP_00418709.1; P17557; YP_173042.1; |
| | | | ZP_00640011.1; CAD72056.1; CAC46203.1; |
| | | | ZP_00049286.2; AAM35807.1; XP_672369.1; |
| | | | AAC23579.1; AAC23578.1; ZP_00164800.1; |
| | | | ZP_00507921.1; AAO11283.1; YP_266230.1; |
| | | | ZP_00559586.1; ZP_00601825.1; |
| | | | ZP_00528415.1; CAG35273.1; NP_102173.1; |
| | | | NP_085655.1; YP_015797.1; CAG35269.1; |
| | | | ZP_00621640.1; AAL87460.1; ZP_00112172.1; |
| | | | ZP_00413882.1; ZP_00130164.2; |
| | | | EAN26936.1; ZP_00579039.1; ZP_00519776.1; |
| | | | AAZ24151.1; YP_113082.1; ZP_00534863.1; |
| | | | ZP_00588083.1; AAK38118.1; ZP_00667695.1; |
| | | | NP_969291.1; ZP_00271173.1; |
| | | | ZP_00513142.1; EAN05956.1; ZP_00532101.1; |
| | | | AAQ00644.1; ZP_00397135.1; YP_155059.1; |
| | | | NP_440110.1; NP_768378.1; YP_009793.1; |
| | | | AAR37813.1; AAV93547.1; NP_953341.1; |
| | | | AAN87044.1; AAK99657.1; AAS52072.1; |
| | | | ZP_00545593.1; ZP_00533303.1; |
| | | | NP_636464.1; YP_244224.1; EAM73436.1; |
| | | | ZP_00526469.1; CAJ06319.1; NP_953750.1; |
| | | | CAG36161.1; ZP_00528447.1; ZP_00051957.1; |
| | | | ZP_00589100.1; ZP_00053708.2; ZP_00570829.1; |
| | | | ZP_00571033.1; ZP_00547004.1; |
| | | | YP_177277.1; AAV46533.1; YP_201341.1 |
| formate-- | metabolisation | NP_939608 | CAI37045.1; YP_253105.1; NP_764963.1; |
| tetrahydrofolate | of | | NP_372256.1; YP_041196.1; YP_186615.1; |
| ligase (EC | formate into | | AAP09070.1; NP_978500.1; YP_036267.1; |
| 6.3.4.3) | formyl- | | ZP_00392371.1; YP_018748.1; NP_345695.1; |
| Formyl | tetrathydrofolate | | ZP_00237595.1; AAK99912.1; ZP_00412295.1; |
| tetrahydrofolate | | | NP_471324.1; ZP_00234496.1; YP_014498.1; |
| synthetase | | | ZP_00231031.1; NP_815430.1; |
| (R75) | | | YP_194410.1; AAB49329.1; AAN58771.1; |
| | | | ZP_00332949.1; NP_267091.1; YP_139285.1; |
| | | | AAV62384.1; ZP_00389385.1; NP_735535.1; |
| | | | AAM99936.1; NP_623926.1; ZP_00576302.1; |
| | | | BAB82175.1; ZP_00538881.1; ZP_00504427.1; |
| | | | NP_803035.1; YP_079490.1; |
| | | | YP_091901.1; AAK34738.1; NP_802315.1; |
| | | | AAL98593.1; YP_061089.1; P21164; AAL97781.1; |
| | | | NP_785345.1; ZP_00366155.1; ZP_00560721.1; |
| | | | ZP_00323289.1; ZP_00559333.1; |
| | | | YP_077015.1; 1FP7; ZP_00064107.1; ZP_00633202.1; |
| | | | EAO23711.1; AAK81137.1; |
| | | | IEG7; AAO36782.1; ZP_00134884.1; AAP33693.1; |
| | | | AAU84895.1; Q07064; AAW88826.1; |
| | | | CAB83907.1; AAF42174.1; CAC46969.1; |
| | | | NP_104026.1; ZP_00243151.1; AAP96104.1; |
| | | | ZP_00366396.1; ZP_00206938.1; BAA25140.1; |
| | | | ZP_00402561.1; ZP_00622670.1; |
| | | | ZP_00566160.1; YP_266693.1; YP_114639.1; |
| | | | EAN06465.1; ZP_00658424.1; ZP_00554273.1; |
| | | | AAV96338.1; ZP_00319191.1; AAL94166.1; |
| | | | ZP_00045954.1; NP_965043.1; |
| | | | ZP_00387414.1; ZP_00379883.1; YP_194023.1; |
| | | | CAH07950.1; NP_229563.1; ZP_00143839.1; |
| | | | YP_134776.1; AAO75844.1; NP_970636.1; |
| | | | YP_054766.1; AAK20249.1; AAQ66392.1; |
| | | | AAK20247.1; AAK20248.1; YP_005676.1; |
| | | | CAC12596.1; NP_465401.1; NP_110608.1; |
| | | | AAK20246.1; ZP_00602143.1; CAJ06565.1; |
| | | | EAN84686.1; XP_563307.1; EAM94020.1; |
| | | | YP_205176.1; ZP_00585967.1; |
| | | | BAD38226.1; AAP55207.1; NP_716196.1; |
| | | | NP_936778.1; YP_130938.1; ZP_00356080.1; |
| | | | AAF96515.1; AAM10111.1; ZP_00581353.1; |
| | | | YP_024022.1; AAL67502.1; AAL67504.1; |
| | | | AAL67501.1; NP_800344.1; AAL67506.1; |
| | | | AAL67503.1; ZP_00209144.1; AAL67500.1; |
| | | | BAB96979.1; BAB96978.1; AAL67505.1; |
| | | | BAB97060.1; BAB96906.1; BAB97012.1; |
| | | | BAB97100.1; BAB97118.1; BAB96908.1; |
| | | | BAB97072.1; BAB97009.1; BAB97061.1; |
| | | | BAB97052.1; BAB97051.1; BAB96990.1; |
| | | | BAB97098.1; CAA58847.1; BAB96987.1; |
| | | | BAB97161.1; BAB97034.1; BAB96918.1; |
| | | | BAB96980.1; BAB97031.1; BAB96887.1; |
| | | | BAB96882.1; BAB97066.1; BAB97152.1; |
| | | | BAB96968.1; BAB96859.1; BAB96821.1; |
| | | | BAB97093.1; BAB97026.1; BAB97015.1; |
| | | | BAB96860.1; AAK20256.1; BAB97110.1; |
| | | | BAB96913.1; CAD39284.1; BAB97046.1; |
| | | | BAB96985.1; AAK20257.1; YP_181413.1; |
| | | | BAB97156.1; BAB96953.1; CAD39277.1; |
| | | | BAB97140.1; BAB97068.1; BAB96976.1; |
| | | | BAB97064.1; BAB97145.1; BAB97132.1; |
| | | | BAB97027.1; BAB97013.1; BAB97113.1; |
| | | | BAB97048.1; BAB97024.1; BAB96920.1; |
| | | | CAD39252.1; BAB97091.1; BAB96984.1; |
| | | | BAB96927.1; BAB96865.1; BAB97018.1; |
| | | | BAB96954.1; BAB97106.1; BAB97041.1; |
| | | | BAB97021.1; BAB96938.1; BAB97025.1; |
| | | | BAB97049.1; BAB97005.1; BAB96910.1; |
| | | | BAB97008.1; BAB97148.1; BAB96988.1; |
| | | | BAB96951.1; BAB96941.1; BAB97155.1; |
| | | | BAB96993.1; BAB96904.1; BAB96875.1; |
| | | | BAB97144.1; BAB97006.1; BAB96956.1; |
| | | | BAB96924.1; BAB97108.1; BAB96930.1; |
| | | | AAK20250.1; BAB97162.1; BAB96872.1; |
| | | | BAB96858.1; CAD39278.1; NP_078054.1; |
| | | | BAB97159.1; BAB96952.1; BAB96948.1; |
| | | | BAB97117.1; BAB97070.1; BAB97104.1; |
| | | | BAB97103.1; BAB97083.1; BAB97055.1; |
| | | | BAB96830.1; BAB97089.1; AAK20254.1; |
| | | | BAB97139.1; BAB97053.1; BAB96997.1; |
| | | | BAB96869.1; BAB96837.1; BAB96925.1; |
| | | | BAB96921.1; BAB96903.1; BAB96900.1; |
| | | | BAB97028.1; BAB97136.1; BAB97126.1; |
| | | | BAB97092.1; AAK20258.1; BAB97069.1; |
| | | | AAK20255.1; BAB96961.1; BAB96957.1; |
| | | | CAD39216.1; AAK20253.1; AAO37738.1; |
| | | | BAB96977.1; BAB96888.1; BAB96893.1; |
| | | | CAD39221.1; AAK20261.1; AAK20251.1; |
| | | | BAB97124.1; BAB97029.1; BAB96940.1; |
| | | | BAB96876.1; BAB96842.1; BAB96831.1; |
| | | | CAD39245.1; AAK20252.1; CAD39266.1; |
| | | | BAB97134.1; BAB97130.1; BAB97099.1; |
| | | | BAB97032.1; BAB96848.1; BAB97017.1; |
| | | | BAB97010.1; BAB96885.1; AAK20268.1; |
| | | | BAB97123.1; BAB97063.1; BAB96971.1; |
| | | | BAB97114.1; BAB97094.1; BAB97056.1; |
| | | | BAB96886.1; BAB96843.1; BAB97138.1; |
| | | | BAB97135.1; BAB97016.1; BAB96998.1; |
| | | | BAB96936.1; BAB96919.1; BAB97160.1; |
| | | | BAB97131.1; BAB97039.1; BAB97033.1; |
| | | | BAB97023.1; BAB97003.1; BAB96983.1; |
| | | | BAB96850.1; BAB96849.1; BAB97030.1; |
| | | | BAB96991.1; BAB96894.1; BAB96863.1; |
| | | | CAD39217.1; BAB97133.1; BAB97112.1; |
| | | | BAB97111.1; BAB97062.1; BAB97045.1; |
| | | | BAB96929.1; BAB96874.1; BAB96827.1; |
| | | | BAB97128.1; BAB97121.1; BAB97096.1; |
| | | | BAB97058.1; BAB97054.1; BAB97047.1; |
| | | | BAB96970.1; BAB96922.1; BAB96828.1; |
| | | | CAD39229.1; BAB97078.1; BAB97011.1; |
| | | | BAB96960.1; BAB96939.1; BAB97154.1; |
| | | | BAB97142.1; BAB96937.1; CAD39275.1; |
| | | | BAB97057.1; BAB97037.1; BAB96992.1; |
| | | | BAB96964.1; BAB96846.1; BAB96838.1; |
| | | | BAB96829.1; BAB97119.1; BAB96931.1; |
| | | | AAO37727.1; BAB96963.1; BAB96890.1; |
| | | | BAB96857.1; BAB96844.1; BAB97129.1; |
| | | | BAB97127.1; BAB97075.1; BAB96975.1; |
| | | | BAB96899.1; BAB96889.1; BAB96881.1; |
| | | | CAD39224.1; BAB97149.1; BAB97073.1; |
| | | | BAB97043.1; BAB97038.1; CAD39240.1; |
| | | | BAB97086.1; BAB97004.1; CAD39283.1; |
| | | | CAD39228.1; CAD39222.1; CAD39220.1; |
| | | | AAK20260.1; BAB97088.1; CAD39272.1; |
| | | | BAB97137.1; BAB97059.1; BAB96965.1; |
| | | | BAB96911.1; BAB96905.1; BAB96841.1; |
| | | | CAD39237.1; BAB97014.1; BAB96898.1; |
| | | | BAB96996.1; BAB96835.1; BAB97084.1; |
| | | | BAB96867.1; BAB96822.1; BAB97125.1; |
| | | | BAB97107.1; BAB96907.1; CAD39249.1; |
| | | | CAD39236.1; CAD39213.1; CAD39212.1; |
| | | | BAB97105.1; BAB96902.1; CAD39279.1; |
| | | | BAB97116.1; BAB96866.1; BAB97102.1; |
| | | | AAK20259.1; BAB97040.1; CAD39256.1; |
| | | | BAB97157.1; BAB97071.1; BAB96972.1; |
| | | | CAD39261.1; AAO37723.1; BAB97101.1; |
| | | | BAB97095.1; BAB96879.1; CAD39248.1; |
| | | | CAD39231.1; CAG36956.1; BAB96955.1; |
| | | | BAB96943.1; CAD39233.1; BAB97081.1; |
| | | | BAB97150.1; BAB96999.1; BAB96949.1; |
| | | | BAB96854.1; BAB96947.1; CAD39263.1; |
| | | | AAK20269.1; BAB96967.1; BAB96962.1; |
| | | | BAB96917.1; CAD39264.1; AAO37741.1; |
| | | | BAB96873.1; BAB96839.1; CAD39269.1; |
| | | | CAD39234.1; BAB97151.1; BAB97085.1; |
| | | | BAB97080.1; BAB96995.1; CAD39280.1; |
| | | | BAB97153.1; CAD39242.1; CAD39215.1; |
| | | | AAO37740.1; BAB96891.1; CAD39243.1; |
| | | | BAB97079.1; BAB97042.1; BAB96840.1; |
| | | | CAD39241.1; CAD39274.1; AAK20262.1; |
| | | | BAB97050.1; CAD39271.1; CAD39265.1; |
| | | | AAO37743.1; AAO37729.1; BAB96923.1; |
| | | | BAB96884.1; BAB96825.1; CAD39251.1; |
| | | | AAO37735.1; CAD39282.1; BAB96861.1; |
| | | | AAO37724.1; CAD39270.1; CAD39235.1; |
| | | | BAB96933.1; BAB96946.1; BAB97146.1; |
| | | | BAB96986.1; BAB96896.1; CAD39232.1; |
| | | | AAO37736.1; BAB96959.1; AAK20267.1; |
| | | | CAD39223.1; CAD39244.1; BAB96966.1; |
| | | | BAB97141.1; CAD39276.1; BAB96935.1; |
| | | | CAD39255.1; CAD39209.1; BAB97115.1; |
| | | | CAD39253.1; AAO37730.1; BAB96926.1; |
| | | | BAB96870.1; AAO37737.1; BAB96958.1; |
| | | | BAB97022.1; CAD39250.1 |
| formyl | converts | NP_600108 | NP_737565.1; ZP_00655917.1; NP_939225.1; |
| tetrahydrofolate | formyl-THF | | BAC71386.1; CAB59679.1; ZP_00292666.1; |
| cyclo ligase | to 5,10- | | YP_121170.1; YP_061656.1; ZP_00545597.1; |
| (EC 6.3.3.2.) | methenyl- | | NP_301256.1; NP_959857.1; ZP_00573419.1; |
| (R76) | THF | | CAI37699.1; NP_214187.1; 1SOU; |
| | | | YP_075021.1; NP_696710.1; ZP_00412260.1; |
| | | | ZP_00134730.1; EAM72866.1; ZP_00575597.1; |
| | | | AAO89633.1; NP_215507.1; NP_854676.1; |
| | | | AAK45268.1; EAN27919.1; ZP_00552794.1; |
| | | | NP_882562.1; ZP_00326738.1; |
| | | | YP_115170.1; NP_886754.1; NP_881632.1; |
| | | | NP_534225.1; NP_440379.1; ZP_00626722.1; |
| | | | ZP_00600182.1; YP_079816.1; YP_148302.1; |
| | | | ZP_00519228.1; NP_439018.1; AAO44764.1; |
| | | | NP_266325.1; ZP_00323162.1; |
| | | | ZP_00156713.1; ZP_00110015.2; ZP_00471373.1; |
| | | | ZP_00534935.1; ZP_00159040.2; ZP_00171408.2; |
| | | | ZP_00398486.1; YP_190665.1; |
| | | | ZP_00396530.1; YP_009590.1; AAQ66843.1; |
| | | | YP_125990.1; AAR37522.1; YP_270316.1; |
| | | | YP_094629.1; YP_122981.1; AAL00707.1; |
| | | | NP_930816.1; BAB82030.1; ZP_00155857.2; |
| | | | YP_222388.1; ZP_00269658.1; AAX87904.1; |
| | | | AAL51496.1; YP_233427.1; CAG35375.1; |
| | | | AAK79064.1; BAB76524.1; NP_794958.1; |
| | | | AAN70768.1; AAO09973.1; AAO79363.1; |
| | | | ZP_00585069.1; AAN48446.1; |
| | | | ZP_00538598.1; ZP_00417043.1; EAO24395.1; |
| | | | ZP_00638539.1; AAQ58176.1; ZP_00403498.1; |
| | | | YP_144877.1; AAX66908.1; CAC41462.1; |
| | | | NP_952189.1; YP_055204.1; ZP_00347044.1; |
| | | | YP_005216.1; 1WKC; YP_156483.1; |
| | | | EAO17392.1; YP_175218.1; ZP_00594221.1; |
| | | | ZP_00319181.1; AAH19921.1; NP_001009349.1; |
| | | | NP_798970.1; ZP_00556764.1; |
| | | | NP_253915.1; ZP_00630769.1; AAT49915.1; |
| | | | ZP_00141705.1; ZP_00264800.1; NP_081105.1; |
| | | | CAC47337.1; ZP_00055330.1; |
| | | | AAT42396.1; NP_102175.1; CAD13617.1; |
| | | | ZP_00062893.1; ZP_00315438.1; AAF95621.1; |
| | | | NP_971601.1; ZP_00588425.1; YP_034143.1; |
| | | | NP_923705.1; CAH06623.1; YP_098246.1; |
| | | | NP_670599.1; NP_311809.2; ZP_00630026.1; |
| | | | YP_263029.1; AAG10441.1; ZP_00592340.1; |
| | | | YP_170963.1; YP_131238.1; |
| | | | NP_614888.1; CAH03038.1; AAL95098.1; |
| | | | BAA28715.1; EAN08520.1; YP_152082.1; |
| | | | NP_726312.1; NP_611785.1; NP_726311.2; |
| | | | YP_071689.1; CAF26595.1; NP_755368.1; |
| | | | NP_681770.1; AAR38106.1; NP_840158.1; |
| | | | ZP_00509367.1; AAH12417.1; NP_104943.1; |
| | | | ZP_00634551.1; EAA26463.1; ZP_00582514.1; |
| | | | AAN58080.1; NP_768167.1; ZP_00211724.1; |
| | | | NP_638609.1; NP_965424.1; XP_308920.2; |
| | | | NP_785167.1; ZP_00004961.1; |
| | | | AAK25207.1; AAF11368.1; BAB05136.1; |
| | | | ZP_00153791.1; NP_949790.1; EAM47244.1; |
| | | | ZP_00380928.1; YP_205487.1; ZP_00243180.1; |
| | | | ZP_00152677.2; ZP_00623093.1; ZP_00529088.1; |
| | | | ZP_00511292.1; ZP_00655348.1; |
| | | | NP_975489.1; NP_806671.1; NP_777982.1; |
| | | | ZP_00388995.1; XP_413857.1; ZP_00123619.1; |
| | | | AAC75949.1; NP_708674.2; AAV63351.1; |
| | | | YP_067406.1; AAK03805.1; AAH89273.1; |
| | | | AAG58038.1; YP_253281.1; XP_478853.1; |
| | | | NP_360331.1; YP_021133.1; ZP_00530394.1; |
| | | | NP_220841.1; NP_470709.1; |
| | | | ZP_00046926.1; ZP_00334623.1; ZP_00143346.1; |
| | | | ZP_00376747.1; EAN07877.1; NP_464861.1; |
| | | | AAB31354.1; YP_013951.1; NP_692845.1; |
| | | | CAD74215.1; ZP_00278951.1; |
| | | | NP_716405.1; NP_980638.1; EAM31460.1; |
| | | | ZP_00463106.1; AAL21936.1; CAE07229.1; |
| | | | CAC05433.1; AAM60972.1; AAO41971.1; |
| | | | YP_085596.1; AAM90961.1; AAV96195.1; |
| | | | XP_510537.1; NP_660736.1; CAB83635.1; |
| | | | YP_246824.1; AAC65661.1; NP_764791.1; |
| | | | NP_878554.1; CAG86858.1; ZP_00386875.1; |
| | | | YP_194357.1; AAW90586.1; ZP_00434550.1; |
| | | | ZP_00492814.1; YP_053491.1; CAG73377.1; |
| | | | AAO35597.1; EAL25167.1; ZP_00499196.1; |
| | | | CAG80466.1; AAW70845.1; |
| | | | YP_045820.1; NP_734906.1; AAM99309.1; |
| | | | YP_038325.1; CAB66452.1; AAV48062.1; |
| | | | AAH24567.1; YP_041023.1; YP_186447.1; |
| | | | NP_372074.1; ZP_00385074.1; NP_390369.1; |
| | | | XP_591041.1; XP_588513.1; AAV88839.1; |
| | | | AAF42416.1; AAP11177.1; ZP_00238535.1; |
| | | | AAM38252.1; YP_153863.1; AAZ19506.1; |
| | | | YP_266014.1; NP_968104.1; NP_816417.1; |
| | | | YP_255152.1; CAA12119.1; CAH89819.1; |
| | | | BAB14383.1; BAC43679.1; BAB14739.1; |
| | | | CAA21728.1; 1YDM; ZP_00544875.1; |
| | | | NP_766349.1; NP_779018.1; AAB84710.1; |
| | | | ZP_00210813.1; ZP_00579599.1; YP_170178.1; |
| | | | AAX78148.1; NP_565139.1; YP_199768.1; |
| | | | AAF73519.1; ZP_00303053.1; XP_511153.1; |
| | | | AAU37082.1; NP_280554.1; EAK93783.1; |
| | | | AAH79691.1; AAS51535.1; AAH94085.1; |
| | | | YP_158939.1; NP_966978.1; CAF99528.1; |
| | | | ZP_00370459.1; ZP_00652045.1; |
| | | | ZP_00548066.1; ZP_00660458.1; XP_395864.2; |
| | | | NP_341992.1; CAF18476.1; NP_299295.1; |
| | | | CAA83541.1; BAB65129.1; ZP_00373105.1; |
| | | | YP_180354.1; NP_220167.1; XP_427228.1; |
| | | | EAL28752.1; NP_011110.1; XP_694566.1; |
| | | | XP_414185.1; CAE66846.1; ZP_00310271.1; |
| | | | CAE21313.1; CAH64402.1; XP_545889.1; |
| | | | XP_598686.1; AAH37852.1; EAN86573.1; |
| | | | AAP98720.1; ZP_00367232.1; |
| | | | ZP_00063393.2; NP_661939.1; AAP05733.1; |
| | | | NP_701792.1; XP_759454.1; AAL64263.1; |
| | | | EAN86747.1; AAF52130.1; XP_310457.2; |
| | | | AAN71569.1; AAP56729.1; NP_147009.1; |
| | | | EAL86873.1; YP_076645.1; ZP_00168659.2; |
| | | | ZP_00598787.1; AAZ13064.1; CAB76079.1; |
| | | | CAJ04645.1; ZP_00632270.1; CAE09241.1; |
| O-acetyl- | transfer of | CAF19359 | NP_737289.1; NP_939004.1; CAI37871.1; |
| homoserine | methyl- | | NP_962391.1; NP_217857.1; YP_119808.1; |
| sulfhydrolase | mercaptane | | ZP_00411842.1; ZP_00381377.1; EAM75402.1; |
| O-succinyl- | on O-acetyl- | | YP_062728.1; AAP21657.1; ZP_00547645.1; |
| homoserine | homoserine | | ZP_00293095.1; ZP_00570235.1; |
| sulfhydrolase | or O- | | ZP_00570237.1; AAK80727.1; YP_146137.1; |
| O-acetyl- | succinyl- | | ZP_00504479.1; NP_464123.1; NP_469947.1; |
| homoserine | homoserine | | YP_013229.1; YP_173935.1; BAB06322.1; |
| methyl- | | | NP_623710.1; AAO77494.1; ZP_00388850.1; |
| sulfhydrolase | | | AAV62566.1; YP_076611.1; ZP_00526270.1; |
| O-succinyl- | | | NP_693970.1; YP_098691.1; AAP51117.1; |
| homoserine | | | CAH07057.1; YP_252508.1; YP_001801.1; |
| methyl- | | | NP_785969.1; AAN49261.1; ZP_00656265.1; |
| sulfhydrolase | | | NP_696109.1; NP_266229.1; ZP_00382492.1; |
| (R77) | | | AAP21659.1; ZP_00319952.1; |
| | | | AAN58864.1; CAA71732.1; ZP_00064071.1; |
| | | | ZP_00358038.1; ZP_00499571.1; ZP_00576077.1; |
| | | | EAM27331.1; YP_133082.1; ZP_00629666.1; |
| | | | ZP_00217525.1; NP_887601.1; |
| | | | AAN68137.1; ZP_00107219.1; ZP_00462048.1; |
| | | | NP_253712.1; ZP_00551474.1; ZP_00141499.1; |
| | | | NP_952236.1; ZP_00506665.1; |
| | | | YP_076510.1; ZP_00160457.2; NP_228690.1; |
| | | | YP_242180.1; YP_269261.1; NP_638415.1; |
| | | | NP_797008.1; ZP_00208451.1; AAL76402.1; |
| | | | ZP_00595723.1; ZP_00512971.1; |
| | | | CAE10117.1; ZP_00242385.1; ZP_00536577.1; |
| | | | AAR38305.1; NP_948106.1; ZP_00398593.1; |
| | | | ZP_00654525.1; ZP_00531189.1; NP_946915.1; |
| | | | NP_930734.1; NP_661504.1; ZP_00005143.1; |
| | | | NP_841729.1; CAE21050.1; |
| | | | CAE07366.1; YP_047868.1; CAD78524.1; |
| | | | ZP_00283981.1; NP_767875.1; NP_949925.1; |
| | | | YP_221536.1; AAL52347.1; AAP99844.1; |
| | | | AAN29722.1; ZP_00589323.1; ZP_00624467.1; |
| | | | CAG81467.1; ZP_00562640.1; EAN27962.1; |
| | | | ZP_00309879.1; ZP_00556008.1; |
| | | | YP_171853.1; ZP_00170753.1; YP_160629.1; |
| | | | EAO18327.1; YP_156395.1; ZP_00151182.1; |
| | | | ZP_00395964.1; CAG37235.1; ZP_00410921.1; |
| | | | ZP_00269047.1; AAM06094.1; NP_772921.1; |
| | | | NP_892760.1; CAG73733.1; NP_766885.1; |
| | | | EAL85880.1; XP_761728.1; AAO77030.1; |
| | | | AAA61543.1; ZP_00169873.1; |
| | | | ZP_00638540.1; ZP_00527619.1; AAV94718.1; |
| | | | EAM43226.1; ZP_00661517.1; CAD15264.1; |
| | | | ZP_00268814.1; ZP_00620303.1; ZP_00634556.1; |
| | | | AAF10450.1; EAA59015.1; |
| | | | AAV31651.1; NP_953471.1; NP_771607.1; |
| | | | EAA67392.1; AAQ59608.1; EAM75775.1; |
| | | | NP_949587.1; ZP_00595151.1; NP_716721.1; |
| | | | ZP_00534594.1; ZP_00305184.1; ZP_00592653.1; |
| | | | AAK02822.1; ZP_00269954.1; ZP_00543698.1; |
| | | | ZP_00552392.1; CAH09043.1; |
| | | | EAA56840.1; CAA22286.1; ZP_00650573.1; |
| | | | CAC45827.1; NP_108558.1; NP_531944.1; |
| | | | YP_004383.1; YP_144026.1; YP_266440.1; |
| | | | NP_693560.1; YP_257642.1; ZP_00134047.2; |
| | | | ZP_00581547.1; CAB99179.2; YP_159786.1; |
| | | | EAM73042.1; CAG36429.1; AAO35727.1; |
| | | | YP_022333.1; ZP_00239222.1; |
| | | | ZP_00269964.1; YP_086673.1; NP_981827.1; |
| | | | AAP12268.1; AAP77233.1; YP_039397.1; |
| | | | ZP_00208374.1; EAN06205.1; CAE10904.1; |
| | | | AAK99898.1; YP_179865.1; ZP_00387486.1; |
| | | | CAH00339.1; CAB73713.1; CAC34631.1; |
| | | | ZP_00370704.1; NP_013406.1; AAV47483.1; |
| | | | ZP_00489181.1; AAS51890.1; NP_947707.1; |
| | | | EAK94256.1; CAG88952.1; AAF01454.1; |
| | | | AAF01453.1; NP_281028.1; CAG58594.1; |
| | | | EAM46849.1; AAV47781.1; ZP_00566262.1; |
| | | | ZP_00402706.1; YP_234755.1; |
| | | | ZP_00334304.1; YP_259190.1; NP_793584.1; |
| | | | EAO18747.1; ZP_00473306.1; AAO36990.1; |
| | | | NP_840779.1; ZP_00333201.1; ZP_00265576.1; |
| | | | NP_251797.1; ZP_00535298.1; |
| | | | ZP_00204956.1; 1Y4I; AAC83351.1; AAO46884.1; |
| | | | AAN32868.1; YP_074661.1; BAC02724.1; |
| | | | ZP_00348658.1; ZP_00593187.1; |
| | | | NP_947695.1; AAV68695.1; AAK29460.1; |
| | | | ZP_00168139.2; YP_114900.1; AAL95612.1; |
| | | | NP_972801.1; YP_046919.1; YP_200029.1; |
| | | | ZP_00317087.1; ZP_00280956.1; ZP_00416859.1; |
| | | | YP_111696.1; ZP_00502001.1; ZP_00466954.1; |
| | | | AAR38140.1; ZP_00448460.1; |
| | | | NP_950100.1; ZP_00395704.1; ZP_00457013.1; |
| | | | NP_717420.1; ZP_00464307.1; AAQ65554.1; |
| | | | ZP_00213081.1; AAQ60395.1; |
| | | | YP_159732.1; ZP_00637444.1; ZP_00425258.1; |
| | | | ZP_00643937.1; BAB55900.1; ZP_00048836.2; |
| | | | NP_767745.1; YP_268443.1; ZP_00583389.1; |
| | | | ZP_00396707.1; ZP_00636123.1; |
| | | | AAV94639.1; EAN05079.1; AAF11730.1; |
| | | | NP_623174.1; ZP_00587584.1; AAZ18645.1; |
| | | | CAE10114.1; ZP_00303125.1; ZP_00623125.1; |
| | | | AAK78087.1; ZP_00655219.1; |
| | | | CAA09983.1; ZP_00624529.1; AAK24209.1; |
| | | | AAL52798.1; AAB03240.1; ZP_00542518.1; |
| | | | NP_531136.1; AAK25130.1; AAN66932.1; |
| | | | P13254; ZP_00400550.1; BAB04518.1; |
| | | | YP_221092.1; NP_945724.1; NP_970501.1; |
| | | | AAU37954.1; NP_981084.1; 1GC2; ZP_00548195.1; |
| | | | NP_635109.1; CAA04124.1; ZP_00236112.1; |
| | | | YP_004765.1; ZP_00055526.1; |
| | | | ZP_00207149.1; AAM05915.1; YP_085976.1; |
| | | | 1E5F; ZP_00308639.1; ZP_00578607.1; |
| | | | EAN29492.1; YP_144422.1; |
| | | | ZP_00507181.1; |
| pyruvate | catalyses the | YP_226326 | |
| kinase (R19) | step from | | |
| | phosphoenolpyruvate | | |
| | to | | |
| | pyruvate | | |
| formyl- | degrades | ADD13491 | |
| tetrahydrofolate | Formyl THF | | |
| deformylase | to formate | | |
| (R79) | and | | |
| | tetrahydrofolate | | |
| phosphoenol- | converts | NP_602055 | |
| pyruvate | oxaloacetate | | |
| carboxykinase | to | | |
| (GTP) (R35) | Phosphoenol- | | |
| | pyruvate | | |

Growth of Escherichia coli and Corynebacterium glutamicum-Media and Culture Conditions

The person skilled in the art is familiar with the cultivation of common microorganisms such as C. glutamicum and E. coli. Thus, a general teaching will be given below as to the cultivation of C. glutamicum. Corresponding information may be retrieved from standard textbooks for cultivation of E. coli.

E. coli strains are routinely grown in MB and LB broth, respectively (Follettie, M. T., Peoples, 0., Agoropoulou, C., and Sinskey, A J. (1993) J. Bacteriol. 175, 4096-4103). Minimal media for E. coli is M9 and modified MCGC (Yoshihama, M., Higashiro, K., Rao, E. A., Akedo, M., Shanabruch, W G., Follettie, M. T., Walker, G. C., and Sinskey, A. J. (1985) J. Bacteriol. 162, 591-507), respectively. Glucose may be added at a final concentration of 1%. Antibiotics may be added in the following amounts (micrograms per millilitre): ampicillin, 50; kanamycin, 25; nalidixic acid, 25. Amino acids, vitamins, and other supplements may be added in the following amounts: methionine, 9.3 mM; arginine, 9.3 mM; histidine, 9.3 mM; thiamine, 0.05 mM. E. coli cells are routinely grown at 37 C, respectively.

Genetically modified Corynebacteria are typically cultured in synthetic or natural growth media. A number of different growth media for Corynebacteria are both well-known and readily available (Lieb et al. (1989) Appl. Microbiol. Biotechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, and thiourea, sulfur containing amino acids like cysteine and other sulfur containing compounds can be used to achieve efficient methionine production. Formate may also be possible as a supplement as are other C1 sources such as methanol or formaldehyde). Particularly suited are methanethiol and its dimer dimethyldisulfide.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121° C.) or by sterile filtration. The components can either be sterilized together or, if necessary, separately.

All media components may be present at the beginning of growth, or they can optionally be added continuously or batch wise. Culture conditions are defined separately for each experiment.

The temperature should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300'rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30 C.

Inoculation of the media is accomplished by either introduction of a saline suspension of C. glutamicum cells from CM plates or addition of a liquid preculture of this bacterium.

The invention will now be illustrated by means of various examples. These examples are however in no way meant to limit the invention in any way.

### EXAMPLES

The embodiments within the specification provide an illustration of embodiments in this disclosure and should not be construed to limit its scope. The skilled artisan readily recognizes that many other embodiments are encompassed by this disclosure. All publications and patents cited and sequences identified by accession or database reference numbers in this disclosure are incorporated by reference in their entirety. To the extent the material incorporated by reference contradicts or is inconsistent with the present specification, the present specification will supersede any such material. The citation of any references herein is not an admission that such references are prior art to the present disclosure.

Unless otherwise indicated, all numbers expressing quantities of ingredients, cell culture, treatment conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims

A) Theoretical prediction of optimal metabolic flux for an organism with increased efficiency of methionine synthesis
Constructing the Metabolic Networks for C. glutamicum and E. coli

C. glutamicum network. The basic metabolic network of the C. glutamicum wild type was set up for utilization of glucose and sulfate as carbon and sulfur source, respectively (http://www.genomejp/kegg/metabolism.html). It includes glucose uptake via a phosphotransferase system (PTS), glycolysis (EMP), pentose phosphate pathway (PPP), tricarboxylic acid (TCA) cycle, anaplerosis and respiratory chain. The assimilation of sulfate comprises uptake and subsequent conversion into hydrogen sulfide (Schiff (1979), Ciba Found Symp, 72, 49-69). In the stoichiometric model the sulfate assimilation pathway was lumped into 2 reactions: the reduction of sulfate to sulfite requiring 2 ATP and I NADPH and the reduction of sulfite to sulfide demanding for 3 NADPH. The complete model consisted of 59 internal and 8 external metabolites. The external metabolites comprise substrates (glucose, sulfate, ammonia, oxygen) and products (biomass, CO₂, methionine, glycine). Glycine was considered as external metabolite, because once formed as by-product it cannot be re-utilized by C. glutamicum (http://www.genomejp/kegg/metabolism.html). In total, the metabolic network contains 62 metabolic reactions, out of which 19 were regarded reversible. For ATP production in the respiratory chain a P/O ratio of 2 (for NADH) and 1 (for FADH) was assumed (Klapa et al. (2003) Eur. J. Biochem., 27017, 3525-3542). The precursor demand for biomass formation was taken from the literature (Marx et al. (1996) Biotechnol. Bioeng., 49 (2), 111-129). The sulfate and ammonia demand for the biomass was calculated from the content of the different amino acids in the biomass. The model for C. glutamicum is shown in FIG. 1 E. coli network. The model construction for the central metabolism of the wild type of E. coli was based on literature (Carlson et al. (2004), Biotechnol. Bioeng., 851, 1-19). and databases (http://www.genome.jp/kegg/metabolism.html). The model for growth and methionine production on glucose and sulfate comprised PTS uptake of glucose, EMP, PPP, TCA cycle, anaplerosis, respiratory chain and sulfate assimilation. The metabolic network contained 64 metabolic reactions, whereby 20 were regarded reversible. Glucose, sulfate, ammonia and oxygen were considered as external substrates, biomass, CO₂ and methionine as external products. For interconversion of NADH and NADPH, a reversible transhydrogenase was considered (Yamaguchi et al. (1995), J. Biol. Chem., 27028, 166653-9). Moreover, it was considered that E. coli activates homoserine via succinylation instead of acetylation (R40) (Sekowska et al. (2000), J. Mol. Micorbiol. Biotechnol., 22, 145-177). Furthermore a glycine cleavage system was considered (R71, R72). For ATP production in the respiratory chain P/O ratios of 2 (for NADH) and 1 (for FADH) were assumed (Carlson et al. (2004), vide supra). The precursor demand for biomass formation was taken from the literature (Edwards et al. (2000); Weber et al., (2002).

Network modifications. In further simulations the stoichiometric networks described above were modified. This involved the deletion or insertion of different reactions and pathways potentially of interest to improve methionine production. Additionally, carbon and sulfur sources were varied to investigate their influence on methionine production.

Metabolic pathway analysis. Metabolic pathway analysis was carried out using METATOOL (Pfeiffer et al., (1999), Bioinformatics, 153, 251-7, Schuster et al. 1999) Trends Biotechnol., 172, 53-60). The version used (meta4.0.1_double.exe) is available in the internet http://www.biozentrum.uni-wuerzburg.de/bio-informatik/corputing/metatool/-pinguin.biologie.uni-iena.de/bioinformatik/networks/). The mathematical details of the algorithm are described in Pfeiffer et al. (vide supra) which is hereby incorporated by reference with respect to the way the METATOOL software is to be used.

Metabolic pathway analysis resulted in several hundreds of elementary flux modes for each situation investigated. For each of these flux modes, the carbon yields of biomass (Y_{X/S}) and methionine (Y_{Met/S}) were calculated as percentage of the carbon that entered the system as substrate. Throughout the work it is given in percent values ((C-mol) (C-mol substrate)⁻¹×100). Accordingly also co-substrates, such as formate or methanethiol and its dimer dimethyl disulfide were considered. Comparative analysis of all elementary modes obtained for a certain network scenario allowed the determination of the theoretical maximum yields Y_{X/S, max} and Y_{Met/S, max}.

### Results and Implications of the Model

Comparison of Methionine Production by C. glutamicum and E. coli

The two most promising organisms for biotechnological production of methionine are C. glutamicum and E. coli. To evaluate the potential of these two organisms, metabolic pathway analysis was carried out as described above.

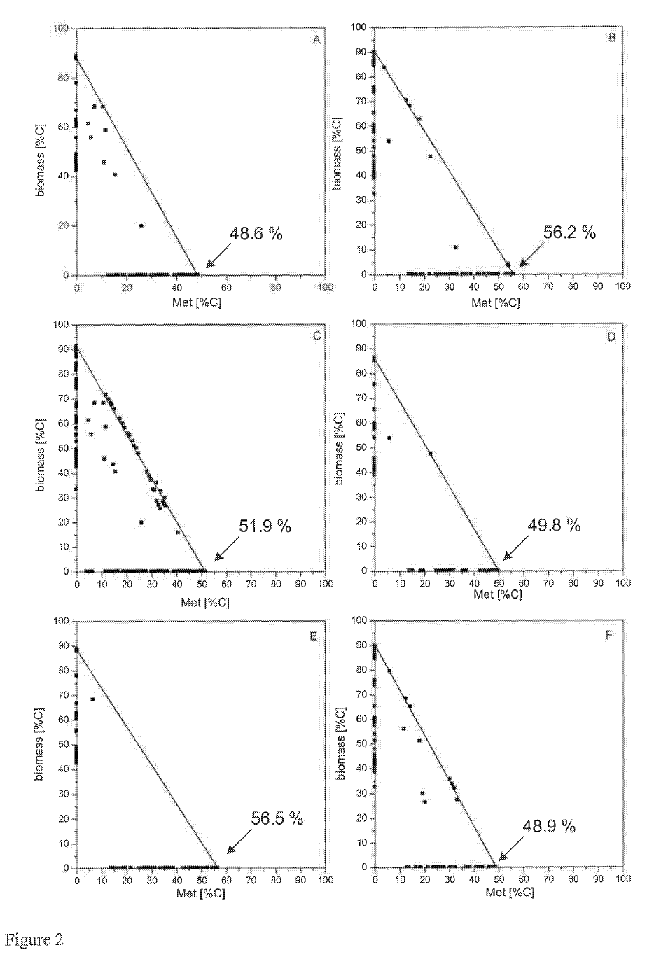

Initially the wild type networks were investigated. As shown for the wild type of C. glutamicum and E. coli, a large number of elementary flux modes with different carbon yields for biomass and methionine was obtained (FIGS. 2 A, B). Among the modes observed, the majority are extreme modes exclusively linked to production of either biomass or methionine. These are given on the two axes of the plot. In addition also flux modes with simultaneous production of biomass and methionine resulted. The maximal theoretical biomass yield was 88.5% for C. glutamicum and thus slightly lower as found for E. coli with 91.7%. Both organisms have a high potential to produce methionine. The maximal theoretical carbon yield for methionine of C. glutamicum was 48.6% (FIG. 2 A). E. coli displays a significantly higher value of 56.2% (FIG. 2 B). The higher potential of the E. coli wild type may indicate advantageous characteristics of its metabolic network. This aspect was studied in additional simulations (see below).

A closer inspection points at two reactions, i.e. the glycine cleavage system and the transhydrogenase, which could be beneficial for increased methionine production. Indeed the optimal solution found for C. glutamicum wild type is linked to substantial formation of glycine, which cannot be re-utilized, whereas no glycine accumulates for optimal methionine production by E. coli wild type. With respect to the high demand of 8 NADPH per methionine, also the availability of the transhydrogenase for interconversion of NADH and NADPH in E. coli could contribute to the higher efficiency observed

To further investigate the importance of these reactions for methionine production, additional simulations were carried out assuming different genetic modifications of the underlying metabolic networks (see below).

Metabolic Fluxes in C. glutamicum and E. coli Under Conditions of Optimal Methionine Production

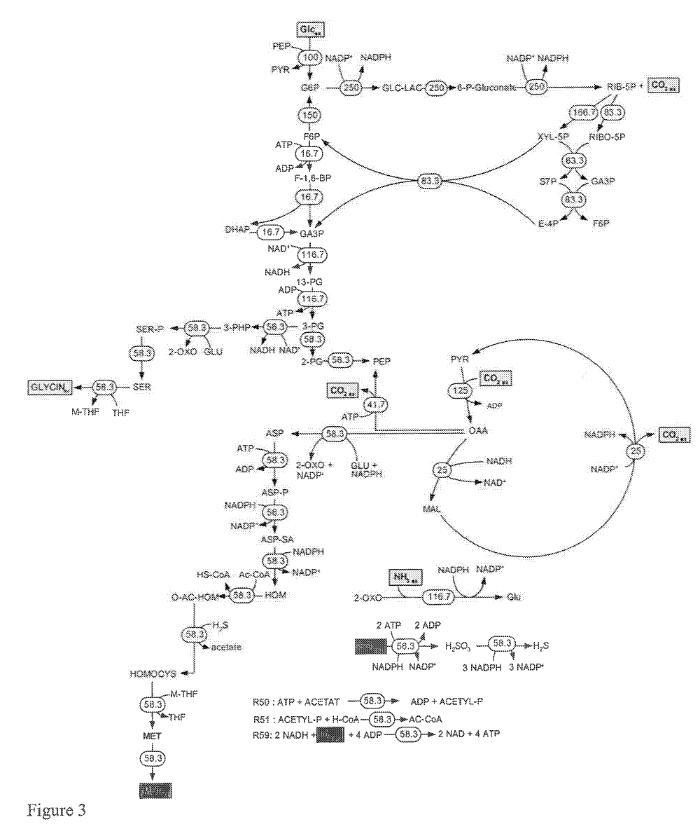

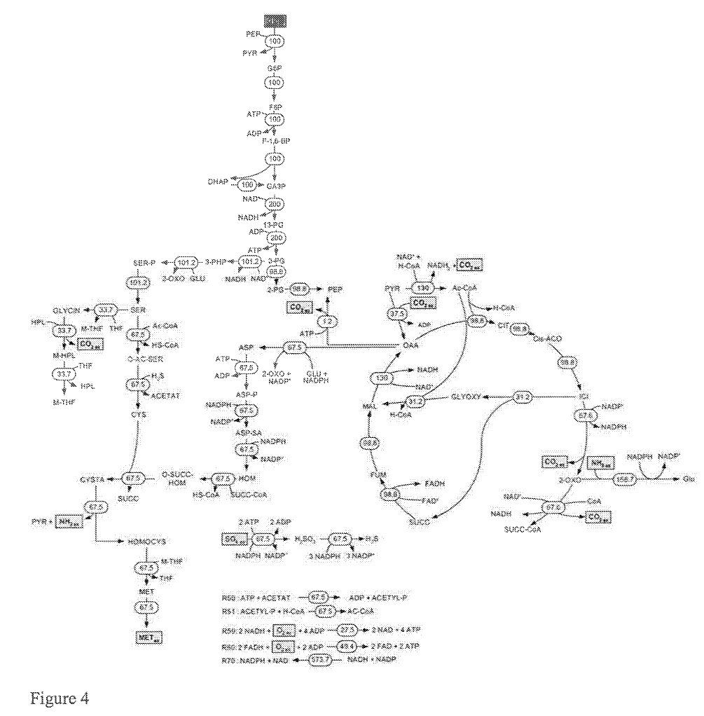

First, the metabolic networks of both organisms were studied in more detail to identify which of the pathways available are involved in optimal methionine production and which pathways should be dispensable. For this purpose, the metabolic flux distribution was calculated for the optimal elementary modes of C. glutamicum and E. coli, i.e. the mode with highest theoretical methionine yield. Hereby all fluxes are given as relative molar values, normalized to the glucose uptake rate, as usually done in metabolic flux analysis. Note that the fluxes (given in mol (mol)⁻¹×100) differ from the carbon yields (in C-mol (C-mol)⁻¹×100) used to describe the maximal performance. Additionally, the reactions from the basic models (FIG. 1) that were inactive in the respective modes were erased from FIGS. 3 and 4. The flux distribution for optimal methionine production in the two organisms differed dramatically (FIGS. 3, 4).

The optimal flux towards methionine in C. glutamicum was 58.3%. For this purpose, C. glutamicum exhibited a very high activity of PPP with a flux through the oxidative reactions of the PPP of 250%. This is probably due to the demand for NADPH as 8 NADPH have to be supplied for methionine synthesis, primarily for sulfur reduction. The flux into the PPP is substantially higher than the uptake flux of glucose. Glucose 6-phosphate isomerase, working in the gluconeogenetic direction, also significantly contributes to the supply of carbon towards the PPP. The TCA cycle is completely turned off, so that isocitrate dehydrogenase does not contribute to NADPH formation. Additionally C. glutamicum employs two important metabolic cycles. The first cycle does only involve 2-oxoglutarate and glutamate, which are interconverted at high flux, to assimilate ammonium and use it for amination reactions required. These are the formation of methionine itself and the formation of serine as donor of the methyl-group for formation of methyl-THF, so that the flux through this cycle is exactly double the methionine flux. The second metabolic cycle comprises the pools of pyruvate, oxaloacetate and malate. It exhibits two major functions: Almost half of the CO₂ lost in the oxidative PPP reenters the metabolic network by the highly active fixation of CO₂ (125% flux). Additionally, the combination of the three enzymes involved in the cycle acts as a transhydrogenase and interconverts NADH into NADPH (25% flux). By this C. glutamicum can, to some extent overcome the lack of a transhydrogenase.

Optimal methionine production in E. coli resulted in a methionine flux of 67.5%. In contrast to C. glutamicum, the PPP was not active, whereas the TCA cycle showed a high flux of almost 100%. However, the TCA cycle was operating in a modified way. The step from succinyl-CoA to succinate is bridged by the corresponding reaction producing succinate in the methionine biosynthesis. Interestingly optimal methionine production required substantial activity of the glyoxylate shunt (31% flux). Most significant is the enormous flux of 574% through transhydrogenase from NADH to NADPH. This underlines the importance of this enzyme for efficient methionine production in E. coli. As shown above, the maximal theoretical methionine yield drops significantly (FIG. 2 D), when this enzyme is deleted. In both organisms pyruvate kinase is dispensable for optimal methionine production. Accordingly, the flux from PEP to pyruvate is exclusively provided by the PTS, coupled to the uptake of glucose. Obviously pyruvate kinase is not required for ATP production. The knockout of this enzyme could be an interesting target, since it might limit the flux towards pyruvate and related over-flow metabolites of the TCA cycle.

Summarizing, the optimal flux distribution of the two organisms was fundamentally different. By using elementary flux mode analysis with respect to methionine synthesis, predictions for genetic modifications can be obtained that should allow to increase efficiency of methionine synthesis.

### Potential Improvement of Methionine Production by Genetic Modifications

To study the influence of some key reactions in more detail, additional simulations with modified metabolic networks were carried out. The implementation of a transhydrogenase into C. glutamicum led to an increased theoretical methionine yield of 51.9% (FIG. 2 C). The knockout of the transhydrogenase in E. coli strongly decreased its potential for methionine production (FIG. 2 D). This underlines the beneficial effect of an active interconversion of NADH into NADPH for methionine production.

The insertion of the glycine cleavage system in C. glutamicum increased the theoretical maximal methionine yield to 56.5% (FIG. 2 E). Similarly, the knockout of either the glycine cleavage system or the transhydrogenase in E. coli resulted in a reduced theoretical maximal methionine yield (FIGS. 2 F, D). Note that the trans-hydrogenase also affects the maximal theoretical biomass yield. Insertion in C. glutamicum leads to an increase, whereas deletion in E. coli causes a decrease of Y_{X/S, max}, respectively.

Concerning the carbon yields, all flux modes were located within a triangle shaped space, which was spanned between the origin and the two extreme flux modes with maximum biomass and methionine formation, respectively (FIGS. 2 A-F). The connection between the two extreme modes hereby displays an optimum line, which gives the maximum methionine yield possible under different regimes of growth. All modes and linear combinations of modes on this line represent interesting solutions for a production process. A real production process will always be linked to a certain formation of biomass.

### Influence of the Sulfur Source on Methionine Production

Potentially positive effects of genetic modifications could be clearly identified. Further simulations were carried out to even more increase the theoretically possible methionine synthesis efficiency. In this regard the effect of alternative nutrients was investigated. Hereby the sulfur source may play a central role. The results obtained are exemplified for C. glutamicum.

The conventional sulfur source is sulfate as also applied in the above pathway analysis for the wild types. Sulfate assimilation is, however, linked to a high demand of 2 ATP and 4 NADPH. Especially the high requirement for reducing power suggests that the reduction state of the sulfur source might be a crucial point. Accordingly, metabolic pathway analysis was carried out using sulfate, thiosulfate, and sulfide as sulfur sources. For utilization of thiosulfate, thiosulfate reductase (Schmidt et al. (1984) vide supra, Heinzinger et al. (1995) J. Bacteriol., 177: 2813-2820, Fong et al. (1993) J. Bacteriol., 175: 6368-6371)) was incorporated into the model. This enzyme allows the cleavage of thiosulfate into sulfite and sulfide and thus reduces the overall demand of NADPH for methionine production by about 25%. It should be noted that, to our knowledge, consumption of both sulfur atoms of thiosulfate has not been shown yet in C. glutamicum. Another possibility to produce sulfide from a more reduced form of sulphur is the so-called anaerobic sulfite reductase (Huang et al. (1991) Journal of Bacteriology. 173(4):1544-53).

It becomes obvious that the sulfur source is a key point concerning the theoretical carbon yield of a production process. Compared to sulfate (FIG. 2 A), the utilization of alternative sulfur sources significantly increases the maximal theoretical yield (FIGS. 3 A, B). The increase from sulfate (48.6%) to thiosulfate (57.8%) to sulfide (63.4%) impressively underlines the high potential of using alternative nutrients for methionine production. Furthermore, it demonstrates the high importance for reducing power (NADPH) for optimal methionine biosynthesis. In C. glutamicum NADPH is mostly produced in the oxidative PPP and to some extend in the TCA cycle with a NADP-dependent isocitrate dehydrogenase. During growth on sulfate the wild type requires 8 moles NADPH per mole methionine synthesized via the direct sulfydrylation and 9 moles NADPH via the transsulfuration pathway (Hwang et al. (2002), J. Bacteriol., 1845, 1277-86). The thiosulfate network requires 6 moles NADPH for methionine production and thus 25% less. The network consuming sulfide will only require 50% of the NADPH demand on sulfate. This stepwise reduction of the NADPH demand by 25% each is linked to a stepwise increase of Y_{Met/S,max} of 9.2% and only 5.6% using sulfide instead of thiosulfate. This could be of importance in later process development as sulfide is highly toxic and volatile.

### Influence of the C₁ Source on Methionine Production

A major target for improvement of C. glutamicum for methionine production is the C₁ metabolism. The optimal production of methionine is linked to the accumulation of equimolar amounts of glycine, which normally cannot be re-utilized (FIG. 3). As shown, this could be overcome by implementation of a glycine cleavage system (FIG. 2 E). An alternative is given by the use of a C₁ carbon source in addition to glucose. In this regard, formate was investigated involving different extensions of the metabolic network. This included the incorporation of an enzyme that catalyzes the formation of 10-formyl-THF from formate, ATP and THF as described for many organisms, e.g. bacilli (E.C. 6.3.4.3). Additionally different steps for conversion of 10-formyl-THF into methyl-THF were implemented. All reactions were lumped together in an overall reaction converting 10-formyl-THF into Methylene-THF linked to oxidation of 1 NADPH and 1 NADH. The utilization of formate plus glucose led to a slight increase of the maximal theoretical methionine yield of 3.3% as compared to the situation with sole use of glucose. Additionally glycine was no more accumulated, when formate was supplied.

### Influence of Combined Use of Different Sulfur and C₁ Sources on Methionine Production

It was shown above that both the C₁- and the sulfur source are important for maximizing maximal theoretical carbon yield in biotechnological methionine production. It therefore appeared interesting to see, if the benefits from C₁ and sulfur sources could be combined. The studies involved the combination of thiosulfate and formate and the combination of sulfide and formate. For the combination of thiosulfate and formate, the maximal theoretical carbon yield increased to 63.0% (FIG. 3 D). This yield is still slightly lower than the maximal theoretical yield of sulfide consumption (FIG. 3 B). However, in contrast to sulfide, formate and thiosulfate are non-hazardous chemicals and probably linked to reduced efforts concerning process safety. Combining the use of sulfide and formate resulted in Y_{Met/S,max} of to 69.6%. This is 6.2% higher than the maximal theoretical yield of sole sulfide consumption.

### Influence of Methanethiol and its Dimer Dimethyl Disulfide as Combined Source for Sulfur and C₁ Carbon on Methionine Production

An interesting possibility of providing reduced sulfur and solving the problem of glycine accumulation is provided by feeding of methanethiol and its dimer dimethyl disulfide. It is known that C. glutamicum can produce methanethiol under certain conditions (Bonnarme et al. (2000), Appl. Environ. Microbiol., 6612, 5514-7). It is assumed here that it is also able to consume methanethiol and its dimer dimethyl disulfide. It is also assumed that the dimer dimethyl disulfide can be cleaved to methanethiol by the mentioned organisms such as but not limited to C. glutamicum or E. coli. A putative reaction was added to the network that uses direct methyl-sulfhydrylation of O-acetyl-homoserine with methanethiol. This new proposed reaction bypasses homocysteine and directly yields methionine. The use of methanethiol and its dimer dimethyl disulfide tremendously increased the maximal theoretical yield of methionine to 83.3% (FIG. 3 F). This shows that this substrate is potentially very useful for biotechnological methionine production. The pathways involved in sulfur metabolism and MTHF-formation would be dispensable for methionine production. In E. coli the maximal theoretical methionine yield on methanethiol and its dimer dimethyl disulfide was 71.4% and thus substantially lower as compared to C. glutamicum. The reason is the requirement for succinyl-CoA for methionine production in this organism. This demands for a high activity of the TCA cycle and corresponding loss of carbon via CO₂. In comparison to C. glutamicum, CO₂ formation in E. coli under these conditions is almost twice as high. Finally the maximal theoretical yield could be further improved by integrating a transhydrogenase into the C. glutamicum model with methanethiol metabolism. Under these conditions C. glutamicum would be able to produce methionine from methanethiol and its dimer dimethyl disulfide and glucose with a maximal carbon yield of 85.5%.

The above analysis has thus shown that particularly the use of glycine or alternative sources of the methyl group in methionine synthesis offer an important potential for optimizing methionine production in C. glutamicum. Furthermore, it could be shown that methionine synthesis in E. coli is more dependent on an active transhydrogenase than C. glutamicum.

B) Genetic Modification of C. glutamicum for Increasing Efficiency of Methionine Synthesis

The goal of the following experiments is to apply the implications of the above theoretic findings for obtaining a C glutamicum organism with increased efficiency of methionine synthesis

### Material and Methods

Protocols for general methods can be found in Handbook on Corynebacterium glutamicum, (2005) eds.: L. Eggeling, M. Bott., Boca Raton, CRC Press, at Martin et al. (Biotechnology (1987) 5, 137-146), Guerrero et al. (Gene (1994), 138, 35-41), Tsuchiya und Morinaga (Biotechnology (1988), 6, 428-430), Eikmanns et al. (Gene (1991), 102, 93-98), EP 0 472 869, U.S. Pat. No. 4,601,893, Schwarzer and Pühler (Biotechnology (1991), 9, 84-87, Reinscheid et al. (Applied and Environmental Microbiology (1994), 60, 126-132), LaBarre et al. (Journal of Bacteriology (1993), 175, 1001-1007), WO 96/15246, Malumbres et al. (Gene (1993), 134, 15-24), in JP-A-10-229891, at Jensen und Hammer (Biotechnology and Bioengineering (1998), 58, 191-195), Makrides (Microbiological Reviews (1996), 60, 512-538) and in well known textbooks of genetic and molecular biology.

### Strains, Media and Plasmids

Strains can be taken e.g. from the following list:

Corynebacterium glutamicum ATCC 13032,
Corynebacterium acetoglutamicum ATCC 15806,
Corynebacterium acetoacidophilum ATCC 13870,
Corynebacterium thermoaminogenes FERM BP-1539,
Corynebacterium melassecola ATCC 17965,
Brevibacterium flavum ATCC 14067,
Brevibacterium lactofermentum ATCC 13869, and

Brevibacterium divaricatum ATCC 14020 or strains which have been derived therefrom such as Corynebacterium glutamicum KFCC 10065

### DSM 17322 or

Corynebacterium glutamicum ATCC21608

### Recombinant DNA Technology

Protocols can be found in: Sambrook, J., Fritsch, E. F., and Maniatis, T., in Molecular Cloning: A Laboratory Manual, 3^{rd} edition (2001) Cold Spring Harbor Laboratory Press, NY, Vol. 1, 2, 3, and Handbook on Corynebacterium glutamicum (2005) eds. L. Eggeling, M. Bott., Boca Raton, CRC Press.

### Quantification of Amino Acids and Methionine Intermediates.

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/1) as non-polar phase (eluent B). The following gradient is applied: Start 0% B; 39 min 39% B; 70 min 64% B; 100% B for 3.5 min; 2 min 0% B for equilibration. Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivatization is done by adding 0.5 μl of 10 mg/ml OPA reagent dissolved in Jan. 45, 1954 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. Note, that the analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) was is as internal standard

### Definition of Recombination Protocol

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid) has integrated into a chromosome by a single homologous recombination event (a cross in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs.

### Construction of the Methionine Producing Strain

### Example 1

### Generation of the Methionine Producing Starting Strain M2014 Strain

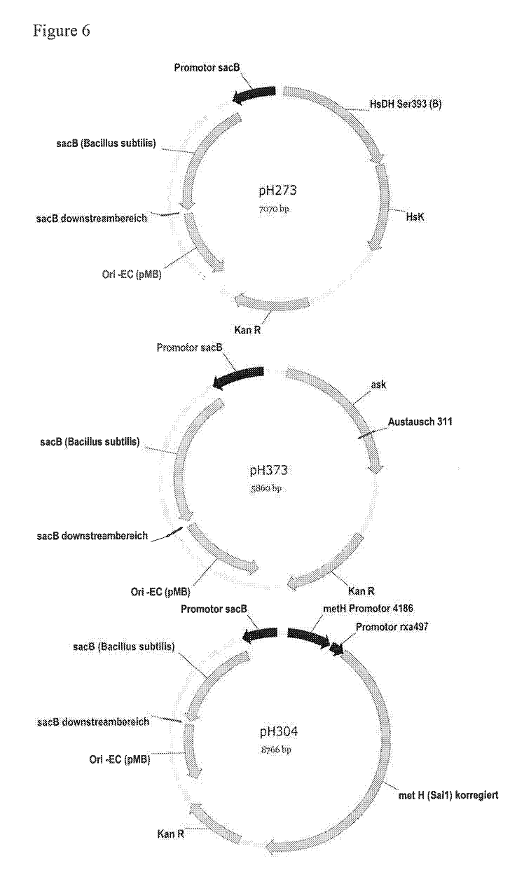

C. glutamicum strain ATCC 13032 was transformed with DNA A (also referred to as pH273) (SEQ ID NO: 1) and “Campbelled in” to yield a “Campbell in” strain. FIG. 6 shows a schematic of plasmid pH273. The “Campbell in” strain was then “Campbelled out” to yield a “Campbell out” strain, M440, which contains a gene encoding a feedback resistant homoserine dehydrogenase enzyme (hom^{fbr}). The resultant homoserine dehydrogenase protein included an amino acid change where S393 was changed to F393 (referred to as Hsdh S393F).

The strain M440 was subsequently transformed with DNA B (also referred to as pH373) (SEQ ID NO:2) to yield a “Campbell in” strain. FIG. 6 depicts a schematic of plasmid pH373. The “Campbell in” strain was then “Campbelled out” to yield a “Campbell out” strain, M603, which contains a gene encoding a feedback resistant aspartate kinase enzyme (Ask^{fbr}) (encoded by lysC). In the resulting aspartate kinase protein, T311 was changed to I311 (referred to as LysC T311I).

It was found that the strain M603 produced about 17.4 mM lysine, while the ATCC13032 strain produced no measurable amount of lysine. Additionally, the M603 strain produced about 0.5 mM homoserine, compared to no measurable amount produced by the ATCC13032 strain, as summarized in Table 3.

TABLE 3
Amounts of homoserine, O-acetylhomoserine, methionine and lysine
produced by strains ATCC13032 and M603

| | | O-acetyl | | |
|---|---|---|---|---|
| | Homoserine | homoserine | Methionine | Lysine |
| Strain | (mM) | (mM) | (mM) | (mM) |
| ATCC13032 | 0.0 | 0.4 | 0.0 | 0.0 |
| M603 | 0.5 | 0.7 | 0.0 | 17.4 |

The strain M603 was transformed with DNA C (also referred to as pH304, a schematic of which is depicted in FIG. 6) (SEQ ID NO:3) to yield a “Campbell in” strain, which was then “Campbelled out” to yield a “Campbell out” strain, M690. The M690 strain contained a PgroES promoter upstream of the metH gene (referred to as P₄₉₇ metH). The sequence of the P₄₉₇ promoter is depicted in SEQ ID NO:11. The M690 strain produced about 77.2 mM lysine and about 41.6 mM homoserine, as shown below in Table 4.

TABLE 4
Amounts of homoserine, O-acetyl homoserine, methionine and lysine
produced by the strains M603 and M690

| | | O-acetyl | | |
|---|---|---|---|---|
| | Homoserine | homoserine | Methionine | Lysine |
| Strain | (mM) | (mM) | (mM) | (mM) |
| M603 | 0.5 | 0.7 | 0.0 | 17.4 |
| M690 | 41.6 | 0.0 | 0.0 | 77.2 |

The M690 strain was subsequently mutagenized as follows: an overnight culture of M690, grown in BHI medium (BECTON DICKINSON), was washed in 50 mM citrate buffer pH 5.5, treated for 20 min at 30° C. with N-methyl-N-nitrosoguanidine (10 mg/ml in 50 mM citrate pH 5.5). After treatment, the cells were again washed in 50 mM citrate buffer pH 5.5 and plated on a medium containing the following ingredients: (all mentioned amounts are calculated for 500 ml medium) 10 g (NH₄)₂SO₄; 0.5 g KH₂PO₄; 0.5 g KH₂PO₄; 0.125 g MgSO₄.7H₂O; 21 g MOPS; 50 mg CaCl₂; 15 mg protocatechuic acid; 0.5 mg biotin; 1 mg thiamine; and 5 g/l D,L-ethionine (SIGMA CHEMICALS, CATALOG #E5139), adjusted to pH 7.0 with KOH. In addition the medium contained 0.5 ml of a trace metal solution composed of: 10 g/l FeSO₄.7H₂O; 1 g/l MnSO₄.H₂O; 0.1 μl ZnSO₄.7H₂O; 0.02 g/l CuSO₄; and 0.002 g/l NiCl₂.6H₂O, all dissolved in 0.1 M HCl. The final medium was sterilized by filtration and to the medium, 40 mls of sterile 50% glucose solution (40 ml) and sterile agar to a final concentration of 1.5% were added. The final agar-containing medium was poured to agar plates and was labeled as minimal-ethionine medium. The mutagenized strains were spread on the plates (minimal-ethionine) and incubated for 3-7 days at 30° C. Clones that grew on the medium were isolated and restreaked on the same minimal-ethionine medium. Several clones were selected for methionine production analysis.

Methionine production was analyzed as follows. Strains were grown on CM-agar medium for two days at 30° C., which contained: 10 g/l D-glucose, 2.5 μl NaCl; 2 g/l urea; 10 g/l Bacto Peptone (DIFCO); 5 g/l Yeast Extract (DIFCO); S g/l Beef Extract (DIFCO); 22 g/l Agar (DIFCO); and which was autoclaved for 20 min at about 121° C.

After the strains were grown, cells were scraped off and resuspended in 0.15 M NaCl. For the main culture, a suspension of scraped cells was added at a starting OD of 600 nm to about 1.5 to 10 ml of Medium II (see below) together with 0.5 g solid and autoclaved CaCO₃ (RIEDEL DE HAEN) and the cells were incubated in a 100 ml shake flask without baffles for 72 h on a orbital shaking platform at about 200 rpm at 30° C. Medium II contained: 40 g/l sucrose; 60 μl total sugar from molasses (calculated for the sugar content); 10 g/l (NH₄)₂SO₄; 0.4 g/l MgSO₄.7H₂O; 0.6 g/l KH₂PO₄; 0.3 mg/l thiamine*HCl; 1 mg/l biotin; 2 mg/l FeSO₄; and 2 mg/l MnSO₄. The medium was adjusted to pH 7.8 with NH₄OH and autoclaved at about 121° C. for about 20 min). After autoclaving and cooling, vitamin B₁₂ (cyanocobalamine) (SIGMA CHEMICALS) was added from a filter sterile stock solution (200 μg/ml) to a final concentration of 100 μg/l.

Samples were taken from the medium and assayed for amino acid content. Amino acids produced, including methionine, were determined using the Agilent amino acid method on an Agilent 1100 Series LC System HPLC. (AGILENT). A pre-column derivatization of the sample with ortho-pthalaldehyde allowed the quantification of produced amino acids after separation on a Hypersil AA-column (AGILENT).

Clones that showed a methionine titer that was at least twice that in M690 were isolated. One such clone, used in further experiments, was named M1197 and was deposited on May 18, 2005, at the DSMZ strain collection as strain number DSM 17322. Amino acid production by this strain was compared to that by the strain M690, as summarized below in Table 5.

TABLE 5
Amounts of homoserine, O-acetylhomoserine, methionine and
lysine produced by strains M690 and M1197

| | | O-acetyl- | | |
|---|---|---|---|---|
| | Homoserine | homoserine | Methionine | Lysine |
| Strain | (mM) | (mM) | (mM) | (mM) |
| M690 | 41.6 | 0.0 | 0.0 | 77.2 |
| M1179 | 26.4 | 1.9 | 0.7 | 79.2 |

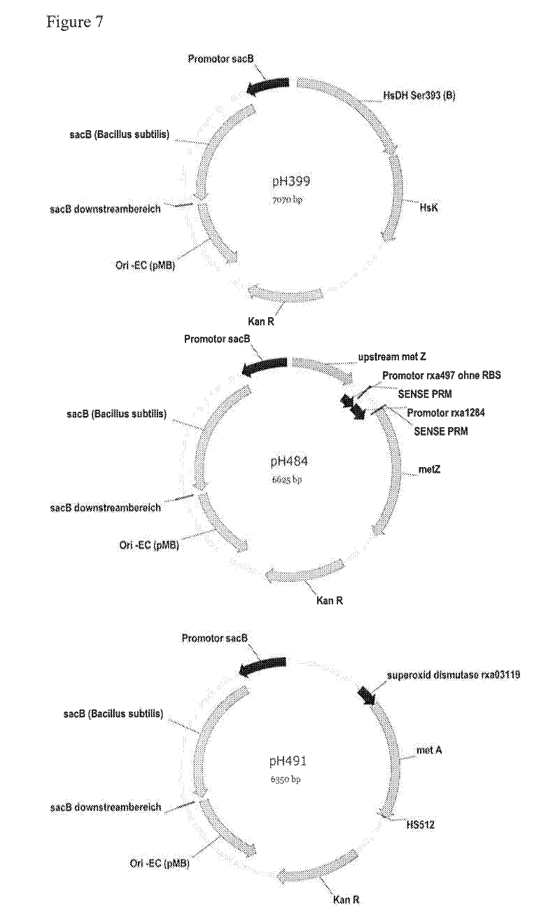

The strain M1197 was transformed with DNA F (also referred to as pH399, a schematic of which is depicted in FIG. 7) (SEQ ID NO:4) to yield a “Campbell in” strain, which was subsequently “Campbelled out” to yield strain M1494. This strain contains a mutation in the gene for the homoserine kinase, which results in an amino acid change in the resulting homoserine kinase enzyme from T190 to A190 (referred to as HskT190A). Amino acid production by the strain M1494 was compared to the production by strain M1197, as summarized below in Table 6.

TABLE 6
Amounts of homoserine, O-acetylhomoserine, methionine and
lysine produced by strains M1197 and M1494

| | | O-acetyl- | | |
|---|---|---|---|---|
| | Homoserine | homoserine | Methionine | Lysine |
| Strain | (mM) | (mM) | (mM) | (mM) |
| M1197 | 26.4 | 1.9 | 0.7 | 79.2 |
| M1494 | 18.3 | 0.2 | 2.5 | 50.1 |

The strain M1494 was transformed with DNA D (also referred to as pH484, a schematic of which is shown in FIG. 7) (SEQ ID NO:5) to yield a “Campbell in” strain, which was subsequently “Campbelled out” to yield the M1990 strain. The M1990 strain overexpresses a metY allele using both a groES-promoter and an EFTU (elongation factor Tu)-promoter (referred to as P₄₉₇ P₁₂₈₄ metY). The sequence of P₄₉₇ P₁₂₈₄ is set forth in SEQ ID NO: 13. Amino acid production by the strain M1494 was compared to the production by strain M1990, as summarized below in Table 7.

TABLE 7
Amounts of homoserine, O-acetylhomoserine, methionine and lysine
produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1494 | 18.3 | 0.2 | 2.5 | 50.1 |
| M1990 | 18.2 | 0.3 | 5.6 | 48.9 |

The strain M1990 was transformed with DNA E (also referred to as pH 491, a schematic of which is depicted in FIG. 7) (SEQ ID NO:6) to yield a “Campbell in” strain, which was then “Campbelled out” to yield a “Campbell out” strain M2014. The M2014 strain overexpresses a metA allele using a superoxide dismutase promoter (referred to as P₃₁₁₉ metA). The sequence of P₃₁₁₉ is set forth in SEQ ID NO:12. Amino acid production by the strain M2014 was compared to the production by strain M2014, as summarized below in Table 8.

TABLE 8
Amounts of homoserine, O-acetylhomoserine, methionine and lysine
produced by strains M1990 and M2014

| | | O-acetyl- | | |
|---|---|---|---|---|
| | Homoserine | homoserine | Methionine | Lysine |
| Strain | (mM) | (mM) | (mM) | (mM) |
| M1990 | 18.2 | 0.3 | 5.6 | 48.9 |
| M2014 | 12.3 | 1.2 | 5.7 | 49.2 |

### Example 2

### Shake Flask Experiments and HPLC Assay

Shake flasks experiments, with the standard Molasses Medium, were performed with strains in duplicate or quadruplicate. Molasses Medium contained in one liter of medium: 40 g glucose; 60 g molasses; 20 g (NH₄)₂ SO₄; 0.4 g MgSO₄.7H₂O; 0.6 g KH₂PO₄; 10 g yeast extract (DIFCO); 5 ml of 400 mM threonine; 2 mgFeSO₄.7H₂O; 2 mg of MnSO₄.H₂O; and 50 g CaCO₃ (Riedel-de Haen), with the volume made up with ddH₂O. The pH was adjusted to 7.8 with 20% NH₄OH, 20 ml of continuously stirred medium (in order to keep CaCO₃ suspended) was added to 250 ml baffled Bellco shake flasks and the flasks were autoclaved for 20 min. Subsequent to autoclaving, 4 ml of “4B solution” was added per liter of the base medium (or 80 μl/flask). The “4B solution” contained per liter: 0.25 g of thiamine hydrochloride (vitamin B1), 50 mg of cyanocobalamin (vitamin B12), 25 mg biotin, 1.25 g pyridoxine hydrochloride (vitamin B6) and was buffered with 12.5 mM KPO₄, pH 7.0 to dissolve the biotin, and was filter sterilized. Cultures were grown in baffled flasks covered with Bioshield paper secured by rubber bands for 48 hours at 28° C. or 30° C. and at 200 or 300 rpm in a New Brunswick Scientific floor shaker. Samples were taken at 24 hours and/or 48 hours. Cells were removed by centrifugation followed by dilution of the supernatant with an equal volume of 60% acetonitrile and then membrane filtration of the solution using Centricon 0.45 μm spin columns. The filtrates were assayed using HPLC for the concentrations of methionine, glycine plus homoserine, O-acetylhomoserine, threonine, isoleucine, lysine, and other indicated amino acids.

For the HPLC assay, filtered supernatants were diluted 1:100 with 0.45 μm filtered 1 mM Na₂EDTA and 1 μl of the solution was derivatized with OPA reagent (AGILENT) in Borate buffer (80 mM NaBO₃, 2.5 mM EDTA, pH 10.2) and injected onto a 200×4.1 mm Hypersil 5μ AA-ODS column run on an Agilent 1100 series HPLC equipped with a G1321A fluorescence detector (AGILENT). The excitation wavelength was 338 nm and the monitored emission wavelength was 425 nm. Amino acid standard solutions were chromatographed and used to determine the retention times and standard peak areas for the various amino acids. Chem Station, the accompanying software package provided by Agilent, was used for instrument control, data acquisition and data manipulation. The hardware was an HIP Pentium 4 computer that supports Microsoft Windows NT 4.0 updated with a Microsoft Service Pack (SP6a).

### Example 3

### Generation of a Microorganism Containing a Deregulated Sulfate Reduction Pathway

Plasmid pOM423 (SEQ ID NO: 7) was used to generate strains that contain a deregulated sulfate reduction pathway. Specifically, an E. coli phage lambda P_{L} and P_{R} divergent promoter construct was used to replace the native sulfate reduction regulon divergent promoters. Strain M2014 was transformed with pOM423 and selected for kanamycin resistance (Campbell in). Following sacB counter-selection, kanamycin sensitive derivatives were isolated from the transformants (Campbell out). These were subsequently analyzed by PCR to determine the promoter structures of the sulfate reduction regulon. Isolates containing the P_{L}-P_{R} divergent promoters were named OM429. Four isolates of OM429 were assayed for sulfate reduction using the DTNB strip test and for methionine production in shake flask assays. To estimate relative sulfide production using the DTNB strip test, a strip of filter paper was soaked in a solution of Elman's reagent (DTNB) and suspended over a shake flask culture of the strain to be tested for 48 hours. Hydrogen sulfide produced by the growing culture reduces the DTNB, producing a yellow color that is roughly proportional to the amount of H₂S generated. Thus, the intensity of the color produced can be used to obtain a rough estimate of the relative sulfate reduction activity of various strains. The results (Table 10) show that two of the four isolates displayed relatively high levels of sulfate reduction. These same two isolates also produced the highest levels of methionine. Cultures were grown for 48 hours in standard molasses medium.

TABLE 10
Methionine production and sulfate reduction by isolates of OM429
in shake flask cultures

| | | Sulfate regulon | Met | DTNB |
|---|---|---|---|---|
| Strain | | promoters | (g/l) | Test |
| M2014 | | Native | 1.1 | − |
| OM429 | -1 | P_{L}/P_{R} | 1.1 | − |
| | -2 | 1.1 | − | |
| | -3 | 1.3 | ++ | |
| | -4 | 1.4 | ++ | |

### Experiment 4

### Strains Containing the E. coli Glycine Cleavage (gcv) Operon

The production of methylene tetrahydrofolate, from serine via the GlyA enzyme (R38) which is necessary for methionine biosynthesis from glucose, also yields glycine as a byproduct. In methionine overproducing strains, the amount of glycine produced will be in excess of the requirement for protein synthesis. Thus, according to the above model, inclusion of the GCS in C. glutamicum should result in enhanced efficiency of methionine synthesis.

In E. coli and B. subtilis, if glycine is present in excess of that required for protein synthesis, it is cleaved to give a second equivalent of methylene tetrahydrofolate by the glycine cleavage enzyme system. In E. coli, the glycine cleavage system involves four different proteins. Three of these are encoded by the gcvTHP operon. The fourth subunit is lipoamide dehydrogenase, which is borrowed from the multi-subunit pyruvate dehydrogenase. C. glutamicum does not appear to have a glycine cleavage system. No homologs of the E. coli Gcv proteins were found in the C. glutamicum genome, although C. glutamicum does have the usual multi-subunit pyruvate dehydrogenase. As a result, methionine production in C. glutamicum results in concomitant glycine production, which appears in culture supernatants. It was thus tried to implement a GCS in C. glutamicum and to recycle glycine into methylene tetrahydrofolate, as is done in E. coli and B. subtilis.

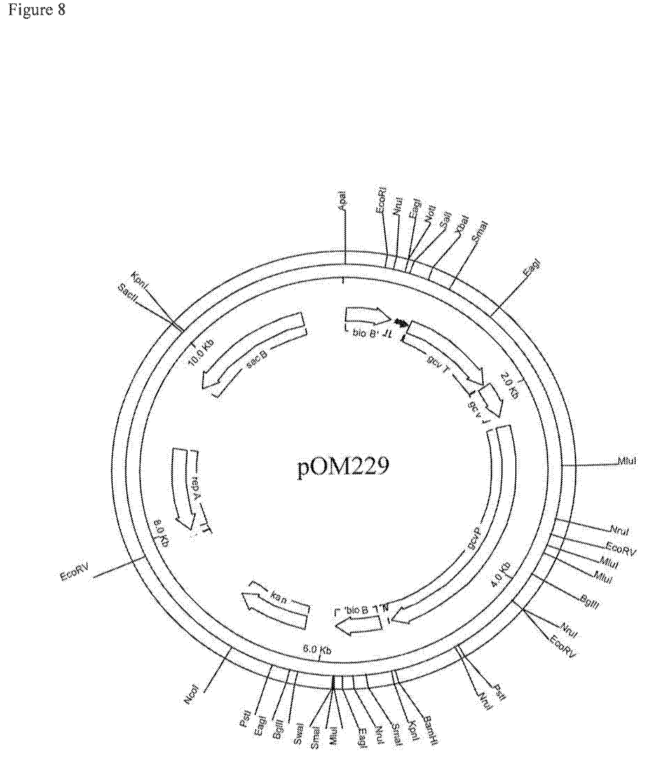

As a first step toward this goal, the E. coli gcvTHP operon was amplified by PCR without its native promoter, and cloned it downstream from the P₄₉₇ promoter in pOM218, which is a low copy E. coli vector designed to integrate expression cassettes at bioB in C. glutamicum. It was assumed that the necessary fourth subunit from pyruvate dehydrogenase can be supplied from the host organism that is C. glutamicum. The resulting plasmid, pOM229 (FIG. 8, SEQ ID No: 8), was transformed into the starter organism, strain M2014 and was successfully Campbelled out to give strains named OM212. These strains were then cultured.

The following medium was used: 40 g/l glucose, 60 g/l molasses with a sugar content of 45%, 10 g/l (NH₄)₂SO₄, 0.4 g/l MgSO₄.7H₂O, 2 mg/l FeSO₄, 2 mg/l MnSO₄, 1.0 mg/l thiamine, 1 mg/l biotin. The pH was adjusted to pH 7.8 with 30% NH₄OH, and the medium autoclaved for 20 minutes. After autoclaving: 200 Mg/l B12, 2 mM L-threonine, 2 ml of 0.5 g/ml CaCO₃ per 20 ml medium. Phosphate buffer pH 7.2 WAS added to 200 mM from a 2 M stock.

In shake flask cultures, one isolate, OM212-1 was analysed as explained above. The results which show an increase in methionine production and a decrease in glycine plus homoserine are shown in Table 11.

TABLE 11
Methionine production by derivatives of M2014 that
contain P₄₉₇ gcvTHP (E. coli)
integrated at bioB, in shake flask cultures grown in molasses
plus CaCO₃ medium.

| | | [Gly + | [O-Ac- | | |
|---|---|---|---|---|---|
| | | Hse] | Hse] | [Lys] | [Met] |
| Strain | New feature | g/l | g/l | g/l | g/l |
| M2014 | parent | 0.66 | 1.4 | 3.3 | 0.64 |
| ″ | ″ | 0.75 | 1.6 | 3.4 | 0.70 |
| | | | | | Av 0.67 |
| OM212-1 | pOM229 | 0.67 | 1.7 | 3.8 | 0.74 |
| ″ | P₄₉₇ gcvTHP | 0.58 | 1.8 | 3.7 | 0.70 |
| | @bioB | | | | Av 0.72 |

It was observed that the carbon yield of strain M2014 was 0.0103 Mol methionine/mol sugar while strain OM212-1 had carbon yield of 0.011 Mol methionine/mol sugar.

In another embodiment the subunit of the glycine cleavage system not coded for by the gcvTHP operon, that is the lpdA gene (SEQ ID No: 10), which encodes lipoamide dehydrogenase is cloned from the host the E. coli. The gene is amplified without its natural promotor and the P₄₉₇ promoter is added instead. The resulting fragment is cloned into the E. coli C. glutamicum shuttle vector pOM229 in addition to the gcvTHP operon.

### Experiment 5

### An In Vivo Assay for a Functional Glycine Cleavage System

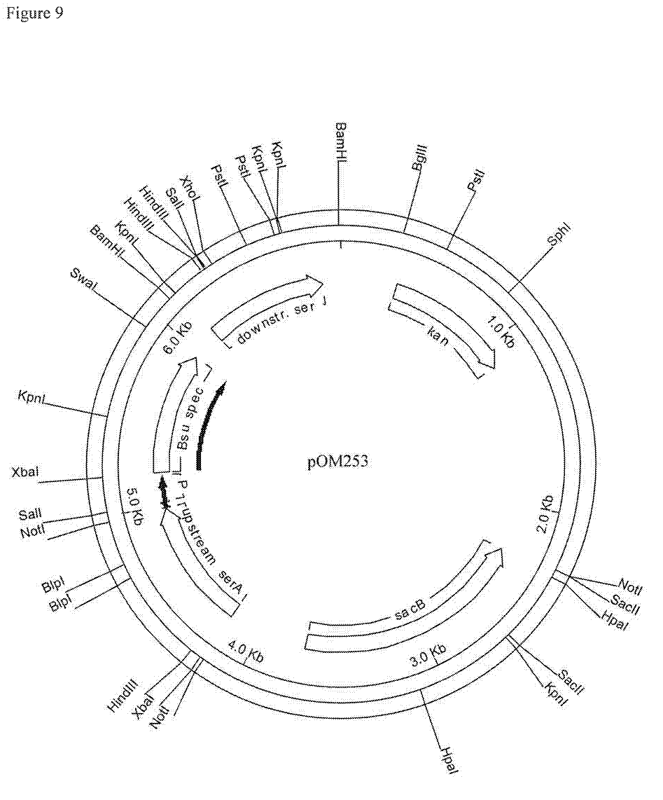

The C. glutamicum serA gene was generated by PCR and cloned into Swa I gapped pC INT to give plasmid pOM238. Next, a blunt fragment containing a gram-positive spectinomycin resistance gene (spc) expressed from C. glutamicum P497, was ligated into Ale I gapped pOM238. An isolate that contained the spc gene in the same orientation as serA was named pOM253 (see FIG. 9, SEQ ID NO:9). pOM253 can be used to create an interruption-deletion in the serA gene of any C. glutamicum strain.

pOM253 was transformed into C. glutamicum strain M2014, selecting for kanamycin resistance, to give “Campbelled in” strain OM264K. OM264K was “Campbelled out” by selecting for sucrose resistance (BHI+5% sucrose) and spectinomycin resistance (BHI+100 mg/l spectinomycin) to give strain OM264, which is a serine, threonine, and biotin auxotroph.

Strain OM264 can be transformed with plasmid pOM229, or another plasmid (or plasmids) that supplies the glycine cleavage pathway (Gcv). If the glycine cleavage pathway is active, then the resulting serA⁻, Gev⁺ strain will be able to grow on minimal medium containing glycine, threonine, and biotin, since methylene tetrahydrofolate will be generated by the glycine cleavage system, and the glyA gene product, serine hydroxymethyl transferase (SHMT), will be able to make serine by running the SHMT reaction in the reverse direction, using glycine and methylene tetrahydrofolage as substrates.

If necessary, a gene encoding lipoamide dehydrogenase, for example, the lpd gene (also called lpdA; Seq No: 10) from E. coli can be cloned and transformed into the above-described strain to supply the necessary fourth subunit for the glycine cleavage system. The genes encoding glycine cleavage systems from organisms other than E. coli can also be cloned by PCR or complementation as described above and used to supply a functional glycine cleavage system in C. glutamicum. For example, the Bacillus subtilis genes, gcvH, gcvPA, gcvPB, gcvT and pdhD, which encode a five subunit glycine cleavage system (the glycine decarboxylase is comprised of two subunits in B. subtilis, encoded by gcvPA and gcvPB, while in E. coli these two functions are combined in to one subunit encoded by gcvP), or any other suitable set of genes could be used. The only requirement is that the system function in C. glutamicum at level sufficient to convert excess glycine (produced as a result of methionine biosynthesis) to methylene tetrahydrofolate.

### Experiment 6

### Knockout of Pyruvate Kinase in C. glutamicum

The elementary mode analysis indicated that a downregulation of pyruvate kinase (R19) may lead to an increased efficiency of methionine synthesis (see e.g. FIG. 3).

To investigate the effect of pyruvate kinase knockout, a lysine-producing strain of C. glutamicum was analyzed. If indeed an increase in lysine production were observed, this should also be indicative of an increased methionine synthesis, as the formation of lysine is preceded by formation of aspartate, aspartate phosphate, etc. An increase in lysine production should therefore be preceded by an increase in e.g. aspartate. As aspartate is also one of the precursors of methionine production, an increased amount of aspartate should also lead to increased methionine synthesis.

A strain comparison between C. glutamicum lysC^{fbr} and C. glutamicum lysC^{fbr} Δpyk was carried out. C. glutamicum lysC^{fbr} is a mutant carrying a point mutation in the gene coding for aspartokinase (Kalinowski et al. (1991), Mol. Microbiol. 5(5), 1197-1204). This strain was then used for deleting the pyruvate kinase (C. glutamicum lysC^{fbr} Δpyk).

Both strains were cultivated in shaker flasks on minimal media and carbon yields determined for biomass, lysine and side products. Based on the mean value of two independent experiments, it was observed that lysine yields for the pyruvate kinase knockout increased from 7.5-12.1%. This corresponds to an increase of approximately 62%.

In conclusion, a pyruvate kinase knockout leads to an increased synthesis of lysine and correspondingly should also lead to increased methionine synthesis. However, using pyruvate kinase knockout for producing methionine would not have been expected to increase methionine synthesis, as methionine itself relies on an active pyruvate kinase if common knowledge about the metabolic networks is taken into account.

### Experiment 7

### Comparison of Uptake in Usage of Different Sulphur Sources

The elementary mode analysis had shown that methionine synthesis efficiency surprisingly was dependent on the reduction state of the sulphur source. As explained above, for each saved NADPH an increase in methionine synthesis efficiency of 4.6% may be expected. However, so far there are only preliminary and incomplete data as to the growth and usage of different sulphur sources by C. glutamicum.

In order to test whether cultivation of C. glutamicum on different carbon sources indeed leads to an increased level of methionine synthesis efficiency, the following experiments were performed.

A C. glutamicum wild-type strain and the ΔmcbR mutant were cultivated on sulfate and thiosulfate in shaker flasks. For that purpose, the corresponding sulphur sources were added in equimolar concentrations to a sulfur-free CG12½ minimal medium.

CG12½-Medien comprises per liter: 20 g glucose, 16 g K₂HPO₄, 4 g KH₂PO₄, 20 g (NH₄)₂SO₄, 300 mg 3,4-dihydroxy benzo acid, 10 mg CaCl₂, 250 mg MgSO₄ 7H₂O, 10 mg FeSO₄.7H₂O, 10 mg MnSO₄.H₂O, 2 mg ZnSO₄.7H₂O, 200 μg CuSO₄.5H₂O, 20 μg NiCl₂.6H₂O, 20 μg Na₂MoO₄.2H₂O, 100 μg cyanocobalamine (Vitamin B₁₂), 300 μg thiamine (vitamin B₁), 4 μg pyridoxal phosphate (vitamin B₆) and 100 μg biotin (vitamin B₇).

In the case of the sulfur-free CG12½ medium all sulfates were replaced by chlorines used in concentrations such that the concentrations of the corresponding cations would not change. The following salts were used: MgCl₂.6H₂O (SO₄²⁻<0.002%, Sigma); ZnCl₂ (SO₄²⁻<0.002%, Sigma); NH₄Cl (SO₄²⁻<0.002%, Fluka); MnCl₄.4H₂O(SO₄²⁻<0.002%, Sigma) and FeCl₂.4H₂O(SO₄²⁻<0.01%, Sigma).

Cultivation of C. glutamicum was carried out in shaker flasks with indentations at 30° C. and 250 upm in shaker cabinets (Multitron, Infors A G, Bottmingen, Switzerland). In order to prevent an oxygen limitation, flasks were filled to a maximum of 10% with medium.

It is known that cysteine synthase CysK (R45 and R45a) and cystathionine-γ-synthase MetB (R46) are overexpressed in C. glutamicum ΔmcbR (Rey et al. (2003) vide supra).

In was found that both strains can grow on sulfate and thiosulfate. The highest growth rate was observed for the wild-type with μₘₐₓ=0.44 h⁻¹ on sulfate. Sulfate thus seems to be the preferred sulfur source for C. glutamicum. Thiosulfate was also used by C. glutamicum, at al lower observed growth rate of μₘₐₓ=0.31 h⁻¹.

However, an increase in biomass was observed for the wild-type from 0.35 gg⁻¹ to 0.60 gg⁻¹ if sulfate was replaced by thiosulfate. In case of the ΔmcbR knockout, the biomass yield increased even from 0.42 gg⁻¹ to 0.51 gg⁻¹ if sulfate was replaced by thiosulfate. This corresponds to an increase in yield of 13% and 21%. Replacing sulfate by thiosulfate thus indeed leads to a reduction in ATP and NADPH which in turn has a positive effect on the carbon yield.

As a reduced amount of sugar/glucose is needed for the production of biomass, more sugar/glucose is available for the production of methionine. Thus, a change from sulfate to thiosulfate should indeed lead to increased yields of methionine synthesis and this effect should be even more pronounced if use of thiosulfate as the sulfur source is combined with an increase of metabolic flux through preferred metabolic pathways by genetic manipulation.

### FIGURE LEGENDS

FIG. 1: Stoichiometric reaction network of the C. glutamicum wild type applied for elementary mode analysis. A double-beaded arrow represents reversible reactions. External metabolites are displayed in grey boxes.

FIG. 2: Metabolic pathway analysis of C glutamicum and E. coli for methionine production: carbon yield for biomass and methionine for the obtained elementary modes of C. glutamicum wild type (A), E. coli wild type (B), C. glutamicum mutant with active transhydrogenase (C), E. coli mutant lacking transhydrogenase (D), C. glutamicum mutant with active glycine cleavage system (E), E. coli mutant lacking glycine cleavage system (F). The number given indicates the maximal theoretical carbon yield for methionine for each scenario. The strait line connects the modes with maximal biomass and maximal methionine yields.

FIG. 3: Flux distribution of the C. glutamicum wild type with maximal theoretical methionine carbon yield. All fluxes are given as relative molar fluxes to the glucose uptake.

FIG. 4: Flux distribution of the E. coli wild type with maximal theoretical methionine carbon yield. All fluxes are given as relative molar fluxes to the glucose uptake.

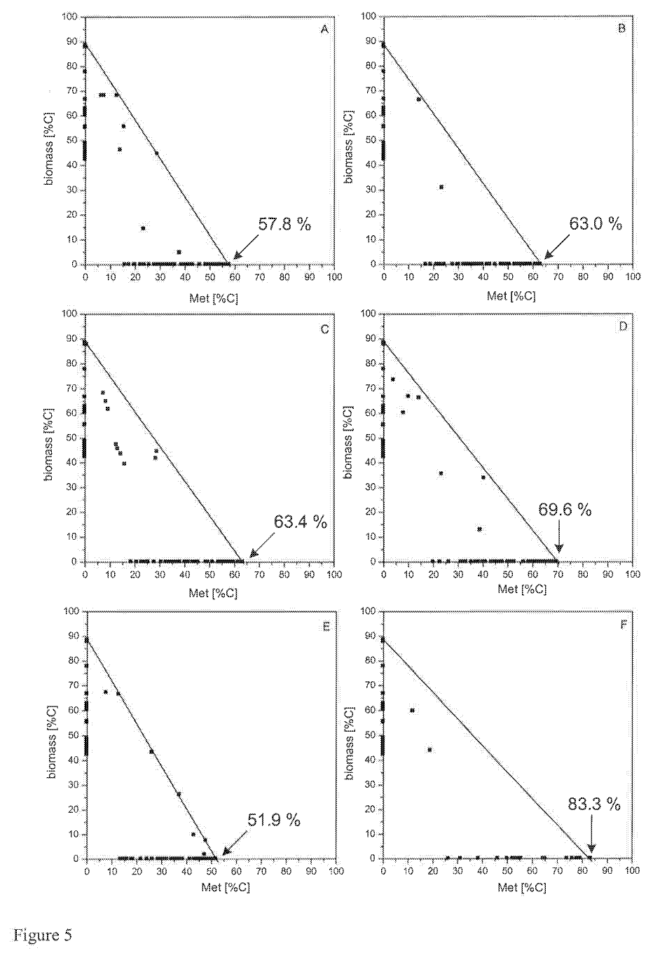

FIG. 5: Metabolic pathway analysis of C. glutamicum for methionine production with different carbon and sulfur sources: carbon yield for biomass and methionine for the obtained elementary modes of C. glutamicum utilizing thiosulfate (A), thiosulfate and formate (B), sulfide (C), sulfide and formate (D), formate (E) and methanethiol or its dimer dimethyl disulfide (F). The number given indicates the maximal theoretical carbon yield for methionine for each scenario. The straight line connects the modes with maximal biomass and maximal methionine yields.

FIG. 6 shows vector pH 273, pH 373 and pH 304

FIG. 7 shows vector pH 399, pH 484 and pH 491

FIG. 8 shows vector pOM 229.

FIG. 9 shows vector pOM 253.

FIG. 10 shows one preferred embodiment of optimized metabolic flux as regards methionine synthesis.

### ABBREVIATIONS

G6P=Glucose-6-phosphate
F6P=Fructose-6-phosphate
F— 16-BP=Fructose-1,6-bisphosphate
ASP=Aspartic acid

### ASP-P=Aspartyl-phosphate

### ASP-SA=Aspartate-semialdehyde

### HOM=Homoserine

### O-AC-HOM=O-acetyl-homoserine

HOMOCYS homocysteine

### 3-PHP=3-Phosphonooxypyruvate

### SER-P=3-Phosphoserine

### SER=Serine

### O-AC-SER=O-acetyl-serine

### CYS=Cysteine

### CYSTA=Cystathionine

GA3P=Glyceraldehyde 3-phosphate
DAHP=Dihydroxyacetone phosphate

### 13-PG=1,3-Bisphospho-glycerate

### 3-PG=3-Phospho-glycerate

### 2-PG=2-Phospho-glycerate

AC-CoA=Acetyl coenzyme A

### PYR=Pyruvate

### PEP=Phosphoenol-pyruvate

CIT=Citric acid

### OAA=Oxaloacetate

Cis-ACO=cis-Aconitate
ICI=Iso-citric acid

### 2-OXO=2-Oxoglutarate

### GLU=Glutamate

SUCC-CoA=Succinyl coenzyme A

### SUCC=Succinate

### FUM=Fumarate

### MAL=Malate

### GLYOXY=Glyoxylate

### H₂SO₃=Sulfite

### H2S=Hydrogen-sulfide

### 6-P-Gluconate=6-Phospho-gluconate

GLC-LAC=6-Phospho-glucono-1,5-lactone
RIB-5P=Ribulose 5-phosphate
RIBO-5P=Ribose 5-phosphate
XYL-5P=Xylulose 5-phosphate
S7P=Sedoheptulose 7-phosphate.
E4P=Erythrose 4-phosphate

### MET=L-Methionine

NADP=oxidized Nicotinamide adenine dinucleotide phosphate
NADPH=reduced Nicotinamide adenine dinucleotide phosphate
ACETAT=acetate

### H-CoA=Coenzyme A

FAD=oxidized Flavin adenine dinucleotide
FADH=reduced Flavin adenine dinucleotide
ATP=Adenosine 5′-triphosphate
ADP=Adenosine 5′-diphosphate
NAD=oxidized Nicotinamide adenine dinucleotide
NADH=reduced Nicotinamide adenine dinucleotide

### M-THF=5-Methyltetrahydrofolate

### THF=Tetrahydrofolate

GDP=Guanosine 5′-diphosphate
GTP=Guanosine 5′-triphosphate

### GLC=Glucose

METex=excreted Methionine

### O2=Oxygen

### NH3=Ammonia

CO2=Carbon dioxide

### SO4=Sulfate

### GLYCINE=Glycine

### HPL=H-protein-lipoyllysine

Methyl-HPL=H-protein-5-aminomethyldihydrolipoyllysine

### Reactions

The following reactions are carried out by enzymes R1 to R80:

### R1: PEP+GLC=PYR+G6P.

### R2: G6P=F6P.

### R3: G6P+NADP=GLC-LAC+NADPH.

### R4: GLC-LAC=6-P-Gluconate.

### R5: 6-P-Gluconate+NADP=RIB-5P+CO2+NADPH.

### R6: RIB-5P=XYL-5P.

### R7: RIB-5P=RIBO-5P.

### R8: S7P+GA3P=RIBO-5P+XYL-5P.

### R9: S7P+GA3P=E-4P+F6P.

### R10: F6P+GA3P=E-4P+XYL-5P.

### R11:ATP+F6P=ADP+F-16-BP.

### R12: F-16-BP=F6P.

### R13: F-16-BP=GA3P+DAHP.

### R14: DAHP=GA3P.

### R15: GA3P+NAD=13-PG+NADH.

### R16: ADP+13-PG=ATP+3-PG.

### R17: 3-PG=2-PG.

### R18: 2-PG=PEP.

### R19: PEP+ADP=PYR+ATP.

### R20: PYR+H-CoA+NAD=AC-CoA+NADH+CO₂.

### R21: AC-CoA+OAA=CIT+H-CoA.

### R22: CIT=Cis-ACO.

### R23: Cis-ACO=ICI.

### R24: ICI+NADP=2-OXO+CO₂+NADPH.

### R25: 2-OXO+NH₃+NADPH=GLU+NADP.

### R26: 2-OXO+NAD+H-CoA=SUCC-CoA+NADH+CO₂.

### R27: SUCC-CoA+GDP=SUCC+H-CoA+GTP.

### R28: SUCC+FAD=FUM+FADH.

### R29: FUM=MAL.

### R30: MAL+NAD=OAA+NADH.

### R31: ICI=GLYOXY+SUCC.

### R32: GLYOXY+AC-CoA=MAL+H-CoA.

### R33: PYR+ATP+CO₂=OAA+ADP.

### R34: PEP+CO₂=OAA.

### R35: OAA+ATP=PEP+ADP+CO₂.

### R36: OAA+ADP=PYR+CO2+ATP.

### R37: OAA+GLU+NADPH=ASP+2-OXO+NADP.

### R38: THF+SER=MTHF+GLYCINE.

### R39: ASP-SA+NADPH=HOM+NADP.

### R40: HOM+SUCC-CoA=O-SUCC-HOM+H-CoA.

### R41: 3-PG+NAD=3-PHP+NADH.

### R42: 3-PHP+GLU=SER-P+2-OXO.

### R43: SER-P=SER.

### R44: SER+AC-CoA=O-AC-SER+H-CoA.

### R45: O-AC-SER+H2S=CYS+ACETAT.

### R45a: H₂S₂O₃+O—Ac-SER=S-Sulfocystein+ACETAT

### R46: CYS+O-SUCC-HOM=CYSTA+SUCC.

### R47: ASP+ATP=ASP-P+ADP.

### R48: ASP-P+NADPH=ASP-SA+NADP.

R49 O-Acetyl-homoserine+H2S=Homocysteine+acetic acid
R50: ATP+ACETAT=ADP+acetyl-phosphate.
R51: acetyl-phosphate+H-CoA=AC-CoA.

### R52: HOMOCYS+MTHF=MET+THF.

### R53: MET=METex.

### R54: CYSTA=HOMOCYS+NH₃+PYR.

### R55: SO4+2 ATP+NADPH=H2SO3+2 ADP+NADP.

### R56: ATP=ADP.

### R57: MAL+NADP=PYR+CO2+NADPH.

### R58: H2SO3+3 NADPH=H2S+3 NADP.

### R59: 2 NADH+O2+4 ADP=2.NAD+4 ATP.

### R60: 2 FADH+O2+2ADP=2FAD+2ATP.

### R61: 6965 NH3+233 SO4+206 G6P+72 F6P+627 RIBO-5P+361 E-4P+129 GA3P+1338 3-PG+720 PEP+2861 PYR+2930 AC-CoA+1481 OAA+1078 2-OXO+16548 NADPM=BIOMASS+16548 NADP+2930H-CoA+1678 CO₂

### R62: ADP+GTP=ATP+GDP.

### R70: NADPH+NAD=NADP+NADH.

### R71: GLYCINE+HPL=Methyl-HPL+CO₂.

### R72: Methyl-HPL+THF=HPL+MTHF+NH4.

R73: I thiosulfate (S₂O₃²⁻)+1 NAD(P)H=1 sulfite+1 sulfide+1 NAD(P)
R74: sulfite+3 NAD(P)H=sulfide+3 NAD(P)
R75: ATP+Formate+THF=ADP+Orthophosphate+10-formyl-THF

### R76: 5,10-Methenyl-THF+NADPH=5,10-Methylene-THF+NADP

R77: O-Acetyl-homoserine+methanethiol=methionine+acetate

### R78: 5,10-Methylene-THF+NADP(H)=Methyl-THF

R79:formyl-tetrahydrofolate=formate+tetrahydrofolate
R80: sulfate+1 NAD(P)H+1 ATP+1 G(A)TP=sulfite+1 NAD(P), 1PPᵢ, 1 G(A)DP+adenylate+P

### R81: 3 NADH+3 NADP⁺+ATP=3 NAD⁺+3 NADPH

### R82: H₂S₂O₃ₑₓₜₑᵣₙₐₗ+ATP=H₂S₂O₃ᵢₙₜₑᵣₙₐₗ+ADP

The wild type C. glutamicum Model (Compare FIG. 1)—Reactions and Enzymes:

R1: Phospho-transferase system

### R2: G6P-isomerase

### R3: G6P-DH

### R4: Lactonase

### R5: Gluconate-DH

### R6: Ribose-5-P-epimerase

### R7: Ribose-5-P-isomerase

### R8: Transketolase 1

### R9: Transaldolase

### R10: Transketolase 2

R11: Phosphofructo kinase

### R12: Fructosebisphosphatase

### R13: Fructosebisphosphate-aldolase

### R14: Triosephosphate-isomerae

R15: 3-phospho glycerate-Kinase

### R16: PG-kinase

### R17: PG-mutase

### R18: PEP-hydrolase

### R19: PYR-kinase

### R20: PYR-DH

### R21: CIT-synthase

### R22: ACO-hydrolase

### R23: ACONITASE

### R24: Isocitrate-DH

### R25: Glutamate-DH

### R26: 2-OXO-DH

### R27: SUCC-CoA-synthase

### R28: SUCC-DH

### R29: FUMARASE

### R30: MAL-DH

### R31: ICI-lyase

### R32: MAL-synthase

### R33: PYR-carboxylase

### R34: PEP-carboxylase

### R35: PEP-carboxykinase

### R36: OAA-decarboxylase

### R37: ASP-transaminase

R38: M-THF synthesis 1

### R39: HOM-DH

### R40: HOM-transacetylase

### R41: PG-DH

### R42: Phosphoserine-transaminase

### R43: Phosphoserine-phosphatase

### R44: Serine-transacetylase

### R45: Cysteine-synthaseR46: Cystathionine-synthase

### R47: Aspartokinase

### R48: ASP-P-DH

R49: O—Ac-HOM sulphhydrylase

### R50: ACETAT-kinase

### R51: Phosphotransacetylase

### R52: MET-synthase (MetE/H)

R53: Methionine exporter
R54: Cystathionine-□-lyase

### R55: ATP-sulfurylase

### R56: ATP-hydrolysis

R57: Malic enzyme

### R58: Sulfite-reductase

R59: Respiratory chain 1
R60: Respiratory chain 2
R61: Biomass formation
R62: GTP-ATP-Phospho transferase

### Reaction Type (Reversible or Irreversible):

### Reversible.

R2r R6r R7r R8r R9r R10r R13r R14r R15r R17r R18r R22r R23r R28r R29r R30r R37r R41r R42r

### Irreversible:

R1 R3 R4 R5 R11 R12 R16 R19 R20 R21 R24 R25 R26 R27 R31 R32 R33 R34 R35 R36 R38 R39 R40 R43 R44 R45 R46 R47 R48 R49 R50 R51 R52 R53 R54 R55 R56 R57 R58 R59 R60 R61 R62

### Metabolites (Internal or External):

### Internal:

G6P F6P F-16-BP ASP ASP-P ASP-SA HOM ATP O-AC-HOM HOMOCYS 3-PHP SER-P SER O-AC-SER CYS CYSTA GA3P DAHP 13-PG 3-PG 2-PG AC-CoA PYR PEP CIT OAA Cis-ACO ICI 2-OXO GLU SUCC-CoA SUCC FUM MAL GLYOXY H2SO3 H2S 6-P-Gluconate GLC-LAC RIB-5P RIBO-5P XYL-5P S7P E-4P MET NADP NADPH acetyl-phosphate ACETAT H-CoA FAD FADH ADP NADH NAD MTHF THF GDP GTP

### External:

BIOMASS GLC METex O2 NH3 CO2 SO4 GLYCINE

### Reaction Stoichiometries:

### R1: PEP+GLC=PYR+G6P.

### R2r: G6F6P.

### R3: G6P+NADP=GLC-LAC+NADPH.

### R4: GLC-LAC=6-P-Gluconate.

### R5: 6-P-Gluconate+NADP=RTB-5P+CO2+NADPH.

### R6r: RIB-5P=XYL-5P.

### R7r: RIB-5P=RIBO-5P.

### R8r: S7P+GA3P=RIBO-5P+XYL-5P.

### R9r: S7P+GA3P=E-4P+F6P.

### R10r: F6P+GA3P=E-4P+XYL-5P.

### R11: ATP+F6P=ADP+F-16-BP.

### R12: F-16-BP=F6P.

### R13r: F-16-BP=GA3P+DAHP;

### R14r: DAHP=GA3P.

### R15r: GA3P+NAD=13-PG+NADH.

### R16: ADP+13-PG=ATP+3-PG.

### R17r: 3-PG=2-PG.

### R18r: 2-PG=PEP.

### R19: PEP+ADP=PYR+ATP.

### R20: PYR+H-CoA+NAD=AC-CoA+NADH+CO2.

### R21: AC-CoA+OAA=CIT+H-CoA.

### R22r: CIT=Cis-ACO.

### R23r: Cis-ACO=ICI.

### R24: ICI+NADP=2-OXO+CO2+NADPH.

### R25: 2-OXO+NH3+NADPH=GLU+NADP.

### R26: 2-OXO+NAD+H-CoA=SUCC-CoA+NADH+CO2.

### R27: SUCC-CoA+GDP=SUCC+H-CoA+GTP.

### R28r: SUCC+FAD=FUM+FADH.

### R29r: FUM=MAL.

### R30r: MAL+NAD=OAA+NADH.

### R31: ICI=GLYOXY+SUCC.

### R32: GLYOXY+AC-CoA=MAL+H-CoA.

### R33: PYR+ATP+CO2=OAA+ADP.

### R34: PEP+CO2=OAA.

### R35: OAA+ATP=PEP+ADP+CO2.

### R36: OAA+ADP=PYR+CO2+ATP.

### R37r: OAA+GLU+NADPH=ASP+2-OXO+NADP.

### R38: THF+SER=MTHF+GLYCINE.

### R39: ASP-SA+NADPH=HOM+NADP.

### R40: HOM+AC-CoA=O-AC-HOM+H-CoA.

### R41r: 3-PG+NAD=3-PHP+NADH.

### R42r: 3-PHP+GLU=SER-P+2-OXO.

### R43: SER-P=SER.

### R44: SER+AC-CoA=O-AC-SER+H-CoA.

### R45: O-AC-SER+H2S=CYS+ACETAT.

### R46: CYS+O-AC-HOM=CYSTA+ACETAT.

### R47: ASP+ATP=ASP-P+ADP.

### R48: ASP-P+NADPH=ASP-SA+NADP.

### R49: O-AC-HOM+H2S=HOMOCYS+ACETAT.

R50: ATP+ACETAT=ADP+acetyl-phosphate.
R51: acetyl-phosphate+H-CoA=AC-CoA.

### R52: HOMOCYS+MTHF=MET+THF.

### R53: MET=METex.

### R54: CYSTA=HOMOCYS+NH3+PYR.

### R55: SO4+2 ATP+NADPH=H2SO3+2 ADP+NADP.

### R56: ATP=ADP.

### R57: MAL+NADP=PYR+CO2+NADPH.

### R58: H2SO3+3 NADPH=H2S+3 NADP.

### R59: 2NADH+O2+4ADP=2NAD+4ATP.

### R60: 2FADH+O2+2ADP=2FAD+2ATP.

### R61: 6231 NH3+233 SO4+205 G6P+308 F6P+879 RIBO-5P+268 E-4P+129 GA3P+1295 3-PG+652 PEP+2604 PYR+3177 AC-CoA+1680 OAA+1224 2-OXO+16429 NADPH=BIOMASS+16429 NADP+3177H-CoA+1227 CO2

R62: ADP+GTP=ATP+GDP.

The Wild Type E. coli Model—Reactions and Enzymes:
R1: Phospho-transferase system

### R2: G6P-isomerase

### R3: G6P-DH

### R4: Lactonase

### R5: Gluconate-DH

### R6: Ribose-5-P-epimerase

### R7: Ribose-5-P-isomerase

### R8: Transketolase 1

### R9: Transaldolase

### R10: Transketolase 2

R11: Phosphofructo kinase

### R12: Fructosebisphosphatase

### R13: Fructosebisphosphate-aldolase

### R14: Triosephosphate-isomerae

R15: 3-phospho glycerate-Kinase

### R16: PG-kinase

### R17: PG-mutase

### R18: PEP-hydrolase

### R19: PYR-kinase

### R20: PYR-DH

### R21: CIT-synthase

### R22: ACO-hydrolase

### R23: ACONITASE

### R24: Isocitrate-DH

### R25: Glutamate-DH

### R26: 2-OXO-DH

### R27: SUCC-CoA-synthase

### R28: SUCC-DH

### R29: FUMARASE

### R30: MAL-DH

### R31: ICI-lyase

### R32: MAL-synthase

### R33: PYR-carboxylase

### R34: PEP-carboxylase

### R35: PEP-carboxykinase

### R36: OAA-decarboxylase

### R37: ASP-transaminase

R38: M-THF synthesis I

### R39: HOM-DH

### R40: HOM-transacetylase

### R41: PG-DH

### R42: Phosphoserine-transaminase

### R43: Phosphoserine-phosphatase

### R44: Serine-transacetylase

### R45: Cysteine-synthase

### R46: Cystathionine-synthase

### R47: Aspartokinase

### R48: ASP-P-DH

### R50: ACETAT-kinase

### R51: Phosphotransacetylase

### R52: MET-synthase (MetE/H)

R53: Methionine exporter
R54: Cystathionine-□-lyase

### R55: ATP-sulfurylase

### R56: ATP-hydrolysis

R57: Malic enzyme

### R58: Sulfite-reductase

R59: Respiratory chain 1
R60: Respiratory chain 2
R61: Biomass formation
R62: GTP-ATP-Phospho transferase

### R70: Transhydrogenase

R71: Glycine cleavage 1
R72: Glycine cleavage 2

### Reaction Type (Reversible or Irreversible):

### Reversible:

R2r R6r R7r R8r R9r R10r R13r R14r R15r R17r R18r R22r R23r R28r R29r R30r R37r R41r R42r R70r

### Irreversible.

R1 R3 R4 R5 R11 R12 R16 R19 R20 R21 R24 R25 R26 R27 R31 R32 R33 R34 R35 R36 R38 R39 R40 R43 R44 R45 R46 R47 R48 R50 R51 R52 R53 R54 R55 R56 R57 R58 R59 R60 R61 R62 R71 R72

### Metabolites (Internal or External):

### Internal:

G6P F6P F-16-BP ASP ASP-P ASP-SA HOM ATP O-SUCC-HOM HOMOCYS 3-PHP SER-P SER O-AC-SER CYS CYSTA GA3P DAHP 13-PG 3-PG 2-PG AC-CoA PYR PEP CIT OAA Cis-ACO ICI 2-OXO GLU SUCC-CoA SUCC FUM MAL GLYOXY H2SO₃H2S 6-P-Gluconate GLC-LAC RIB-5P RIBO-5P XYL-5P S7P E-4P MET NADP NADPH H-CoA FAD FADH ADP NADH NAD MTHF THF GDP GTP ACETAT acetyl-phosphate HPL methyl-HPL GLYCINE

### External:

BIOMASS GLC METex O2 NH3 CO2 SO4

### Reaction Stoichiometries:

### R1: PEP+GLC=PYR+G6P.

### R2r: G6P=F6P.

### R3: G6P+NADP=GLC-LAC+NADPH.

### R4: GLC-LAC=6-P-Gluconate.

### R5: 6-P-Gluconate+NADP=RIB-5P+CO2+NADPH.

### R6r: RIB-5P=XYL-5P.

### R7r: RIB-5P=RIBO-5P.

### R8r: S7P+GA3P=RIBO-5P+XYL-5P.

### R9r: S7P+GA3P=E-4P+F6P.

### R10r: F6P+GA3P=E-4P+XYL-5P.

### R11: ATP+F6P=ADP+F-16-BP.

### R12: F-16-BP=F6P.

### R13r: F-16-BP=GA3P+DAHP.

### R14r: DAHP=GA3P.

### R15r: GA3P+NAD=13-PG+NADH.

### R16: ADP+13-PG=ATP+3-PG.

### R17r: 3-PG=2-PG.

### R18r: 2-PG=PEP.

### R19: PEP+ADP=PYR+ATP.

### R20: PYR+H-CoA+NAD=AC-CoA+NADH+CO2.

### R21: AC-CoA+OAA=CIT+H-CoA.

### R22r: CIT=Cis-ACO.

### R23r: Cis-ACO=ICI.

### R24: ICI+NADP=2-OXO+CO₂+NADPH.

### R25: 2-OXO+NH₃+NADPH=GLU+NADP.

### R26: 2-OXO+NAD+H-CoA=SUCC-CoA+NADH+CO₂.

### R27: SUCC-CoA+GDP=SUCC+H-CoA+GTP.

### R28r: SUCC+FAD=FUM+FADH.

### R29r: FUM=MAL.

### R30r: MAL+NAD=OAA+NADH.

### R31: ICI=GLYOXY+SUCC.

### R32: GLYOXY+AC-CoA=MAL+H-CoA.

### R33: PYR+ATP+CO2=OAA+ADP.

### R34: PEP+CO2=OAA.

### R35: OAA+ATP=PEP+ADP+CO₂.

### R36: OAA+ADP=PYR+CO2+ATP.

### R37r: OAA+GLU+NADPH=ASP+2-OXO+NADP.

### R38: THF+SER=MTHF+GLYCINE.

### R39: ASP-SA+NADPH=HOM+NADP.

### R40: HOM+SUCC-CoA=O-SUCC-HOM+H-CoA.

### R41r: 3-PG+NAD=3-PHP+NADH.

### R42r: 3-PHP+GLU=SER-P+2-OXO.

### R43: SER-P=SER.

### R44: SER+AC-CoA=O-AC-SER+H-CoA.

### R45: O-AC-SER+H2S=CYS+ACETAT.

### R46: CYS+O-SUCC-HOM=CYSTA+SUCC.

### R47: ASP+ATP=ASP-P+ADP.

### R48: ASP-P+NADPH=ASP-SA+NADP.

### R52: HOMOCYS+MTHF=MET+THF.

### R53: MET=METex.

### R54: CYSTA=HOMOCYS+NH3+PYR.

### R55: SO4+2 ATP+NADPH=H2SO3+2 ADP+NADP.

### R56: ATP=ADP.

### R57: MAL+NADP=PYR+CO₂+NADPH.

### R58: H₂SO3+3 NADPH=H2S+3 NADP.

### R59: 2 NADH+O2+4 ADP=2 NAD+4 ATP.

### R60: 2 FADH+O2+2 ADP=2 FAD+2 ATP.

### R61: 6965 NH3+233 SO4+206 G6P+72 F6P+627 RIBO-5P+361 E-4P+129 GA3P+1338 3-PG+720 PEP+2861 PYR+2930 AC-CoA+1481 OAA+1078 2-OXO+16548 NADPH=BIOMASS+16548 NADP+2930H-CoA+1678 CO2

### R62: ADP+GTP=ATP+GDP.

R50: ATP+ACETAT=ADP+acetyl-phosphate.
R51: acetyl-phosphate+H-CoA=AC-CoA.

### R70r: NADPH+NAD=NADP+NADH.

### R71: GLYCINE+HPL=Methyl-HPL+CO2.

R72: Methyl-HPL+THF=HPL+MTHF+NH3.

1. A method for determining an organism with increased efficiency for methionine synthesis, wherein the method comprises the steps of
a. parameterizing, by means of a plurality of parameters, the metabolic flux of an initial methionine synthesizing organism based on pre-known metabolic pathways related to methionine synthesis;
b. determining a theoretic model of an organism with increased efficiency for methionine synthesis by modifying at least one of the plurality of parameters and/or introducing at least one further such parameter in such a manner as to increase the efficiency of methionine synthesis compared to the initial methionine synthesizing organism.

2. A device of determining an organism with increased efficiency for methionine synthesis, the device comprising a processor adapted to carry out the following method steps
a. parameterizing, by means of a plurality of parameters, the metabolic flux of an initial methionine synthesizing wild type organism based on pre-known metabolic pathways related to methionine synthesis;
b. determining a theoretic model of an organism with increased efficiency for methionine synthesis by modifying at least one of the plurality of parameters and/or introducing at least one further such parameter in such a manner as to increase the efficiency of methionine synthesis compared to the initial methionine synthesizing organism.

3. A computer-readable medium, in which a computer program of determining an organism with increased efficiency for methionine synthesis is stored which, when being executed by a processor, is adapted to carry out the following method steps
a. parameterizing, by means of a plurality of parameters, the metabolic flux of an initial methionine synthesizing organism based on pre-known metabolic pathways related to methionine synthesis;
b. determining a theoretic model of an organism with increased efficiency for methionine synthesis by modifying at least one of the plurality of parameters and/or introducing at least one further such parameter in such a manner as to increase the efficiency of methionine synthesis compared to the initial methionine synthesizing organism.

4. A program element of determining an organism with increased efficiency for methionine synthesis which, when being executed by a processor, is adapted to carry out the following method steps.
a. parameterizing, by means of a plurality of parameters, the metabolic flux of an initial methionine synthesizing organism based on pre-known metabolic pathways related to methionine synthesis;
b. determining a theoretic model of an organism with increased efficiency for methionine synthesis by modifying at least one of the plurality of parameters and/or introducing at least one further such parameter in such a manner as to increase the efficiency of methionine synthesis compared to the initial methionine synthesizing organism.

5. A method for producing an organism being selected from the group of prokaryotes, lower eukaryotes and plants with increased efficiency of methionine synthesis compared to the starting organisms comprising the following steps:
a. parameterizing, by means of a plurality of parameters, the metabolic flux of an initial methionine synthesizing organism based on pre-known metabolic pathways related to methionine synthesis;
b. determining a theoretic model of an organism with increased efficiency for methionine synthesis by modifying at least one of the plurality of parameters and/or introducing at least one further such parameter in such a manner as to increase the efficiency of methionine synthesis compared to the initial methionine synthesizing organism.
c. genetically modifying a starting organism in such a manner as to modify at least one existing metabolic pathway in the organisms such that the metabolic flux of the organism is approximated to the theoretical model of the organism and/or
d. genetically modifying a starting organism in such a manner as to introduce at least one exogenous metabolic pathway into the organisms such that the metabolic flux of the organism is approximated to the theoretical model of the organism and/or
e. providing at least one external metabolites in an amount sufficient to channel the metabolic flux through the metabolic pathways, modified in step c and/or introduced in step d.

6. The method according to claim 5, wherein the metabolic flux through at least one of the existing metabolic pathways selected from the group consisting of
phosphotransferase system (PTS)
pentose phosphate pathway (PPP)
glycolysis (EMP)
tricarboxylic acid cycle (TCA)
glyoxylate shunt (GS)
anaplerosis (AP)
respiratory chain (RC)
sulfur assimilation (SA)
methionine synthesis (MS)
serine/cysteine/glycine synthesis (SCGS)
glycine cleavage system (GCS)
transhydrogenase conversion (THGC)
pathway 1 (P1)
pathway 2 (P2)
pathway 3 (P3)
pathway 4 (P4)
pathway 5 (P5)
pathway 6 (P6)
pathway 7 (P7)
pathway 8 (P8)
is modified by genetic modification of the organisms, and/or
the metabolic flux through at least one of the exogenous metabolic pathways selected from the group consisting of
Glycine cleavage system (GCS)
transhydrogenase conversion (THGC)
Thiosulfate Reductase System (TRS)
Sulfite Reductase System (SRS)
Sulfate Reductase System (SARS)
Formate converting system (FCS)
Methanethiol converting system (MCS)
is introduced by genetic modification of the organisms, and/or
the organisms are cultivated in the presence of external metabolites selected from the group consisting of
sulfate
sulfite
sulfide
thiosulfate
C1-metabolites such as formate, formaldehyde, methanol, methanethiol or its dimer dimethyl-disulfide

7. A method for producing an organism being selected from the group of prokaryotes, lower eukaryotes and plants with increased efficiency of methionine synthesis compared to the starting organisms comprising the following steps:
a. modifying the metabolic flux through at least one of the metabolic pathways selected from the group consisting of:
phosphotransferase system (PTS)
pentose phosphate pathway (PPP)
glycolysis (EMP)
tricarboxylic acid cycle (TCA)
glyoxylate shunt (GS)
anaplerosis (AP)
respiratory chain (RC)
sulfur assimilation (SA)
methionine synthesis (MS)
serine/cysteine/glycine synthesis (SCGS)
glycine cleavage system (GCS)
transhydrogenase conversion (THGC)
pathway 1 (P1)
pathway 2 (P2)
pathway 3 (P3)
pathway 4 (P4)
pathway 5 (P5)
pathway 6 (P6)
pathway 7 (P7)
pathway 8 (P8)
by genetic modification of the organism, and/or
b. introducing a metabolic flux through at least one of the exogenous metabolic pathways selected from the group consisting of
Glycine cleavage system (GCS)
transhydrogenase conversion (THGC)
Thiosulfate Reductase System (TRS)
Sulfite Reductase System (SRS)
Sulfate Reductase System (SRS)
Formate converting system (FCS)
Methanethiol converting system (MCS)
by genetic modification of the organism, and/or
c. cultivating the organisms in the presence of at least one external metabolite selected from the group consisting of:
sulfate
sulfite
sulfide
thiosulfate
organic sulfur sources
C1-metabolites such as formate, formaldehyde, methanol, methanethiol or its dimer dimethyldisulfide.

8. An organism being selected from the group of prokaryotes, lower eukaryotes and plants with increased efficiency of methionine synthesis compared to the starting organisms obtainable by the methods of claim 5.

9. The organism according to claim 8 or 23, wherein the organism is selected from the group consisting of microorganisms of the genus Corynebacterium, of the genus Brevibacterium, of the genus Escherichia, yeasts and plants.

10. A method for producing a microorganism of the genus Corynebacterium with increased efficiency of methionine production comprising the following steps
a. increasing and/or introducing the metabolic flux through at least one of the pathways selected from the group consisting of:
phosphotransferase system (PTS) and/or
pentose phosphate pathway (PPP) and/or
sulfur assimilation (SA) and/or
anaplerosis (AP) and/or
methionine synthesis (MS) and/or
serine glycine synthesis (SCGS) and/or
glycine cleavage system (GCS) and/or
transhydrogenase conversion (THGC) and/or
pathway 1 (P1) and/or
pathway 2 (P2) and/or
Thiosulfate Reductase System (TRS) and/or
Sulfite Reductase System (SRS) and/or
Sulfate Reductase System (SARS) and/or
Formate converting system (FCS) and/or
Methanethiol converting system (MCS) and/or
by genetic modification of the organism compared to the starting organism, and/or
b. at least partially decreasing the metabolic flux through at least one of the pathways selected from the group consisting of:
glycolysis (EMP) and/or
tricarboxylic acid cycle (TCA) and/or
glyoxylate shunt (GS) and/or
respiratory chain (RC) and/or
R19 and/or
R35 and/or
R79 and/or
pathway 3 (P3) and/or
pathway 4 (P4) and/or
pathway 7 (P7) and/or
by genetic modification of the organism compared to the starting.

11. The method according to claim 10 wherein the amount and/or activity of enzymes selected from the group consisting of:
R1 in order to produce more G6P and/or
R3 in order to produce more GLC-LAC and/or
R4 in order to produce more 6-P-Gluconate and/or
R5 in order to produce more RIB-5P and/or
R6 in order to produce more XYL-5P and/or
R7 in order to produce more RIBO-5P and/or
R8 in order to produce more S7P and GA3P and/or
R9 in order to produce more E-4p and F6P and/or
R10 in order to produce more F6P and GA3P and/or
R2 in order to produce more G6P and/or
R55 in order to produce more H2SO3 and/or
R58 in order to produce more H2S and/or
R71 in order to produce more M-HPL and/or
R72 in order to produce more Methylene-THF and/or
R70 in order to produce more NADPH and/or
R81 in order to produce more NADPH and/or
R25 in order to produce more Glu and/or
R33 and/or R36 in order to produce more OAA and/or
R30 in order to produce more MAL and/or
R57 in order to produce more Pyr and/or
R73 in order to metabolize thiosulfate to sulfide and sulfite and/or
R82 in order to import more external thiosulfate into the cell and/or
R74 in order to metabolize sulfite to sulfide and/or
R75 in order to produce more 10-formyl-THF and/or
R76 in order to produce more Methylene-THF and/or
R78 in order to produce more Methyl-THF and/or
R77 in order to methyl-sulfhydrylate O-Acetyl-homoserine with methanethiol and/or
R80 in order to metabolise sulfate into sulfite and/or
R47 and/or
R48 and/or
R39 and/or
R46 and/or
R49 and/or
R52 and/or
R52 and/or
R54
is increased and/or introduced compared to the starting organism, and/or the amount and/or activity of enzymes selected from the group consisting of
R11 in order to produce less F-1,6-BP and/or
R13 in order to produce less DHAP and GA3P and/or
R14 in order to produce less GA3P and/or
R15 in order to produce less 1,3-PG and/or
R16 in order to produce less 3-PG and/or
R17 in order to produce less 2-PG and/or
R18 in order to produce less PEP and/or
R19 in order to produce less Pyr and/or
R20 in order to produce less Ac-CoA and/or
R21 in order to produce less CIT and/or
R22 in order to produce less Cis-ACO and/or
R23 in order to produce less ICI and/or
R24 in order to produce less 2-OXO and/or
R26 in order to produce less SUCC-CoA and/or
R27 in order to produce less SUCC and/or
R28 in order to produce less FUM and/or
R29 in order to produce less MAL and/or
R30 in order to produce less OAA and/or
R21 in order to produce less CIT and/or
R22 in order to produce less Cis-ACO and/or
R23 in order to produce less ICI and/or
R31 in order to produce less GLYOXY and SUCC and/or
R32 in order to produce less MAL and/or
R28 in order to produce less FUM and/or
R29 in order to produce less MAL and/or
R30 in order to produce less OAA and/or
R60 and/or
R56 and/or
R62 and/or
R61 and/or
R19 and/or
R35 and/or
R79
is/are at least partially reduced compared to the starting organism.

12. The method according to claim 11 wherein the amount and/or activity of enzymes selected from the group consisting of:
R3 in order to produce more GLC-LAC and/or
R4 in order to produce more 6-P-Gluconate and/or
R5 in order to produce more RIB-5P and/or
R10 in order to produce more F6P and GA3P and/or
R2 in order to produce more G6P and/or
R55 in order to produce more H2SO3 and/or
R58 in order to produce more H2S and/or
R71 in order to produce more M-HPL and/or
R72 in order to produce more Methylene-THF and/or
R70 in order to produce more NADPH and/or
R81 in order to produce more NADPH and/or
R25 in order to produce more Glu and/or
R33 and/or R36 in order to produce more OAA and/or
R30 in order to produce more MAL and/or
R57 in order to produce more Pyr and/or
R73 in order to metabolize thiosulfate to sulfide and sulfite and/or
R82 in order to import more external thiosulfate into the cell and/or
R75 in order to produce 10-formyl-THF and/or
R76 in order to produce more Methylene-THF and/or
R78 in order to produce more Methyl-THF and/or
R77 in order methyl-sulfhydrylate O-Acetyl-homoserine with methanethiol and/or
R47 and/or
R48 and/or
R39 and/or
R46 and/or
R49 and/or
R52 and/or
R52 and/or
R54 and or
R80 in order to metabolise sulfate into sulfite
are increased and/or introduced compared to the starting organism, and/or the amount and/or activity of enzymes selected from the group consisting of:
R11 in order to produce less F-1,6-BP and/or
R19 in order to produce less Pyr and/or
R20 in order to produce less Ac-CoA and/or
R21 in order to produce less CIT and/or
R24 in order to produce less 2-OXO and/or
R26 in order to produce less SUCC-CoA and/or
R27 in order to produce less SUCC and/or
R31 in order to produce less GLYOXY and SUCC and/or
R32 in order to produce less MAL and/or
R19 in order to produce less Pyruvate and/or
R35 in order to produce less PEP and/or
R79 in order to produce less THF
are at least partially reduced compared to the starting organism.

13. The method of claim 11 wherein
the amount and/or activity of enzymes selected from the group consisting of
R3 in order to produce more GLC-LAC and/or
R4 in order to produce more 6-P-Gluconate and/or
R5 in order to produce more RIB-5P and/or
R10 in order to produce more F6P and GA3P and/or
R2 in order to produce more G6P and
R55 in order to produce more H2SO3 and/or
R58 in order to produce more H2S and
R71 in order to produce more M-HPL and/or
R72 in order to produce more Methylene-THF and/or
R78 in order to produce more Methyl-THF and
R70 in order to produce more NADPH and/or
R81 in order to produce more NADPH and/or
R25 in order to produce more Glu and/or
R33 and/or R36 in order to produce more OAA and/or
R30 in order to produce more MAL and/or
R57 in order to produce more Pyr and/or
R73 in order to metabolize thiosulfate to sulfide and sulfite and
R82 in order to import more external thiosulfate into the cell and/or
R75 in order to produce 10-formyl-THF and/or
R76 in order to produce Methylene-THF and
R77 in order to methyl-sulfhydrylate O-Acetyl-homoserine with methanethiol and/or
R47 and/or
R48 and/or
R39 and/or
R46 and/or
R49 and/or
R52 and/or
R52 and/or
R54 and/or
R80 in order to metabolise sulfate into sulfite
are increased and/or introduced compared to the starting organism, and/or:
the amount and/or activity of enzymes selected from the group consisting of:
R11 in order to produce less F-1,6-BP and/or
R19 in order to produce less Pyr and/or
R20 in order to produce less Ac-CoA and/or
R21 in order to produce less CIT and/or
R24 in order to produce less 2-OXO and/or
R26 in order to produce less SUCC-CoA and/or
R27 in order to produce less SUCC and/or
R31 in order to produce less GLYOXY and SUCC and/or
R32 in order to produce less MAL and
R19 in order to produce less Pyruvate and
R35 in order to produce less PEP and
R79 in order to produce less THF
are at least partially reduced compared to the starting organism.

14. A microorganism of the genus Corynebacterium obtainable by any of the methods according to claim 10 preferably selected from the group consisting of Corynebacterium acetoacidophilum, C. acetoglutamicum, C. acetophilum, C. ammoniagenes, C. glutamicum, C. lilium, C. nitrilophilus or C. spec. and preferably Corynebacterium glutamicum ATCC 13032, Corynebacterium acetoglutamicum ATCC 15806, Corynebacterium acetoacidophilum ATCC 13870, Corynebacterium thermoaminogenes FERM BP-1539, Corynebacterium melassecola ATCC 17965, Corynebacterium glutamicum KFCC 10065 or Corynebacterium glutamicum ATCC21608 and Corynebacterium glutamicum DSM 17322.

15. A method for producing a microorganism of the genus Escherichia with increased efficiency of methionine production comprising the following steps increasing and/or introducing the metabolic flux through at least one of the pathways selected from the group consisting of:
phosphotransferase system (PTS) and/or
glyoclysis (EMP) and/or
tricarboxylic acid cycle (TCA) and/or
glyoxylate shunt (GS) and/or
pathway 1 (P1) and/or
sulfur assimilation (SA) and/or
anaplerosis (AP) and/or
methionine synthesis (MS) and/or
serine/cysteine/glycine (SCGS) and/or
glycine cleavage system (GCS) and/or
transhydrogenase conversion (THGC) and/or
Thiosulfate Reductase System (TRS) and/or
Sulfite Reductase System (SRS) and/or
Sulfate Reductase System (SARS) and/or
Formate converting system (FCS) and/or
Methanethiol converting system (MCS) and/or
Serine/cysteine/glycine synthesis (SCGS)
compared to the starting by genetic modification of the organism, and/or
at least partially decreasing the metabolic flux through at least one of the pathways selected from the group consisting of:
pentose phosphate pathway (PPP) and/or
R19 in order to produce less Pyruvate and/or
R35 in order to produce less PEP and/or
R79 in order to produce less THF
pathway 3 (P3) and/or
pathway 4 (P4) and/or
pathway 7 (P7)
compared to the starting by genetic modification of the organism.

16. The method according to claim 15 wherein the amount and/or activity of enzymes selected from the group consisting of:
R1 in order to produce more G6P
R2 in order to produce more F6P and/or
R11 in order to produce more F-1,6-BP and/or
R13 in order to produce more DHAP and GA3P and/or
R14 in order to produce more GA3P and/or
R15 in order to produce more 1,3-PG and/or
R16 in order to produce more 3-PG and/or
R17 in order to produce more 2-PG and/or
R18 in order to produce more PEP and/or
R19 in order to produce more Pyr and/or
R20 in order to produce more Ac-CoA and/or
R21 in order to produce more CIT and/or
R22 in order to produce more Cis-ACO and/or
R23 in order to produce more ICI and/or
R24 in order to produce more 2-OXO and/or
R26 in order to produce more SUCC-CoA and/or
R27 in order to produce more SUCC and/or
R28 in order to produce more FUM and/or
R29 in order to produce more MAL and/or
R30 in order to produce more OAA and/or
R21 in order to produce more CIT and/or
R22 in order to produce more Cis-ACO and/or
R23 in order to produce more ICI and/or
R31 in order to produce more GLYOXY and SUCC and/or
R32 in order to produce more MAL and/or
R28 in order to produce more FUM and/or
R29 in order to produce more MAL and/or
R30 in order to produce more OAA and/or
R25 in order to produce more Glu and/or
R55 in order to produce more H2SO3 and/or
R58 in order to produce more H2S and/or
R71 in order to produce more M-HPL and/or
R72 in order to produce more Methylene-THF and/or
R78 in order to produce more Methyl-THF and/or
R70 in order to produce more NADPH and/or
R81 in order to produce more NADPH and/or
R73 in order to metabolize thiosulfate to sulfide and sulfite and/or
R82 in order to import more external thiosulfate into the cell and/or
R74 in order to metabolize sulfite to sulfide and/or
R75 in order to produce more 10-formyl-THF and/or
R76 in order to produce more Methylene-THF from 10-formyl-THF and/or
R77 in order to methyl-sulfhydrylate O-Acetyl-homoserine with methanethiol and/or
R80 in order to metabolise sulfate into sulfite and/or
R44 in order to produce more O—Ac-SER and/or
R45 in order to produce more CYS
is increased and/or introduced compared to the starting organism, and/or
the amount and/or activity of enzymes selected from the group consisting of:
R3 in order to produce less GLC-LAC and/or
R4 in order to produce less 6-P-Gluconate and/or
R5 in order to produce less RIB-5P and/or
R6 in order to produce less XYL-5P and/or
R7 in order to produce less RIBO-5P and/or
R8 in order to produce less S7P and GA3P and/or
R9 in order to produce less E-4p and F6P and/or
R10 in order to produce less F6P and GA3P and/or
R2 in order to produce less G6P and/or
R49 in order to produce less HOMOCYS and/or
R19 in order to produce less Pyruvate and/or
R35 in order to produce less PEP and/or
R79 in order to produce less THF and/or
R56 and/or
R62 and/or
R61
is/are at least partially reduced compared to the starting organism.

17. The method according to claim 16 wherein
the amount and/or activity of enzymes selected from the group consisting of:
R1 in order to produce more G6P and/or
R2 in order to produce more F6P and/or
R11 in order to produce more F-1,6-BP and/or
R19 in order to produce more Pyr and/or
R20 in order to produce more Ac-CoA and/or
R21 in order to produce more CIT and/or
R24 in order to produce more 2-OXO and/or
R26 in order to produce more SUCC-CoA and/or
R31 in order to produce more GLYOXY and SUCC and/or
R32 in order to produce more MAL and/or
R25 in order to produce more Glu and/or
R55 in order to produce more H₂SO₃ and/or
R58 in order to produce more H2S and/or
R71 in order to produce more M-HPL and/or
R72 in order to produce more Methylene-THF and/or
R78 in order to produce more Methyl-THF and/or
R70 in order to produce more NADPH and/or
R81 in order to produce more NADPH and/or
R73 in order to metabolize thiosulfate to sulfide and sulfite and/or
R82 in order to import more external thiosulfate into the cell and/or
R74 in order to metabolize sulfite to sulfide and/or
R75 in order to produce more 10-formyl-THF and/or
R76 in order to produce more Methylene-THF and/or
R77 in order to methyl-sulfhydrylate O-Acetyl-homoserine with methanethiol and/or
R80 to metabolise sulfate into sulfite and/or
R44 in order to produce more O—Ac-SER and/or
R45 in order to produce more CYS
is/are increased and/or introduced compared to the starting organism, and/or the amount and/or activity of enzymes selected from the group consisting of:
R3 in order to produce less GLC-LAC and/or
R4 in order to produce less 6-P-Gluconate and/or
R5 in order to produce less RIB-5P and/or
R10 in order to produce less F6P and GA3P and/or
R19 in order to produce less Pyruvate and/or
R35 in order to produce less PEP and/or
R79 in order to produce less THF
is/are at least partially reduced compared to the starting organism

18. A microorganism of the genus Escherichia obtainable by the methods of claim 15 preferably selected from the group consisting of E. coli.

19. The organism according to any one of claim 8, 14, 18, and 23 wherein methionine is produced with a molar ratio of methionine to glucose input of at least 10%, of at least 20%, of at least 30%, of at least 40%, at least 45%, at least 50%, at least 55%, at least 60%, at least 65%, at least 70%, at least 75%, at least 80% or at least 85%.

20. The use of organism of any one of claim 8, 14, 18, and 23 for producing methionine.

21. A method of producing methionine comprising the following steps:
a. cultivating an organism according to any one of claim 8, 14, 18, and 23; and
b. isolating methionine

22. The method according to claim 21 wherein cultivation is performed in a suitable medium and optionally thiosulfate, sulfite, sulfide and/or C1-compounds such as formate or methanethiol.

23. An organism being selected from the group of prokaryotes, lower eukaryotes and plants with increased efficiency of methionine synthesis compared to the starting organisms obtainable by the methods of claim 7.


SEQUENCE LISTING (13 sequences)

SEQ ID NO: 1 - DNA, 7070 bp, Artificial
  plasmid pH273
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctcctctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 2 - DNA, 7070 bp, Artificial; identical to SEQ ID NO: 1
  plasmid pH373

SEQ ID NO: 3 - DNA, 8766 bp, Artificial
  plasmid pH304
tcgagaggcctgacgtcgggcccggtaccacgcgtcatatgactagttcggacctagggatatcgtcgacatcgatgctcttctgcgttaattaacaattgggatctctcaactaatgcagcgatgcgttctttccagaatgctttcatgacagggatgctgtcttgatcaggcaggcgtctgtgctggatgccgaagctggatttattgtcgcctttggaggtgaagttgacgctcactcgagaatcatcggccaaccatttggcattgaatgttctaggttcggaggcggaggttttctcaattagtgcgggatcgagccactgcgcccgcaggtcatcgtctccgaagagcttccacactttttcgaccggcaggttaagggttttggaggcattggccgcgaacccatcgctggtcatcccgggtttgcgcatgccacgttcgtattcataaccaatcgcgatgccttgagcccaccagccactgacatcaaagttgtccacgatgtgctttgcgatgtgggtgtgagtccaagaggtggcttttacgtcgtcaagcaattttagccactcttcccacggctttccggtgccgttgaggatagcttcaggggacatgcctggtgttgagccttgcggagtggagtcagtcatgcgaccgagactagtggcgctttgggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 4 - DNA, 7070 bp, Artificial
  plasmid pH399
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctccgctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 5 - DNA, 6625 bp, Artificial
  plasmid pH484
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 6 - DNA, 6350 bp, Artificial
  plasmid pH491
tcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 7 - DNA, 7520 bp, Artificial
  plasmid pOM423
ctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccggtaccacgcgtgagttctttgagttcctgtggggtgaacttgacctgtgctgggccacgacgtccgaaaacgtgcacttcagtggccttgttttctttgagggagtcgtagacgttgtcggaaatttcggtgactttgagctcgtcgcctgtcttagccaggatgcgggctacgtcgaggccgacgttaccaacgccgataacagcgacggactgtgcagacagatcccaggagcgctcgaagcgtgggttgccgtcgtagaagccaacgaactcgccggcaccgaaggagccttctgcttcaattccggggatgttgaggtcgcggtctgcaactgcgccggtggagaacacgactgcatcgtagtagtcgcggagttcttcgacggtgatgtctttgccgatttcaatgttaccgagcaggcgcaggcgtggcttgtccaacacgttgtgcagggacttaacgatgcccttgatgcgtgggtggtctggagcaacgccgtaacggatgagtccgaacggtgcaggcatttgctcgaaaaggtcaacgaacacttcgcgctcttcattgcggatgaggaggtcggatgcgtaaatgccagcagggccagctccgatgacggctacgcgcaggggagttgtcataatattacacctccttagataacaattgattgaatgtatgcaaataaatgcccttcttcagggcttaatttttaagagcgtcaccttcatggtggtcagtgcgtcctgctgatgtgctcagtatcaccgccagtggtatttatgtcaacaccgccagagataatttatcaccgcagatggttatctgtatgttttttatatgaatttattttttgcaggggggcattgtttggtaggtgagagatcaattctgcgtcgactcatacgttaaatctatcaccgcaagggataaatatctaacaccgtgcgtgttgactattttacctctggcggtgataatggttgcatgtactaaggaggattaattaatgacaacaaccaccggaagtgcccggccagcacgtgccgccaggaagcctaagcccgaaggccaatggaaaatcgacggcaccgagccgcttaaccatgccgaggaaattaagcaagaagaacccgcttttgctgtcaagcagcgggtcattgatatttactccaagcagggtttttcttccattgcaccggatgacattgccccacgctttaagtggttgggcatttacacccagcgtaagcaggatctgggcggtgaactgaccggtcagcttcctgatgatgagctgcaggatgagtacttcatgatgcgtgtgcgttttgatggcggactggcttcccctgagcgcctgcgtgccgtgggtgaaatttctagggattatgctcgttccaccgcggacttcaccgaccgccagaacattcagctgcactggattcgtattgaagatgtgcctgcgatctgggagaagctagaaaccgtcggactgtccaccatgcttggttgcggtgacgttccacgtgttatcttgggctccccagtttctggcgtagctgctgaagagctgatcgatgccaccccggctatcgatgcgattcgtgagcgctacctagacaaggaagagttccacaaccttcctcgtaaggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagct

SEQ ID NO: 8 - DNA, 11558 bp, Artificial
  plasmid pOM229
ggatcggcggccagggccctcatggatgaccatccccggcaccatccttgacaccgcccgcacccaagttctggaacagggaattggccttaatcagcagcagttgatggaggttctcaccttgcctgaagagcaaatcccagacttgatggaattagcccaccaggttcggttgaagtggtgtggggaagaaatcgaggtcgagggcattatttccctcaaaactggcggttgccctgaagattgtcatttctgctcacagtctgggttgtttgaatcgccggtgcgttcggtgtggctggatattccgaatctggttgaagccgctaaacagaccgcaaaaactggcgctaccgaattctgtatcgtcgccgcagtcaaggggcctgatgagaggctcatgacccagctggaggaagcagtcctcgcgattcactctgaagttgaaattgaagtcgcagcatcgatcggaacgttaaataaggaacaggtggatcgcctcgctgctgccggcgtgcaccgctagcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggaggtgaaagtatggcacagcagacccctttgtacgaacaacacacgctttgcggcgctcgcatggtggatttccacggctggatgatgccgctgcattacggttcgcaaatcgacgaacatcatgcggtacgtaccgatgccggaatgtttgatgtgtcacatatgaccatcgtcgatcttcgcggcagccgcacccgggagtttctgcgttatctgctggcgaacgatgtggcgaagctcaccaaaagcggcaaagccctttactcggggatgttgaatgcctctggcggtgtgatagatgacctcatcgtctactactttactgaagatttcttccgcctcgttgttaactccgccacccgcgaaaaagacctctcctggattacccaacacgctgaacctttcggcatcgaaattaccgttcgtgatgacctttccatgattgccgtgcaagggccgaatgcgcaggcaaaagctgccacactgtttaatgacgcccagcgtcaggcggtggaagggatgaaaccgttctttggcgtgcaggcgggcgatctgtttattgccaccactggttataccggtgaagcgggctatgaaattgcgctgcccaatgaaaaagcggccgatttctggcgtgcgctggtggaagcgggtgttaagccatgtggcttgggcgcgcgtgacacgctgcgtctggaagcgggcatgaatctttatggtcaggagatggacgaaaccatctctcctttagccgccaacatgggctggaccatcgcctgggaaccggcagatcgtgactttatcggtcgtgaagccctggaagtgcagcgtgagcatggtacagaaaaactggttggtctggtgatgaccgaaaaaggcgtgctgcgtaatgaactgccggtacgctttaccgatgcgcagggcaaccagcatgaaggcattatcaccagcggtactttctccccgacgctgggttacagcattgcgctggcgcgcgtgccggaaggtattggcgaaacggcgattgtgcaaattcgcaaccgtgaaatgccggttaaagtgacaaaacctgtttttgtgcgtaacggcaaagccgtcgcgtgatttacttttttggagattgattgatgagcaacgtaccagcagaactgaaatacagcaaagaacacgaatggctgcgtaaagaagccgacggcacttacaccgttggtattaccgaacatgctcaggagctgttaggcgatatggtgtttgttgacctgccggaagtgggcgcaacggttagcgcgggcgatgactgcgcggttgccgaatcggtaaaagcggcgtcagacatttatgcgccagtaagcggtgaaatcgtggcggtaaacgacgcactgagcgattccccggaactggtgaacagcgaaccgtatgcaggcggctggatctttaaaatcaaagccagcgatgaaagcgaactggaatcactgctggatgcgaccgcatacgaagcattgttagaagacgagtaacggctttattcctcttctgcgggagaggatcagggtgaggaaaatttatgcctcaccctcactctcttcgtaaggagagaggttcacaattcactgcacgtttcaggaaccatcgctcatgacacagacgttaagccagcttgaaaacagcggcgcttttattgaacgccatatcggaccggacgccgcgcaacagcaagaaatgctgaatgccgttggtgcacaatcgttaaacgcgctgaccggccagattgtgccgaaagatattcaacttgcgacaccaccgcaggttggcgcaccggcgaccgaatacgccgcactggcagaactcaaggctattgccagtcgcaataaacgcttcacgtcttacatcggcatgggttacaccgccgtgcagctaccgccggttatcctgcgtaacatgctggaaaatccgggctggtataccgcgtacactccgtatcaacctgaagtctcccagggccgccttgaagcactgctcaacttccagcaggtaacgctggatttgactggactggatatggcctctgcttctcttctggacgaggccaccgctgccgccgaagcaatggcgatggcgaaacgcgtcagcaaactgaaaaatgccaaccgcttcttcgtggcttccgatgtgcatccgcaaacgctggatgtggtccgtactcgtgccgaaacctttggttttgaagtgattgtcgatgacgcgcaaaaagtgctcgaccatcaggacgtcttcggcgtgctgttacagcaggtaggcactaccggtgaaattcacgactacactgcgcttattagcgaactgaaatcacgcaaaattgtggtcagcgttgccgccgatattatggcgctggtgctgttaactgcgccgggtaaacagggcgcggatattgtttttggttcggcgcaacgcttcggcgtgccgatgggctacggtggcccacacgcggcattctttgcggcgaaagatgaatacaaacgctcaatgccgggccgtattatcggtgtatcgaaagatgcagctggcaataccgcgctgcgcatggcgatgcagactcgcgagcaacatatccgccgtgagaaagcgaactccaacatttgtacttcccaggtactgctggcaaacatcgccagcctgtatgccgtttatcacggcccggttggcctgaaacgtatcgctaaccgcattcaccgtctgaccgatatcctggcggcgggcctgcaacaaaaaggtctgaaactgcgccatgcgcactatttcgacaccttgtgtgtggaagtggccgacaaagcgggcgtactgacgcgtgccgaagcggctgaaatcaacctgcgtagcgatattctgaacgcggttgggatcacccttgatgaaacaaccacgcgtgaaaacgtaatgcagcttttcaacgtgctgctgggcgataaccacggcctggacatcgacacgctggacaaagacgtggctcacgacagccgctctatccagcctgcgatgctgcgcgacgacgaaatcctcacccatccggtgtttaatcgctaccacagcgaaaccgaaatgatgcgctatatgcactcgctggagcgtaaagatctggcgctgaatcaggcgatgatcccgctgggttcctgcaccatgaaactgaacgccgccgccgagatgatcccaatcacctggccggaatttgccgaactgcacccgttctgcccgccggagcaggccgaaggttatcagcagatgattgcgcagctggctgactggctggtgaaactgaccggttacgacgccgtttgtatgcagccgaactctggcgcacagggcgaatacgcgggcctgctggcgattcgtcattatcatgaaagccgcaacgaagggcatcgcgatatctgcctgatcccggcttctgcgcacggaactaaccccgcttctgcacatatggcaggaatgcaggtggtggttgtggcgtgtgataaaaacggcaacatcgatctgactgatctgcgcgcgaaagcggaacaggcgggcgataacctctcctgtatcatggtgacttatccttctacccacggcgtgtatgaagaaacgatccgtgaagtgtgtgaagtcgtgcatcagttcggcggtcaggtttaccttgatggcgcgaacatgaacgcccaggttggcatcacctcgccgggctttattggtgcggacgtttcacaccttaacctacataaaactttctgcattccgcacggcggtggtggtccgggtatgggaccgatcggcgtgaaagcgcatttggcaccgtttgtaccgggtcatagcgtggtgcaaatcgaaggcatgttaacccgtcagggcgcggtttctgcggcaccgttcggtagcgcctctatcctgccaatcagctggatgtacatccgcatgatgggcgcagaagggctgaaaaaagcaagccaggtggcaatcctcaacgccaactatattgccagccgcctgcaggatgccttcccggtgctgtataccggtcgcgacggtcgcgtggcgcacgaatgtattctcgatattcgcccgctgaaagaagaaaccggcatcagcgagctggatattgccaagcgcctgatcgactacggtttccacgcgccgacgatgtcgttcccggtggcgggtacgctgatggttgaaccgactgaatctgaaagcaaagtggaactggatcgctttatcgacgcgatgctggctatccgcgcagaaattgaccaggtgaaagccggtgtctggccgctggaagataacccgctggtgaacgcgccgcacattcagagcgaactggtcgccgagtgggcgcatccgtacagccgtgaagttgcggtattcccggcaggtgtggcagacaaatactggccgacagtgaaacgtctggatgatgtttacggcgaccgtaacctgttctgctcctgcgtaccgattagcgaataccagtaattcactgattcgactattttctaaaggcgcttcggcgcctttttagtcagatgacaaagtacaaaagtgctcagacagtcccctcgccggatccgccctcccgcaaagcgtgcgggagggcggtacctgcgcgttcctatttccctgaagttgtcaccactcatacatgggaagagcgccgcgaaactttgcgcctggtggcagaagctggaatggaagtctgttccggcggaatcttaggaatgggcgaaactttagagcagcgcgccgagtttgccgtgcagctggcggagcttgatccgcacgaagtccccatgaacttccttgatcctcgcccgggcaccccatttgccgatagggaattgatggacagccgtgacgctctgcgctctattggtgcgttccgccttgcgatgcctcacaccatgcttcgttttgctggcggtcgcgagctgactttgggcgacaagggttccgagcaagccctcctgggaggcatcaatgcgatgatcgtcggaaactacctgactacgctcggccgcccaatggaagatgacctcgacatgatggatcgtctccagctgcccatcaaagtccttaataaggacgcgtcccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagtttaaactgcggatcagtgagggtttgtaactgcgggtcaaggatctggatttcgatcacggcacgatcatcgtgcgggagggcaagggctccaaggatcgggccttgatgttacccgagagcttggcacccagcctgcgcgagcaggggaattgatccggtggatgaccttttgaatgacctttaatagattatattactaattaattggggaccctagaggtccccttttttattttaaaaattttttcacaaaacggtttacaagcataacgggttttgctgcccgcaaacgggctgttctggtgttgctagtttgttatcagaatcgcagatccggcttcaggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttaaatgcaccaaaaactcgtaaaagctctgatgtatctatcttttttacaccgttttcatctgtgcatatggacagttttccctttgatatctaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatgcttactttgcatgtcactcaaaaattttgcctcaaaactggtgagctgaatttttgcagttaaagcatcgtgtagtgtttttcttagtccgttacgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgctatgtggtgctatctgactttttgctgttcagcagttcctgccctctgattttccagtctgaccacttcggattatcccgtgacaggtcattcagactggctaatgcacccagtaaggcagcggtatcatcaacaggcttagtttaaacccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaa

SEQ ID NO: 9 - DNA, 6995 bp, Artificial
  plasmid pOM253
aaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttgggtctagaggaggtgaaacaatgtcccagaatggccgtccagtagtcctcatcgccgataagcttgcgcagtccactgttgacgcgcttggagatgcagtagaagtccgttgggttgacggacctaaccgcccagaactgcttgatgcagttaaggaagcggacgcactgctcgtgcgttctgctaccactgtcgatgctgaagtcatcgccgctgcccctaacttgaagatcgtcggtcgtgccggcgtgggcttggacaacgttgacatccctgctgccactgaagctggcgtcatggttgctaacgcaccgacctctaatattcactccgcttgtgagcacgcaatttctttgctgctgtctactgctcgccagatccctgctgctgatgcgacgctgcgtgagggcgagtggaagcggtcttctttcaacggtgtggaaattttcggaaaaactgtcggtatcgtcggttttggccacattggtcagttgtttgctcagcgtcttgctgcgtttgagaccaccattgttgcttacgatccttacgctaaccctgctcgtgcggctcagctgaacgttgagttggttgagttggatgagctgatgagccgttctgactttgtcaccattcaccttcctaagaccaaggaaactgctggcatgtttgatgcgcagctccttgctaagtccaagaagggccagatcatcatcaacgctgctcgtggtggccttgttgatgagcaggctttggctgatgcgattgagtccggtcacatctgcggccgcacagcgatcccagaggaaatatcctctggggtcgctgtgtcgaccttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctagaggagggattcatcatgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataaaaaaattgaaaaaatggtggaaacacttttttcaatttttttgttttattatttaatatttgggaaatattcattctaattggtaatcagattttagaaaacaataaacccttgcatagggggatcgatatccgtttaggctgggcggatccgccctcccgcacgctttgcgggagggcggtaccaggggtgcttctactgaagaggctcaggatcgtgcgggtactgacgttgctgattctgtgctcaaggcgctggctggcgagttcgtggcggatgctgtgaacgtttccggtggtcgcgtgggcgaagaggttgctgtgtggatggatctggctcgcaagcttggtcttcttgctggcaagcttgtcgacgccgccccagtctccattgaggttgaggctcgaggcgagctttcttccgagcaggtcgatgcacttggtttgtccgctgttcgtggtttgttctccggaattatcgaagagtccgttactttcgtcaacgctcctcgcattgctgaagagcgtggcctggacatctccgtgaagaccaactctgagtctgttactcaccgttccgtcctgcaggtcaaggtcattactggcagcggcgcgagcgcaactgttgttggtgccctgactggtcttgagcgcgttgagaagatcacccgcatcaatggccgtggcctggatctgcgcgcagagggtctgaacctcttcctgcagtacactgacgctcctggtgcactgggtaccgttggtaccaagctgggtgctgctggcatcaacatcgaggctgctgcgttgactcaggctgagaagggtgacggcgctgtcctgatcctgcgtgttgagtccgctgtctctgaagagctggaagctgaaatcaacgctgagttgggtgctacttccttccaggttgatcttgactaattagagatccatttgcttgaaccgccttcccatctttgaattcattcaaggtggtaaggcggttttcgctcttttaatacagttttaatggtagatttgggatccctc

SEQ ID NO: 10 - DNA, 1425 bp, Escherichia coli
atgagtactgaaatcaaaactcaggtcgtggtacttggggcaggccccgcaggttactccgctgccttccgttgcgctgatttaggtctggaaaccgtaatcgtagaacgttacaacacccttggcggtgtttgcctgaacgtcggctgtatcccttctaaagcactgctgcacgtagcaaaagttatcgaagaagccaaagcgctggctgaacacggtatcgtcttcggcgaaccgaaaaccgatatcgacaagattcgtacctggaaagagaaagtgatcaatcagctgaccggtggtctggctggtatggcgaaaggccgcaaagtcaaagtggtcaacggtctgggtaaattcaccggggctaacaccctggaagttgaaggtgagaacggcaaaaccgtgatcaacttcgacaacgcgatcattgcagcgggttctcgcccgatccaactgccgtttattccgcatgaagatccgcgtatctgggactccactgacgcgctggaactgaaagaagtaccagaacgcctgctggtaatgggtggcggtatcatcggtctggaaatgggcaccgtttaccacgcgctgggttcacagattgacgtggttgaaatgttcgaccaggttatcccggcagctgacaaagacatcgttaaagtcttcaccaagcgtatcagcaagaaattcaacctgatgctggaaaccaaagttaccgccgttgaagcgaaagaagacggcatttatgtgacgatggaaggcaaaaaagcacccgctgaaccgcagcgttacgacgccgtgctggtagcgattggtcgtgtgccgaacggtaaaaacctcgacgcaggcaaagcaggcgtggaagttgacgaccgtggtttcatccgcgttgacaaacagctgcgtaccaacgtaccgcacatctttgctatcggcgatatcgtcggtcaaccgatgctggcacacaaaggtgttcacgaaggtcacgttgccgctgaagttatcgccggtaagaaacactacttcgatccgaaagttatcccgtccatcgcctataccgaaccagaagttgcatgggtgggtctgactgagaaagaagcgaaagagaaaggcatcagctatgaaaccgccaccttcccgtgggctgcttctggtcgtgctatcgcttccgactgcgcagacggtatgaccaagctgattttcgacaaagaatctcaccgtgtgatcggtggtgcgattgtcggtactaacggcggcgagctgctgggtgaaatcggcctggcaatcgaaatgggttgtgatgctgaagacatcgcactgaccatccacgcgcacccgactctgcacgagtctgtgggcctggcggcagaagtgttcgaaggtagcattaccgacctgccgaacccgaaagcgaagaagaagtaa

SEQ ID NO: 11 - DNA, 184 bp, Artificial
  promoter P497
ggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatc

SEQ ID NO: 12 - DNA, 192 bp, Artificial
  promoter P3119
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 13 - DNA, 363 bp, Artificial
  promoter P497_P1284
cggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca